(19) United States
(12) Patent Application Publication
(10) Pub. No.: US 2021/0252011 A1
(43) Pub. Date: Aug. 19, 2021

(54) TREATMENT OF GLIOBLASTOMA WITH FASN INHIBITORS

(71) Applicants: FORMA Therapeutics, Inc., Watertown, MA (US); Cornell University, Ithaca, NY (US)

(72) Inventors: Wei LU, Newton, MA (US); Matthew W. MARTIN, Arlington, MA (US); Erik WILKER, Lexington, MA (US); Howard FINE, New York, NY (US)

(21) Appl. No.: 15/733,029

(22) PCT Filed: Oct. 25, 2018
(86) PCT No.: PCT/US2018/057559

§ 371 Date: Apr. 24, 2020

Related U.S. Application Data
(60) Provisional application No. 62/577,098, filed on Oct. 25, 2017.

(51) Int. Cl. A61K 31/55; A61K 31/496; A61K 9/00; A61P 35/00

(52) U.S. Cl. CPC A61K 31/55; A61P 35/00; A61K 9/0053; A61K 31/496

(57) ABSTRACT

The present disclosure relates to methods of treating glioblastoma by administering to a patient in need thereof a FASN inhibitor.

This application claims priority to U.S. Provisional Application Ser. No. 62/577,098, filed on Oct. 25, 2017, the contents of which are incorporated by reference in its entirety.

### BACKGROUND

A glioma is a tumor that grows from glial cells in the brain. Glial cells help maintain the blood-brain barrier and provide nerve cells with nutrients. One form of glioma, glioblastoma, also known as glioblastoma multiforme (GBM), is the most common and aggressive malignant brain tumor in adults. The average annual incidence rate is 3.2 per 100,000 population (Davis, M. E. (2016). Glioblastoma: Overview of Disease and Treatment. Clin. J. Oncol. Nurs. 20, S2-8). The current standard treatment includes surgical resection followed by concurrent radiation with temozolomide (TMZ), an alkylating chemotherapy agent, and then adjuvant chemotherapy with TMZ. Despite surgical resection and multimodality therapy, 70% GBM patients will experience disease progression within one year of diagnosis, with a five-year survival rate of less than 5% (Davis 2016). Although radiotherapy became the conventional treatment for glioblastoma over four decades ago, improvements in therapy have been marginal and have extended median survival from approximately 9 months to 15 months (Fine, H. A. (2015). New Strategies in Glioblastoma: Exploiting the New Biology. Clin. Cancer Res. 21 (9), 1984-1988). Hence, GBM is a disease with unmet medical need.

### BRIEF DESCRIPTION OF THE DRAWINGS

FIG. 1 depicts inhibition of GSC827 neurosphere growth by Compound A.

FIG. 2 depicts inhibition of GSC923 neurosphere growth by Compound A.

FIG. 3 depicts inhibition of GSC827 neurosphere growth by Compound B.

FIG. 4 depicts inhibition of GSC923 neurosphere growth by Compound B.

FIG. 5 depicts percent survival of mice treated with vehicle or Compound A in an orthotopic GBM patient-derived xenograft (PDX) ST112.

FIG. 6 depicts percent survival of mice treated with vehicle or Compound A in an orthotopic GBM patient-derived xenograft (PDX) ST610.

### DEFINITIONS

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The abbreviations used herein have their conventional meaning within the chemical and biological arts. The chemical structures and formulae set forth herein are constructed according to the standard rules of chemical valency known in the chemical arts.

As used above, and throughout this disclosure, the following terms, unless otherwise indicated, shall be understood to have the following meanings. If a definition is missing, convention definition as known to one skilled in the art controls.

The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, the term “administration” typically refers to the administration of a composition or compound to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, administration of a particular compound may be achieved by administration of a composition that includes or otherwise delivers the compound to the subject or system (or to a relevant part thereof or site therein). Thus, in some embodiments, administration of a compound may be achieved by administration of a composition comprising the compound. Alternatively or additionally, in some embodiments, administration of a compound may be achieved by administration of a composition that achieves delivery of the compound (e.g., of a composition that includes a prodrug or other variant of the compound that is metabolized to the compound upon administration of the composition).

As used herein, the term “patient” or “subject” refers to any organism to which provided compound or compounds described herein are administered in accordance with the present invention e.g., for experimental, diagnostic, prophylactic, and/or therapeutic purposes, and includes mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the present invention, the mammal is a human. In some embodiments, a subject may be suffering from, and/or susceptible to a disease, disorder, and/or condition.

The term “FASN” refers all classes, types, subtypes, isotypes, segments, variants, and mutant forms of fatty acid synthase.

As used herein, the term “inhibitor” refers to an agent, condition, or event whose presence, level, degree, type, or form correlates with decreased level or activity of another agent (i.e., the inhibited agent, or target). In general, an inhibitor may be or include an agent of any chemical class including, for example, small molecules, polypeptides, nucleic acids, carbohydrates, lipids, metals, and/or any other entity, condition or event that shows the relevant inhibitory activity. In some embodiments, an inhibitor may be direct (in which case it exerts its influence directly upon its target, for example by binding to the target); in some embodiments, an inhibitor may be indirect (in which case it exerts its influence by interacting with and/or otherwise altering a regulator of the target, so that level and/or activity of the target is reduced).

The terms “effective amount” or “therapeutically effective amount” refer to a sufficient amount of the agent to provide the desired biological result. That result can be reduction and/or alleviation of the signs, symptoms, or causes of a disease, or any other desired alteration of a biological system. For example, an “effective amount” for therapeutic use is the amount of the composition comprising a compound as disclosed herein required to provide a clinically significant decrease in a disease, disorder, and/or condition. An appropriate “effective” amount in any individual case may be determined by one of ordinary skill in the art using routine experimentation. Thus, the expression “effective amount” generally refers to the quantity for which the active substance has therapeutic effects.

“Mammal” means humans and other mammalian animals.

As used herein, the term “prevent” or “prevention,” when used in connection with the occurrence of a disease, disorder, and/or condition, refers to reducing the risk of developing the disease, disorder, and/or condition and/or to delaying onset of one or more characteristics or symptoms of the disease, disorder, or condition. Prevention may be considered complete when onset of a disease, disorder, or condition has been delayed for a predefined period of time.

As used herein, the terms “treat” or “treatment” refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

By “pharmaceutically acceptable is meant a material which is not biologically or otherwise undesirable—the material may be administered to an individual without causing any undesirable biological effects or interacting in a deleterious manner with any of the components of the composition in which it is contained.

“Carrier materials” or what are also referred to as “excipients” include any commonly used excipients in pharmaceutics and should be selected on the basis of compatibility and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may comprise, e.g., acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, and the like. See, e.g., Hoover, John E., Remington's Pharmaceutical Sciences, Mack Publishing Co., Easton, Pa. 1975.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocyclyl,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In some embodiments, aliphatic groups contain 1-4 aliphatic carbon atoms. In some embodiments, aliphatic groups contain 1-3 aliphatic carbon atoms, and in yet other embodiments, aliphatic groups contain 1-2 aliphatic carbon atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, “alkyl” means a straight chain or branched saturated chain having from 1 to 10 carbon atoms. Representative saturated alkyl groups include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, n-hexyl and the like, and longer alkyl groups, such as heptyl, and octyl and the like. An alkyl group can be unsubstituted or substituted. Alkyl groups containing three or more carbon atoms may be straight, branched or cyclized. As used herein, “lower alkyl” means an alkyl having from 1 to 6 carbon atoms.

As used herein, an “alkenyl” includes an unbranched or branched hydrocarbon chain having one or more double bonds therein. The double bond of an alkenyl group can be unconjugated or conjugated to another unsaturated group. Illustrative alkenyl groups include, but are not limited to, (C₂-C₈) alkenyl groups, such as ethylenyl, vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl and the like. An alkenyl group can be unsubstituted or substituted.

As used herein, “alkynyl” includes an unbranched or branched hydrocarbon chain having one or more triple bonds therein. The triple bond of an alkynyl group can be unconjugated or conjugated to another unsaturated group. Suitable alkynyl groups include, but are not limited to, (C₂-C₆) alkynyl groups, such as ethynyl, propynyl, butynyl, pentynyl, hexynyl, methylpropynyl, 4-methyl-1-butynyl, 4-propyl-2-pentynyl, 4-butyl-2-hexynyl and the like. An alkynyl group can be unsubstituted or substituted.

The terms “trifluoromethyl,” “sulfonyl,” and “carboxyl” include CF₃, SO₂, and CO₂H, respectively.

The term “hydroxyl” means an OH group.

The term alkyl hydroxyl or hydroxyalkyl means an alkyl group as defined above, where the alkyl group has an OH group disposed thereon.

The term “alkoxy” as used herein includes —O-(alkyl), wherein alkyl is defined above.

The term “aminoalkyl” as used herein means a group having one or more nitrogen atoms and one or more alkyl groups as defined above on the nitrogen.

“Aralkyl” or “arylalkyl” means an aryl-alkyl-group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Heteroarylalkyl” means a heteroaryl moiety as defined herein linked via an alkyl moiety (defined above) to a parent core. Non-limiting examples of suitable heteroaryls include 2-pyridinylmethyl, quinolinylmethyl, and the like.

“Heterocyclylalkyl” means a heterocyclyl moiety as defined herein linked via an alkyl moiety (defined above) to a parent core. Non-limiting examples of suitable heterocyclylalkyls include piperidinylmethyl, piperazinylmethyl and the like.

It should also be noted that any carbon as well as heteroatom with unsatisfied valences in the text, schemes, examples and Tables herein is assumed to have the sufficient number of hydrogen atom(s) to satisfy the valences.

When any variable (e.g., aryl, heterocycle, R², etc.) occurs more than one time in any constituent or in the Formulas, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

The term “deuterium” as used herein means a stable isotope of hydrogen having odd numbers of protons and neutrons.

The term “halo” as used herein means a substituent having at least one halogen selected from fluorine, chlorine, bromine, and iodine.

The term “cyano” as used herein means a substituent having a carbon atom joined to a nitrogen atom by a triple bond.

The term “amino” as used herein means a substituent containing at least one nitrogen atom.

The term “(amino)alkoxy” as used herein means a substituent having at least one amino group and at least one alkoxy group.

The term “aryloxy” as used herein means a substituent of the form Ar—O— where Ar is an aryl group as defined herein.

The term “methylenedioxy” as used herein means a functional group with the structural formula —O—CH₂—O— which is connected to the molecule by two chemical bonds via the oxygens.

As used herein, “alkoxyalkyl” means -(alkyl)-O-(alkyl), wherein each “alkyl” is independently an alkyl group defined above.

The term “(alkoxyalkyl)amino” as used herein means a substituent having at least one alkoxyalkyl group as defined above and at least one amino group as defined above.

As used herein, the term “aryl” used alone or as part of a larger moiety as in “aralkyl,” “aralkoxy,” or “aryloxyalkyl,” refers to a monocyclic or fused polycyclic, aromatic carbocycle (ring structure having ring atoms that are all carbon) having from 6 to 24 ring atoms, and which includes monocyclic and bicyclic ring systems having a total of five to 10 ring members, wherein at least one ring in the system is aromatic and wherein each ring in the system contains three to seven ring members. The term “aryl” may be used interchangeably with the term “aryl ring”. In some embodiments, an 8-10 membered bicyclic aryl group is an optionally substituted naphthyl ring. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. Illustrative examples of aryl groups include, but are not limited to, the following moieties:

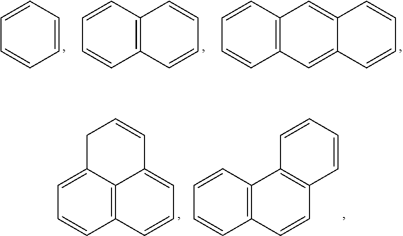

and the like. Illustrative substituted aryls include:

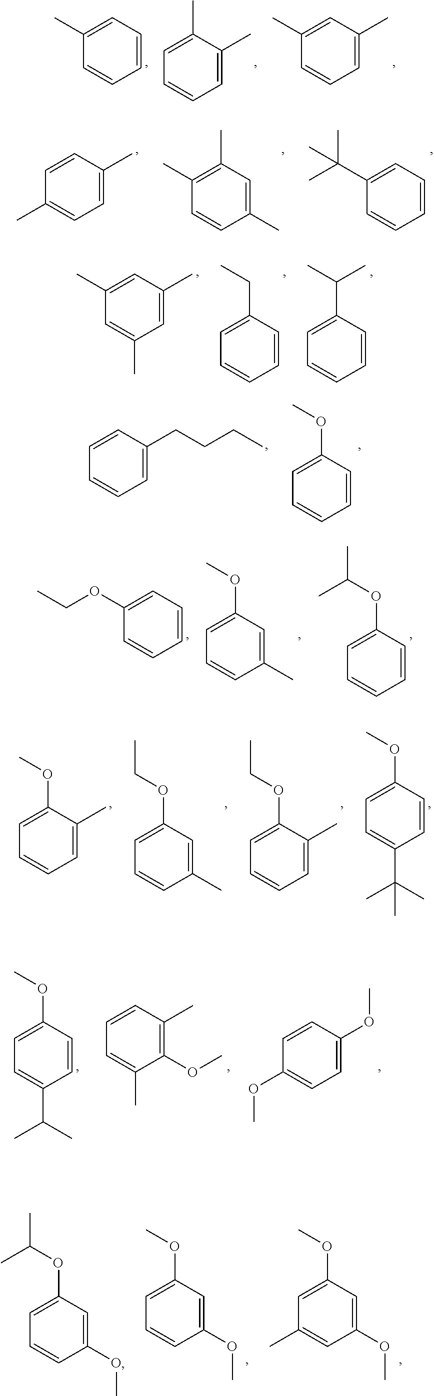

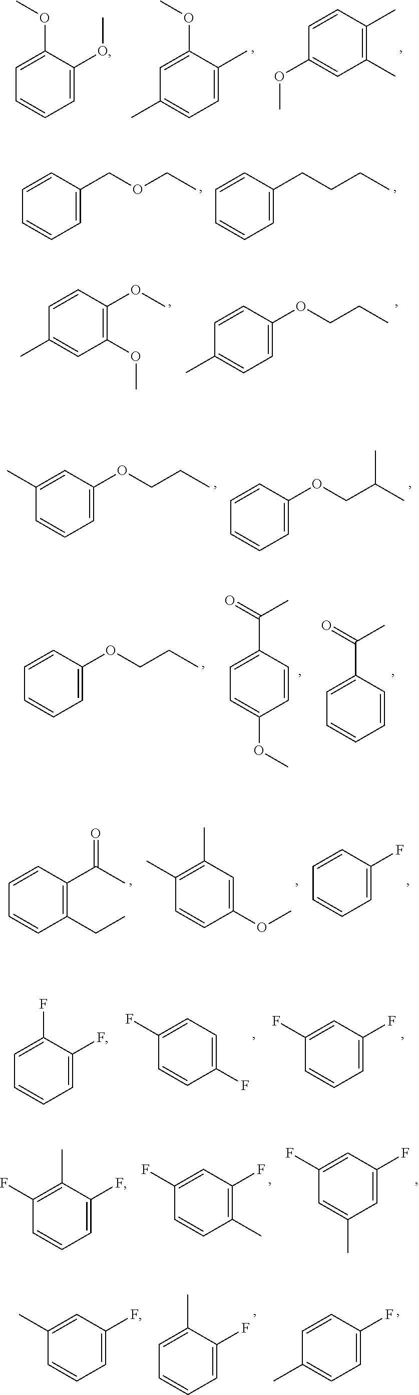

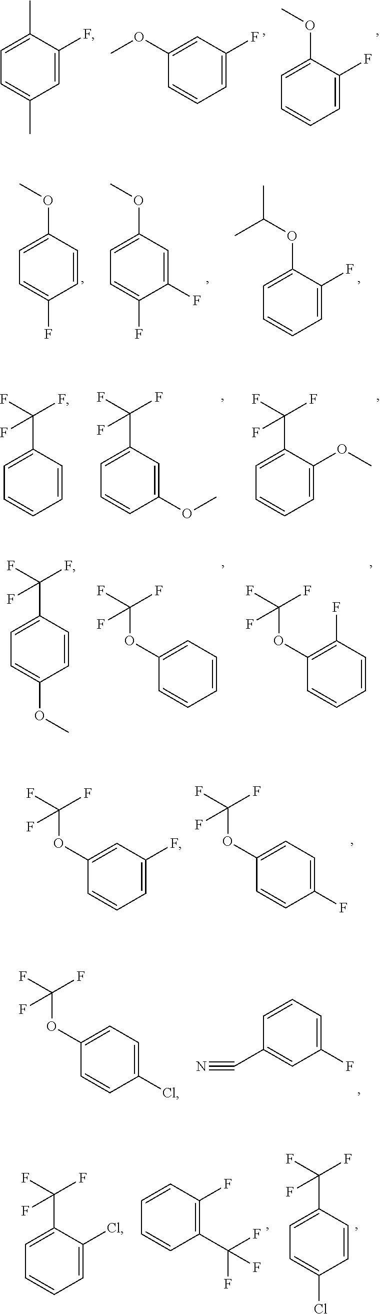

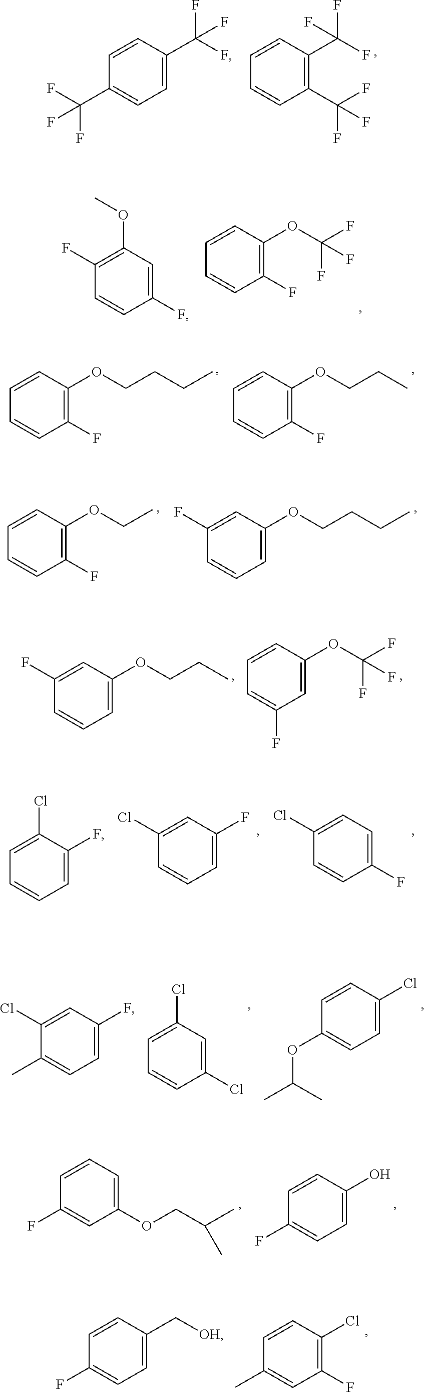

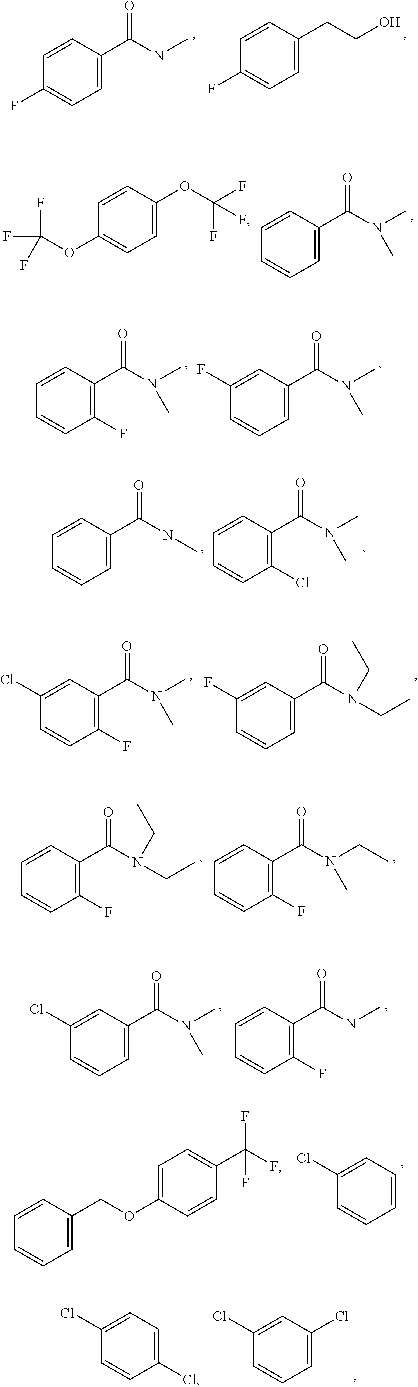

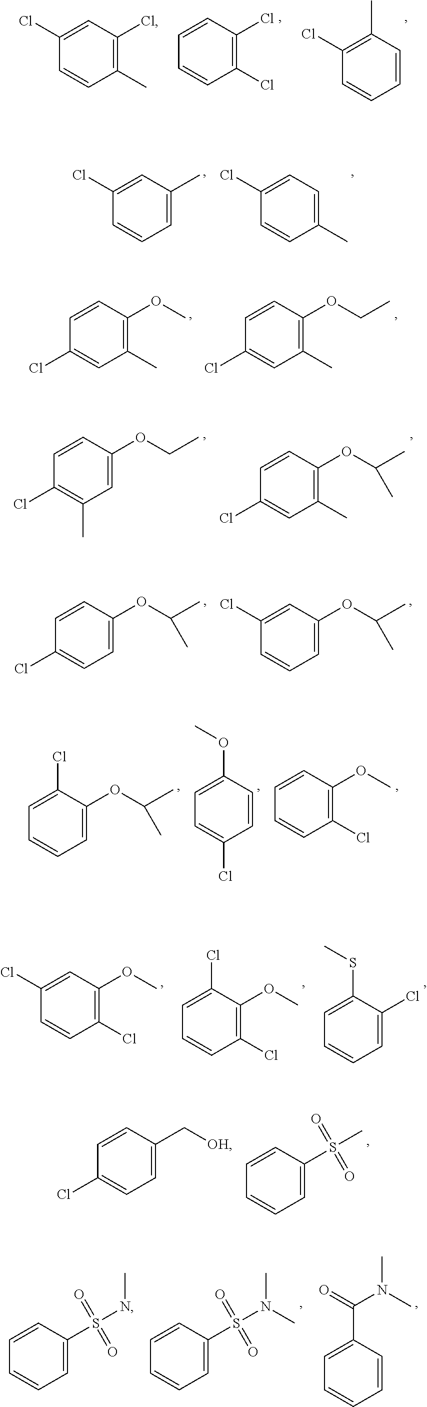

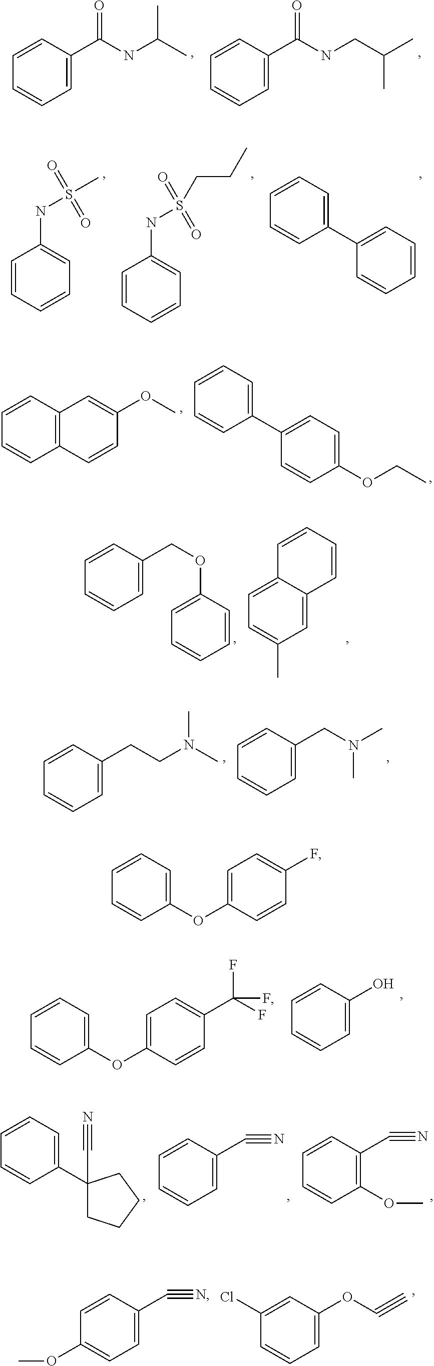

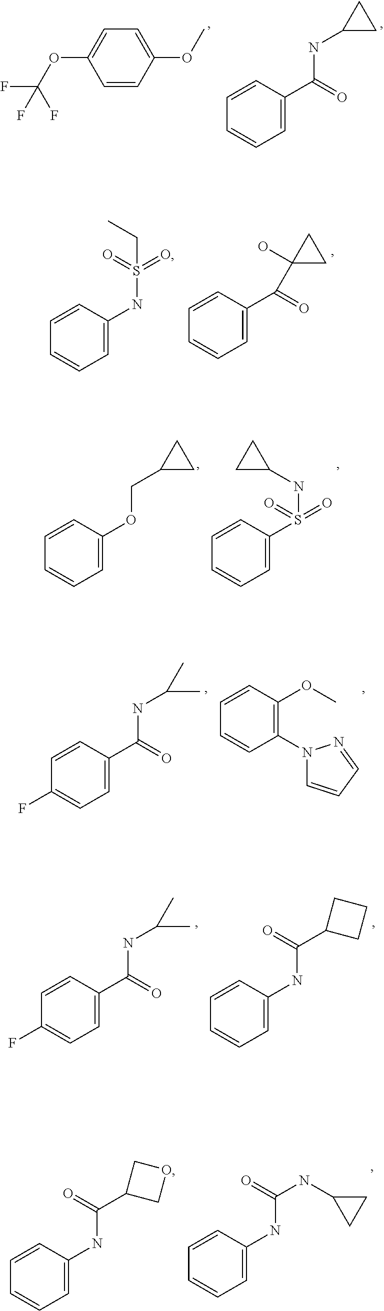

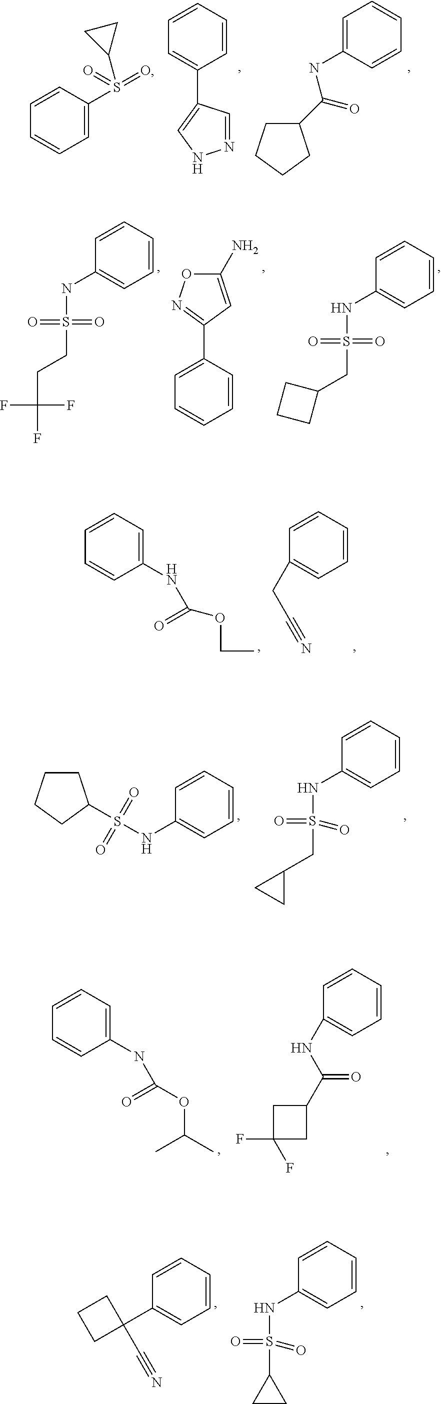

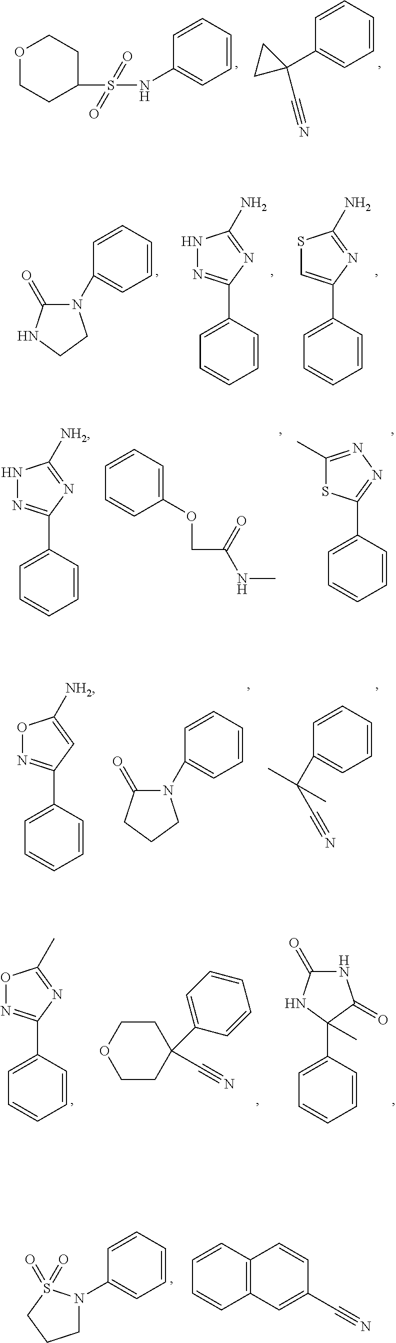

and the like.

The terms “heteroaryl” and “heteroar-,” used alone or as part of a larger moiety, e.g., “heteroaralkyl,” or “heteroaralkoxy,” refer to groups having from 5 to 24 ring atoms, and includes those having 5 to 10 ring atoms, preferably 5, 6, or 9 ring atoms; having 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted. Illustrative examples of heteroaryl and substituted heteroaryl groups include, but are not limited to the following moieties:

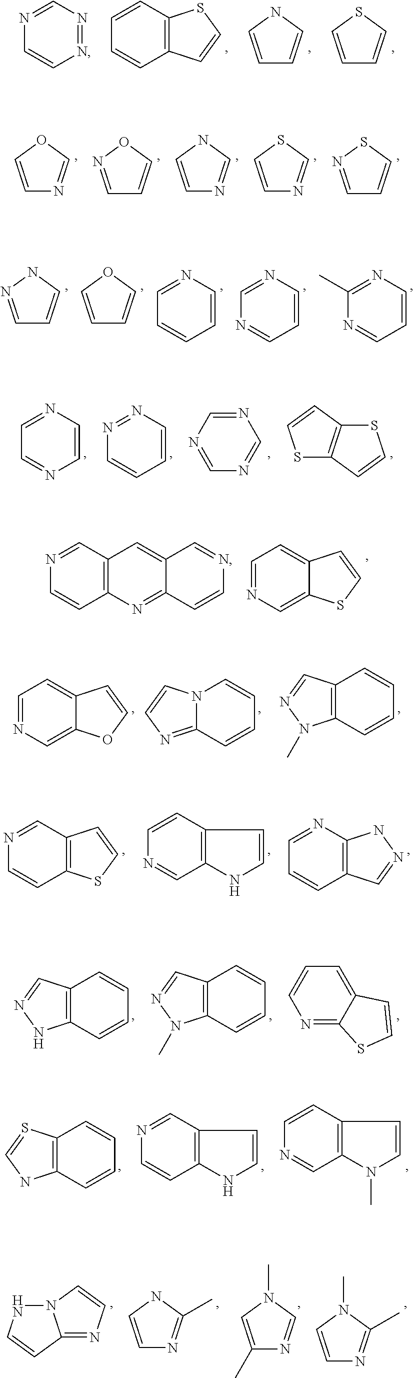

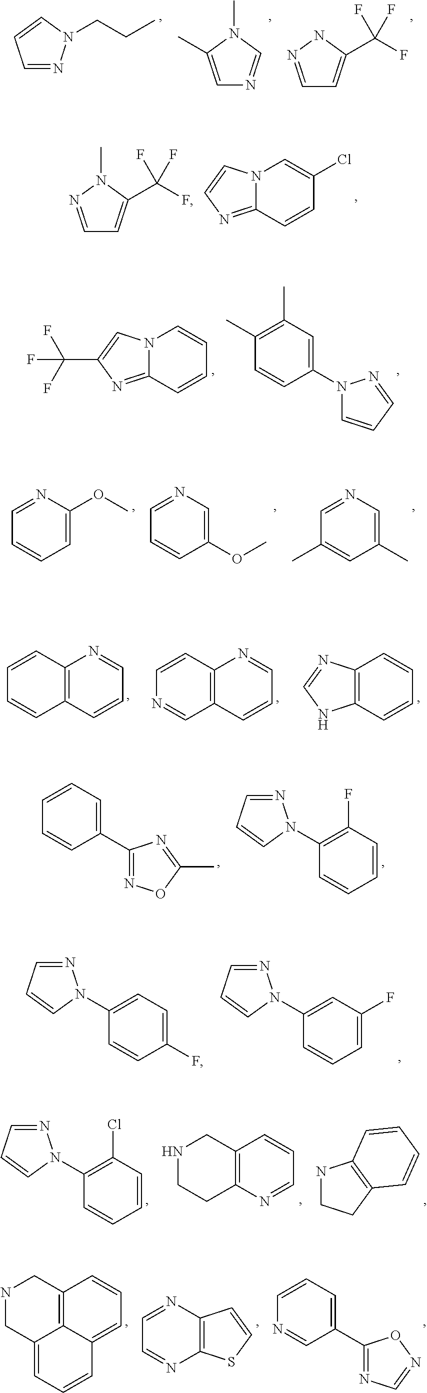

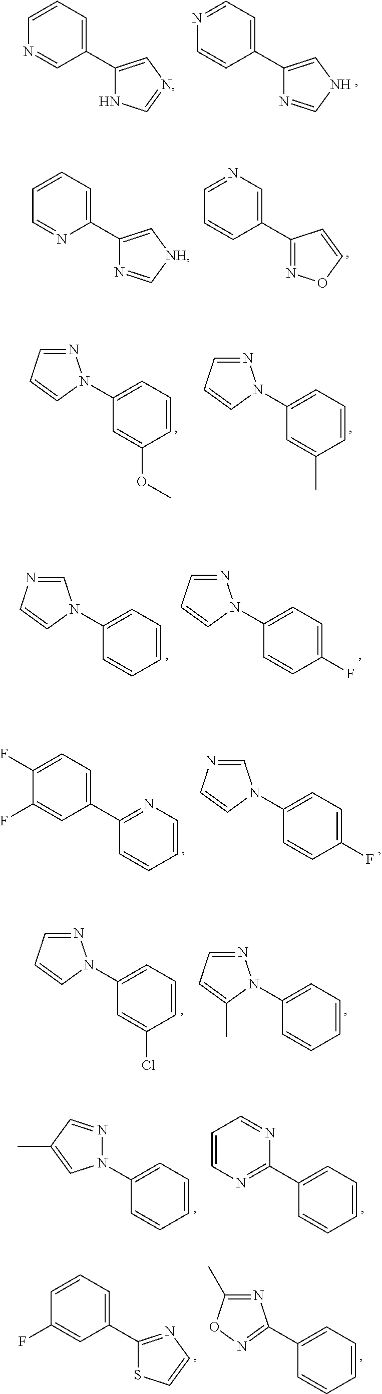

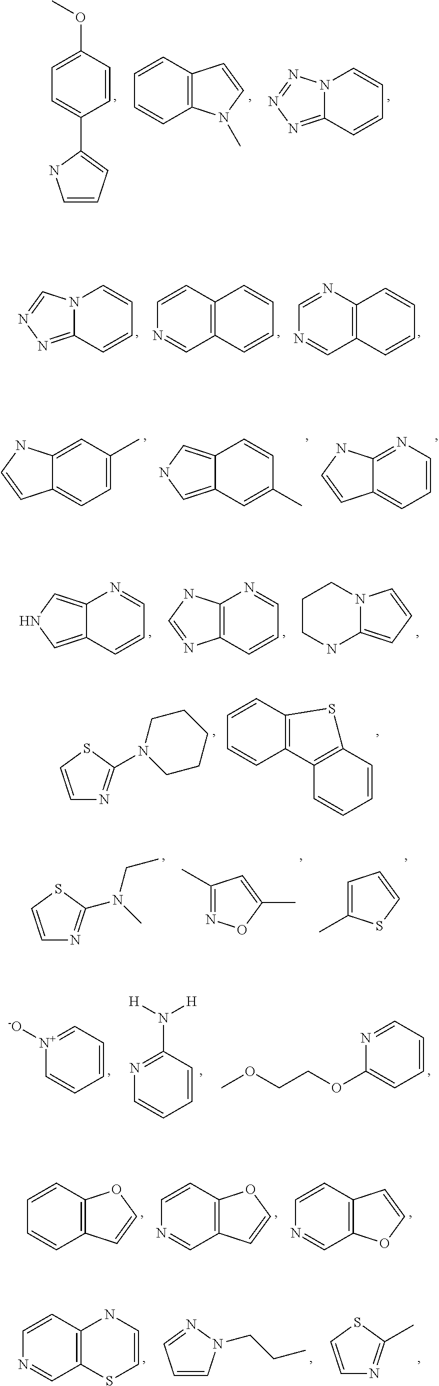

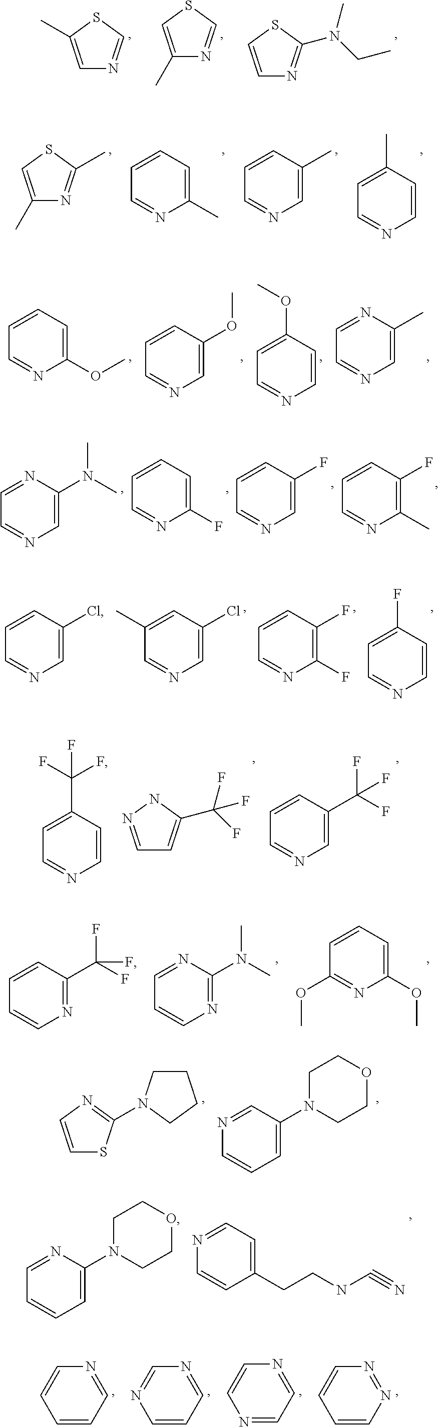

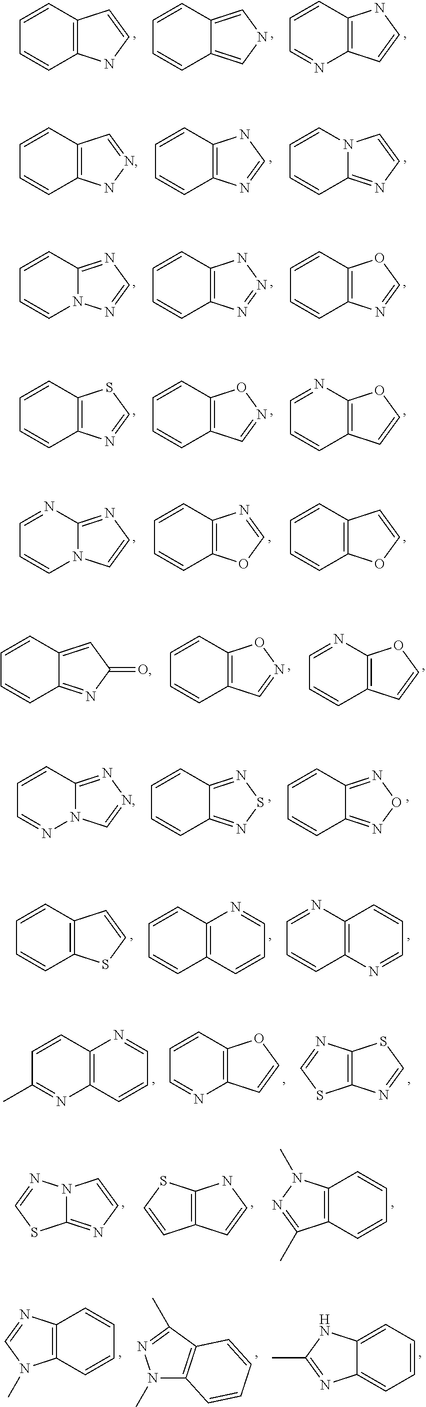

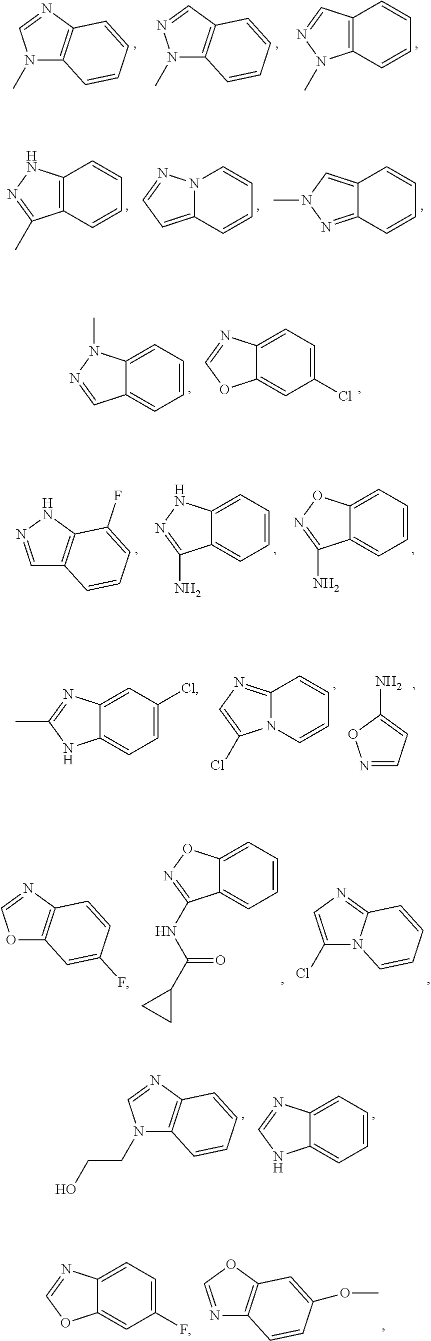

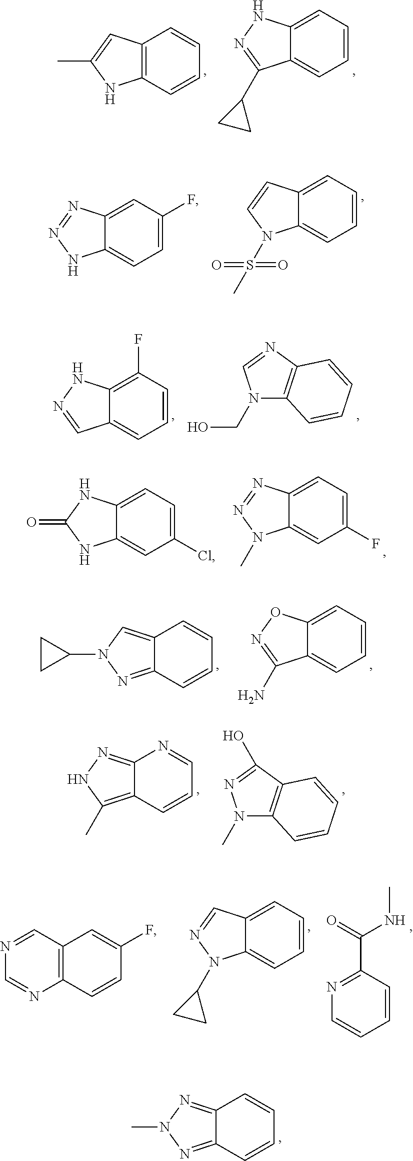

and the like.

As used herein, the term “cycloalkyl” refers to a saturated or partially saturated, monocyclic or fused or spiro polycyclic, carbocycle having from 3 to 24 ring atoms. In some embodiments, “cycloaliphatic” (or “carbocyclyl” or “cycloalkyl”) refers to a monocyclic C₃-C₇ hydrocarbon that is completely saturated or that contains one or more units of unsaturation, but which is not aromatic, that has a single point of attachment to the rest of the molecule. Illustrative examples of cycloalkyl groups include, but are not limited to, the following moieties:

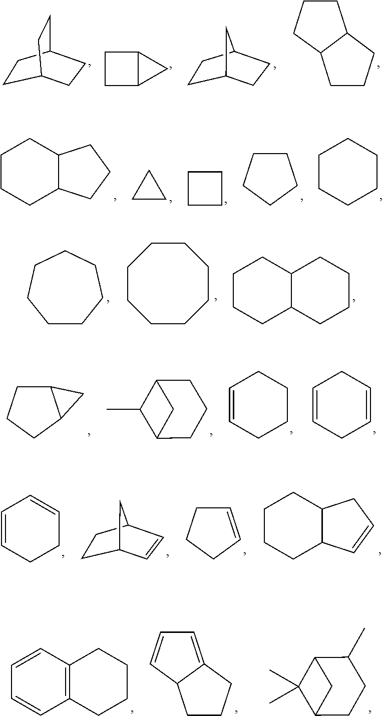

and the like.

As used herein, the term “heterocycloalkyl” refers to a monocyclic, or fused or spiro, polycyclic, ring structure that is saturated or partially saturated and has from 3 to ring atoms selected from C atoms and N, O, and S heteroatoms. In some embodiments, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Illustrative examples of heterocycloalkyl and substituted heterocycloalkyl groups include, but are not limited to:

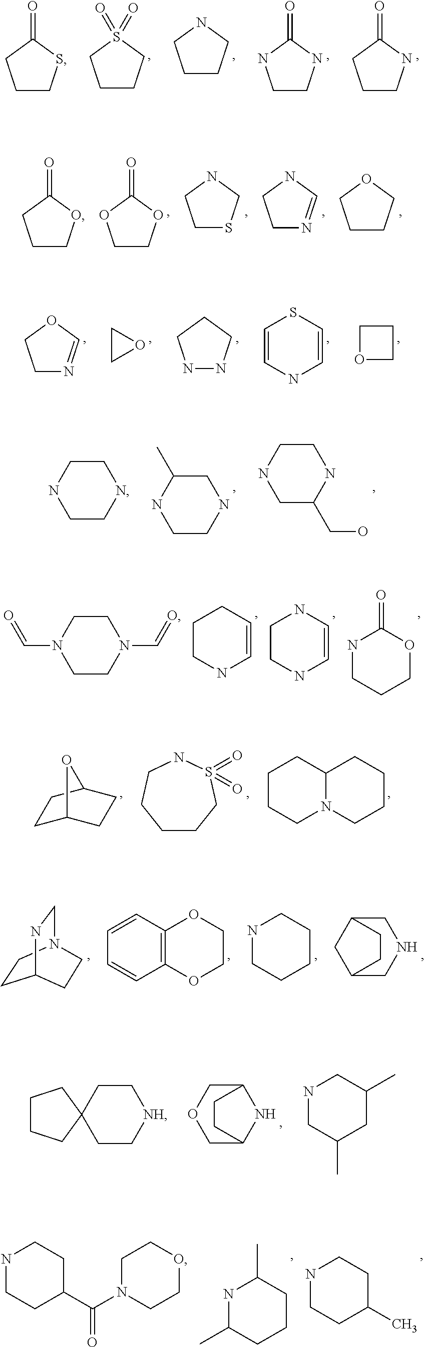

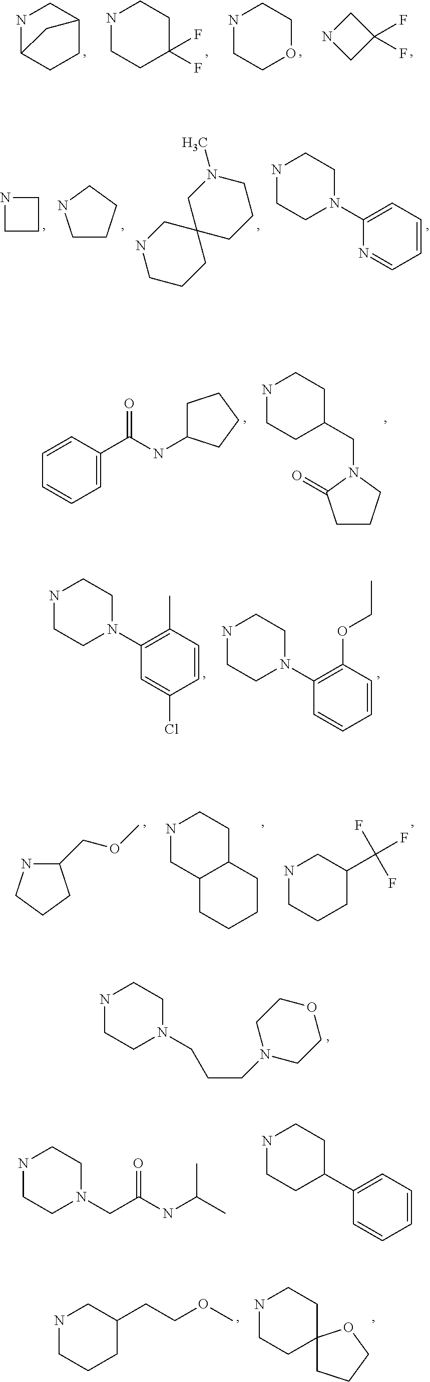

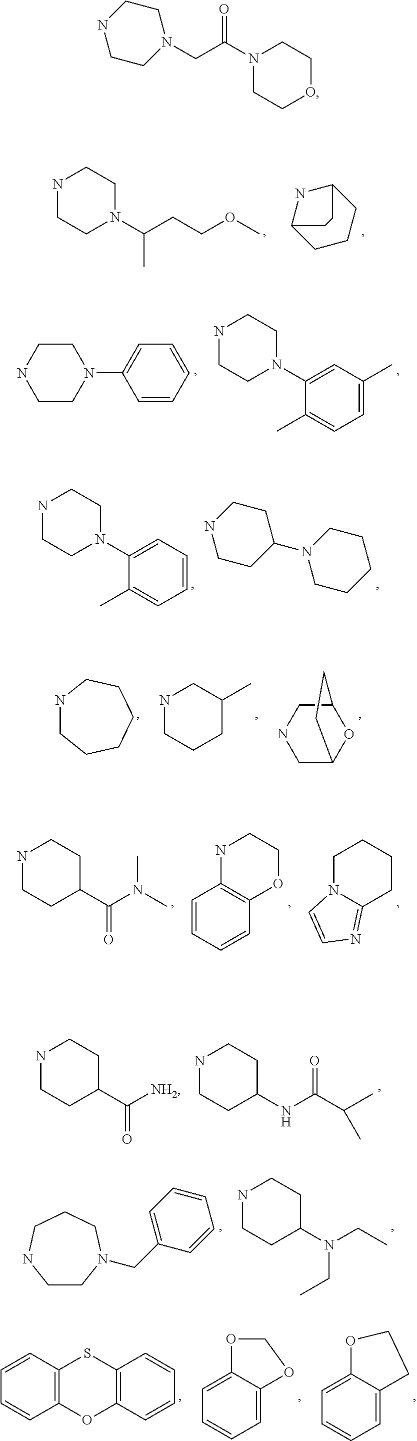

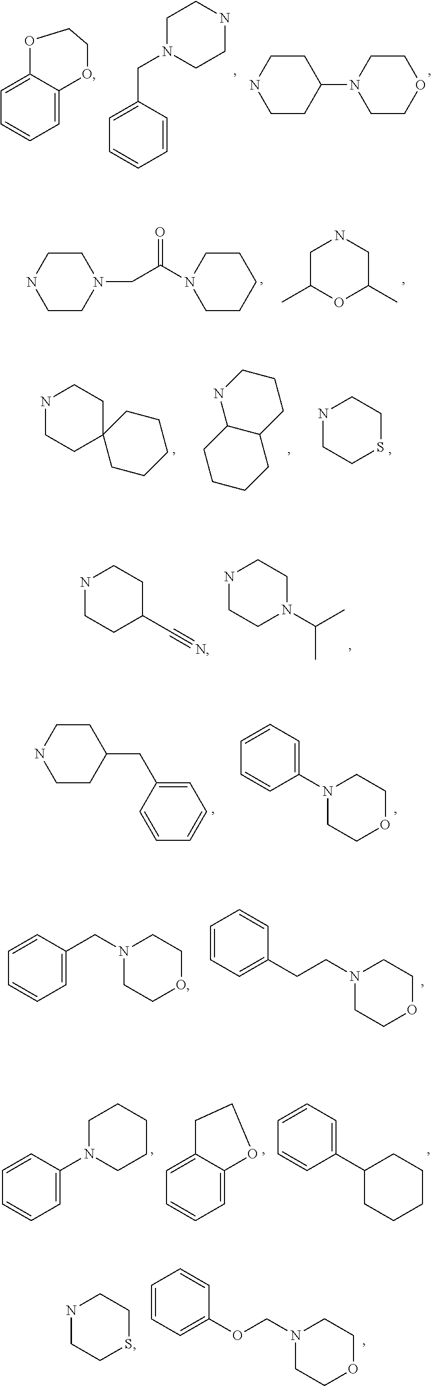

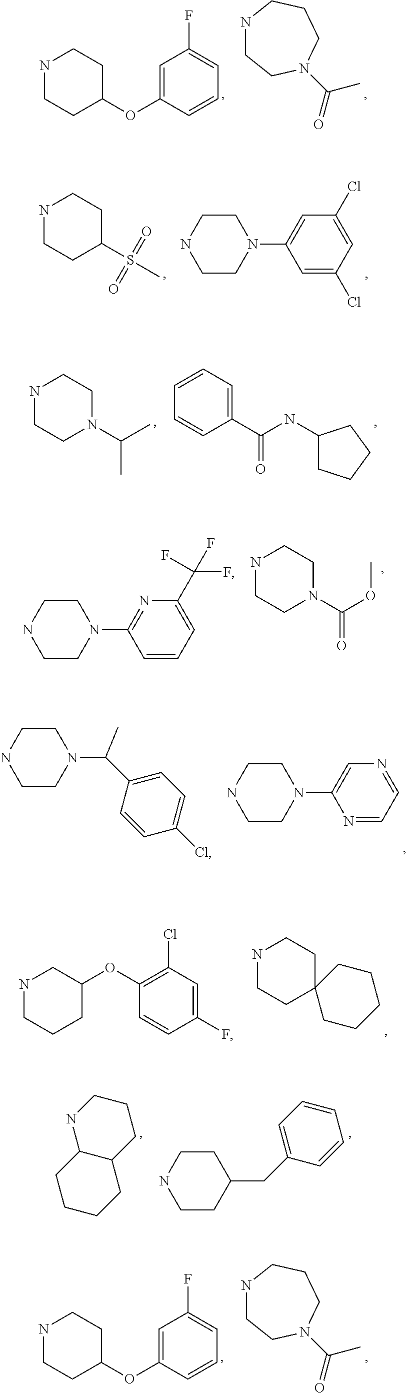

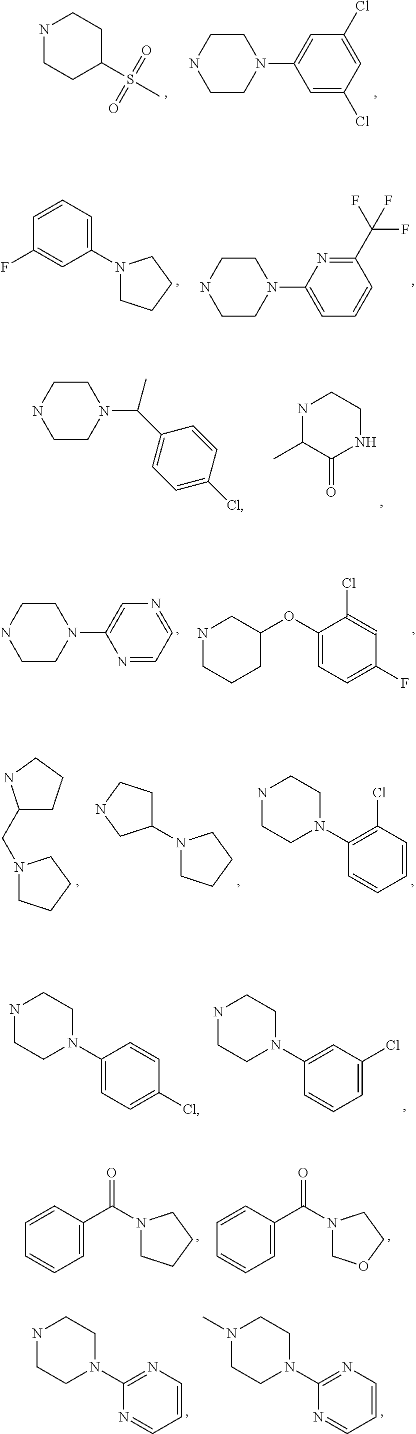

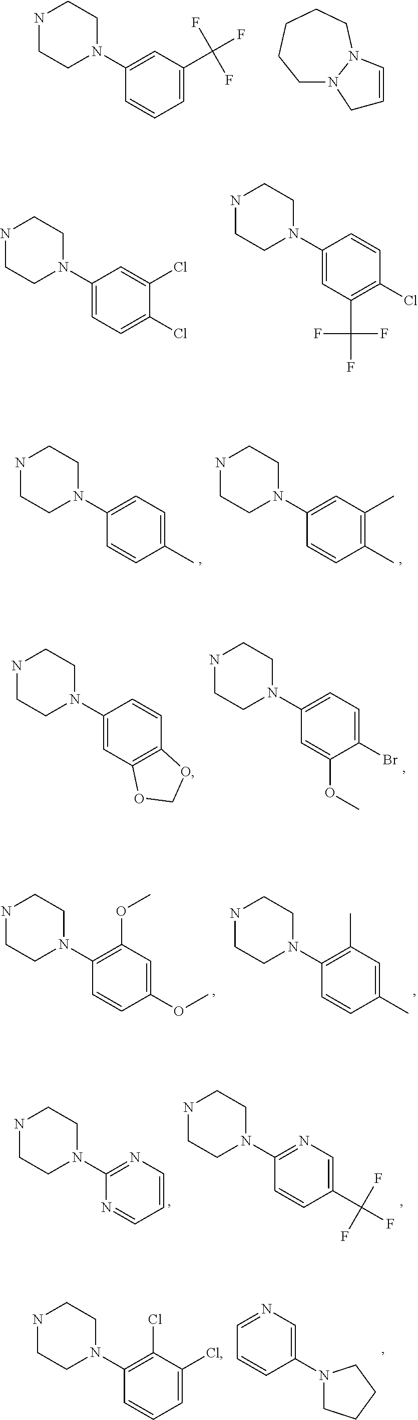

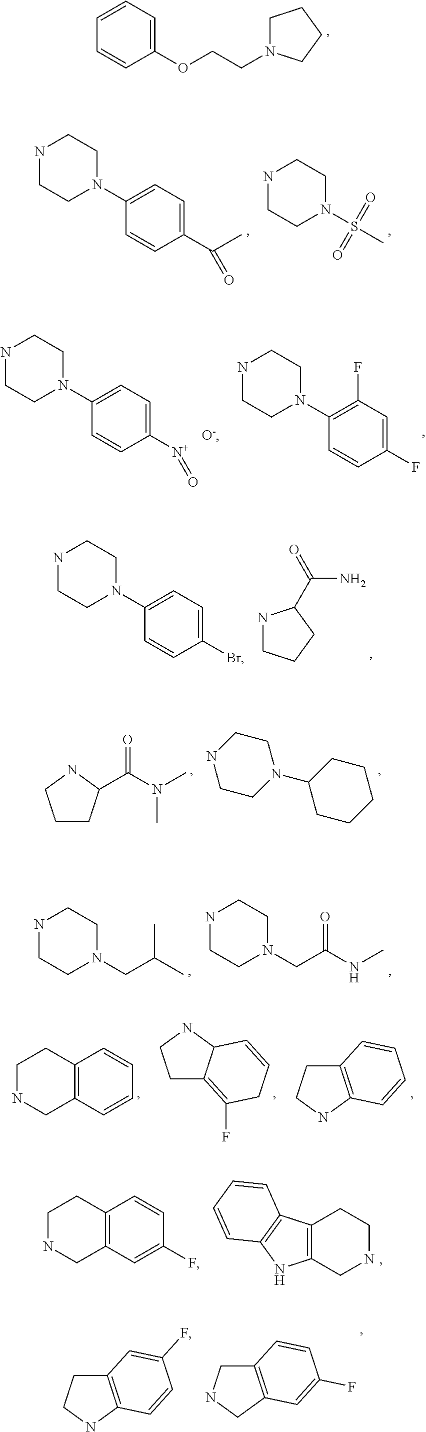

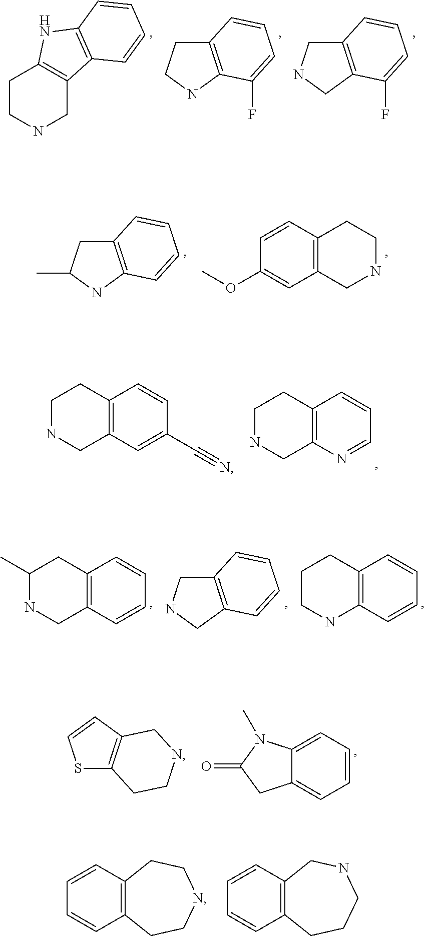

and the like.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference.

Numerical ranges, as used herein, are intended to include sequential integers. For example, a range expressed as “from 0 to 4” would include 0, 1, 2, 3 and 4.

As used herein, the terms “monocyclic, bicyclic, or tricyclic aryl, heteroaryl, cycloalkyl, or heterocycloalkyl,” mean any ring of a bicyclic or tricyclic structure may independently be aryl, heteroaryl, cycloalkyl, or heterocycloalkyl in an ortho or ortho and peri fused system.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

As used herein, the term “optionally substituted” means that the specified group is unsubstituted or substituted by one or more substituents.

When a multifunctional moiety is shown, the point of attachment to the core may be identified by a line. For e.g. (cycloalkyloxy)alkyl- refers to alkyl being the point of attachment to the core while cycloalkyl is attached to alkyl via the oxy group. In the absence of a line, attachment at any position may be assumed.

It must be noted that, as used in the specification and the appended claims, the singular forms “a,” “an” and “the” include plural referents unless the context clearly dictates otherwise.

When used as a therapeutic agent the inhibitors of the FASN (FASN) described herein may be administered with one or more physiologically acceptable excipients. A physiologically acceptable carrier or excipient is a formulation to which the compound can be added to dissolve it or otherwise facilitate its administration.

The dosage forms of the present invention, may contain a mixture of one or more compounds of this invention, and may include additional materials known to those skilled in the art as pharmaceutical excipients. Stabilizing additives may be incorporated into the delivery agent solution. With some drugs, the presence of such additives promotes the stability and dispersibility of the agent in solution. The stabilizing additives may be employed at a concentration ranging from about 0.1 and 5% (W/V), preferably about 0.5% (W/V). Suitable, but non-limiting, examples of stabilizing additives include gum acacia, gelatin, methyl cellulose, polyethylene glycol, carboxylic acids and salts thereof, and polylysine. The preferred stabilizing additives are gum acacia, gelatin and methyl cellulose.

Acidifying agents (acetic acid, glacial acetic acid, citric acid, fumaric acid, hydrochloric acid, diluted hydrochloric acid, malic acid, nitric acid, phosphoric acid, diluted phosphoric acid, sulfuric acid, tartaric acid); Aerosol propellants (butane, dichlorodifluoro-methane, dichlorotetrafluoroethane, isobutane, propane, trichloromonofluoromethane); Air displacements (carbon dioxide, nitrogen); Alcohol denaturants (denatonium benzoate, methyl isobutyl ketone, sucrose octacetate); Alkalizing agents (strong ammonia solution, ammonium carbonate, diethanolamine, diisopropanolamine, potassium hydroxide, sodium bicarbonate, sodium borate, sodium carbonate, sodium hydroxide, trolamine); Anticaking agents (see glidant); Antifoaming agents (dimethicone, simethicone); Antimicrobial preservatives (benzalkonium chloride, benzalkonium chloride solution, benzelthonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, thymol); Antioxidants (ascorbic acid, acorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, tocopherols excipient); Buffering agents (acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate); Capsule lubricants (see tablet and capsule lubricant); Chelating agents (edetate disodium, ethylenediaminetetraacetic acid and salts, edetic acid); Coating agents (sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyethylene glycol, polyvinyl acetate phthalate, shellac, sucrose, titanium dioxide, carnauba wax, microcystalline wax, zein); Colorants (caramel, red, yellow, black or blends, ferric oxide); Complexing agents (ethylenediaminetetraacetic acid and salts (EDTA), edetic acid, gentisic acid ethanolmaide, oxyquinoline sulfate); Desiccants (calcium chloride, calcium sulfate, silicon dioxide); Emulsifying and/or solubilizing agents (acacia, cholesterol, diethanolamine (adjunct), glyceryl monostearate, lanolin alcohols, lecithin, mono- and di-glycerides, monoethanolamine (adjunct), oleic acid (adjunct), oleyl alcohol (stabilizer), poloxamer, polyoxyethylene 50 stearate, polyoxyl 35 caster oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, soritan monooleate, sorbitan monopalmitate, sorbitan monostearate, stearic acid, trolamine, emulsifying wax); Filtering aids (powdered cellulose, purified siliceous earth); Flavors and perfumes (anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin); Glidants and/or anticaking agents (calcium silicate, magnesium silicate, colloidal silicon dioxide, talc); Humectants (glycerin, hexylene glycol, propylene glycol, sorbitol); Plasticizers (castor oil, diacetylated monoglycerides, diethyl phthalate, glycerin, mono- and di-acetylated monoglycerides, polyethylene glycol, propylene glycol, triacetin, triethyl citrate); Polymers (e.g., cellulose acetate, alkyl celloloses, hydroxyalkylcelloloses, acrylic polymers and copolymers); Solvents (acetone, alcohol, diluted alcohol, amylene hydrate, benzyl benzoate, butyl alcohol, carbon tetrachloride, chloroform, corn oil, cottonseed oil, ethyl acetate, glycerin, hexylene glycol, isopropyl alcohol, methyl alcohol, methylene chloride, methyl isobutyl ketone, mineral oil, peanut oil, polyethylene glycol, propylene carbonate, propylene glycol, sesame oil, water for injection, sterile water for injection, sterile water for irrigation, purified water); Sorbents (powdered cellulose, charcoal, purified siliceous earth); Carbon dioxide sorbents (barium hydroxide lime, soda lime); Stiffening agents (hydrogenated castor oil, cetostearyl alcohol, cetyl alcohol, cetyl esters wax, hard fat, paraffin, polyethylene excipient, stearyl alcohol, emulsifying wax, white wax, yellow wax); Suspending and/or viscosity-increasing agents (acacia, agar, alginic acid, aluminum monostearate, bentonite, purified bentonite, magma bentonite, carbomer 934p, carboxymethylcellulose calcium, carboxymethylcellulose sodium, carboxymethycellulose sodium 12, carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, propylene glycol alginate, silicon dioxide, colloidal silicon dioxide, sodium alginate, tragacanth, xanthan gum); Sweetening agents (aspartame, dextrates, dextrose, excipient dextrose, fructose, mannitol, saccharin, calcium saccharin, sodium saccharin, sorbitol, solution sorbitol, sucrose, compressible sugar, confectioner's sugar, syrup); Tablet binders (acacia, alginic acid, sodium carboxymethylcellulose, microcrystalline cellulose, dextrin, ethylcellulose, gelatin, liquid glucose, guar gum, hydroxypropyl methylcellulose, methycellulose, polyethylene oxide, povidone, pregelatinized starch, syrup); Tablet and/or capsule diluents (calcium carbonate, dibasic calcium phosphate, tribasic calcium phosphate, calcium sulfate, microcrystalline cellulose, powdered cellulose, dextrates, dextrin, dextrose excipient, fructose, kaolin, lactose, mannitol, sorbitol, starch, pregelatinized starch, sucrose, compressible sugar, confectioner's sugar); Tablet disintegrants (alginic acid, microcrystalline cellulose, croscarmellose sodium, corspovidone, polacrilin potassium, sodium starch glycolate, starch, pregelatinized starch); Tablet and/or capsule lubricants (calcium stearate, glyceryl behenate, magnesium stearate, light mineral oil, polyethylene glycol, sodium stearyl fumarate, stearic acid, purified stearic acid, talc, hydrogenated vegetable oil, zinc stearate); Tonicity agent (dextrose, glycerin, mannitol, potassium chloride, sodium chloride); Vehicle: flavored and/or sweetened (aromatic elixir, compound benzaldehyde elixir, iso-alcoholic elixir, peppermint water, sorbitol solution, syrup, tolu balsam syrup); Vehicle: oleaginous (almond oil, corn oil, cottonseed oil, ethyl oleate, isopropyl myristate, isopropyl palmitate, mineral oil, light mineral oil, myristyl alcohol, octyldodecanol, olive oil, peanut oil, persic oil, seame oil, soybean oil, squalane); Vehicle: solid carrier (sugar spheres); Vehicle: sterile (bacteriostatic water for injection, bacteriostatic sodium chloride injection); Viscosity-increasing (see suspending agent); Water repelling agent (cyclomethicone, dimethicone, simethicone); and Wetting and/or solubilizing agent (benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysorbate 80, sodium lauryl sulfate, sorbitan monolaureate, sorbitan monooleate, sorbitan monopalmitate, sorbitan monostearate, tyloxapol) may be used as excipients. This list is not meant to be exclusive, but instead merely representative of the classes of excipients and the particular excipients which may be used in dosage forms of the present invention.

The compounds of Formulas I and subgenera thereof can form salts which are also within the scope of this invention. Reference to a compound of the formulae herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of the formulae contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the a formula may be formed, for example, by reacting a compound of a formula with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Compounds of the various Formulas, and salts, solvates, esters and prodrugs thereof, may exist in their tautomeric form (for example, as an amide or imino ether). All such tautomeric forms are contemplated herein as part of the present invention.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

The symbol “ [symbol] ”, except when used as a bond to depict unknown or mixed stereochemistry, denotes the point of attachment of a chemical moiety to the remainder of a molecule or chemical formula.

### DETAILED DESCRIPTION OF CERTAIN EMBODIMENTS

The present disclosure encompasses the recognition that there remains a need for therapeutics useful for the treatment of glioblastoma. The present disclosure provides, among other things, insight regarding particular compounds useful for the treatment of glioblastoma.

The present disclosure provides surprising evidence that, despite a lack of other FASN inhibitors suitable for use as chemotherapeutics for the treatment of glioblastoma, compounds described herein show significant biological benefits in model systems of glioblastoma. The present disclosure therefore provides methods of treating individuals suffering from or susceptible to glioblastoma by administration of a compound as described herein.

Other studies have postulated a relationship between FASN and gliomas or glioma stem cells using the FASN inhibitors Orlistat, Cerulenin, and C75 (Grube, S. (2014). Overexpression of fatty acid synthase in human gliomas correlates with the WHO tumor grade and inhibition with Orlistat reduces cell viability and triggers apoptosis. J. Neurooncol. 118, 277-287; Yasumoto, Y. (2015) Inhibition of Fatty Acid Synthase Decreases Expression of Stemness Markers in Glioma Stem Cells. Plos One DOI: 10.1371/journal.pone.0147717). However, these compounds have known shortcomings that significantly limit their use as therapeutics for treating gliomas. For example, Cerulenin and C75 are chemically unstable, lack good potency and/or selectivity for FASN, or have other liabilities that make them poor thereapeutics (Grube 2014). Orlistat, while approved for the treatment of obesity, does not possess a favorable bioavailability for systemic use as chemotherapy (Grube 2014). In order to reach the brain, a key feature of a chemotherapeutic to be administered orally or intravenously is the ability to cross the blood-brain barrier.

Regardless, prior to the teachings described herein, those of ordinary skill in the art understood that FASN inhibitor compounds used to target glioblastoma may present unique efficacy challenges or other defects. Against this backdrop, the present disclosure presents surprising evidence of the usefulness and effectiveness of provided compounds as described herein in the treatment of glioblastoma.

Applicants have found that provided compounds blocked the neurosphere growth of patient-derived glioma stem cells. Furthermore, in vivo activity of provided compounds is shown by prolonging the survival of two orthotopic GBM patient-derived xenograft mouse model. In some embodiments, provided compounds are capable of crossing the blood-brain barrier.

The present disclosure encompasses the recognition that in order to treat glioblastoma, it is useful for a compound to have favorable brain penetration, particularly where the compound is to be administered systemically (e.g. intravenously or orally). In some embodiments, a compound used in accordance with provided methods is characterized in that, when measured 2 or 8 hours after administration according to the procedure of ensuing Example 2, it has a ratio of concentration in the brain to plasma greater than about 0.01, greater than about 0.05, greater than about 0.1, greater than about 0.15, or greater than about 0.20. In some embodiments, the ratio is greater than about 0.1.

In some embodiments, a compound used in accordance with provided methods is characterized in that, when measured according to the procedure of ensuing Example 2, it has a free brain compound concentration equal to or above its cellular or enzymatic IC50 concentration.

In some embodiments, FASN inhibitors are those disclosed in WO 2014164749 and WO2014164767.

The compounds disclosed herein can be prepared in a manner similar to the procedures described in WO2014164749 and WO2014164767, the entire contents of each of which are hereby incorporated by reference.

In some embodiments, FASN inhibitors comprise a compound of formula I:

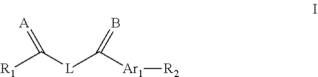

or a pharmaceutically acceptable salt thereof, wherein
R₁ is a C₁-C₃ hydroxyl-alkyl either unsubstituted or substituted with —CH₃ or —CH_{z}F_{3-z}, 5 membered cycloalkyl either unsubstituted or substituted with substituents selected from the group consisting of —Rₚ, —ORₚ, and —NRₚRₚ₁, or 3 or 4 membered cycloalkyl or heterocycloalkyl wherein (i) the heteroatom ring member of the 3 or 4 membered heterocycloalkyl is independently selected from O, S, or N, and (ii) the 3 or 4 membered cycloalkyl or heterocycloalkyl is either unsubstituted or optionally substituted with substituents selected from the group consisting of —Rₐ, —ORₐ, and —NRₐRₐ₁;
L is a 5-10 membered monocyclic or bicyclic alkyl or heteroalkyl wherein (i) the heteroatom ring members of the 5-10 membered monocyclic or bicyclic heteroalkyl are independently selected from O, S, or N, and (ii) each of the 5-10 membered monocyclic or bicyclic alkyl or heteroalkyl is either unsubstituted or optionally substituted with one or more —R_{b};
A and B are independently O or S;
Ar₁ is a 4-10 membered monocyclic or bicyclic aryl, heteroaryl or heterocycloalkyl, wherein (i) the 4-10 membered monocyclic or bicyclic heteroaryl or heterocycloalkyl has 1, 2, 3, or 4 heteroatoms which are independently selected from N, S or O, and (ii) each of said 4-10 membered monocyclic or bicyclic aryl, heteroaryl, or heterocycloalkyl is either unsubstituted or optionally independently substituted with 1 or more substituents which can be the same or different and are independently —R_{q};
R₂ is H or a 4-15 membered monocyclic, bicyclic, or tricyclic aryl, heteroaryl, cycloalkyl, or heterocycloalkyl, wherein (i) the 4-15 membered monocyclic, bicyclic, or tricyclic heteroaryl or heterocycloalkyl has 1, 2, 3, 4, 5, 6, 7, or 8 heteroatoms which are independently selected from N, S or O, and (ii) the aryl, heteroaryl, cycloalkyl, or heterocycloalkyl is optionally substituted with r instances of Rₑ;
each Rₚ and Rₚ₁ is independently H, C₁-C₄ alkyl, or C₃-C₄ cycloalkyl;
each Rₐ and Rₐ₁ is independently H, C₁-C₄ alkyl, or C₃-C₄ cycloalkyl;
each R_{b} is independently halo, C₁-C₄ alkyl, C₁-C₃ hydroxyl-alkyl, or C₃-C₄ cycloalkyl;
each Rₑ is independently halo, cyano, nitro, hydroxyl, hydroxyl-alkyl, hydroxylcycloalkyl, hydroxyl, heterocycloalkyl, hydroxyl-aryl, hydroxyl-heteroaryl, amino, aminoalkyl, (amino)alkoxy, —CONH₂, —C(O)NH(alkyl), —C(O)N(alkyl)₂, —C(O)NH(aryl), —C(O)N(aryl)₂, —CH_{z}F_{3-z}, —OCH_{z}F_{3-z}, alkyl, alkoxy, alkenyl, alkynyl, aryloxy, (alkoxyalkyl)amino, cycloalkyl, heterocycloalkyl, (heterocycloalkyl)alkyl, aryl, heteroaryl, —O(alkyl), —O(cycloalkyl), —O(heterocycloalkyl), —O(aryl), —O(heteroaryl), —ONH₂, —C(O)NH(alkyl), —C(O)N(aryl)₂, —C(O)NH(cycloalkyl), —NH(CO)cycloalkyl, —NH(SO₂), —NH(SO₂)alkyl, —NH(SO₂)aryl, —NH(SO₂)heteroaryl, —N(SO₂)cycloalkyl, —C(O)N(alkyl)₂, (aryl)alkyl, (heteroaryl)alkyl, —S(O)₂-alkyl, —S(O)₂-aryl, —S(O)₂-cycloalkyl, —C(O)N(alkyl)₂, —C(O)alkyl, —NH—C(O)-alkyl, —NH—C(O)-cycloalkyl, —NH—C(O)-heterocycloalkyl, —NH—C(O)-heterocycloalkyl-R_{d}, —NH—C(O)—R_{d}, —NH—C(O)-aryl, —NH—C(O)—NH-alkyl, —NH—C(O)—NH-cycloalkyl, —NH₂(CO)cycloalkyl, —NH—C(O)—NH-aryl, —NH—C(O)—O-alkyl, —NH—C(O)—NH-cycloalkyl, —NH—C(O)—O-cycloalkyl, —N(R_{d})—C(O)-alkyl, —N(R_{d})—C(O)-aryl, —N(R_{d})—S(O₂)cycloalkyl, —S(O₂)NH₂, —S(O₂)NH(alkyl), —S(O₂)N(R_{d})cycloalkyl, —S(O₂)N(alkyl)₂, —C(O)N(H)(alkyl), —C(O)N(R_{d})(cycloalkyl), methylenedioxy, —CH_{z}F_{3-z}, or —OCH_{z}F_{3-z}, wherein each instance of alkyl, aryl, heteroaryl, cycloalkyl, or heterocycloalkyl is optionally substituted with r instances of R_{b};
each R_{q} is independently halo, alkyl, —CH_{z}F_{3-z}, cyano, nitro, hydroxyl, hydroxylalkyl, amino, aminoalkyl, (amino)alkoxy, —CONH₂, —C(O)NH(alkyl), —C(O)N(alkyl)₂, —C(O)NH(aryl), —C(O)N(aryl)₂, —OCH_{z}F_{3-z}, alkyl, alkenyl, alkynyl, alkoxy, (alkoxyalkyl)amino, —N(R_{c})—C(O)-alkyl, —N(R_{c})—C(O)-aryl, cycloalkyl, heterocycloalkyl, aryl, or heteroaryl;
each Rₑ is independently H, halo, C₁-C₄ alkyl, or C₃-C₄ cycloalkyl;
each R_{d} is independently H, halo, C₁-C₄ alkyl, or C₃-C₄ cycloalkyl;
with the proviso that no two adjacent ring heteroatoms are both S or both O;
m is 0, 1, 2, 3, or 4;
n is 0, 1, 2, 3, or 4;
each r is independently 1, 2, 3, or 4
and z is 0, 1, 2, 3, or 4;
or solvates, esters, prodrugs or isomers thereof.

In some embodiments, FASN inhibitors comprise a compound of formula I-A:

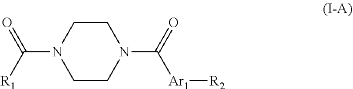

wherein:
Ar₁, R₁, R₂, Rₐ, Rₐ₁, R_{d}, Rₑ, Rₚ, Rₚ₁, R_{q}, m, r, and z are defined above and described in classes and subclasses herein, both singly and in combination;
or pharmaceutically acceptable salts, solvates, esters, prodrugs or isomers thereof.

In some embodiments, FASN inhibitors comprise a compound of formula I-B:

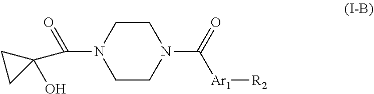

wherein:
Ar₁, R₂, R_{c}, R_{d}, Rₑ, R_{q}, m, r, and z are defined above and described in classes and subclasses herein, both singly and in combination;
or solvates, esters, prodrugs or isomers thereof.

In some embodiments, FASN inhibitors comprise a compound of formula

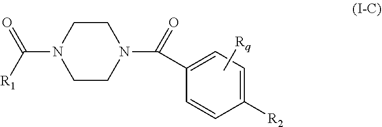

wherein:
R₁, R₂, Rₐ, Rₐ₁, R_{c}, R_{d}, Rₑ, Rₚ, Rₚ₁, R_{q}, r, and z are defined above and described in classes and subclasses herein, both singly and in combination; R_{q} is H, halo, C₁-C₄ alkyl, or C₃-C₄ cycloalkyl;
or solvates, esters, prodrugs or isomers thereof.

In some embodiments, FASN inhibitors comprise a compound of formula I-C′:

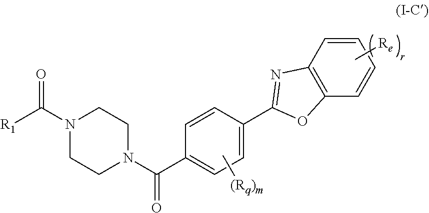

wherein:
R₁, Rₐ, Rₐ₁, R_{c}, R_{d}, Rₑ, Rₚ, Rₚ₁, R_{q}, m, r, and z are defined above and described in classes and subclasses herein, both singly and in combination;
or solvates, esters, prodrugs or isomers thereof.

In some embodiments, FASN inhibitors comprise a compound of formula I-C″:

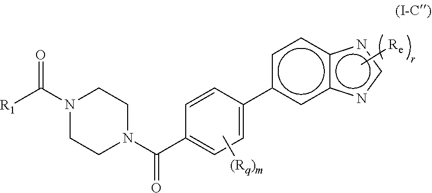

wherein:
R₁, Rₐ, Rₐ₁, R_{c}, R_{d}, Rₑ, Rₚ, Rₚ₁, R_{q}, m, r, and z are defined above and described in classes and subclasses herein, both singly and in combination;
or solvates, esters, prodrugs or isomers thereof.

In some embodiments, FASN inhibitors comprise a compound of formula I-D:

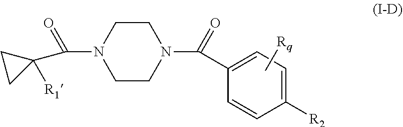

wherein:
R₁′ is OH or NH₂;
Ar₁, R₂, R_{c}, R_{d}, Rₑ, R_{q}, m, r, and z are defined above and described in classes and subclasses herein, both singly and in combination;
or solvates, esters, prodrugs or isomers thereof.

In some embodiments, FASN inhibitors comprise a compound of formula I-E:

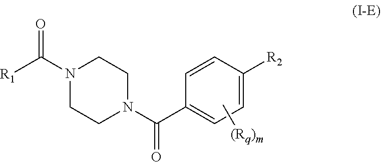

wherein:
R₁, R₂, Rₐ, Rₐ₁, R_{c}, R_{d}, Rₑ, Rₚ, Rₚ₁, R_{q}, m, r, and z are defined above and described in classes and subclasses herein, both singly and in combination;
or solvates, esters, prodrugs or isomers thereof.

In some embodiments, FASN inhibitors comprise a compound of formula I-F:

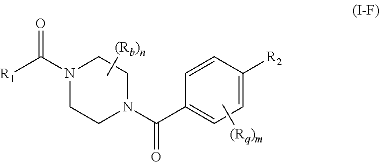

R₁, R₂, Rₚ, Rₚ₁, Rₐ, Rₐ₁, R_{b}, R_{c}, R_{d}, Rₑ, R_{q}, n, m, r, and z are defined above and described in classes and subclasses herein, both singly and in combination;
or solvates, esters, prodrugs or isomers thereof.

In some embodiments, FASN inhibitors comprise a compound of formula I-F′:

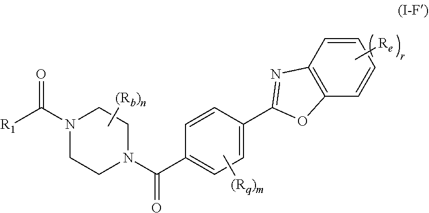

wherein:
R₁, Rₚ, Rₚ₁, Rₐ, Rₐ₁, R_{c}, R_{d}, Rₑ, R_{q}, n, m, r, and z are defined above and described in classes and subclasses herein, both singly and in combination;
or solvates, esters, prodrugs or isomers thereof.

In some embodiments, FASN inhibitors comprise a compound of formula I-F″:

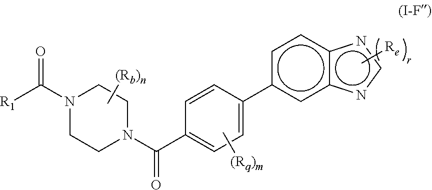

wherein:
R₁, Rₚ, Rₚ₁, Rₐ, Rₐ₁, R_{b}, R_{c}, R_{d}, Rₑ, R_{q}, n, m, r, and z are defined above and described in classes and subclasses herein, both singly and in combination;
or solvates, esters, prodrugs or isomers thereof.

In some embodiments, FASN inhibitors comprise a compound of formula I-G:

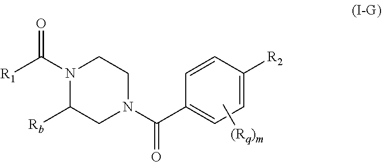

wherein:
R₁, Rₚ, Rₚ₁, Rₐ, Rₐ₁, R_{b}, R_{c}, R_{d}, Rₑ, R_{q}, n, m, r, and z are defined above and described in classes and subclasses herein, both singly and in combination;
or solvates, esters, prodrugs or isomers thereof.

In some embodiments, FASN inhibitors comprise a compound of formula I-G′:

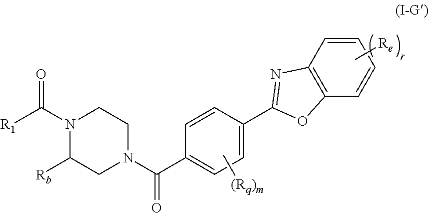

wherein:
R₁, Rₚ, Rₚ₁, Rₐ, Rₐ₁, R_{c}, R_{d}, Rₑ, R_{q}, n, m, r, and z are defined above and described in classes and subclasses herein, both singly and in combination;
or solvates, esters, prodrugs or isomers thereof.

In some embodiments, FASN inhibitors comprise a compound of formula I-G″:

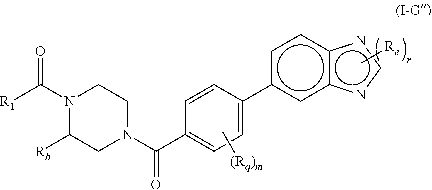

wherein:
R₁, Rₚ, Rₚ₁, Rₐ, Rₐ₁, R_{b}, R_{c}, R_{d}, Rₑ, R_{q}, n, m, r, and z are defined above and described in classes and subclasses herein, both singly and in combination;
or solvates, esters, prodrugs or isomers thereof.

In some embodiments, FASN inhibitors comprise a compound of formula I-H:

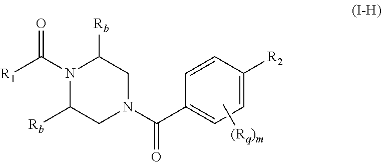

wherein:
R₁, Rₚ, Rₚ₁, Rₐ, Rₐ₁, R_{b}, R_{c}, R_{d}, Rₑ, R_{q}, n, m, r, and z are defined above and described in classes and subclasses herein, both singly and in combination;
or solvates, esters, prodrugs or isomers thereof.

In some embodiments, FASN inhibitors comprise a compound of formula I-H′:

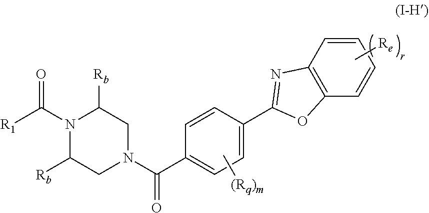

wherein:
R₁, Rₚ, Rₚ₁, Rₐ, Rₐ₁, R_{c}, R_{d}, Rₑ, R_{q}, n, m, r, and z are defined above and described in classes and subclasses herein, both singly and in combination;
or solvates, esters, prodrugs or isomers thereof.

In some embodiments, FASN inhibitors comprise a compound of formula I-H″:

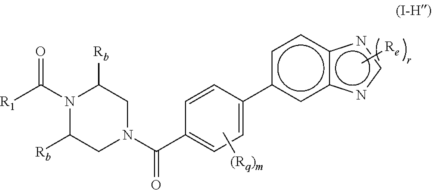

wherein:
R₁, Rₚ, Rₚ₁, Rₐ, Rₐ₁, R_{b}, R_{c}, R_{d}, Rₑ, R_{q}, n, m, r, and z are defined above and described in classes and subclasses herein, both singly and in combination;
or solvates, esters, prodrugs or isomers thereof.

In some embodiments, FASN inhibitors comprise a compound of formula II:

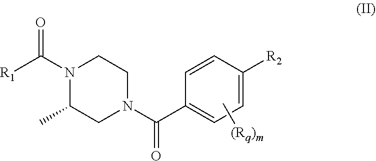

wherein:
R₁, R₂, Rₚ, Rₚ₁, Rₐ, Rₐ₁, R_{c}, R_{d}, Rₑ, R_{q}, m, r, and z are defined above and described in classes and subclasses herein, both singly and in combination;
or solvates, esters, prodrugs or isomers thereof.

In some embodiments, FASN inhibitors comprise a compound of formula III:

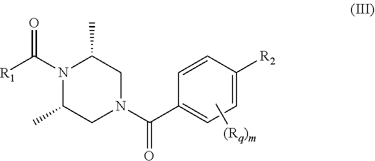

wherein:
R₁, R₂, Rₚ, Rₚ₁, Rₐ, Rₐ₁, R_{c}, R_{d}, Rₑ, R_{q}, m, r, and z are defined above and described in classes and subclasses herein, both singly and in combination;
or solvates, esters, prodrugs or isomers thereof.

In some embodiments, R₂ is selected from the group consisting of

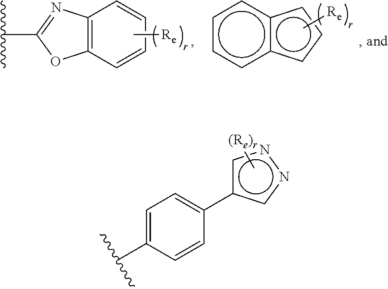

wherein:
R₁, Rₚ, Rₚ₁, Rₐ, Rₐ₁, R_{c}, R_{d}, Rₑ, R_{q}, n, m, r, and z are defined above and described in classes and subclasses herein, both singly and in combination;
or solvates, esters, prodrugs or isomers thereof.

In some embodiments, R₂ is

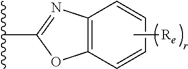

wherein:
R₁, Rₚ, Rₚ₁, Rₐ, Rₐ₁, R_{c}, R_{d}, Rₑ, R_{q}, n, m, r, and z are defined above and described in classes and subclasses herein, both singly and in combination;
or solvates, esters, prodrugs or isomers thereof.

In some embodiments, R₂ is

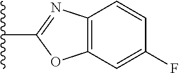

wherein:
R₁, Rₚ, Rₚ₁, Rₐ, Rₐ₁, R_{c}, R_{d}, Rₑ, R_{q}, n, m, r, and z are defined above and described in classes and subclasses herein, both singly and in combination;
or solvates, esters, prodrugs or isomers thereof.

In some embodiments, R₂ is

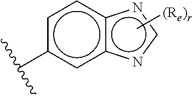

wherein:
R₁, Rₚ, Rₚ₁, Rₐ, Rₐ₁, R_{c}, R_{d}, Rₑ, R_{q}, n, m, r, and z are defined above and described in classes and subclasses herein, both singly and in combination;
or solvates, esters, prodrugs or isomers thereof.

In some embodiments, R₂ is

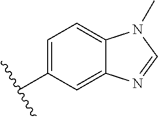

wherein:
R₁, Rₚ, Rₚ₁, Rₐ, Rₐ₁, R_{c}, R_{d}, Rₑ, R_{q}, n, m, r, and z are defined above and described in classes and subclasses herein, both singly and in combination;
or solvates, esters, prodrugs or isomers thereof.

In some embodiments, R₂ is

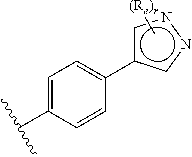

wherein:
R₁, Rₚ, Rₚ₁, Rₐ, Rₐ₁, R_{c}, R_{d}, Rₑ, R_{q}, n, m, r, and z are defined above and described in classes and subclasses herein, both singly and in combination;
or solvates, esters, prodrugs or isomers thereof.

In some embodiments, R₂ is

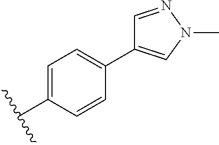

wherein:
R₁, Rₚ, Rₚ₁, Rₐ, Rₐ₁, R_{c}, R_{d}, Rₑ, R_{q}, n, m, r, and z are defined above and described in classes and subclasses herein, both singly and in combination;
or solvates, esters, prodrugs or isomers thereof.

The following embodiments are directed to any Formulas described herein, as applicable. For any moieties that are not specifically defined, the previous definitions control. Further, the moieties aryl, heteroaryl, and heterocycloalkyl in these embodiments can be independently unsubstituted or optionally substituted or optionally fused as described earlier.

In some embodiments, R₁ is C₁-C₃ hydroxyl-alkyl either unsubstituted or substituted with —CH₃ or —CH_{z}F_{3-z}, and A, B, L, Ar₁, R₂, Rₚ, Rₚ₁, Rₐ, Rₐ₁, R_{b}, R_{c}, R_{d}, R_{q} and z are as defined above and described in classes and subclasses herein, both singly and in combination.

In some embodiments, R₁ is a 5 membered cycloalkyl either unsubstituted or substituted with hydroxyl, and A, B, L, Ar₁, R₂, Rₚ, Rₚₜ, Rₐ, Rₐ₁, R_{b}, R_{c}, R_{d}, R_{q} and z are as defined above and described in classes and subclasses herein, both singly and in combination.

In some embodiments, R₁ is a 3 or 4 membered cycloalkyl, and A, B, L, Ar₁, R₂, Rₚ, Rₚ₁, Rₐ, Rₐ₁, R_{b}, R_{c}, R_{d}, R_{q} and z are as defined above and described in classes and subclasses herein, both singly and in combination.

In some embodiments, R₁ is a 3 or 4 membered heterocycloalkyl, and A, B, L, Ar₁, R₂, Rₚ, Rₚ₁, Rₐ, Rₐ₁, R_{b}, R_{c}, R_{d}, R_{q} and z are as defined above and described in classes and subclasses herein, both singly and in combination.

In some embodiments, R₁ is

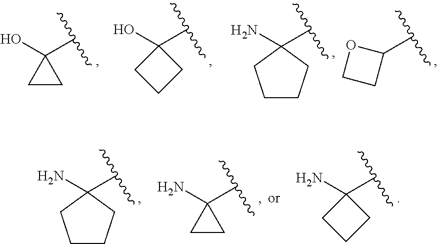

and A, B, L, Ar₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments R₁ is selected from the group consisting of:

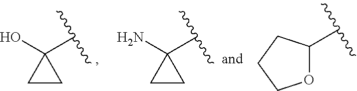 [SMILES: *C1(O)CC1 ; *C1(N)CC1 ; *C1CCCO1]

and A, B, L, Ar₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments R₁ is:

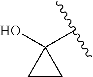 [SMILES: *C1(O)CC1]

and A, B, L, Ar₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments R₁ is

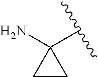 [SMILES: *C1(N)CC1]

and A, B, L, Ar₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments R₁ is:

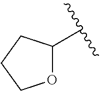 [SMILES: *C1CCCO1]

and A, B, L, Ar₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₁ is

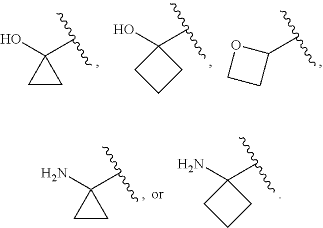

and A, B, L, Ar₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₁ is

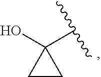 [SMILES: *C1(O)CC1]

and A, B, L, Ar₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₁ is

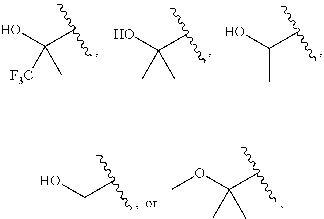

and A, B, L, Ar₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, A and B are O, and L, Ar₁, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, A and B are S, and L, Ar₁, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, either A or B is O, the other is S, and L, Ar₁, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, L is a 5-10 membered monocyclic alkyl, and A, B, Ar₁, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, L is a 5-10 membered bicyclic alkyl, and A, B, Ar₁, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, L is a 5-10 membered monocyclic heteroalkyl, and A, B, Ar₁, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, L is a 5-10 membered bicyclic heteroalkyl, and A, B, Ar₁, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, L is

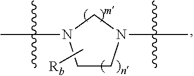

m′ is 1, 2, or 3, n′ is 0, 1, 2, or 3, and A, B, Ar₁, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, L is

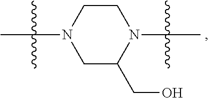 [SMILES: *N1CCN(*)C(CO)C1]

and A, B, Ar₁, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, L is

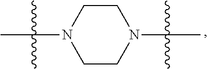 [SMILES: *N1CCN(*)CC1]

and A, B, Ar₁, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Ar₁ is an aryl, and A, B, L, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Ar₁ is a heteroaryl, and A, B, L, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Ar₁ is a 5-10 membered monocyclic aryl, and A, B, L, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Ar₁ is a 5-10 membered bicyclic aryl, and A, B, L, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Ar₁ is a 5-10 membered monocyclic heteroaryl, and A, B, L, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Ar₁ is a 5-10 membered bicyclic heteroaryl, and A, B, L, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Ar₁ is a substituted or unsubstituted 5 membered monocyclic aryl or heteroaryl and said heteroaryl has 1 or 2 heteroatoms which are independently S or N, and A, B, L, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Ar₁ is a substituted or unsubstituted form of

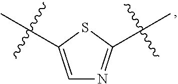 [SMILES: *c1cnc(*)s1]

and A, B, L, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Ar₁ is a substituted or unsubstituted 6 membered monocyclic aryl or heteroaryl and said heteroaryl has 1 or 2 heteroatoms which are N, and A, B, L, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Ar₁ is a substituted or unsubstituted form of

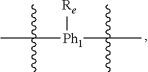

Ph₁ is phenyl, pyridinyl, pyrazinyl, or pyrimidinyl, Rₑ is H, halo, or C₁-C₃ alkyl, and A, B, L, R₁, R₂, Rₐ, Rₐ₁, R_{b}, R_{c}, R_{d}, Rₚ, Rₚ₁, R_{q}, n, m, r, and z are as defined above and described in classes and subclasses herein, both singly and in combination.

In some embodiments, Ar₁ is a substituted or unsubstituted form of

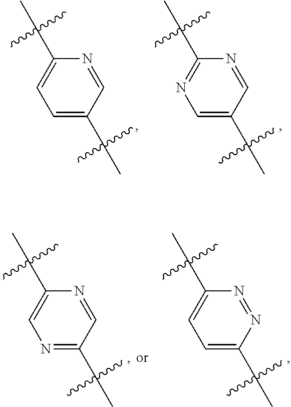 [SMILES: *c1ccc(*)nc1 ; *c1cnc(*)nc1 ; *c1cnc(*)cn1 ; *c1ccc(*)nn1]

and A, B, L, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Ar₁ is a substituted or unsubstituted 6 membered monocyclic aryl, and A, B, L, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Ar₁ is

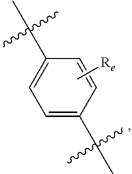

Rₑ is H, halo, or C₁-C₃ alkyl, and A, B, L, R₁, R₂, Rₐ, Rₐ₁, R_{b}, R_{c}, R_{d}, Rₚ, Rₚ₁, R_{q}, n, m, r, and z are as defined above and described in classes and subclasses herein, both singly and in combination.

In some embodiments, Ar₁ is

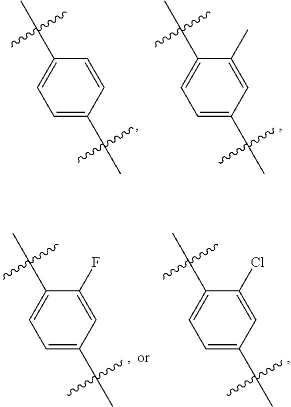

and A, B, L, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Ar₁ is a substituted or unsubstituted 9 membered 6,5-bicyclic heteroaryl and said heteroaryl has 1, 2, or 3 heteroatoms which are independently O, S or N, and A, B, L, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Ar₁ is a substituted or unsubstituted form of

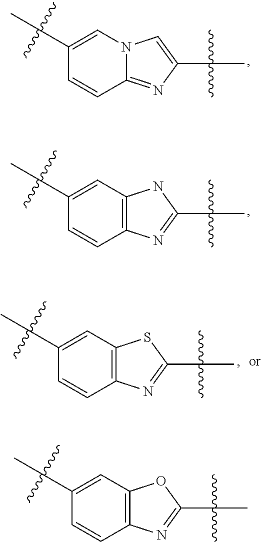 [SMILES: *c1ccc2nc(*)cn2c1 ; *c1ccc2nc(*)[nH]c2c1 ; *c1ccc2nc(*)sc2c1 ; *c1ccc2nc(*)oc2c1]

and said heteroaryl has 1, 2, or 3 heteroatoms which are independently S or N, and A, B, L, R₁, R₂, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₂ is a substituted or unsubstituted aryl, and A, B, L, Ar₁, R₁, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₂ is a substituted or unsubstituted heteroaryl, and A, B, L, Ar₁, R₁, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₂ is a substituted or unsubstituted cycloalkyl, and A, B, L, Ar₁, R₁, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₂ is a substituted or unsubstituted heterocycloalkyl, and A, B, L, Ar₁, R₁, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₂ is a substituted or unsubstituted monocyclic or bicyclic 5-10 membered aryl or heteroaryl, and A, B, L, Ar₁, R₁, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₂ is a unsubstituted or substituted monocylic 6 membered aryl, and A, B, L, Ar₁, R₁, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₂ is

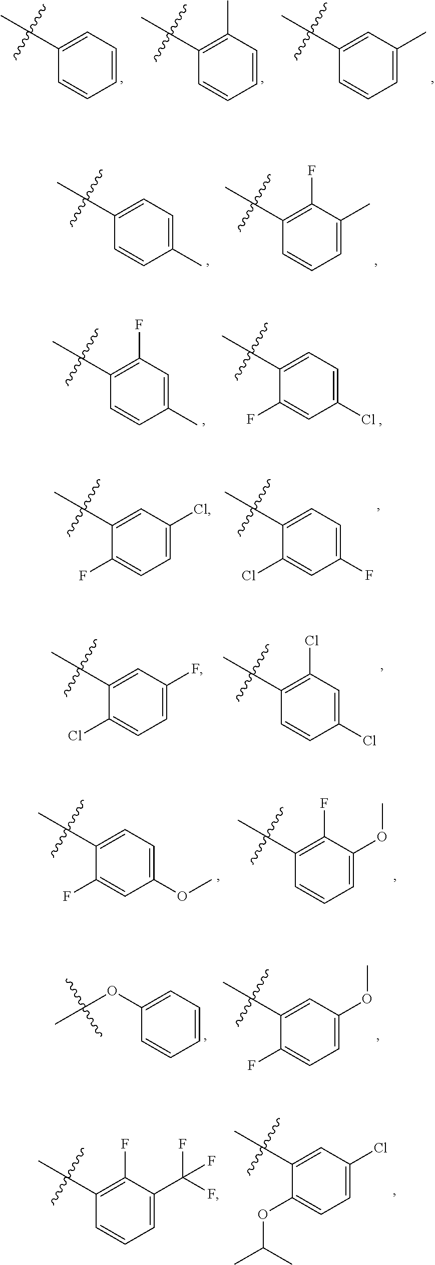

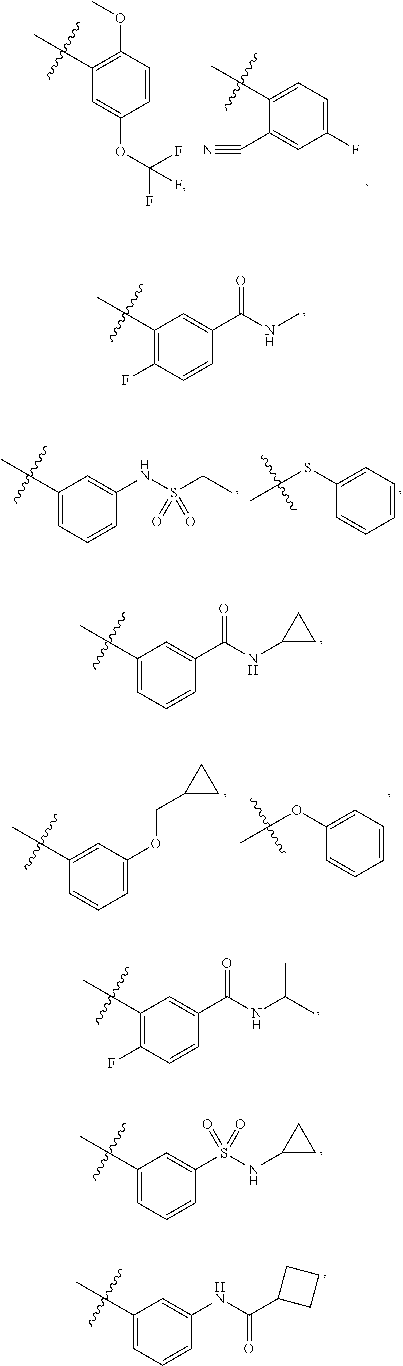

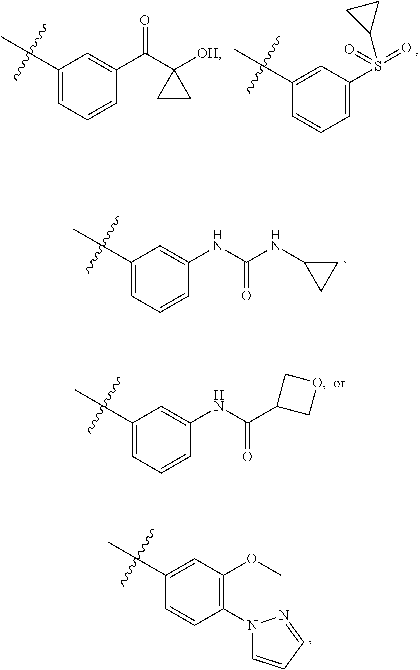 [SMILES: *c1cccc(C(=O)C2(O)CC2)c1 ; *c1cccc(S(=O)(=O)C2CC2)c1 ; "," ; *c1cccc(NC(=O)NC2CC2)c1 ; *c1cccc(NC(=O)C2COC2)c1 ; *c1ccc(-n2cccn2)c(OC)c1]

and A, B, L, Ar₁, R₁, Rₐ, Rₐ₁, R_{b}, R_{c}, R_{d}, Rₚ, Rₚ₁, R_{q}, n, m, and z are as defined above and described in classes and subclasses herein, both singly and in combination.

In some embodiments, R₂ is a substituted or unsubstituted bicyclic 8-10 membered aryl or 8-10 membered heteroaryl, and A, B, L, Ar₁, R₁, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₂ is a substituted or unsubstituted 8 membered 5,5 bicyclic heteroaryl and said heteroaryl has 1, 2, 3, or 4 heteroatoms and said heteroatoms are independently O, S, or N, and A, B, L, Ar₁, R₁, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₂ is a substituted or unsubstituted form of

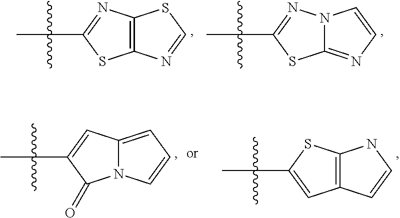 [SMILES: *c1nc2scnc2s1 ; *c1nn2ccnc2s1 ; *C1=Cc2cccn2C1=O ; *c1cc2cc[nH]c2s1]

and A, B, L, Ar₁, R₁, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₂ is a substituted or unsubstituted 9 membered 6,5 bicyclic heteroaryl and said heteroaryl has 1, 2, 3, or 4 heteroatoms and said heteroatoms are independently O, S, or N, and A, B, L, Ar₁, R₁, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₂ is a substituted or unsubstituted form of

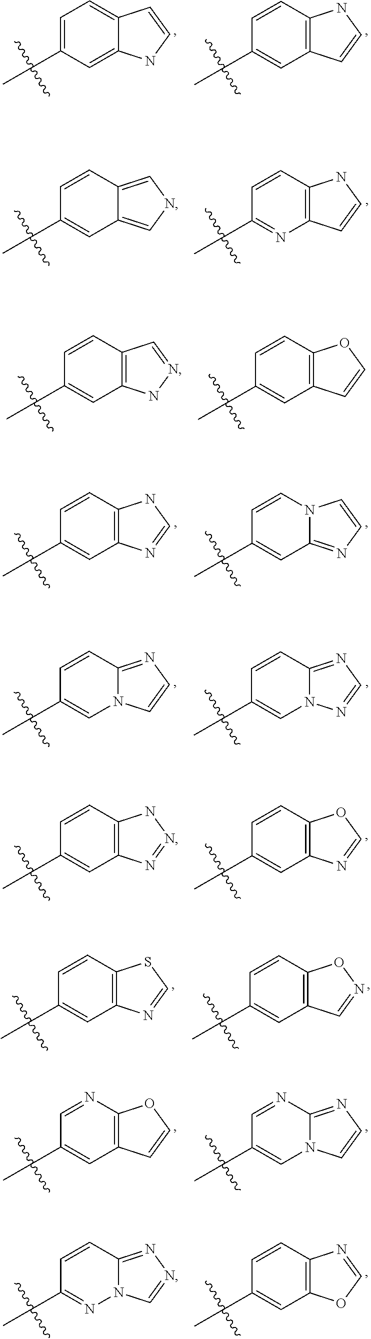

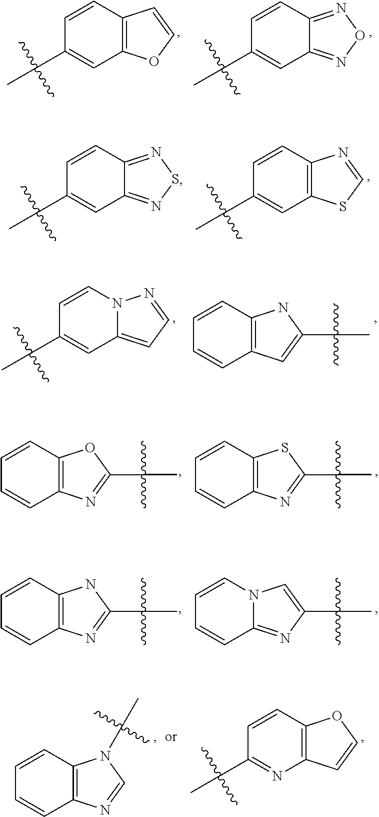

and A, B, L, Ar₁, R₁, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₂ is a substituted or unsubstituted 10 membered 6,6 bicyclic aryl or heteroaryl and said heteroaryl has 1, 2, 3, or 4 heteroatoms and said heteroatoms are O, S, or N, and A, B, L, Ar₁, R₁, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₂ is a substituted or unsubstituted form of

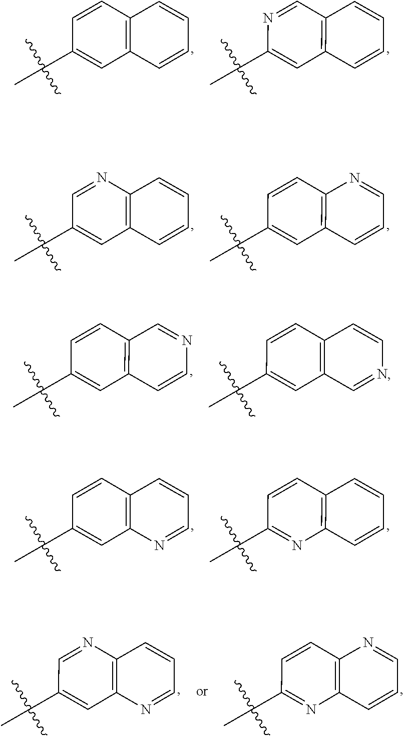

and A, B, L, Ar₁, R₁, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₂ is optionally substituted benzoxazolyl, and A, B, L, Ar₁, R₁, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination. In some embodiments, R₂ is benzoxazolyl substituted with halogen, and A, B, L, Ar₁, R₁, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, R₂ is optionally substituted benzimidazolyl, and A, B, L, Ar₁, R₁, Rₐ, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Rₚ is H, and A, B, L, Ar₁, R₁, R₂, Rₐ, Rₐ₁, R_{b}, R_{c}, R_{d}, Rₑ, Rₚ₁, R_{q}, n, m, r, and z as defined above and described in classes and subclasses herein, both singly and in combination.

In some embodiments, Rₚ is halo, and A, B, L, Ar₁, R₁, R₂, Rₐ, Rₐ₁, R_{b}, R_{c}, R_{d}, Rₑ, Rₚ₁, R_{q}, n, m, r, and z are as defined above and described in classes and subclasses herein, both singly and in combination.

In some embodiments, Rₚ is C₁-C₄ alkyl, and A, B, L, Ar₁, R₁, R₂, Rₐ, Rₐ₁, R_{b}, R_{c}, R_{d}, Rₑ, Rₚ₁, R_{q}, n, m, r, and z are as defined above and described in classes and subclasses herein, both singly and in combination.

In some embodiments, Rₚ is C₃-C₄ cycloalkyl, and A, B, L, Ar₁, R₁, R₂, Rₐ, Rₐ₁, R_{b}, R_{c}, R_{d}, Rₑ, Rₚ₁, R_{q}, n, m, r, and z are as defined above and described in classes and subclasses herein, both singly and in combination.

In some embodiments, Rₚ₁ is H, and A, B, L, Ar₁, R₁, R₂, Rₐ, Rₐ₁, R_{b}, R_{c}, R_{d}, Rₑ, Rₚ, R_{q}, n, m, r, and z are as defined above and described in classes and subclasses herein, both singly and in combination.

In some embodiments, Rₚ₁ is halo, and A, B, L, Ar₁, R₁, R₂, Rₐ, Rₐ₁, R_{b}, R_{c}, R_{d}, Rₑ, Rₚ, R_{q}, n, m, r, and z are as defined above and described in classes and subclasses herein, both singly and in combination.

In some embodiments, Rₚ₁ is C₁-C₄ alkyl, and A, B, L, Ar₁, R₁, R₂, Rₐ, Rₐ₁, R_{b}, R_{c}, R_{d}, Rₑ, Rₚ, R_{q}, n, m, r, and z are as defined above and described in classes and subclasses herein, both singly and in combination.

In some embodiments, Rₚ₁ is C₃-C₄ cycloalkyl, and A A, B, L, Ar₁, R₁, R₂, Rₐ, Rₐ₁, R_{b}, R_{c}, R_{d}, Rₑ, Rₚ, R_{q}, n, m, r, and z are as defined above and described in classes and subclasses herein, both singly and in combination.

In some embodiments, Rₐ is H, and A, B, L, Ar₁, R₁, R₂, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Rₐ is halo, and A, B, L, Ar₁, R₁, R₂, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Rₐ is C₁-C₄ alkyl, and A, B, L, Ar₁, R₁, R₂, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Rₐ is C₃-C₄ cycloalkyl, and A, B, L, Ar₁, R₁, R₂, Rₐ₁, R_{b}, R_{c}, R_{d}, Rₑ, Rₚ, Rₚ₁, R_{q}, n, m, r, and z are as defined above and described in classes and subclasses herein, both singly and in combination.

In some embodiments, Rₐ₁ is H, and A, B, L, Ar₁, R₁, R₂, Rₐ, R_{b}, R_{c}, R_{d}, Rₑ, Rₚ, Rₚ₁, R_{q}, n, m, r, and z are as defined above and described in classes and subclasses herein, both singly and in combination.

In some embodiments, Rₐ₁ is halo, and A, B, L, Ar₁, R₁, R₂, Rₐ, R_{b}, R_{c}, R_{d}, Rₑ, Rₚ, Rₚ₁, R_{q}, n, m, r, and z are as defined above and described in classes and subclasses herein, both singly and in combination.

In some embodiments, Rₐ₁ is C₁-C₄ alkyl, and A, B, L, Ar₁, R₁, R₂, Rₐ, R_{b}, R_{c}, R_{d}, Rₑ, Rₚ, Rₚ₁, R_{q}, n, m, r, and z are as defined above and described in classes and subclasses herein, both singly and in combination.

In some embodiments, Rₐ₁ is C₃-C₄ cycloalkyl, and A, B, L, Ar₁, R₁, R₂, Rₐ, R_{b}, R_{c}, R_{d}, Rₑ, Rₚ, Rₚ₁, R_{q}, n, m, r, and z are as defined above and described in classes and subclasses herein, both singly and in combination.

In some embodiments, R_{b} is H, and A, B, L, Ar₁, R₁, R₂, Rₐ, Rₐ₁, R_{c}, R_{d}, Rₑ, Rₚ, Rₚ₁, R_{q}, n, m, r, and z are as defined above and described in classes and subclasses herein, both singly and in combination.

In some embodiments, R_{b} is halo, and A, B, L, Ar₁, R₁, R₂, Rₐ, Rₐ₁, R_{c}, R_{d}, Rₑ, Rₚ, Rₚ₁, R_{q}, n, m, r, and z are as defined above and described in classes and subclasses herein, both singly and in combination.

In some embodiments, R_{b} is C₁-C₄ alkyl, and A, B, L, Ar₁, R₁, R₂, Rₐ, Rₐ₁, R_{c}, R_{d}, Rₑ, Rₚ, Rₚ₁, R_{q}, n, m, r, and z are as defined above and described in classes and subclasses herein, both singly and in combination.

In some embodiments, R_{b} is C₁-C₃ hydroxyl-alkyl, and A, B, L, Ar₁, R₁, R₂, Rₐ, Rₐ₁, R_{c}, R_{d}, Rₑ, Rₚ, Rₚ₁, R_{q}, n, m, r, and z are as defined above and described in classes and subclasses herein, both singly and in combination.

In some embodiments, R_{b} is C₃-C₄ cycloalkyl, and A, B, L, Ar₁, R₁, R₂, Rₐ, Rₐ₁, R_{c}, R_{d}, Rₑ, Rₚ, Rₚ₁, R_{q}, n, m, r, and z are as defined above and described in classes and subclasses herein, both singly and in combination.

In some embodiments, R_{c} is H, and A, B, L, Ar₁, R₁, R₂, Rₐ, Rₐ₁, R_{b}, R_{d}, Rₑ, Rₚ, Rₚ₁, R_{q}, n, m, r, and z are as defined above and described in classes and subclasses herein, both singly and in combination.

In some embodiments, R_{c} is halo, and A, B, L, Ar₁, R₁, R₂, Rₐ, Rₐ₁, R_{b}, R_{d}, Rₑ, Rₚ, Rₚ₁, R_{q}, n, m, r, and z are as defined above and described in classes and subclasses herein, both singly and in combination.

In some embodiments, R_{c} is C₁-C₄ alkyl, and A, B, L, Ar₁, R₁, R₂, Rₐ, Rₐ₁, R_{b}, R_{d}, Rₑ, Rₚ, Rₚ₁, R_{q}, n, m, r, and z are as defined above and described in classes and subclasses herein, both singly and in combination.

In some embodiments, R_{c} is C₃-C₄ cycloalkyl, and A, B, L, Ar₁, R₁, R₂, Rₐ, Rₐ₁, R_{b}, R_{d}, Rₑ, Rₚ, Rₚ₁, R_{q}, n, m, r, and z are as defined above and described in classes and subclasses herein, both singly and in combination.

In some embodiments, R_{d} is H, and A, B, L, Ar₁, R₁, R₂, Rₐ, Rₐ₁, R_{b}, R_{c}, Rₑ, Rₚ, Rₚ₁, R_{q}, n, m, r, and z are as defined above and described in classes and subclasses herein, both singly and in combination.

In some embodiments, R_{d} is halo, and A, B, L, Ar₁, R₁, R₂, Rₐ, Rₐ₁, R_{b}, R_{c}, Rₑ, Rₚ, Rₚ₁, R_{q}, n, m, r, and z are as defined above and described in classes and subclasses herein, both singly and in combination.

In some embodiments, R_{d} is C₁-C₄ alkyl, and A, B, L, Ar₁, R₁, R₂, Rₐ, Rₐ₁, R_{b}, R_{c}, Rₑ, Rₚ, Rₚ₁, R_{q}, n, m, r, and z are as defined above and described in classes and subclasses herein, both singly and in combination.

In some embodiments, R_{d} is C₃-C₄ cycloalkyl, and A, B, L, Ar₁, R₁, R₂, Rₐ, Rₐ₁, R_{b}, R_{c}, Rₑ, Rₚ, Rₚ₁, R_{q}, n, m, r, and z are as defined above and described in classes and subclasses herein, both singly and in combination.

In some embodiments, R_{q} is H, and A, B, L, Ar₁, R₁, R₂, Rₐ, Rₐ₁, R_{b}, R_{c}, Rₑ, Rₚ, Rₚ₁, R_{d}, n, m, r, and z are as defined above and described in classes and subclasses herein, both singly and in combination.

In some embodiments, R_{q} is halo, and A, B, L, Ar₁, R₁, R₂, Rₐ, Rₐ₁, R_{b}, R_{c}, Rₑ, Rₚ, Rₚ₁, R_{d}, n, m, r, and z are as defined above and described in classes and subclasses herein, both singly and in combination.

In some embodiments, R_{q} is C₁-C₄ alkyl, and A, B, L, Ar₁, R₁, R₂, Rₐ, Rₐ₁, R_{b}, R_{c}, Rₑ, Rₚ, Rₚ₁, R_{d}, n, m, r, and z are as defined above and described in classes and subclasses herein, both singly and in combination.

In some embodiments, R_{q} is C₃-C₄ cycloalkyl, and A, B, L, Ar₁, R₁, R₂, Rₐ, Rₐ₁, R_{b}, R_{c}, Rₑ, Rₚ, Rₚ₁, R_{d}, n, m, r, and z are as defined above and described in classes and subclasses herein, both singly and in combination.

In some embodiments, z is 0, and A, B, L, Ar₁, R₁, R₂, Rₐ, Rₐ₁, R_{b}, R_{c}, Rₑ, Rₚ, Rₚ₁, R_{q}, R_{d}, n, m, and r are as defined above and described in classes and subclasses herein, both singly and in combination.

In some embodiments, z is 1 and A, B, L, Ar₁, R₁, R₂, Rₐ, Rₐ₁, R_{b}, R_{c}, Rₑ, Rₚ, Rₚ₁, R_{q}, R_{d}, n, m, and r are as defined above and described in classes and subclasses herein, both singly and in combination.

In some embodiments, z is 2 and A, B, L, Ar₁, R₁, R₂, Rₐ, Rₐ₁, R_{b}, R_{c}, Rₑ, Rₚ, Rₚ₁, R_{q}, R_{d}, n, m, and r are as defined above and described in classes and subclasses herein, both singly and in combination.

In some embodiments of methods provided herein, for a compound as described in the formulae above, R₂ is not a substituted or unsubstituted form of

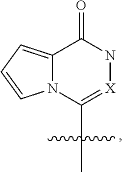

where X is N or CH.

In some embodiments of methods provided herein, for a compound as described in the formulae above, when Ar₁ is

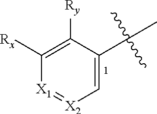

connected to

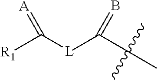

at position 1, and X₁ and X₂ are independently N or C—R_{z}, and R_{y} and R_{z} are any substituent, then Rₓ does not include alkynyl, alkenyl, aryl, 5-14 membered heterocyclic, 5-14 membered heteroaromatic, or 4-9 membered carbocyclic.

In some embodiments of methods provided herein, for a compound as described in the formulae above when R₂ is

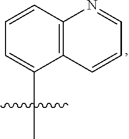 [SMILES: *c1cccc2ncccc12]

Ar₁ is not a substituted or unsubstituted form of

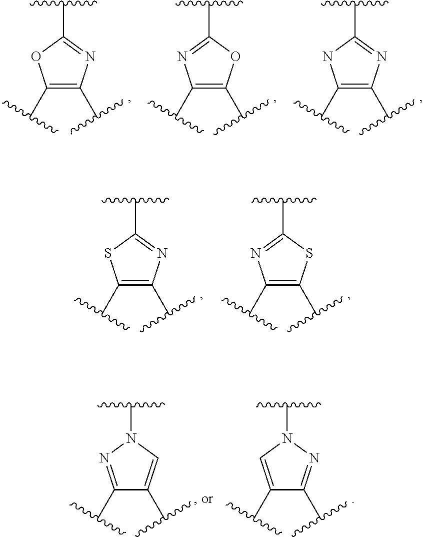

In some embodiments of methods provided herein, for a compound as described in the formulae above, when Ar₁ is a substituted or unsubstituted form of a 5 membered heteroaryl, Ar₁ is

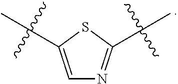 [SMILES: *c1cnc(*)s1]

In some embodiments, n is 0. In some embodiments, n is 1. In some embodiments n is 2. In some embodiments, n is 3. In some embodiments, n is 4.

In some embodiments, each Rb is independently C1-4 alkyl. In some embodiments, Rb is methyl. In some embodiments, Rb is ethyl. In some embodiments, Rb is methyl and n is 1. In some embodiments, Rb is methyl and n is 2.

In some embodiments, m is 0. In some embodiments, m is 1. In some embodiments m is 2. In some embodiments, m is 3. In some embodiments m is 4.

In some embodiments, Rq is halogen. In some embodiments, Rq is fluoro. In some embodiments, Rq is chloro. In some embodiments, m is 1 and Rq is fluoro.

In some embodiments, FASN inhibitors useful in provided methods are selected from the group consisting of

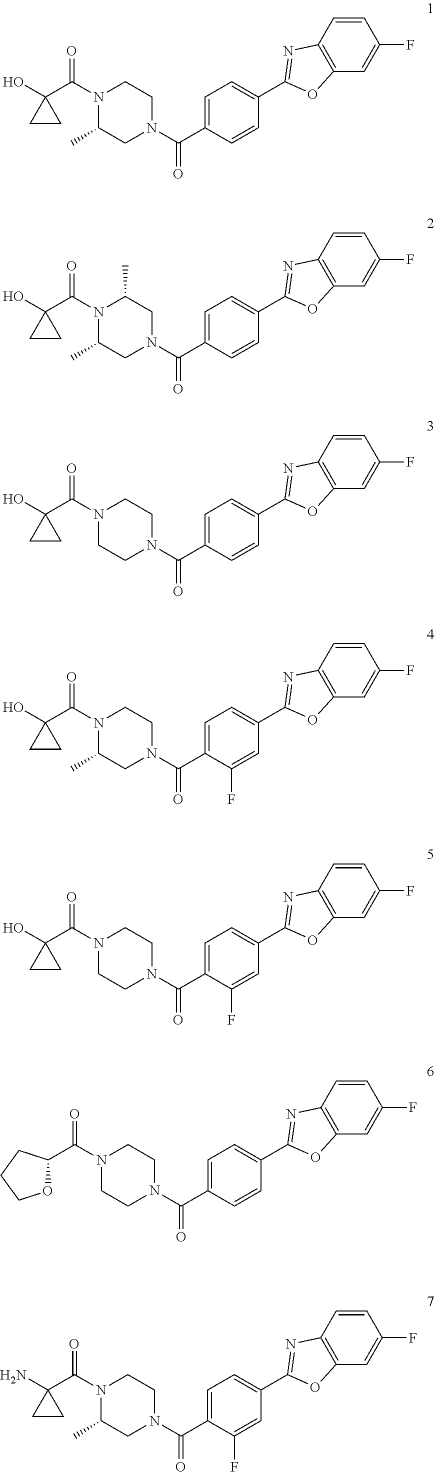

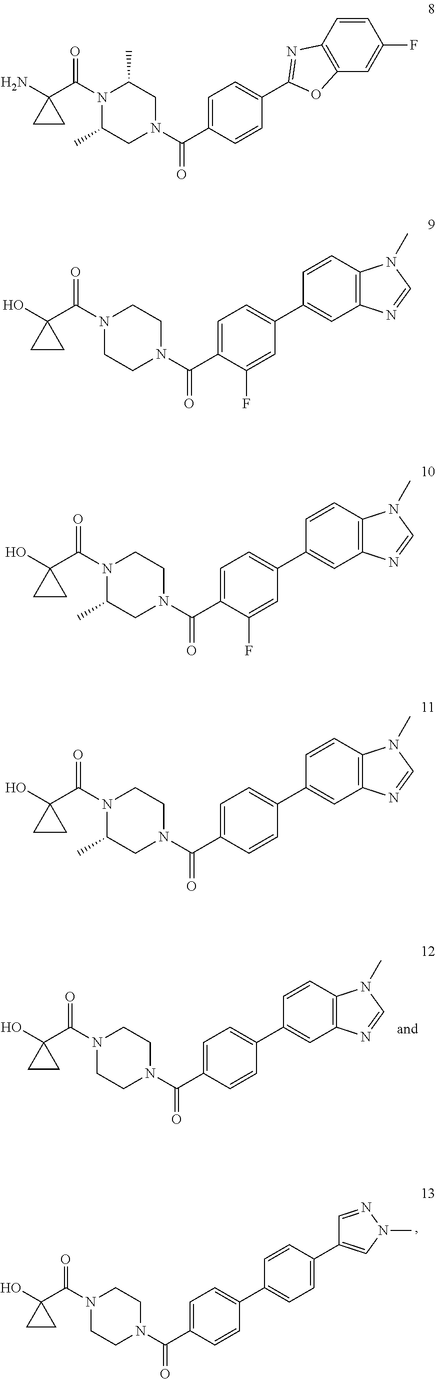 [SMILES: C[C@@H]1CN(C(=O)c2ccc(-c3nc4ccc(F)cc4o3)cc2)C[C@H](C)N1C(=O)C1(N)CC1 ; Cn1cnc2cc(-c3ccc(C(=O)N4CCN(C(=O)C5(O)CC5)CC4)c(F)c3)ccc21 ; C[C@H]1CN(C(=O)c2ccc(-c3ccc4c(c3)ncn4C)cc2F)CCN1C(=O)C1(O)CC1 ; C[C@H]1CN(C(=O)c2ccc(-c3ccc4c(c3)ncn4C)cc2)CCN1C(=O)C1(O)CC1 ; Cn1cnc2cc(-c3ccc(C(=O)N4CCN(C(=O)C5(O)CC5)CC4)cc3)ccc21 ; Cn1cc(-c2ccc(-c3ccc(C(=O)N4CCN(C(=O)C5(O)CC5)CC4)cc3)cc2)cn1]

or pharmaceutically acceptable salts thereof.

In some embodiments, a FASN inhibitor useful in provided methods is

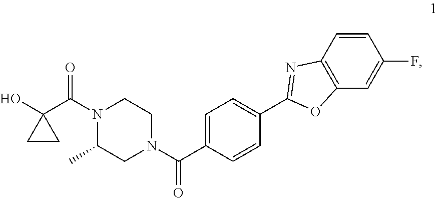 [SMILES: C[C@H]1CN(C(=O)c2ccc(-c3nc4ccc(F)cc4o3)cc2)CCN1C(=O)C1(O)CC1]

or pharmaceutically acceptable salts thereof.

In some embodiments, a FASN inhibitor useful in provided methods is

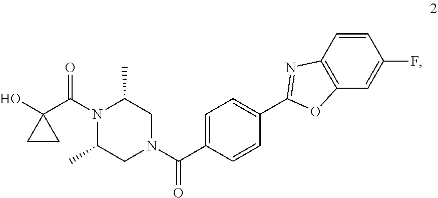 [SMILES: C[C@@H]1CN(C(=O)c2ccc(-c3nc4ccc(F)cc4o3)cc2)C[C@H](C)N1C(=O)C1(O)CC1]

or pharmaceutically acceptable salts thereof.

In some embodiments, a FASN inhibitor useful in provided methods is

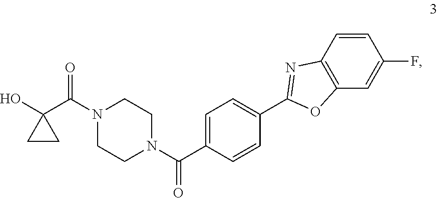 [SMILES: O=C(c1ccc(-c2nc3ccc(F)cc3o2)cc1)N1CCN(C(=O)C2(O)CC2)CC1]

or pharmaceutically acceptable salts thereof.

In some embodiments, a FASN inhibitor useful in provided methods is

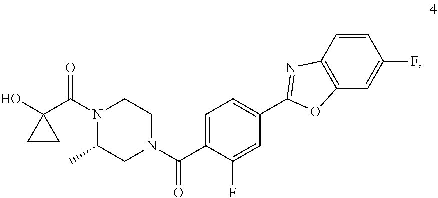 [SMILES: C[C@H]1CN(C(=O)c2ccc(-c3nc4ccc(F)cc4o3)cc2F)CCN1C(=O)C1(O)CC1]

or pharmaceutically acceptable salts thereof.

In some embodiments, a FASN inhibitor useful in provided methods is

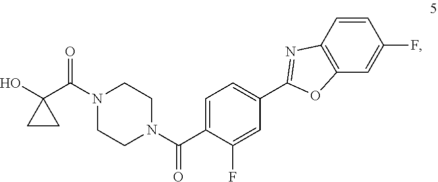 [SMILES: O=C(c1ccc(-c2nc3ccc(F)cc3o2)cc1F)N1CCN(C(=O)C2(O)CC2)CC1]

or pharmaceutically acceptable salts thereof.

In some embodiments, a FASN inhibitor useful in provided methods is

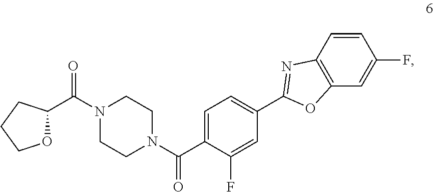 [SMILES: O=C(c1ccc(-c2nc3ccc(F)cc3o2)cc1F)N1CCN(C(=O)[C@H]2CCCO2)CC1]

or pharmaceutically acceptable salts thereof.

In some embodiments, a FASN inhibitor useful in provided methods is

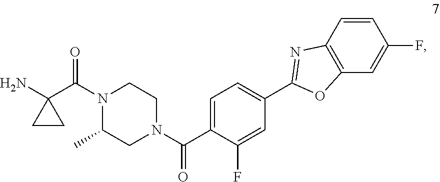 [SMILES: C[C@H]1CN(C(=O)c2ccc(-c3nc4ccc(F)cc4o3)cc2F)CCN1C(=O)C1(N)CC1]

or pharmaceutically acceptable salts thereof.

In some embodiments, a FASN inhibitor useful in provided methods is

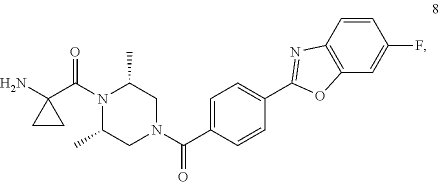 [SMILES: C[C@@H]1CN(C(=O)c2ccc(-c3nc4ccc(F)cc4o3)cc2)C[C@H](C)N1C(=O)C1(N)CC1]

or pharmaceutically acceptable salts thereof.

In some embodiments, a FASN inhibitor useful in provided methods is

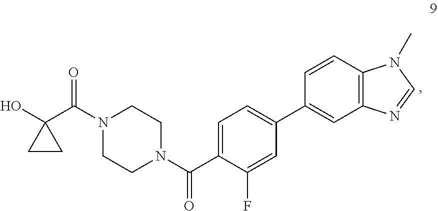 [SMILES: Cn1cnc2cc(-c3ccc(C(=O)N4CCN(C(=O)C5(O)CC5)CC4)c(F)c3)ccc21]

or pharmaceutically acceptable salts thereof.

In some embodiments, a FASN inhibitor useful in provided methods is

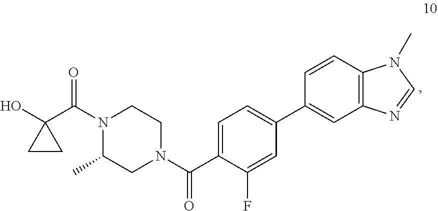 [SMILES: C[C@H]1CN(C(=O)c2ccc(-c3ccc4c(c3)ncn4C)cc2F)CCN1C(=O)C1(O)CC1]

or pharmaceutically acceptable salts thereof.

In some embodiments, a FASN inhibitor useful in provided methods is

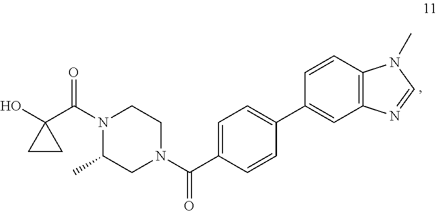 [SMILES: C[C@H]1CN(C(=O)c2ccc(-c3ccc4c(c3)ncn4C)cc2)CCN1C(=O)C1(O)CC1]

or pharmaceutically acceptable salts thereof.

In some embodiments, a FASN inhibitor useful in provided methods is

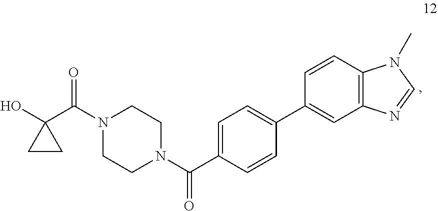 [SMILES: Cn1cnc2cc(-c3ccc(C(=O)N4CCN(C(=O)C5(O)CC5)CC4)cc3)ccc21]

or pharmaceutically acceptable salts thereof.

In some embodiments, a FASN inhibitor useful in provided methods is

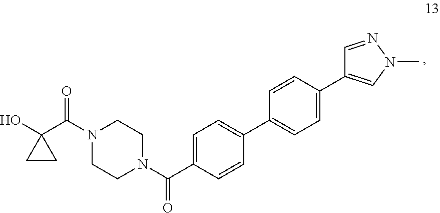 [SMILES: Cn1cc(-c2ccc(-c3ccc(C(=O)N4CCN(C(=O)C5(O)CC5)CC4)cc3)cc2)cn1]

or pharmaceutically acceptable salts thereof.

Certain embodiments of the present disclosure refer to Compound A and Compound B, which are compounds of Formula I-F and Formula I-F′. In some embodiments, Compound A and Compound B are selected from one of Compounds 1-8 shown above.

### Uses for the Treatment of Glioblastoma

Glioblastoma (GBM) is considered a tumor type highly dependent on lipid metabolism, a mechanism that provides lipid building blocks for cell membranes, provide acyl chain for protein translational modifications, and promotes oncogenic signaling. High de novo lipogenesis (DNL) activity inversely correlates with overall survival of GBM patients (Geng et al., (2016). Inhibition of SOAT1 Suppresses Glioblastoma Growth via Blocking SREBP-1-Mediated Lipogenesis. Clin. Cancer Res. Off. J. Am. Assoc. Cancer Res. 22, 5337-5348.). It has been reported that the blood brain barrier may restrict the passage of long fatty acids into the brain (Edmond et al., (1998). Fatty acid transport and utilization for the developing brain. J. Neurochem. 70, 1227-1234). CD36 is a fatty acid transporter that facilitates fatty acid uptake in human cancers. GBM tumors showed lower CD36 expression levels comparing to other human cancer types. GBM is highly dependent on DNL and may have a lesser potential of escape via the uptake and utilization of exogenous fatty acids.

Glioma stem cells (GSCs) are a subpopulation of brain tumor cells that express CD133 and have self renewal and greater tumorigenic activity (Lathia et al., (2015). Cancer stem cells in glioblastoma. Genes Dev. 29, 1203-1217). Accumulating evidence suggests that GSCs are the major culprit for the resistance to radiation and chemotherapy. In both human glioma xenografts and primary patient GBM specimens, GSCs survived radiation by preferentially activating the DNA damage checkpoint and repairing radiation-induced DNA damage when compared to CD133-negative gliomas cells (Bao et al., (2006). Glioma stem cells promote radioresistance by preferential activation of the DNA damage response. Nature 444, 756-760). In a genetically engineered murine glioma model, GSCs were resistant to TMZ treatment and became a new pool of tumor cells that caused the mortality of glioma bearing mice. Eradication of GSCs led to prolonged survival in the tumor bearing mice, suggesting targeting GSCs could offer potential benefit for GBM patients (Chen et al., (2012). A restricted cell population propagates glioblastoma growth after chemotherapy. Nature 488, 522-526). DNL is also believed to support glioma stem cell growth and activity (Yasumoto et al., 2016). Inhibition of lipid metabolism targets, such as Fatty Acid Synthase (FASN), Fatty acyl-CoA synthetase (ACSVL3), or transcription factor Sterol Regulatory Element-Binding Protein-1 (SREBP-1), suppresses the growth and stem cell markers in GSCs (Geng et al., 2016; Sun et al., (2014). Lipid metabolism enzyme ACSVL3 supports glioblastoma stem cell maintenance and tumorigenicity. BMC Cancer 14, 401; Yasumoto et al., 2016).

FASN is the enzyme that controls the last step of the biosynthesis of palmitate, the end product of DNL. FASN is overexpressed in many cancers, including GBM, and DNL has been postulated to provide lipid building blocks for cell membranes, provide acyl moiety for protein translational modifications, and promote oncogenic signaling. Furthermore, DNL is important to maintain the stemness of GSCs, a subpopulation of brain tumor cells that have been shown to confer resistance to GBM standard-of-care therapies, such as temozolomide and ionizing radiation. In addition, intracellular synthesis of fatty acids is a requirement for M2 macrophage differentiation as well as for sustaining the immunosuppressive mechanisms of myeloid-derived suppressor cells (MDSC). M2 macrophages and MDSCs block the anti-tumor function of T cells and represent major populations of GBM-infiltrating innate immune cells in humans. Targeting M2-like tumor-associated macrophages (TAMs) and MDSCs is a strategy that has shown anti-tumor activity and survival benefit in preclinical studies in murine glioma models. Without wishing to be bound by any particular theory, Applicant proposes that FASN inhibitors are useful to treat GBM by: 1) inhibiting the growth of differentiated GBM cells with inherent elevated DNL activity, 2) perturbing the “sternness” state of GSCs that are dependent on DNL, and 3) restoring anti-tumor T cell responses by inhibiting TAM and MDSC activities.

Compounds of the present disclosure potently inhibited fatty acid synthesis in a human cancer line at concentrations commensurate with inhibition of proliferation and induction of apoptosis (data not shown). In addition, compounds of the present disclosure potently blocked the neurosphere growth of patient-derived GSCs, blocked CSF-1 dependent M2 macrophage differentiation (data not shown), and relieved MDSC-mediated suppression of T cell proliferation in vitro (data not shown).

In some embodiments, the present disclosure provides methods of treating glioblastoma comprising the step of administering to a patient in need thereof a compound as provided herein.

In some embodiments, the present disclosure provides an improvement to methods for inhibiting FASN, the improvement comprising administering a FASN inhibitor that crosses the blood brain barrier. In certain embodiments, such FASN inhibitor compounds are of Formula I and other classes, subclasses, and species described herein.

In some embodiments, a FASN inhibitor is selected from compounds shown in Table A, which lists the IUPAC names and the structures of certain exemplary compounds.

TABLE A

| NAME | Structure |
|---|---|
| (4-([1,1′-biphenyl]-4- carbonyl)piperazin-1-yl)(1- hydroxycyclopropyl)methanone | 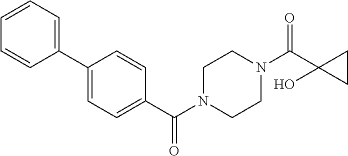 [SMILES: O=C(c1ccc(-c2ccccc2)cc1)N1CCN(C(=O)C2(O)CC2)CC1] |
| 1-(4-([1,1′-biphenyl]-4- carbonyl)piperazin-1-yl)-2-hydroxy- 2-methylpropan-1-one | 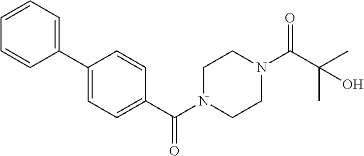 [SMILES: CC(C)(O)C(=O)N1CCN(C(=O)c2ccc(-c3ccccc3)cc2)CC1] |
| 1-(4-([1,1′-biphenyl]-4- carbonyl)piperazin-1-yl)-2- hydroxyethan-1-one | 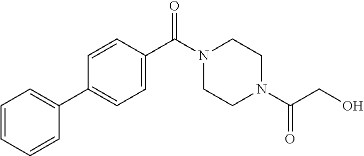 [SMILES: O=C(CO)N1CCN(C(=O)c2ccc(-c3ccccc3)cc2)CC1] |
| (4-([1,1′-biphenyl]-4- carbonyl)piperazin-1-yl)(1- hydroxycyclopentyl)methanone | 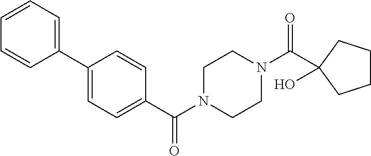 [SMILES: O=C(c1ccc(-c2ccccc2)cc1)N1CCN(C(=O)C2(O)CCCC2)CC1] |
| 1-(4-([1,1′-biphenyl]-4- carbonyl)piperazin-1-yl)-3,3,3- trifluoro-2-hydroxy-2- methylpropan-1-one | 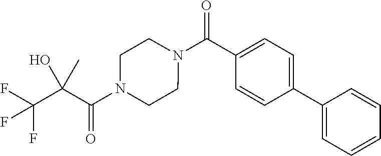 [SMILES: CC(O)(C(=O)N1CCN(C(=O)c2ccc(-c3ccccc3)cc2)CC1)C(F)(F)F] |
| (4-(1-hydroxycyclopropane-1- carbonyl)piperazin-1-yl)(3′-methyl- [1,1′-biphenyl]-4-yl)methanone | 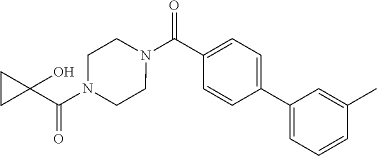 [SMILES: Cc1cccc(-c2ccc(C(=O)N3CCN(C(=O)C4(O)CC4)CC3)cc2)c1] |
| (4-(1-hydroxycyclopropane-1- carbonyl)piperazin-1-yl)(4′-methyl- [1,1′-biphenyl]-4-yl)methanone | 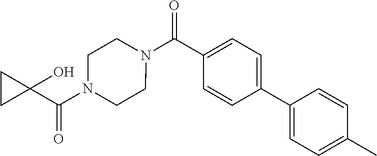 [SMILES: Cc1ccc(-c2ccc(C(=O)N3CCN(C(=O)C4(O)CC4)CC3)cc2)cc1] |
| (4-(2′-fluoro-3′-methoxy-[1,1′- biphenyl]-4-carbonyl)piperazin-1- yl)(1- hydroxycyclopropyl)methanone | 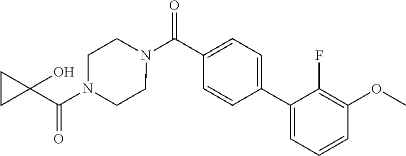 [SMILES: COc1cccc(-c2ccc(C(=O)N3CCN(C(=O)C4(O)CC4)CC3)cc2)c1F] |
| (4-(2′-fluoro-4′-methyl-[1,1′- biphenyl]-4-carbonyl)piperazin-1- yl)(1- hydroxycyclopropyl)methanone | 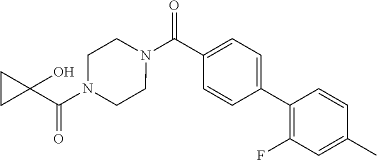 [SMILES: Cc1ccc(-c2ccc(C(=O)N3CCN(C(=O)C4(O)CC4)CC3)cc2)c(F)c1] |
| (4-(2′-fluoro-4′-methoxy-[1,1′- biphenyl]-4-carbonyl)piperazin-1- yl)(1- hydroxycyclopropyl)methanone | 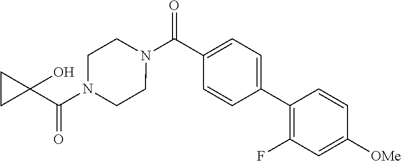 [SMILES: COc1ccc(-c2ccc(C(=O)N3CCN(C(=O)C4(O)CC4)CC3)cc2)c(F)c1] |
| (4-(2′-chloro-4′-fluoro-[1,1′- biphenyl]-4-carbonyl)piperazin-1- yl)(1- hydroxycyclopropyl)methanone | 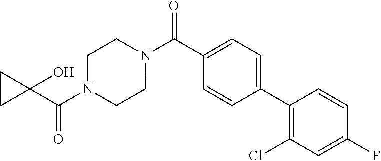 [SMILES: O=C(c1ccc(-c2ccc(F)cc2Cl)cc1)N1CCN(C(=O)C2(O)CC2)CC1] |
| (4-(3′-chloro-4′-fluoro-[1,1′- biphenyl]-4-carbonyl)piperazin-1- yl)(1- hydroxycyclopropyl)methanone | 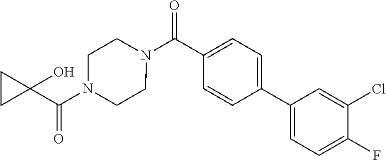 [SMILES: O=C(c1ccc(-c2ccc(F)c(Cl)c2)cc1)N1CCN(C(=O)C2(O)CC2)CC1] |
| (4-(4′-chloro-2′-fluoro-[1,1′- biphenyl]-4-carbonyl)piperazin-1- yl)(1- hydroxycyclopropyl)methanone | 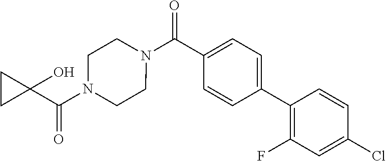 [SMILES: O=C(c1ccc(-c2ccc(Cl)cc2F)cc1)N1CCN(C(=O)C2(O)CC2)CC1] |
| (4-(2′,4′-dichloro-[1,1′-biphenyl]-4- carbonyl)piperazin-1-yl)(1- hydroxycyclopropyl)methanone | 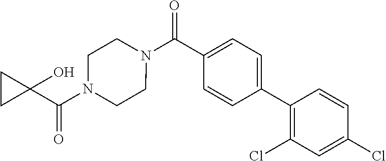 [SMILES: O=C(c1ccc(-c2ccc(Cl)cc2Cl)cc1)N1CCN(C(=O)C2(O)CC2)CC1] |
| (4-(5′-fluoro-2′-methyl-[1,1′- biphenyl]-4-carbonyl)piperazin-1- yl)(1- hydroxycyclopropyl)methanone | 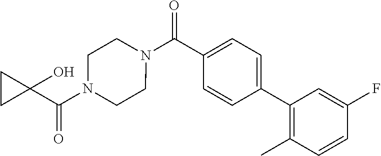 [SMILES: Cc1ccc(F)cc1-c1ccc(C(=O)N2CCN(C(=O)C3(O)CC3)CC2)cc1] |
| (4-(3′-fluoro-4′-methyl-[1,1′- biphenyl]-4-carbonyl)piperazin-1- yl)(1- hydroxycyclopropyl)methanone | 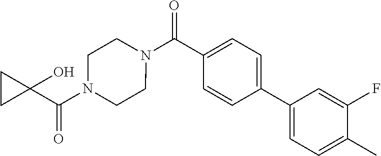 [SMILES: Cc1ccc(-c2ccc(C(=O)N3CCN(C(=O)C4(O)CC4)CC3)cc2)cc1F] |
| (4-(2′,3′-dichloro-[1,1′-biphenyl]-4- carbonyl)piperazin-1-yl)(1- hydroxycyclopropyl)methanone | 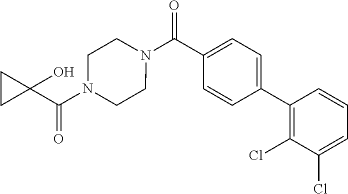 [SMILES: O=C(c1ccc(-c2cccc(Cl)c2Cl)cc1)N1CCN(C(=O)C2(O)CC2)CC1] |
| (4-(2′,5′-dichloro-[1,1′-biphenyl]-4- carbonyl)piperazin-1-yl)(1- hydroxycyclopropyl)methanone | 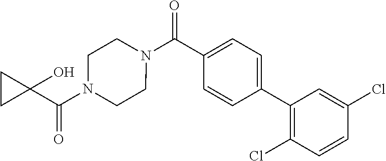 [SMILES: O=C(c1ccc(-c2cc(Cl)ccc2Cl)cc1)N1CCN(C(=O)C2(O)CC2)CC1] |
| (4-(4′-chloro-3′-fluoro-[1,1′- biphenyl]-4-carbonyl)piperazin-1- yl)(1- hydroxycyclopropyl)methanone | 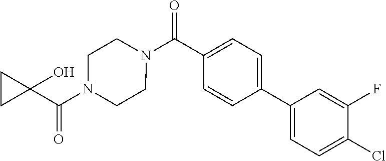 [SMILES: O=C(c1ccc(-c2ccc(Cl)c(F)c2)cc1)N1CCN(C(=O)C2(O)CC2)CC1] |
| (4-(2′,5′-difluoro-[1,1′-biphenyl]-4- carbonyl)piperazin-1-yl)(1- hydroxycyclopropyl)methanone | 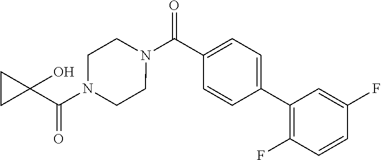 [SMILES: O=C(c1ccc(-c2cc(F)ccc2F)cc1)N1CCN(C(=O)C2(O)CC2)CC1] |
| (4-(3′,4′-dichloro-[1,1′-biphenyl]-4- carbonyl)piperazin-1-yl)(1- hydroxycyclopropyl)methanone | 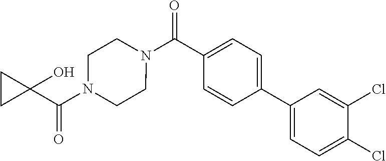 [SMILES: O=C(c1ccc(-c2ccc(Cl)c(Cl)c2)cc1)N1CCN(C(=O)C2(O)CC2)CC1] |
| (4-(2′,3′-difluoro-[1,1′-biphenyl]-4- carbonyl)piperazin-1-yl)(1- hydroxycyclopropyl)methanone | 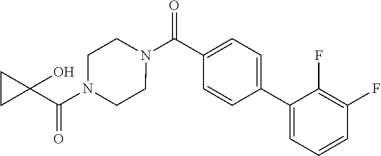 [SMILES: O=C(c1ccc(-c2cccc(F)c2F)cc1)N1CCN(C(=O)C2(O)CC2)CC1] |
| (4-(1-hydroxycyclopropane-1- carbonyl)piperazin-1-yl)(4-(1- methyl-1H-indol-5- yl)phenyl)methanone | 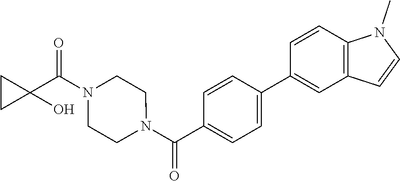 [SMILES: Cn1ccc2cc(-c3ccc(C(=O)N4CCN(C(=O)C5(O)CC5)CC4)cc3)ccc21] |
| (4-(1-hydroxycyclopropane-1- carbonyl)piperazin-1-yl)(2′- methoxy-4′-(1H-pyrazol-1-yl)-[1,1′- biphenyl]-4-yl)methanone | 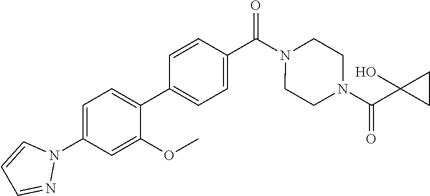 [SMILES: COc1cc(-n2cccn2)ccc1-c1ccc(C(=O)N2CCN(C(=O)C3(O)CC3)CC2)cc1] |
| (4-(2′-fluoro-5′-methyl-[1,1′- biphenyl]-4-carbonyl)piperazin-1- yl)(1- hydroxycyclopropyl)methanone | 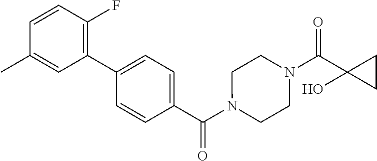 [SMILES: Cc1ccc(F)c(-c2ccc(C(=O)N3CCN(C(=O)C4(O)CC4)CC3)cc2)c1] |
| (4-(2′-chloro-5′-(trifluoromethyl)- [1,1′-biphenyl]-4- carbonyl)piperazin-1-yl)(1- hydroxycyclopropyl)methanone | 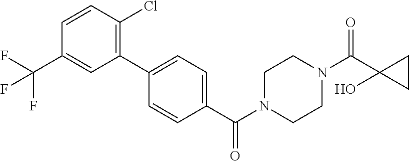 [SMILES: O=C(c1ccc(-c2cc(C(F)(F)F)ccc2Cl)cc1)N1CCN(C(=O)C2(O)CC2)CC1] |
| (4-(4′-chloro-2′-methoxy-[1,1′- biphenyl]-4-carbonyl)piperazin-1- yl)(1- hydroxycyclopropyl)methanone | 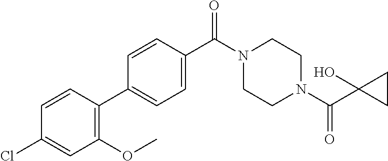 [SMILES: COc1cc(Cl)ccc1-c1ccc(C(=O)N2CCN(C(=O)C3(O)CC3)CC2)cc1] |
| (4-(3′-chloro-2′-methoxy-[1,1′- biphenyl]-4-carbonyl)piperazin-1- yl)(1- hydroxycyclopropyl)methanone | 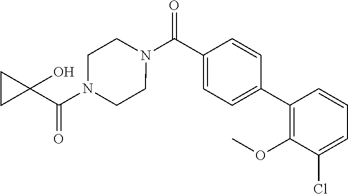 [SMILES: COc1c(Cl)cccc1-c1ccc(C(=O)N2CCN(C(=O)C3(O)CC3)CC2)cc1] |
| (4-(4′,5′-difluoro-2′-methoxy-[1,1′- biphenyl]-4-carbonyl)piperazin-1- yl)(1- hydroxycyclopropyl)methanone | 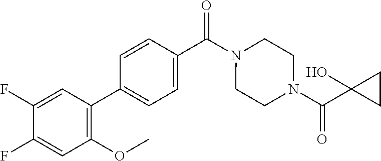 [SMILES: COc1cc(F)c(F)cc1-c1ccc(C(=O)N2CCN(C(=O)C3(O)CC3)CC2)cc1] |
| (4-(2′-fluoro-3′-(trifluoromethyl)- [1,1′-biphenyl]-4- carbonyl)piperazin-1-yl)(1- hydroxycyclopropyl)methanone | 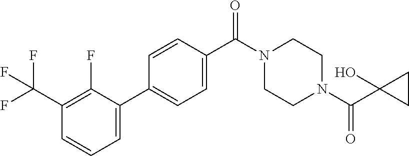 [SMILES: O=C(c1ccc(-c2cccc(C(F)(F)F)c2F)cc1)N1CCN(C(=O)C2(O)CC2)CC1] |
| (4-(4′-chloro-2′-(trifluoromethyl)- [1,1′-biphenyl]-4- carbonyl)piperazin-1-yl)(1- hydroxycyclopropyl)methanone | 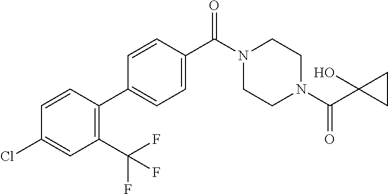 [SMILES: O=C(c1ccc(-c2ccc(Cl)cc2C(F)(F)F)cc1)N1CCN(C(=O)C2(O)CC2)CC1] |
| (4-(2′-chloro-4′-methoxy-[1,1′- biphenyl]-4-carbonyl)piperazin-1- yl)(1- hydroxycyclopropyl)methanone | 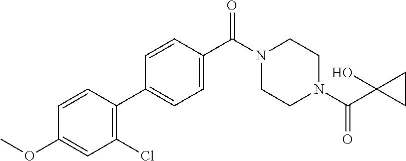 [SMILES: COc1ccc(-c2ccc(C(=O)N3CCN(C(=O)C4(O)CC4)CC3)cc2)c(Cl)c1] |
| (4-(5′-chloro-2′-isopropoxy-[1,1′- biphenyl]-4-carbonyl)piperazin-1- yl)(1- hydroxycyclopropyl)methanone | 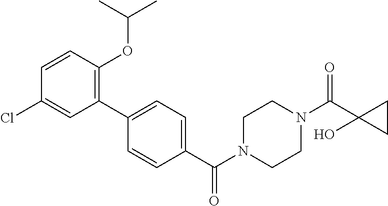 [SMILES: CC(C)Oc1ccc(Cl)cc1-c1ccc(C(=O)N2CCN(C(=O)C3(O)CC3)CC2)cc1] |
| (4-(3′-fluoro-5′-(trifluoromethyl)- [1,1′-biphenyl]-4- carbonyl)piperazin-1-yl)(1- hydroxycyclopropyl)methanone | 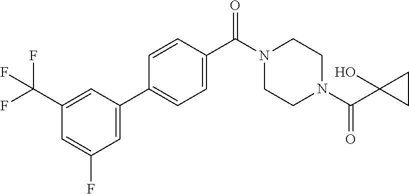 [SMILES: O=C(c1ccc(-c2cc(F)cc(C(F)(F)F)c2)cc1)N1CCN(C(=O)C2(O)CC2)CC1] |
| (4-(1-hydroxycyclopropane-1- carbonyl)piperazin-1-yl)(2′- methoxy-5′-(trifluoromethoxy)- [1,1′-biphenyl]-4-yl)methanone | 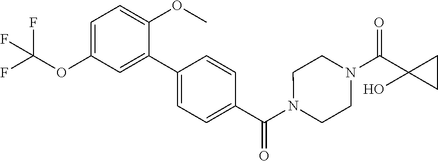 [SMILES: COc1ccc(OC(F)(F)F)cc1-c1ccc(C(=O)N2CCN(C(=O)C3(O)CC3)CC2)cc1] |
| (4-(3′-fluoro-2′-methoxy-[1,1′- biphenyl]-4-carbonyl)piperazin-1- yl)(1- hydroxycyclopropyl)methanone | 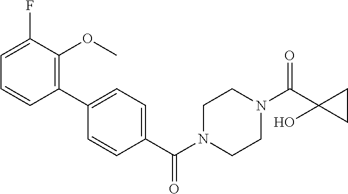 [SMILES: COc1c(F)cccc1-c1ccc(C(=O)N2CCN(C(=O)C3(O)CC3)CC2)cc1] |
| (4-(1-hydroxycyclopropane-1- carbonyl)piperazin-1-yl)(4-(3- methoxypyridin-4- yl)phenyl)methanone | 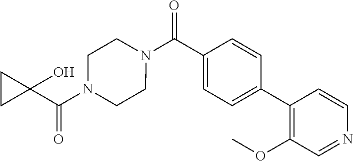 [SMILES: COc1cnccc1-c1ccc(C(=O)N2CCN(C(=O)C3(O)CC3)CC2)cc1] |
| (4-(1-hydroxycyclopropane-1- carbonyl)piperazin-1-yl)(4- (quinolin-3-yl)phenyl)methanone | 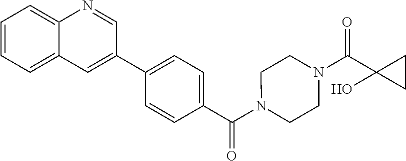 [SMILES: O=C(c1ccc(-c2cnc3ccccc3c2)cc1)N1CCN(C(=O)C2(O)CC2)CC1] |
| (4-(1-hydroxycyclopropane-1- carbonyl)piperazin-1-yl)(4- (isoquinolin-6- yl)phenyl)methanone | 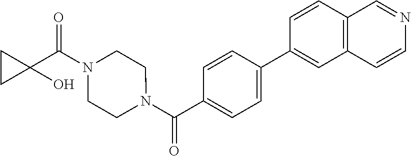 [SMILES: O=C(c1ccc(-c2ccc3cnccc3c2)cc1)N1CCN(C(=O)C2(O)CC2)CC1] |
| (4-(1-hydroxycyclopropane-1- carbonyl)piperazin-1-yl)(4- (imidazo[1,2-a]pyridin-6- yl)phenyl)methanone | 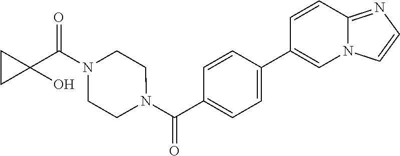 [SMILES: O=C(c1ccc(-c2ccc3nccn3c2)cc1)N1CCN(C(=O)C2(O)CC2)CC1] |
| (4-(4-(benzo[d]thiazol-5- yl)benzoyl)piperazin-1-yl)(1- hydroxycyclopropyl)methanone | 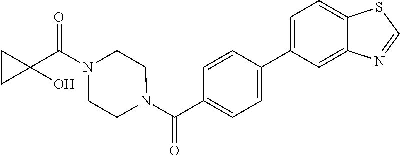 [SMILES: O=C(c1ccc(-c2ccc3scnc3c2)cc1)N1CCN(C(=O)C2(O)CC2)CC1] |
| (4-(1-hydroxycyclopropane-1- carbonyl)piperazin-1-yl)(4-(6- (trifluoromethyl)pyridin-3- yl)phenyl)methanone | 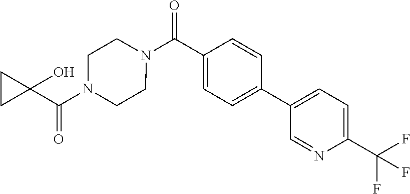 [SMILES: O=C(c1ccc(-c2ccc(C(F)(F)F)nc2)cc1)N1CCN(C(=O)C2(O)CC2)CC1] |
| (4-(5′-chloro-2′-fluoro-[1,1′]- biphenyl]-4-carbonyl)piperazin-1- yl)(1- hydroxycyclopropyl)methanone | 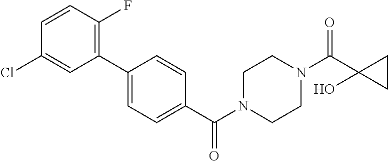 [SMILES: O=C(c1ccc(-c2cc(Cl)ccc2F)cc1)N1CCN(C(=O)C2(O)CC2)CC1] |
| (4-(1-hydroxycyclopropane-1- carbonyl)piperazin-1-yl)(4-(6- methylpyridin-3- yl)phenyl)methanone | 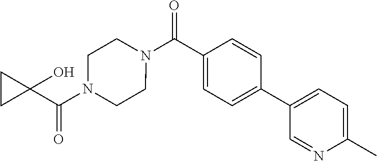 [SMILES: Cc1ccc(-c2ccc(C(=O)N3CCN(C(=O)C4(O)CC4)CC3)cc2)cn1] |
| (4-(4-(2,6-dimethoxypyridin-3- yl)benzoyl)piperazin-1-yl)(1- hydroxycyclopropyl)methanone | 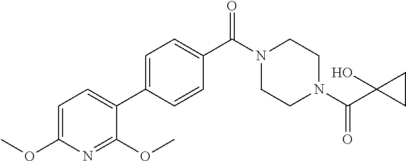 [SMILES: COc1ccc(-c2ccc(C(=O)N3CCN(C(=O)C4(O)CC4)CC3)cc2)c(OC)n1] |
| (4-(4-(5-fluoropyridin-3- yl)benzoyl)piperazin-1-yl)(1- hydroxycyclopropyl)methanone | 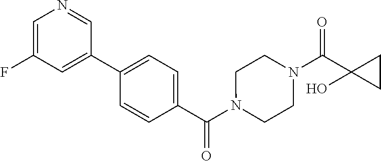 [SMILES: O=C(c1ccc(-c2cncc(F)c2)cc1)N1CCN(C(=O)C2(O)CC2)CC1] |
| (4-(1-hydroxycyclopropane-1- carbonyl)piperazin-1-yl)(4-(1- methyl-1H-indol-2- yl)phenyl)methanone | 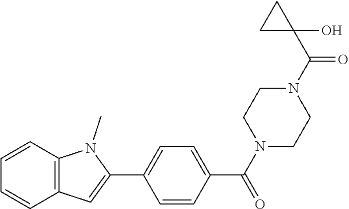 [SMILES: Cn1c(-c2ccc(C(=O)N3CCN(C(=O)C4(O)CC4)CC3)cc2)cc2ccccc21] |
| (4-(2′-chloro-5′-fluoro-[1,1′- biphenyl]-4-carbonyl)piperazin-1- yl)(1- hydroxycyclopropyl)methanone | 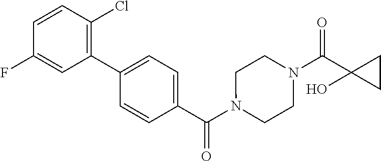 [SMILES: O=C(c1ccc(-c2cc(F)ccc2Cl)cc1)N1CCN(C(=O)C2(O)CC2)CC1] |
| (4-(2′-fluoro-5′-methoxy-[1,1′- biphenyl]-4-carbonyl)piperazin-1- yl)(1- hydroxycyclopropyl)methanone | 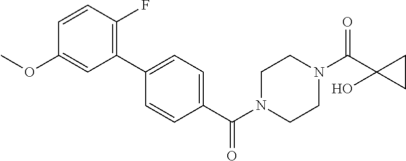 [SMILES: COc1ccc(F)c(-c2ccc(C(=O)N3CCN(C(=O)C4(O)CC4)CC3)cc2)c1] |
| (4-(2′-fluoro-3′-methyl-[1,1′- biphenyl]-4-carbonyl)piperazin-1- yl)(1- hydroxycyclopropyl)methanone | 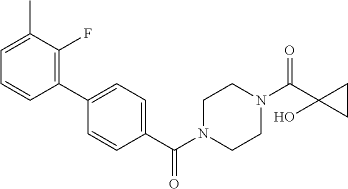 [SMILES: Cc1cccc(-c2ccc(C(=O)N3CCN(C(=O)C4(O)CC4)CC3)cc2)c1F] |
| (4-(4′-fluoro-2′-(trifluoromethyl)- [1,1′-biphenyl]-4- carbonyl)piperazin-1-yl)(1- hydroxycyclopropyl)methanone | 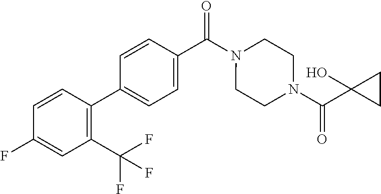 [SMILES: O=C(c1ccc(-c2ccc(F)cc2C(F)(F)F)cc1)N1CCN(C(=O)C2(O)CC2)CC1] |
| (4-(2′-chloro-5′-(hydroxymethyl)- [1,1′-biphenyl]-4- carbonyl)piperazin-1-yl)(1- hydroxycyclopropyl)methanone | 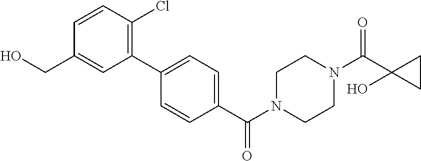 [SMILES: O=C(c1ccc(-c2cc(CO)ccc2Cl)cc1)N1CCN(C(=O)C2(O)CC2)CC1] |
| (4-(1-hydroxycyclopropane-1- carbonyl)piperazin-1-yl)(4-(2- methyl-2H-indazol-6- yl)phenyl)methanone | 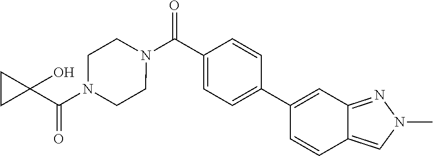 [SMILES: Cn1cc2ccc(-c3ccc(C(=O)N4CCN(C(=O)C5(O)CC5)CC4)cc3)cc2n1] |
| (4-(1-hydroxycyclopropane-1- carbonyl)piperazin-1-yl)(4-(1- methyl-1H-indazol-7- yl)phenyl)methanone | 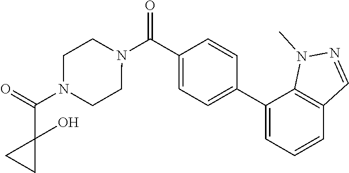 [SMILES: Cn1ncc2cccc(-c3ccc(C(=O)N4CCN(C(=O)C5(O)CC5)CC4)cc3)c21] |
| (4-(4-(1H-indol-4- yl)benzoyl)piperazin-1-yl)(1- hydroxycyclopropyl)methanone | 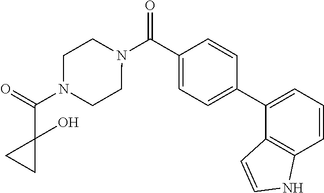 [SMILES: O=C(c1ccc(-c2cccc3[nH]ccc23)cc1)N1CCN(C(=O)C2(O)CC2)CC1] |
| N-cyclopropyl-4′-(4-(1- hydroxycyclopropane-1- carbonyl)piperazine-1-carbonyl)- [1,1′-biphenyl]-3-carboxamide | 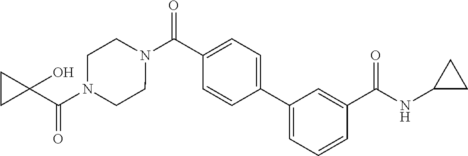 [SMILES: O=C(NC1CC1)c1cccc(-c2ccc(C(=O)N3CCN(C(=O)C4(O)CC4)CC3)cc2)c1] |
| N-(4′-(4-(1-hydroxycyclopropane-1- carbonyl)piperazine-1-carbonyl)- [1,1′-biphenyl]-3- yl)ethanesulfonamide | 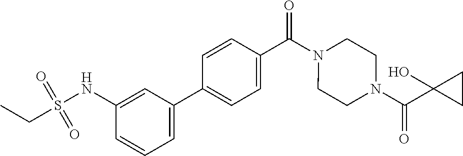 [SMILES: CCS(=O)(=O)Nc1cccc(-c2ccc(C(=O)N3CCN(C(=O)C4(O)CC4)CC3)cc2)c1] |
| 6-fluoro-4′-(4-(1- hydroxycyclopropane-1- carbonyl)piperazine-1-carbonyl)-N- methyl-[1,1′-biphenyl]-3- carboxamide | 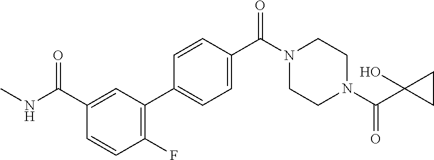 [SMILES: CNC(=O)c1ccc(F)c(-c2ccc(C(=O)N3CCN(C(=O)C4(O)CC4)CC3)cc2)c1] |
| 6-fluoro-4′-(4-(1- hydroxycyclopropane-1- carbonyl)piperazine-1-carbonyl)-N- isopropyl-[1,1′-biphenyl]-3- carboxamide | 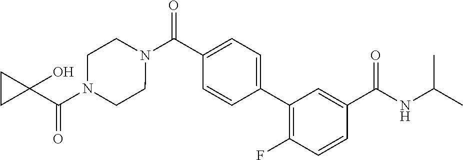 [SMILES: CC(C)NC(=O)c1ccc(F)c(-c2ccc(C(=O)N3CCN(C(=O)C4(O)CC4)CC3)cc2)c1] |
| (4-(2′,4′-dichloro-3′-methoxy-[1,1′- biphenyl]-4-carbonyl)piperazin-1- yl)(1- hydroxycyclopropyl)methanone | 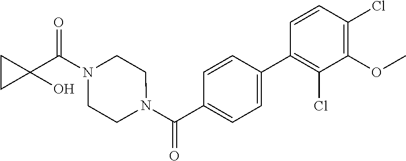 [SMILES: COc1c(Cl)ccc(-c2ccc(C(=O)N3CCN(C(=O)C4(O)CC4)CC3)cc2)c1Cl] |
| (4-(2′-fluoro-3′-isopropoxy-[1,1′- biphenyl]-4-carbonyl)piperazin-1- yl)(1- hydroxycyclopropyl)methanone | 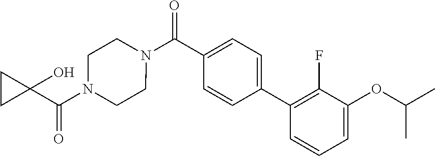 [SMILES: CC(C)Oc1cccc(-c2ccc(C(=O)N3CCN(C(=O)C4(O)CC4)CC3)cc2)c1F] |
| (4-(2′-fluoro-3′-(trifluoromethoxy)- 1′,2′,3′,4′,5′,6′-hexahydro-[1,1′- biphenyl]-4-carbonyl)piperazin-1- yl)(1- hydroxycyclopropyl)methanone | 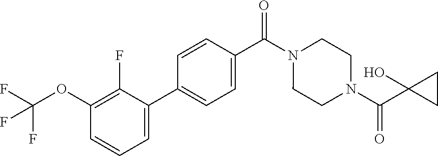 [SMILES: O=C(c1ccc(-c2cccc(OC(F)(F)F)c2F)cc1)N1CCN(C(=O)C2(O)CC2)CC1] |
| (4-(3′-(cyclopropylmethoxy)-(1,1′- biphenyl]-4-carbonyl)piperazin-1- yl)(1- hydroxycyclopropyl)methanone | 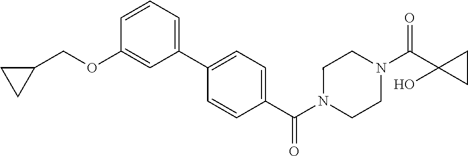 [SMILES: O=C(c1ccc(-c2cccc(OCC3CC3)c2)cc1)N1CCN(C(=O)C2(O)CC2)CC1] |
| 4-fluoro-4′-(4-(1- hydroxycyclopropane-1- carbonyl)piperazine-1-carbonyl)- [1,1′-biphenyl]-2-carbonitrile | 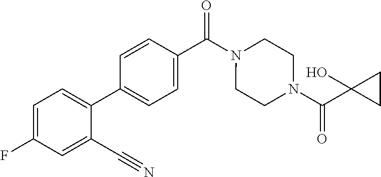 [SMILES: N#Cc1cc(F)ccc1-c1ccc(C(=O)N2CCN(C(=O)C3(O)CC3)CC2)cc1] |
| (4-(1-hydroxycyclopropane-1- carbonyl)piperazin-1-yl)(4-(1- methyl-1H-indazol-6- yl)phenyl)methanone | 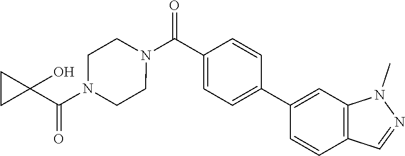 [SMILES: Cn1ncc2ccc(-c3ccc(C(=O)N4CCN(C(=O)C5(O)CC5)CC4)cc3)cc21] |
| (4-(1-hydroxycyclopropane-1- carbonyl)piperazin-1-yl)(4-(1- methyl-1H-indazol-5- yl)phenyl)methanone | 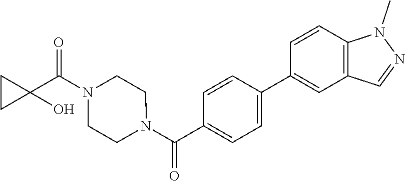 [SMILES: Cn1ncc2cc(-c3ccc(C(=O)N4CCN(C(=O)C5(O)CC5)CC4)cc3)ccc21] |
| (4-(1-hydroxycyclopropane-1- carbonyl)piperazin-1-yl)(6-(1- methyl-1H-indol-5-yl)pyridin-3- yl)methanone | 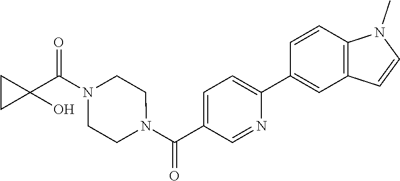 [SMILES: Cn1ccc2cc(-c3ccc(C(=O)N4CCN(C(=O)C5(O)CC5)CC4)cn3)ccc21] |
| (4-(1-hydroxycyclopropane-1- carbonyl)piperazin-1-yl)(5-(1- methyl-1H-indol-5-yl)pyridin-2- yl)methanone | 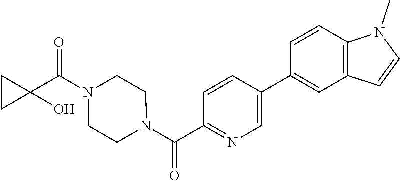 [SMILES: Cn1ccc2cc(-c3ccc(C(=O)N4CCN(C(=O)C5(O)CC5)CC4)nc3)ccc21] |
| (4-(2-fluoro-4-(1-methyl-1H-indol- 5-yl)benzoyl)piperazin-1-yl)(1- hydroxycyclopropyl)methanone | 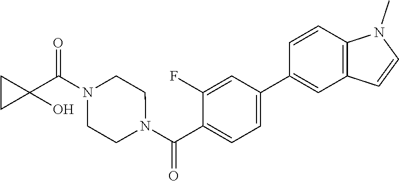 [SMILES: Cn1ccc2cc(-c3ccc(C(=O)N4CCN(C(=O)C5(O)CC5)CC4)c(F)c3)ccc21] |
| (4-(2-fluoro-4-(quinolin-6- yl)benzoyl)piperazin-1-yl)(1- hydroxycyclopropyl)methanone | 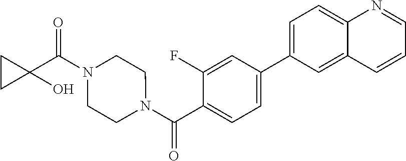 [SMILES: O=C(c1ccc(-c2ccc3ncccc3c2)cc1F)N1CCN(C(=O)C2(O)CC2)CC1] |
| 1-(4-([1,1′-biphenyl]-4- carbonyl)piperazin-1-yl)-2- methoxy-2-methylpropan-1-one | 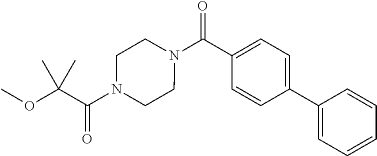 [SMILES: COC(C)(C)C(=O)N1CCN(C(=O)c2ccc(-c3ccccc3)cc2)CC1] |
| (S)-1-(4-([1,1′-biphenyl]-4- carbonyl)piperazin-1-yl)-2- hydroxypropan-1-one | 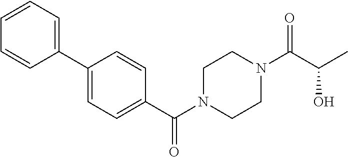 [SMILES: C[C@H](O)C(=O)N1CCN(C(=O)c2ccc(-c3ccccc3)cc2)CC1] |
| (4-(4-(3,4-dihydroquinolin-3- yl)benzoyl)piperazin-1-yl)(1- hydroxycyclobutyl)methanone | 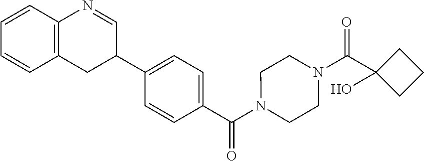 [SMILES: O=C(c1ccc(C2C=Nc3ccccc3C2)cc1)N1CCN(C(=O)C2(O)CCC2)CC1] |
| (4-(2-chloro-4-(quinolin-6- yl)benzoyl)piperazin-1-yl)(1- hydroxycyclopropyl)methanone | 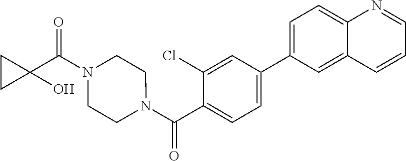 [SMILES: O=C(c1ccc(-c2ccc3ncccc3c2)cc1Cl)N1CCN(C(=O)C2(O)CC2)CC1] |
| (4-(2-chloro-4-(1-methyl-1H-indol- 5-yl)benzoyl)piperazin-1-yl)(1- hydroxycyclopropyl)methanone | 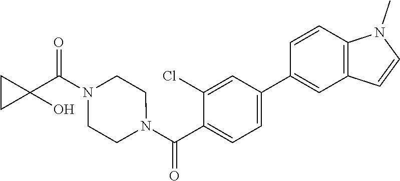 [SMILES: Cn1ccc2cc(-c3ccc(C(=O)N4CCN(C(=O)C5(O)CC5)CC4)c(Cl)c3)ccc21] |
| (4-(1-hydroxycyclopropane-1- carbonyl)piperazin-1-yl)(6- (quinolin-6-yl)pyridin-3- yl)methanone | 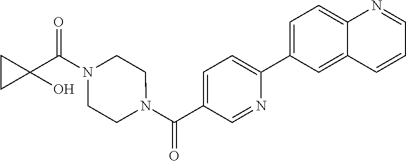 [SMILES: O=C(c1ccc(-c2ccc3ncccc3c2)nc1)N1CCN(C(=O)C2(O)CC2)CC1] |
| (4-(1-hydroxycyclopropane-1- carbonyl)piperazin-1-yl)(4-methyl- 2-(1-methyl-1H-indol-5-yl)thiazol-5- yl)methanone | 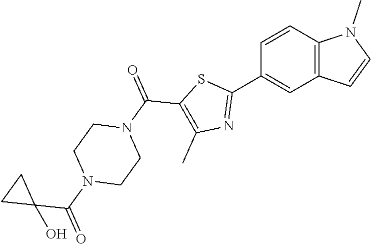 [SMILES: Cc1nc(-c2ccc3c(ccn3C)c2)sc1C(=O)N1CCN(C(=O)C2(O)CC2)CC1] |
| (4-(1-hydroxycyclopropane-1- carbonyl)piperazin-1-yl)(4-methyl- 2-(quinolin-3-yl)thiazol-5- yl)methanone | 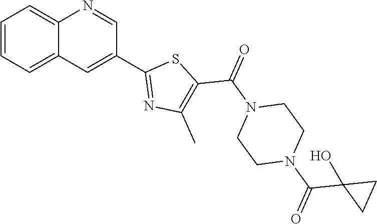 [SMILES: Cc1nc(-c2cnc3ccccc3c2)sc1C(=O)N1CCN(C(=O)C2(O)CC2)CC1] |
| (2-(benzo[d]thiazol-5-yl)-4- methylthiazol-5-yl)(4-(1- hydroxycyclopropane-1- carbonyl)piperazin-1-yl)methanone | 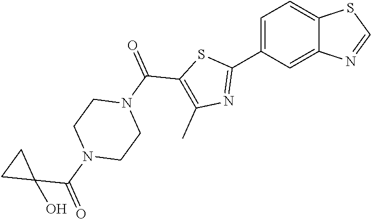 [SMILES: Cc1nc(-c2ccc3scnc3c2)sc1C(=O)N1CCN(C(=O)C2(O)CC2)CC1] |
| (4-(3-fluoro-4-(quinolin-3- yl)benzoyl)piperazin-1-yl)(1- hydroxycyclopropyl)methanone | 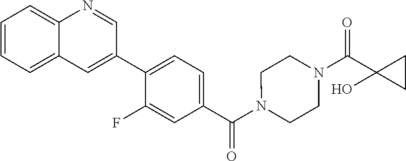 [SMILES: O=C(c1ccc(-c2cnc3ccccc3c2)c(F)c1)N1CCN(C(=O)C2(O)CC2)CC1] |
| (4-(3-fluoro-4-(quinolin-6- yl)benzoyl)piperazin-1-yl)(1- hydroxycyclopropyl)methanone | 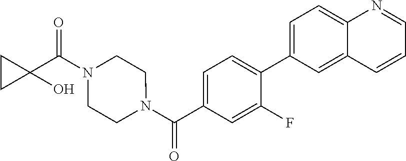 [SMILES: O=C(c1ccc(-c2ccc3ncccc3c2)c(F)c1)N1CCN(C(=O)C2(O)CC2)CC1] |
| (4-(4-(benzo[d]thiazol-5-yl)-3- fluorobenzoyl)piperazin-1-yl)(1- hydroxycyclopropyl)methanone | 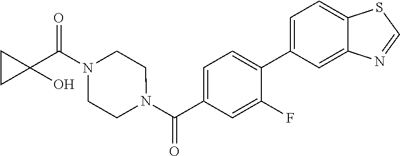 [SMILES: O=C(c1ccc(-c2ccc3scnc3c2)c(F)c1)N1CCN(C(=O)C2(O)CC2)CC1] |
| (4-(1-hydroxycyclopropane-1- carbonyl)piperazin-1-yl)(3-methyl- 4-(1-methyl-1H-indol-5- yl)phenyl)methanone | 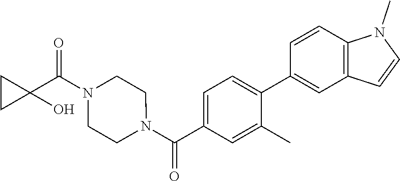 [SMILES: Cc1cc(C(=O)N2CCN(C(=O)C3(O)CC3)CC2)ccc1-c1ccc2c(ccn2C)c1] |
| (4-(1-hydroxycyclopropane-1- carbonyl)piperazin-1-yl)(3-methyl- 4-(quinolin-3-yl)phenyl)methanone | 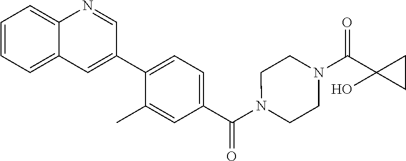 [SMILES: Cc1cc(C(=O)N2CCN(C(=O)C3(O)CC3)CC2)ccc1-c1cnc2ccccc2c1] |
| (4-(1-hydroxycyclopropane-1- carbonyl)piperazin-1-yl)(3-methyl- 4-(quinolin-6-yl)phenyl)methanone | 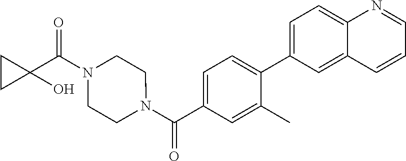 [SMILES: Cc1cc(C(=O)N2CCN(C(=O)C3(O)CC3)CC2)ccc1-c1ccc2ncccc2c1] |
| (4-(4-(benzo[d]thiazol-5-yl)-3- methylbenzoyl)piperazin-1-yl)(1- hydroxycyclopropyl)methanone | 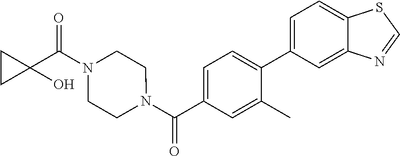 [SMILES: Cc1cc(C(=O)N2CCN(C(=O)C3(O)CC3)CC2)ccc1-c1ccc2scnc2c1] |
| (4-(2-chloro-4-(quinolin-3- yl)benzoyl)piperazin-1-yl)(1- hydroxycyclopropyl)methanone | 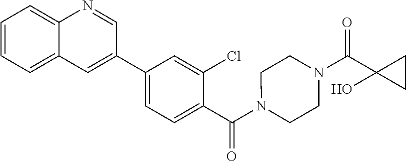 [SMILES: O=C(c1ccc(-c2cnc3ccccc3c2)cc1Cl)N1CCN(C(=O)C2(O)CC2)CC1] |
| (4-(4-(benzo[d]thiazol-5-yl)-2- chlorobenzoyl)piperazin-1-yl)(1- hydroxycyclopropyl)methanone | 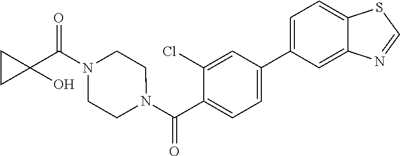 [SMILES: O=C(c1ccc(-c2ccc3scnc3c2)cc1Cl)N1CCN(C(=O)C2(O)CC2)CC1] |
| (4-(3-chloro-4-(1-methyl-1H-indol- 5-yl)benzoyl)piperazin-1-yl)(1- hydroxycyclopropyl)methanone | 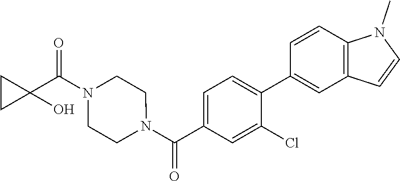 [SMILES: Cn1ccc2cc(-c3ccc(C(=O)N4CCN(C(=O)C5(O)CC5)CC4)cc3Cl)ccc21] |
| (4-(3-chloro-4-(quinolin-3- yl)benzoyl)piperazin-1-yl)(1- hydroxycyclopropyl)methanone | 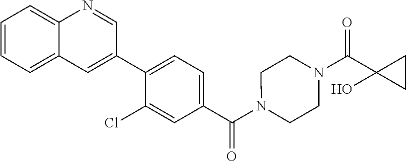 [SMILES: O=C(c1ccc(-c2cnc3ccccc3c2)c(Cl)c1)N1CCN(C(=O)C2(O)CC2)CC1] |
| (4-(3-chloro-4-(quinolin-6- yl)benzoyl)piperazin-1-yl)(1- hydroxycyclopropyl)methanone | 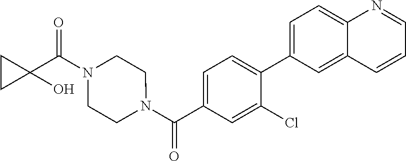 [SMILES: O=C(c1ccc(-c2ccc3ncccc3c2)c(Cl)c1)N1CCN(C(=O)C2(O)CC2)CC1] |
| (4-(4-(benzo[d]thiazol-5-yl)-3- chlorobenzoyl)piperazin-1-yl)(1- hydroxycyclopropyl)methanone | 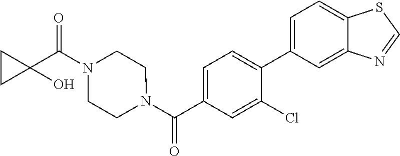 [SMILES: O=C(c1ccc(-c2ccc3scnc3c2)c(Cl)c1)N1CCN(C(=O)C2(O)CC2)CC1] |
| (4-(1-hydroxycyclopropane-1- carbonyl)piperazin-1-yl)(4-(1- methyl-1H-indol-5-yl)-2- (trifluoromethyl)phenyl)methanone | 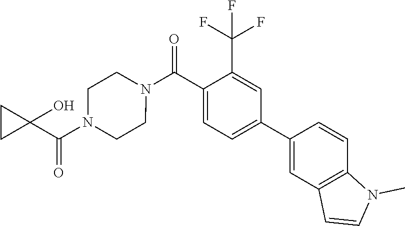 [SMILES: Cn1ccc2cc(-c3ccc(C(=O)N4CCN(C(=O)C5(O)CC5)CC4)c(C(F)(F)F)c3)ccc21] |
| (4-(1-hydroxycyclopropane-1- carbonyl)piperazin-1-yl)(4- (quinolin-6-yl)-2- (trifluoromethyl)phenyl)methanone | 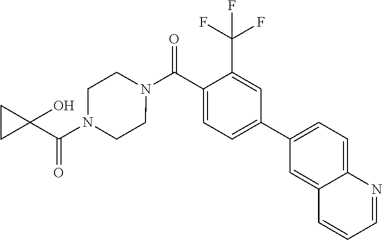 [SMILES: O=C(c1ccc(-c2ccc3ncccc3c2)cc1C(F)(F)F)N1CCN(C(=O)C2(O)CC2)CC1] |
| (4-(1-hydroxycyclopropane-1- carbonyl)piperazin-1-yl)(3- methoxy-4-(quinolin-6- yl)phenyl)methanone | 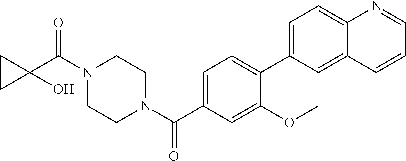 [SMILES: COc1cc(C(=O)N2CCN(C(=O)C3(O)CC3)CC2)ccc1-c1ccc2ncccc2c1] |
| (4-(1-hydroxycyclopropane-1- carbonyl)piperazin-1-yl)(2- methoxy-4-(quinolin-3- yl)phenyl)methanone | 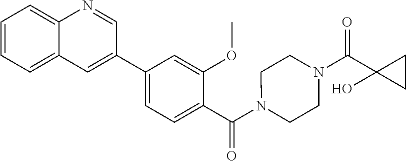 [SMILES: COc1cc(-c2cnc3ccccc3c2)ccc1C(=O)N1CCN(C(=O)C2(O)CC2)CC1] |
| (4-(1-hydroxycyclopropane-1- carbonyl)piperazin-1-yl)(2- methoxy-4-(quinolin-6- yl)phenyl)methanone | 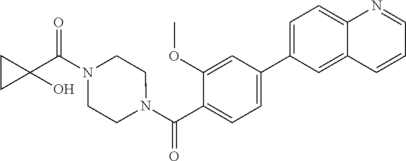 [SMILES: COc1cc(-c2ccc3ncccc3c2)ccc1C(=O)N1CCN(C(=O)C2(O)CC2)CC1] |
| (4-(4-(benzo[d]thiazol-5-yl)-2- methoxybenzoyl)piperazin-1-yl)(1- hydroxycyclopropyl)methanone | 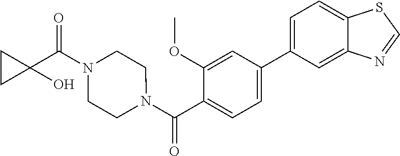 [SMILES: COc1cc(-c2ccc3scnc3c2)ccc1C(=O)N1CCN(C(=O)C2(O)CC2)CC1] |
| (4-(1-hydroxycyclopropane-1- carbonyl)piperazin-1-yl)(5- (quinolin-6-yl)pyrimidin-2- yl)methanone | 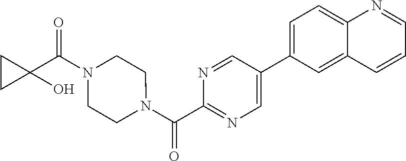 [SMILES: O=C(c1ncc(-c2ccc3ncccc3c2)cn1)N1CCN(C(=O)C2(O)CC2)CC1] |
| (4-(1-hydroxycyclopropane-1- carbonyl)piperazin-1-yl)(5-(1- methyl-1H-indol-5-yl)pyrazin-2- yl)methanone | 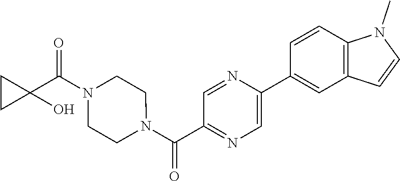 [SMILES: Cn1ccc2cc(-c3cnc(C(=O)N4CCN(C(=O)C5(O)CC5)CC4)cn3)ccc21] |
| (4-(1-hydroxycyclopropane-1- carbonyl)piperazin-1-yl)(5- (quinolin-3-yl)pyrazin-2- yl)methanone | 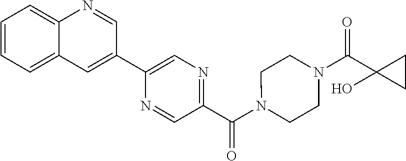 [SMILES: O=C(c1cnc(-c2cnc3ccccc3c2)cn1)N1CCN(C(=O)C2(O)CC2)CC1] |
| (4-(1-hydroxycyclopropane-1- carbonyl)piperazin-1-yl)(5- (quinolin-6-yl)pyrazin-2- yl)methanone | 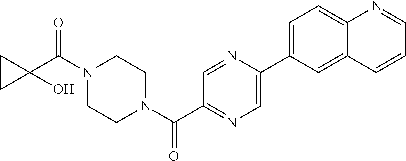 [SMILES: O=C(c1cnc(-c2ccc3ncccc3c2)cn1)N1CCN(C(=O)C2(O)CC2)CC1] |
| (4-([1,1′-biphenyl]-4- carbonyl)piperazin-1-yl)(1- hydroxycyclobutyl)methanone | 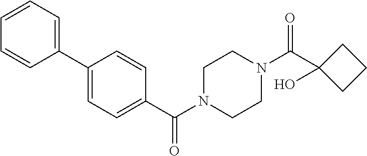 [SMILES: O=C(c1ccc(-c2ccccc2)cc1)N1CCN(C(=O)C2(O)CCC2)CC1] |
| (4-(1-hydroxycyclobutane-1- carbonyl)piperazin-1-yl)(4- (isoquinolin-6- yl)phenyl)methanone | 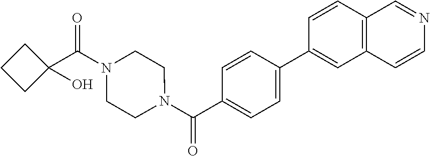 [SMILES: O=C(c1ccc(-c2ccc3cnccc3c2)cc1)N1CCN(C(=O)C2(O)CCC2)CC1] |
| ((2R,5S)-4-(1-hydroxycyclopropane- 1-carbonyl)-2,5-dimethylpiperazin- 1-yl)(4-(isoquinolin-6- yl)phenyl)methanone | 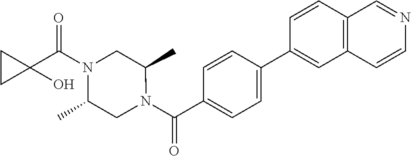 [SMILES: C[C@@H]1CN(C(=O)C2(O)CC2)[C@@H](C)CN1C(=O)c1ccc(-c2ccc3cnccc3c2)cc1] |
| (4-(4-(1H-indol-5- yl)benzoyl)piperazin-1-yl)(1- hydroxycyclopropyl)methanone | 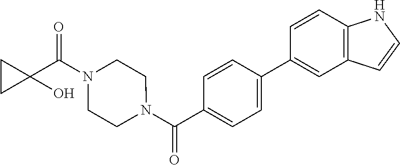 [SMILES: O=C(c1ccc(-c2ccc3[nH]ccc3c2)cc1)N1CCN(C(=O)C2(O)CC2)CC1] |
| oxetan-2-yl(4-(4-(quinolin-3- yl)benzoyl)piperazin-1- yl)methanone | 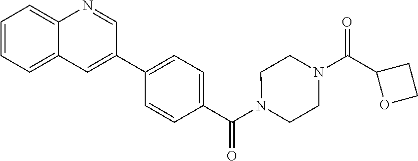 [SMILES: O=C(c1ccc(-c2cnc3ccccc3c2)cc1)N1CCN(C(=O)C2CCO2)CC1] |
| (4-(4-(1H-indol-5- yl)benzoyl)piperazin-1-yl)(oxetan-2- yl)methanone | 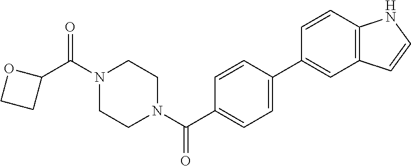 [SMILES: O=C(c1ccc(-c2ccc3[nH]ccc3c2)cc1)N1CCN(C(=O)C2CCO2)CC1] |
| (4-(2-fluoro-4-(quinolin-3- yl)benzoyl)piperazin-1-yl)(1- hydroxycyclopropyl)methanone | 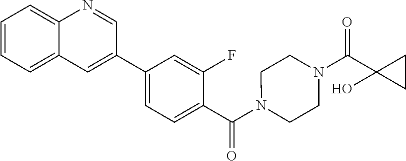 [SMILES: O=C(c1ccc(-c2cnc3ccccc3c2)cc1F)N1CCN(C(=O)C2(O)CC2)CC1] |
| (4-(4-(isoquinolin-6- yl)benzoyl)piperazin-1-yl)(oxetan-2- yl)methanone | 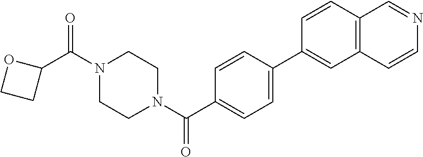 [SMILES: O=C(c1ccc(-c2ccc3cnccc3c2)cc1)N1CCN(C(=O)C2CCO2)CC1] |
| (4-(4-(1,5-naphthyridin-3- yl)benzoyl)piperazin-1-yl)(1- hydroxycyclopropyl)methanone | 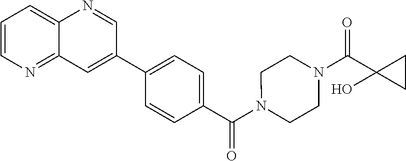 [SMILES: O=C(c1ccc(-c2cnc3cccnc3c2)cc1)N1CCN(C(=O)C2(O)CC2)CC1] |
| (4-(4-(benzofuran-5- yl)benzoyl)piperazin-1-yl)(1- hydroxycyclopropyl)methanone | 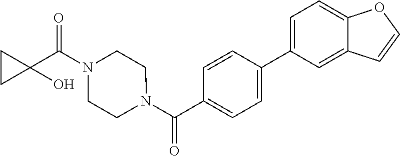 [SMILES: O=C(c1ccc(-c2ccc3occc3c2)cc1)N1CCN(C(=O)C2(O)CC2)CC1] |
| (4-(1-hydroxycyclopropane-1- carbonyl)piperazin-1-yl)(4- phenoxyphenyl)methanone | 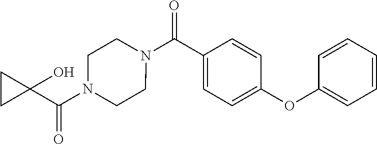 [SMILES: O=C(c1ccc(Oc2ccccc2)cc1)N1CCN(C(=O)C2(O)CC2)CC1] |
| (4-(2-fluoro-4-(7-fluoro-1H-indol-5- yl)benzoyl)piperazin-1-yl)(1- hydroxycyclopropyl)methanone | 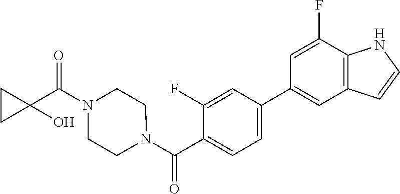 [SMILES: O=C(c1ccc(-c2cc(F)c3[nH]ccc3c2)cc1F)N1CCN(C(=O)C2(O)CC2)CC1] |
| (4-(3-chloro-4-(1-(trifluoromethyl)- 1H-indol-5-yl)benzoyl)piperazin-1- yl)(1- hydroxycyclopropyl)methanone | 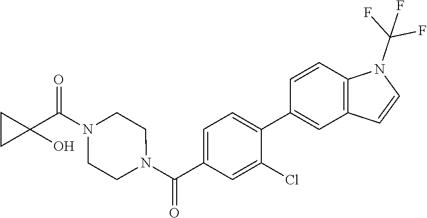 [SMILES: O=C(c1ccc(-c2ccc3c(ccn3C(F)(F)F)c2)c(Cl)c1)N1CCN(C(=O)C2(O)CC2)CC1] |
| (4-(4-(benzo[d]oxazol-5- yl)benzoyl)piperazin-1-yl)(1- hydroxycyclopropyl)methanone | 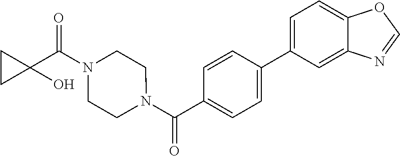 [SMILES: O=C(c1ccc(-c2ccc3ocnc3c2)cc1)N1CCN(C(=O)C2(O)CC2)CC1] |
| (4-(4-(benzo[d]thiazol-6- yl)benzoyl)piperazin-1-yl)(1- hydroxycyclopropyl)methanone | 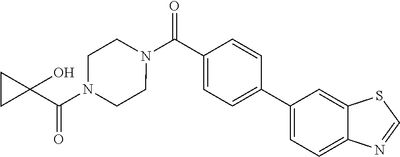 [SMILES: O=C(c1ccc(-c2ccc3ncsc3c2)cc1)N1CCN(C(=O)C2(O)CC2)CC1] |
| (4-(3-chloro-4-(1-isopropyl-1H- indol-5-yl)benzoyl)piperazin-1-yl)(1- hydroxycyclopropyl)methanone | 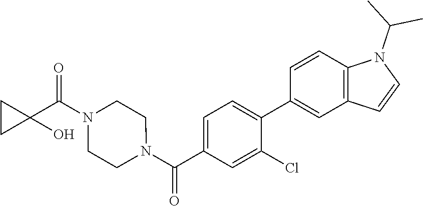 [SMILES: CC(C)n1ccc2cc(-c3ccc(C(=O)N4CCN(C(=O)C5(O)CC5)CC4)cc3Cl)ccc21] |
| (4-(4-(1H-benzo[d]imidazol-5- yl)benzoyl)piperazin-1-yl)(1- hydroxycyclopropyl)methanone | 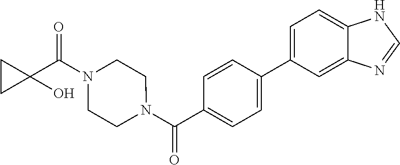 [SMILES: O=C(c1ccc(-c2ccc3[nH]cnc3c2)cc1)N1CCN(C(=O)C2(O)CC2)CC1] |
| 5-(4-(4-(1-hydroxycyclopropane-1- carbonyl)piperazine-1- carbonyl)phenyl)-1H-indole-3- carbonitrile | 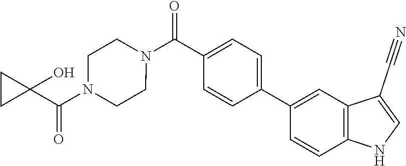 [SMILES: N#Cc1c[nH]c2ccc(-c3ccc(C(=O)N4CCN(C(=O)C5(O)CC5)CC4)cc3)cc12] |
| (4-(2-fluoro-4-(2-hydroxyquinolin- 3-yl)benzoyl)piperazin-1-yl)(1- hydroxycyclopropyl)methanone | 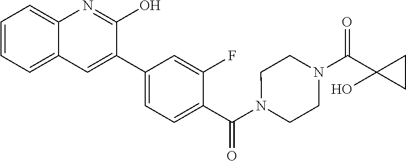 [SMILES: O=C(c1ccc(-c2cc3ccccc3nc2O)cc1F)N1CCN(C(=O)C2(O)CC2)CC1] |
| (4-(4-(benzo[d]oxazol-2- yl)benzoyl)piperazin-1-yl)(1- hydroxycyclopropyl)methanone | 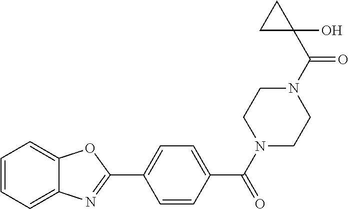 [SMILES: O=C(c1ccc(-c2nc3ccccc3o2)cc1)N1CCN(C(=O)C2(O)CC2)CC1] |
| (4-(2-fluoro-4-(2-methoxyquinolin- 3-yl)benzoyl)piperazin-1-yl)(1- hydroxycyclopropyl)methanone | 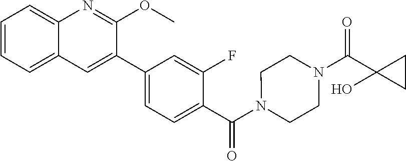 [SMILES: COc1nc2ccccc2cc1-c1ccc(C(=O)N2CCN(C(=O)C3(O)CC3)CC2)c(F)c1] |
| (4-(4-(4-chloroquinolin-3- yl)benzoyl)piperazin-1-yl)(1- hydroxycyclopropyl)methanone | 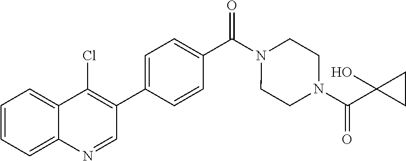 [SMILES: O=C(c1ccc(-c2cnc3ccccc3c2Cl)cc1)N1CCN(C(=O)C2(O)CC2)CC1] |
| (4-(4-(7-fluoro-1H-indol-5- yl)benzoyl)piperazin-1-yl)(1- hydroxycyclopropyl)methanone | 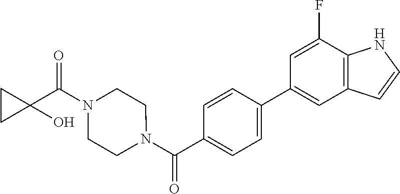 [SMILES: O=C(c1ccc(-c2cc(F)c3[nH]ccc3c2)cc1)N1CCN(C(=O)C2(O)CC2)CC1] |
| (4-(1-hydroxycyclopropane-1- carbonyl)piperazin-1-yl)(4- (quinolin-2-yl)phenyl)methanone | 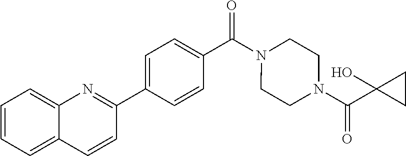 [SMILES: O=C(c1ccc(-c2ccc3ccccc3n2)cc1)N1CCN(C(=O)C2(O)CC2)CC1] |
| (4-(4-(benzo[d]thiazol-2- yl)benzoyl)piperazin-1-yl)(1- hydroxycyclopropyl)methanone | 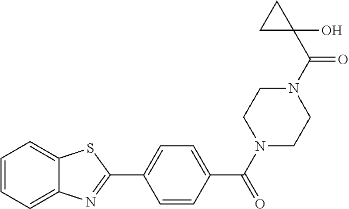 [SMILES: O=C(c1ccc(-c2nc3ccccc3s2)cc1)N1CCN(C(=O)C2(O)CC2)CC1] |
| (4-(1-hydroxycyclopropane-1- carbonyl)piperazin-1-yl)(4-(2- methoxyquinolin-6- yl)phenyl)methanone | 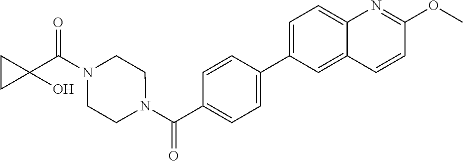 [SMILES: COc1ccc2cc(-c3ccc(C(=O)N4CCN(C(=O)C5(O)CC5)CC4)cc3)ccc2n1] |
| (4-(1-hydroxycyclopropane-1- carbonyl)piperazin-1-yl)(4-(1- (trifluoromethyl)-1H-indol-5- yl)phenyl)methanone | 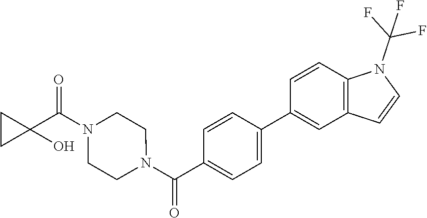 [SMILES: O=C(c1ccc(-c2ccc3c(ccn3C(F)(F)F)c2)cc1)N1CCN(C(=O)C2(O)CC2)CC1] |
| (4-(1-hydroxycyclopropane-1- carbonyl)piperazin-1-yl)(4-(6- methoxynaphthalen-2- yl)phenyl)methanone | 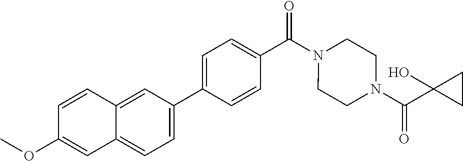 [SMILES: COc1ccc2cc(-c3ccc(C(=O)N4CCN(C(=O)C5(O)CC5)CC4)cc3)ccc2c1] |
| (4-(4-(5-chlorobenzo[d]oxazol-2- yl)benzoyl)piperazin-1-yl)(1- hydroxycyclopropyl)methanone | 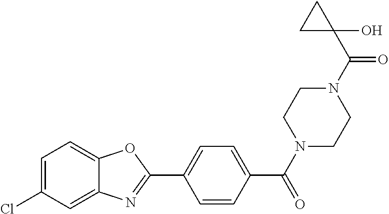 [SMILES: O=C(c1ccc(-c2nc3cc(Cl)ccc3o2)cc1)N1CCN(C(=O)C2(O)CC2)CC1] |
| (4-(4-(6-chlorobenzo[d]oxazol-2- yl)benzoyl)piperazin-1-yl)(1- hydroxycyclopropyl)methanone | 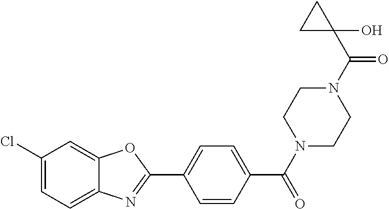 [SMILES: O=C(c1ccc(-c2nc3ccc(Cl)cc3o2)cc1)N1CCN(C(=O)C2(O)CC2)CC1] |
| (4-(4-(5-fluorobenzo[d]oxazol-2- yl)benzoyl)piperazin-1-yl)(1- hydroxycyclopropyl)methanone | 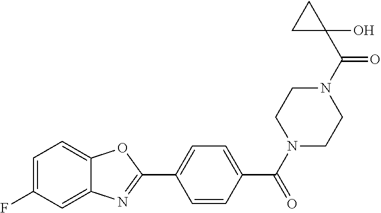 [SMILES: O=C(c1ccc(-c2nc3cc(F)ccc3o2)cc1)N1CCN(C(=O)C2(O)CC2)CC1] |
| (4-(4-(6-fluorobenzo[d]oxazol-2- yl)benzoyl)piperazin-1-yl)(1- hydroxycyclopropyl)methanone | 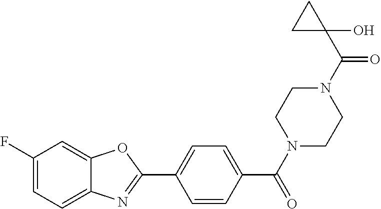 [SMILES: O=C(c1ccc(-c2nc3ccc(F)cc3o2)cc1)N1CCN(C(=O)C2(O)CC2)CC1] |
| (4-(4-(1H-benzo[d]imidazol-2- yl)benzoyl)piperazin-1-yl)(1- hydroxycyclopropyl)methanone | 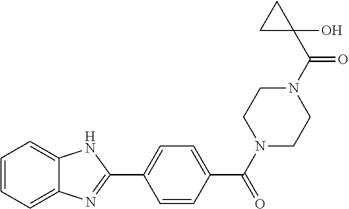 [SMILES: O=C(c1ccc(-c2nc3ccccc3[nH]2)cc1)N1CCN(C(=O)C2(O)CC2)CC1] |
| 5-(4-(4-(1-hydroxycyclopropane-1- carbonyl)piperazine-1- carbonyl)phenyl)-1-isopropyl-1H- indole-3-carbonitrile | 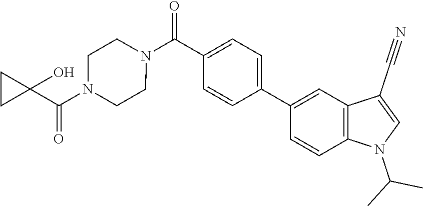 [SMILES: CC(C)n1cc(C#N)c2cc(-c3ccc(C(=O)N4CCN(C(=O)C5(O)CC5)CC4)cc3)ccc21] |
| 5-(2-chloro-4-(4-(1- hydroxycyclopropane-1- carbonyl)piperazine-1- carbonyl)phenyl)-1H-indole-3- carbonitrile | 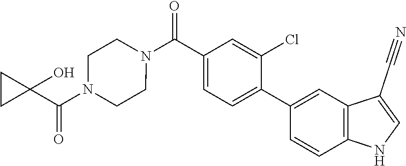 [SMILES: N#Cc1c[nH]c2ccc(-c3ccc(C(=O)N4CCN(C(=O)C5(O)CC5)CC4)cc3Cl)cc12] |
| 5-(3-chloro-4-(4-(1- hydroxycyclopropane-1- carbonyl)piperazine-1- carbonyl)phenyl)-1H-indole-3- carbonitrile | 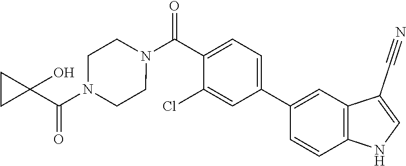 [SMILES: N#Cc1c[nH]c2ccc(-c3ccc(C(=O)N4CCN(C(=O)C5(O)CC5)CC4)c(Cl)c3)cc12] |
| 6-(4-(4-(1-hydroxycyclopropane-1- carbonyl)piperazine-1- carbonyl)phenyl)quinolin-2(1H)- one | 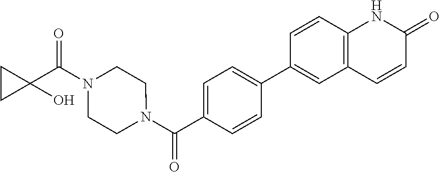 [SMILES: O=C(c1ccc(-c2ccc3[nH]c(=O)ccc3c2)cc1)N1CCN(C(=O)C2(O)CC2)CC1] |
| (4-(1-hydroxycyclopropane-1- carbonyl)piperazin-1-yl)(4- (pyrazolo[1,5-a]pyridin-2- yl)phenyl)methanone | 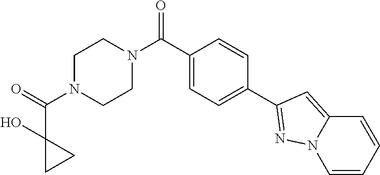 [SMILES: O=C(c1ccc(-c2cc3ccccn3n2)cc1)N1CCN(C(=O)C2(O)CC2)CC1] |
| (4-(1-hydroxycyclopropane-1- carbonyl)piperazin-1-yl)(4-(1- isopropyl-1H-indazol-5- yl)phenyl)methanone | 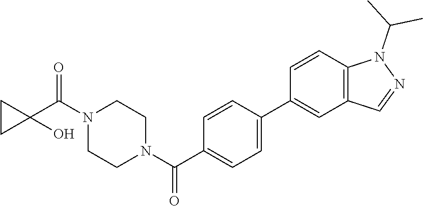 [SMILES: CC(C)n1ncc2cc(-c3ccc(C(=O)N4CCN(C(=O)C5(O)CC5)CC4)cc3)ccc21] |
| (4-(1-hydroxycyclopropane-1- carbonyl)piperazin-1-yl)(6-(2- methoxyquinolin-6-yl)pyridin-3- yl)methanone | 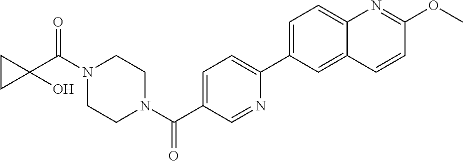 [SMILES: COc1ccc2cc(-c3ccc(C(=O)N4CCN(C(=O)C5(O)CC5)CC4)cn3)ccc2n1] |
| (4-(1-hydroxycyclopropane-1- carbonyl)piperazin-1-yl)(5-(2- methoxyquinolin-6-yl)pyridin-2- yl)methanone | 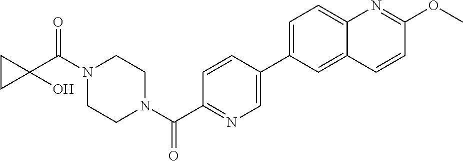 [SMILES: COc1ccc2cc(-c3ccc(C(=O)N4CCN(C(=O)C5(O)CC5)CC4)nc3)ccc2n1] |
| (4-(1-hydroxycyclopropane-1- carbonyl)piperazin-1-yl)(6- phenylimidazo[1,2-a]pyridin-2- yl)methanone | 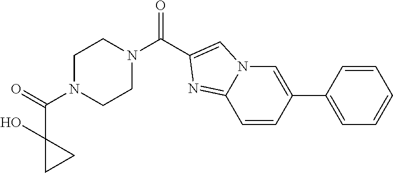 [SMILES: O=C(c1cn2cc(-c3ccccc3)ccc2n1)N1CCN(C(=O)C2(O)CC2)CC1] |
| (6-(4-fluorophenyl)imidazo[1,2- a]pyridin-2-yl)(4-(1- hydroxycyclopropane-1- carbonyl)piperazin-1-yl)methanone | 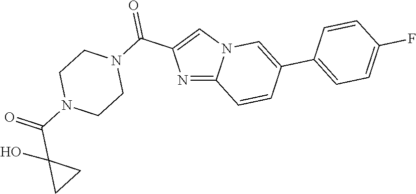 [SMILES: O=C(c1cn2cc(-c3ccc(F)cc3)ccc2n1)N1CCN(C(=O)C2(O)CC2)CC1] |
| (4-(5-(4-chloro-2- fluorophenyl)picolinoyl)piperazin- 1-yl)(1- hydroxycyclopropyl)methanone | 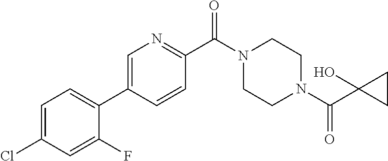 [SMILES: O=C(c1ccc(-c2ccc(Cl)cc2F)cn1)N1CCN(C(=O)C2(O)CC2)CC1] |
| (4-(6-(4-chloro-2- fluorophenyl)nicotinoyl)piperazin- 1-yl)(1- hydroxycyclopropyl)methanone | 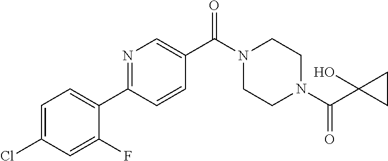 [SMILES: O=C(c1ccc(-c2ccc(Cl)cc2F)nc1)N1CCN(C(=O)C2(O)CC2)CC1] |
| (5-(4-chloro-2- fluorophenyl)pyrimidin-2-yl)(4-(1- hydroxycyclopropane-1- carbonyl)piperazin-1-yl)methanone | 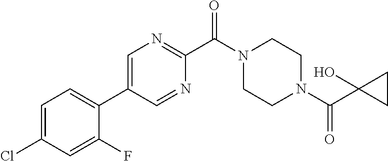 [SMILES: O=C(c1ncc(-c2ccc(Cl)cc2F)cn1)N1CCN(C(=O)C2(O)CC2)CC1] |
| (4-(2-fluoro-4-(6-fluoroquinolin-2- yl)benzoyl)piperazin-1-yl)(1- hydroxycyclopropyl)methanone | 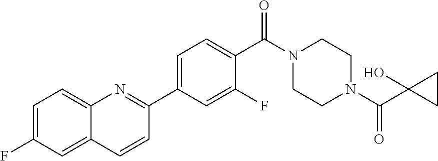 [SMILES: O=C(c1ccc(-c2ccc3cc(F)ccc3n2)cc1F)N1CCN(C(=O)C2(O)CC2)CC1] |
| (4-(2-fluoro-4-(6-methoxyquinolin- 2-yl)benzoyl)piperazin-1-yl)(1- hydroxycyclopropyl)methanone | 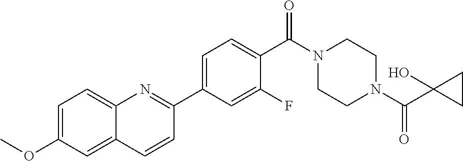 [SMILES: COc1ccc2nc(-c3ccc(C(=O)N4CCN(C(=O)C5(O)CC5)CC4)c(F)c3)ccc2c1] |
| (4-(2,3-difluoro-4-(quinolin-2- yl)benzoyl)piperazin-1-yl)(1- hydroxycyclopropyl)methanone | 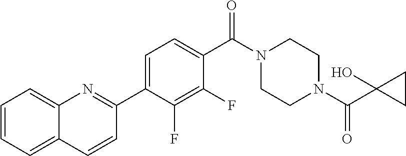 [SMILES: O=C(c1ccc(-c2ccc3ccccc3n2)c(F)c1F)N1CCN(C(=O)C2(O)CC2)CC1] |
| (4-(2,3-difluoro-4-(6-fluoroquinolin- 2-yl)benzoyl)piperazin-1-yl)(1- hydroxycyclopropyl)methanone | 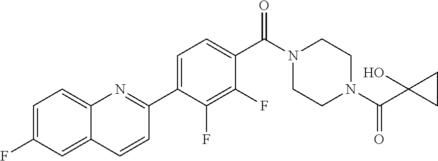 [SMILES: O=C(c1ccc(-c2ccc3cc(F)ccc3n2)c(F)c1F)N1CCN(C(=O)C2(O)CC2)CC1] |
| (4-(2,3-difluoro-4-(6- methoxyquinolin-2- yl)benzoyl)piperazin-1-yl)(1- hydroxycyclopropyl)methanone | 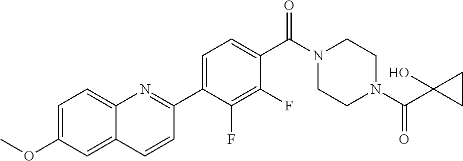 [SMILES: COc1ccc2nc(-c3ccc(C(=O)N4CCN(C(=O)C5(O)CC5)CC4)c(F)c3F)ccc2c1] |
| (4-(4-(6-fluoroquinolin-2- yl)benzoyl)piperazin-1-yl)(1- hydroxycyclopropyl)methanone | 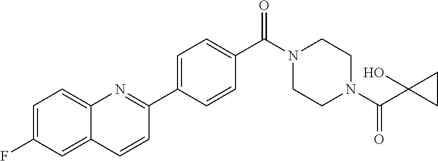 [SMILES: O=C(c1ccc(-c2ccc3cc(F)ccc3n2)cc1)N1CCN(C(=O)C2(O)CC2)CC1] |
| (4-(1-hydroxycyclopropane-1- carbonyl)piperazin-1-yl)(4-(6- methoxyquinolin-2- yl)phenyl)methanone | 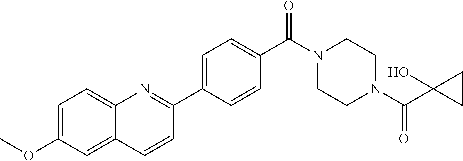 [SMILES: COc1ccc2nc(-c3ccc(C(=O)N4CCN(C(=O)C5(O)CC5)CC4)cc3)ccc2c1] |
| (4-(1-hydroxycyclopropane-1- carbonyl)piperazin-1-yl)(4- (imidazo[1,2-a]pyridin-2- yl)phenyl)methanone | 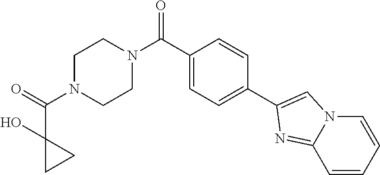 [SMILES: O=C(c1ccc(-c2cn3ccccc3n2)cc1)N1CCN(C(=O)C2(O)CC2)CC1] |
| (4-(4-(benzo[c][1,2,5]oxadiazol-5- yl)benzoyl)piperazin-1-yl)(1- hydroxycyclopropyl)methanone | 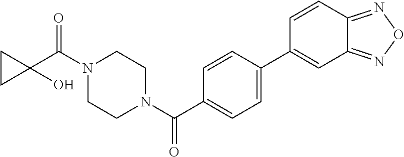 [SMILES: O=C(c1ccc(-c2ccc3nonc3c2)cc1)N1CCN(C(=O)C2(O)CC2)CC1] |
| N-(4′-(4-(1-hydroxycyclopropane-1- carbonyl)piperazine-1-carbonyl)- [1,1′-biphenyl]-3- yl)benzenesulfonamide | 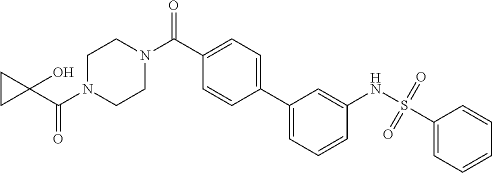 [SMILES: O=C(c1ccc(-c2cccc(NS(=O)(=O)c3ccccc3)c2)cc1)N1CCN(C(=O)C2(O)CC2)CC1] |
| N-(4′-(4-(1-hydroxycyclopropane-1- carbonyl)piperazine-1-carbonyl)- 2,3,4,5-tetrahydro-[1,1′-biphenyl]- 3-yl)methanesulfonamide | 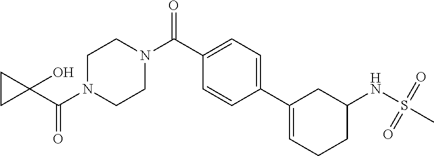 [SMILES: CS(=O)(=O)NC1CCC=C(c2ccc(C(=O)N3CCN(C(=O)C4(O)CC4)CC3)cc2)C1] |
| N-(4′-(4-(1-hydroxycyclopropane-1- carbonyl)piperazine-1-carbonyl)- [1,1′-biphenyl]-3-yl)propane-2- sulfonamide | 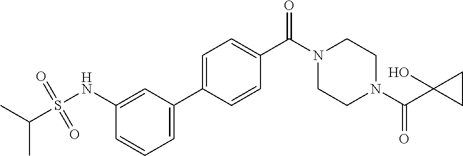 [SMILES: CC(C)S(=O)(=O)Nc1cccc(-c2ccc(C(=O)N3CCN(C(=O)C4(O)CC4)CC3)cc2)c1] |
| N-(4′-(4-(1-hydroxycyclopropane-1- carbonyl)piperazine-1-carbonyl)- 3′,4′-dihydro-[1,1′-biphenyl]-3- yl)cyclopropanesulfonamide | 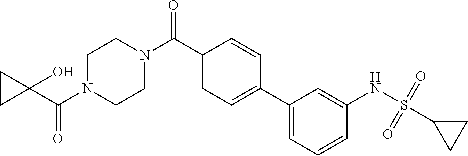 [SMILES: O=C(C1C=CC(c2cccc(NS(=O)(=O)C3CC3)c2)=CC1)N1CCN(C(=O)C2(O)CC2)CC1] |
| (4-(1-hydroxycyclopropane-1- carbonyl)piperazin-1-yl)(4- (isoquinolin-1- yl)phenyl)methanone | 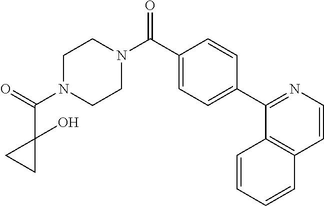 [SMILES: O=C(c1ccc(-c2nccc3ccccc23)cc1)N1CCN(C(=O)C2(O)CC2)CC1] |
| (4-(1-hydroxycyclopropane-1- carbonyl)piperazin-1-yl)(4-(7- (trifluoromethyl)quinolin-4- yl)phenyl)methanone | 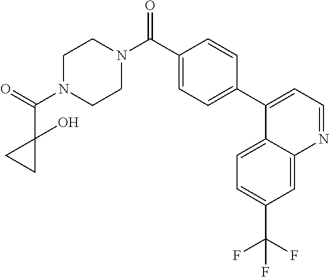 [SMILES: O=C(c1ccc(-c2ccnc3cc(C(F)(F)F)ccc23)cc1)N1CCN(C(=O)C2(O)CC2)CC1] |
| (4-(1-hydroxycyclopropane-1- carbonyl)piperazin-1-yl)(4-(6- methoxy-4-methylquinolin-2- yl)phenyl)methanone | 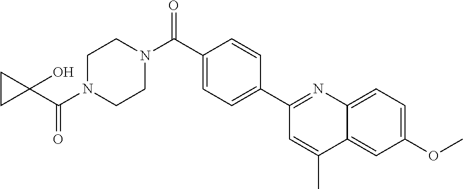 [SMILES: COc1ccc2nc(-c3ccc(C(=O)N4CCN(C(=O)C5(O)CC5)CC4)cc3)cc(C)c2c1] |
| (4-(4-(5-chloro-1H- benzo[d]imidazol-2- yl)benzoyl)piperazin-1-yl)(1- hydroxycyclopropyl)methanone | 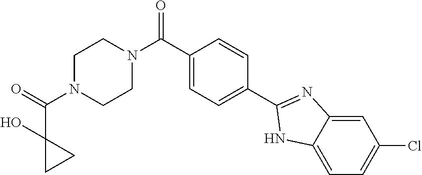 [SMILES: O=C(c1ccc(-c2nc3cc(Cl)ccc3[nH]2)cc1)N1CCN(C(=O)C2(O)CC2)CC1] |
| (4-(4-(4-chloro-1H- benzo[d]imidazol-2- yl)benzoyl)piperazin-1-yl)(1- hydroxycyclopropyl)methanone | 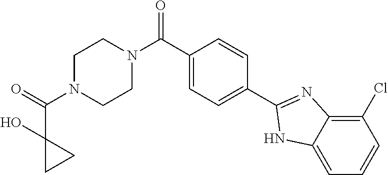 [SMILES: O=C(c1ccc(-c2nc3c(Cl)cccc3[nH]2)cc1)N1CCN(C(=O)C2(O)CC2)CC1] |
| (4-(4-(5,6-difluoro-1H- benzo[d]imidazol-2- yl)benzoyl)piperazin-1-yl)(1- hydroxycyclopropyl)methanone | 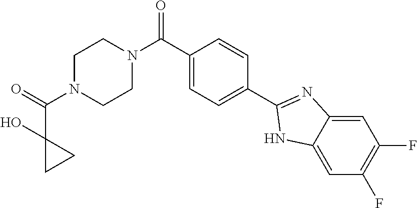 [SMILES: O=C(c1ccc(-c2nc3cc(F)c(F)cc3[nH]2)cc1)N1CCN(C(=O)C2(O)CC2)CC1] |
| (4-(1-hydroxycyclopropane-1- carbonyl)piperazin-1-yl)(4-(5- methoxy-1H-benzo[d]imidazol-2- yl)phenyl)methanone | 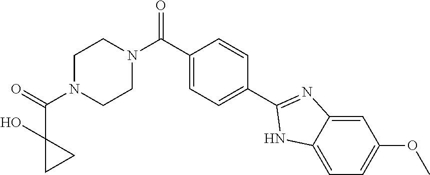 [SMILES: COc1ccc2[nH]c(-c3ccc(C(=O)N4CCN(C(=O)C5(O)CC5)CC4)cc3)nc2c1] |
| (4-(4-(5-fluoro-1H- benzo[d]imidazol-2- yl)benzoyl)piperazin-1-yl)(1- hydroxycyclopropyl)methanone | 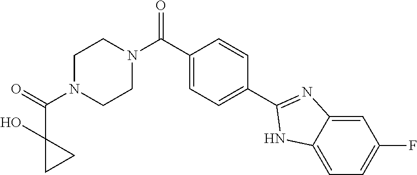 [SMILES: O=C(c1ccc(-c2nc3cc(F)ccc3[nH]2)cc1)N1CCN(C(=O)C2(O)CC2)CC1] |
| (4-(4-(1,3-dimethyl-1H-indazol-5- yl)benzoyl)piperazin-1-yl)(1- hydroxycyclopropyl)methanone | 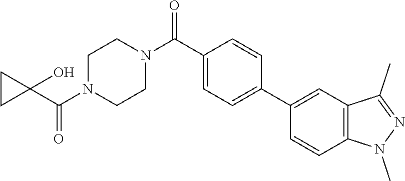 [SMILES: Cc1nn(C)c2ccc(-c3ccc(C(=O)N4CCN(C(=O)C5(O)CC5)CC4)cc3)cc12] |
| 6-(4-(4-(1-hydroxycyclopropane-1- carbonyl)piperazine-1- carbonyl)phenyl)benzo[d]oxazol- 2(3H)-one | 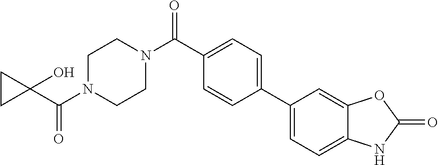 [SMILES: O=C(c1ccc(-c2ccc3[nH]c(=O)oc3c2)cc1)N1CCN(C(=O)C2(O)CC2)CC1] |
| 5-(4-(4-(1-hydroxycyclopropane-1- carbonyl)piperazine-1- carbonyl)phenyl)benzo[d]oxazol- 2(3H)-one | 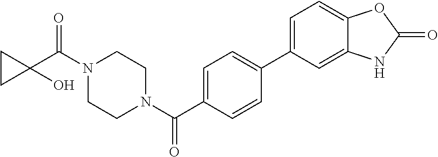 [SMILES: O=C(c1ccc(-c2ccc3oc(=O)[nH]c3c2)cc1)N1CCN(C(=O)C2(O)CC2)CC1] |
| (4-(1-hydroxycyclopropane-1- carbonyl)piperazin-1-yl)(4-(3- methylbenzo[d]isoxazol-6- yl)phenyl)methanone | 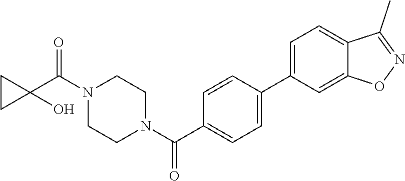 [SMILES: Cc1noc2cc(-c3ccc(C(=O)N4CCN(C(=O)C5(O)CC5)CC4)cc3)ccc12] |
| (4-(4-(1H-benzo[d]imidazol-4- yl)benzoyl)piperazin-1-yl)(1- hydroxycyclopropyl)methanone | 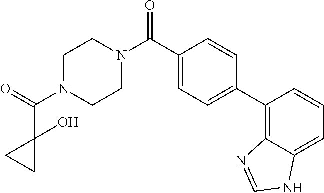 [SMILES: O=C(c1ccc(-c2cccc3[nH]cnc23)cc1)N1CCN(C(=O)C2(O)CC2)CC1] |
| (4-(4-(benzo[d]oxazol-4- yl)benzoyl)piperazin-1-yl)(1- hydroxycyclopropyl)methanone | 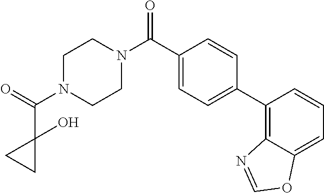 [SMILES: O=C(c1ccc(-c2cccc3ocnc23)cc1)N1CCN(C(=O)C2(O)CC2)CC1] |
| (4-(1-hydroxycyclopropane-1- carbonyl)piperazin-1-yl)(4- (quinolin-5-yl)phenyl)methanone | 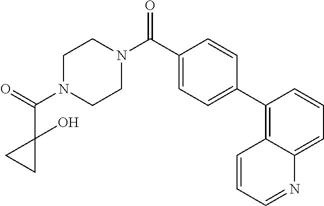 [SMILES: O=C(c1ccc(-c2cccc3ncccc23)cc1)N1CCN(C(=O)C2(O)CC2)CC1] |
| (4-(4-(1H-indazol-3- yl)benzoyl)piperazin-1-yl)(1- hydroxycyclopropyl)methanone | 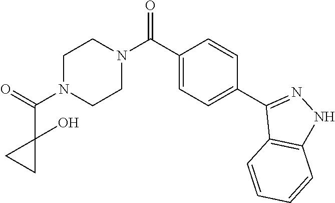 [SMILES: O=C(c1ccc(-c2n[nH]c3ccccc23)cc1)N1CCN(C(=O)C2(O)CC2)CC1] |
| (4-(4-(furo[3,2-b]pyridin-5- yl)benzoyl)piperazin-1-yl)(1- hydroxycyclopropyl)methanone | 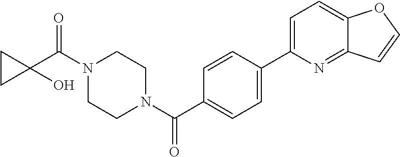 [SMILES: O=C(c1ccc(-c2ccc3occc3n2)cc1)N1CCN(C(=O)C2(O)CC2)CC1] |
| (4-(4-(furo[3,2-c]pyridin-4- yl)benzoyl)piperazin-1-yl)(1- hydroxycyclopropyl)methanone | 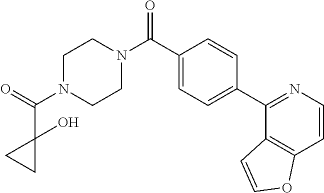 [SMILES: O=C(c1ccc(-c2nccc3occc23)cc1)N1CCN(C(=O)C2(O)CC2)CC1] |
| (4-(4-([1,2,4]triazolo[4,3- b]pyridazin-6-yl)benzoyl)piperazin- 1-yl)(1- hydroxycyclopropyl)methanone | 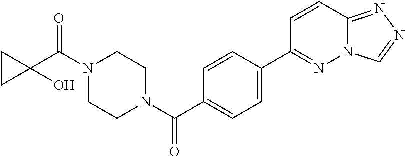 [SMILES: O=C(c1ccc(-c2ccc3nncn3n2)cc1)N1CCN(C(=O)C2(O)CC2)CC1] |
| (4-(1-hydroxycyclopropane-1- carbonyl)piperazin-1-yl)(4- (imidazo[1,2-a]pyrazin-6- yl)phenyl)methanone | 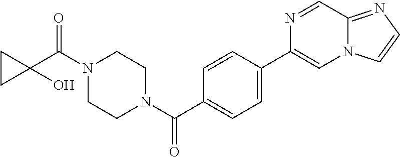 [SMILES: O=C(c1ccc(-c2cn3ccnc3cn2)cc1)N1CCN(C(=O)C2(O)CC2)CC1] |
| (4-(4-(1H-pyrrolo[3,2-b]pyridin-5- yl)benzoyl)piperazin-1-yl)(1- hydroxycyclopropyl)methanone | 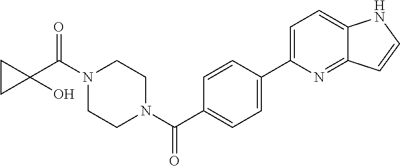 [SMILES: O=C(c1ccc(-c2ccc3[nH]ccc3n2)cc1)N1CCN(C(=O)C2(O)CC2)CC1] |
| (4-(1-hydroxycyclopropane-1- carbonyl)piperazin-1-yl)(4- (imidazo[1,2-b]pyridazin-6- yl)phenyl)methanone | 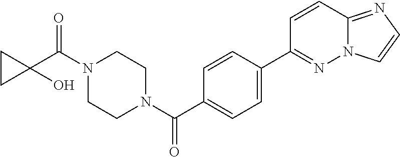 [SMILES: O=C(c1ccc(-c2ccc3nccn3n2)cc1)N1CCN(C(=O)C2(O)CC2)CC1] |
| (4-(1-hydroxycyclopropane-1- carbonyl)piperazin-1-yl)(4-(1- methyl-1H-benzo[d]imidazol-5- yl)phenyl)methanone | 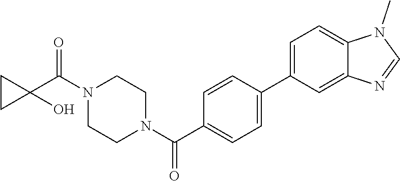 [SMILES: Cn1cnc2cc(-c3ccc(C(=O)N4CCN(C(=O)C5(O)CC5)CC4)cc3)ccc21] |
| (4-(4-(benzo[c][1,2,5]thiadiazol-4- yl)benzoyl)piperazin-1-yl)(1- hydroxycyclopropyl)methanone | 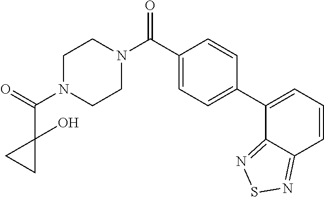 [SMILES: O=C(c1ccc(-c2cccc3nsnc23)cc1)N1CCN(C(=O)C2(O)CC2)CC1] |
| (4-(1-hydroxycyclopropane-1- carbonyl)piperazin-1-yl)(4-(2- (trifluoromethyl)imidazo[1,2- a]pyridin-6-yl)phenyl)methanone | 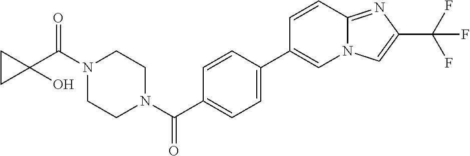 [SMILES: O=C(c1ccc(-c2ccc3nc(C(F)(F)F)cn3c2)cc1)N1CCN(C(=O)C2(O)CC2)CC1] |
| (4-(4-(6-chloroimidazo[1,2- a]pyridin-3-yl)benzoyl)piperazin-1- yl)(1- hydroxycyclopropyl)methanone | 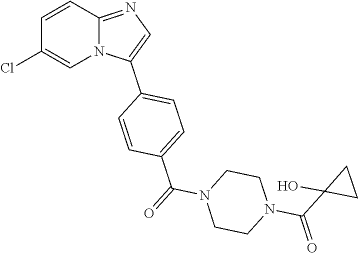 [SMILES: O=C(c1ccc(-c2cnc3ccc(Cl)cn23)cc1)N1CCN(C(=O)C2(O)CC2)CC1] |
| (4-(1-hydroxycyclopropane-1- carbonyl)piperazin-1-yl)(4-(2- methyl-2H-indazol-5- yl)phenyl)methanone | 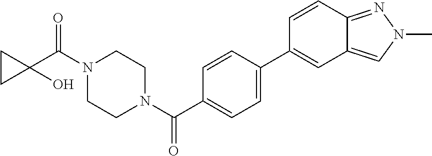 [SMILES: Cn1cc2cc(-c3ccc(C(=O)N4CCN(C(=O)C5(O)CC5)CC4)cc3)ccc2n1] |
| (4-(1-hydroxycyclopropane-1- carbonyl)piperazin-1-yl)(4- (imidazo[1,2-a]pyrimidin-7- yl)phenyl)methanone | 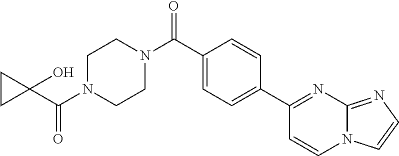 [SMILES: O=C(c1ccc(-c2ccn3ccnc3n2)cc1)N1CCN(C(=O)C2(O)CC2)CC1] |
| 5-(4-(4-(1-hydroxycyclopropane-1- carbonyl)piperazine-1- carbonyl)phenyl)-1H-indole-2- carbonitrile | 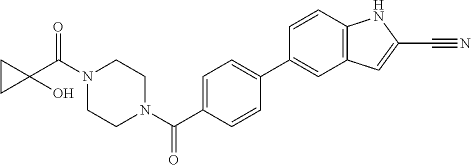 [SMILES: N#Cc1cc2cc(-c3ccc(C(=O)N4CCN(C(=O)C5(O)CC5)CC4)cc3)ccc2[nH]1] |
| 2-(4-(4-(1-hydroxycyclopropane-1- carbonyl)piperazine-1- carbonyl)phenyl)benzo[d]oxazole- 6-carbonitrile | 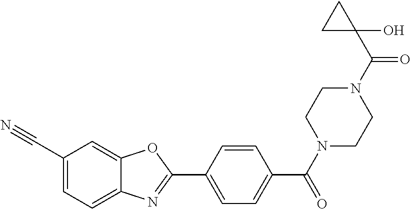 [SMILES: N#Cc1ccc2nc(-c3ccc(C(=O)N4CCN(C(=O)C5(O)CC5)CC4)cc3)oc2c1] |
| (4-(4-(1H-3l2- benzo[c][1,2,5]thiadiazol-5- yl)benzoyl)piperazin-1-yl)(1- hydroxycyclopropyl)methanone | 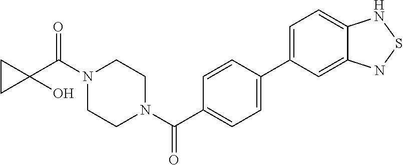 [SMILES: O=C(c1ccc(-c2ccc3c(c2)NSN3)cc1)N1CCN(C(=O)C2(O)CC2)CC1] |
| (4-(4-(2H-benzo[d][1,2,3]triazol-5- yl)benzoyl)piperazin-1-yl)(1- hydroxycyclopropyl)methanone | 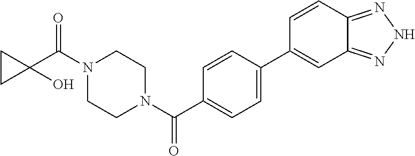 [SMILES: O=C(c1ccc(-c2ccc3n[nH]nc3c2)cc1)N1CCN(C(=O)C2(O)CC2)CC1] |
| (4-(1-hydroxycyclopropane-1- carbonyl)piperazin-1-yl)(2- phenylbenzo[d]thiazol-6- yl)methanone | 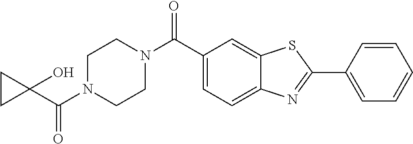 [SMILES: O=C(c1ccc2nc(-c3ccccc3)sc2c1)N1CCN(C(=O)C2(O)CC2)CC1] |
| (2-(4-fluorophenyl)benzo[d]thiazol- 6-yl)(4-(1-hydroxycyclopropane-1- carbonyl)piperazin-1-yl)methanone | 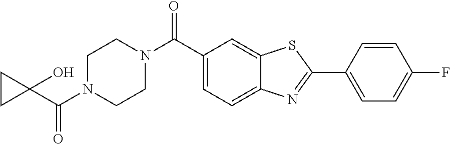 [SMILES: O=C(c1ccc2nc(-c3ccc(F)cc3)sc2c1)N1CCN(C(=O)C2(O)CC2)CC1] |
| 2-(4-(4-(1-hydroxycyclopropane-1- carbonyl)piperazine-1- carbonyl)phenyl)benzo[d]oxazole- 5-carbonitrile | 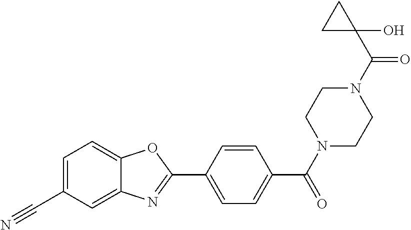 [SMILES: N#Cc1ccc2oc(-c3ccc(C(=O)N4CCN(C(=O)C5(O)CC5)CC4)cc3)nc2c1] |
| 3-(4-(4-(1-hydroxycyclopropane-1- carbonyl)piperazine-1- carbonyl)phenyl)quinolin-2(1H)- one | 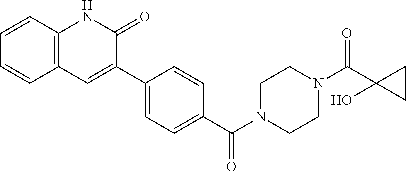 [SMILES: O=C(c1ccc(-c2cc3ccccc3[nH]c2=O)cc1)N1CCN(C(=O)C2(O)CC2)CC1] |
| (4-(2-chloro-4-(imidazo[1,2- a]pyridin-2-yl)benzoyl)piperazin-1- yl)(1- hydroxycyclopropyl)methanone | 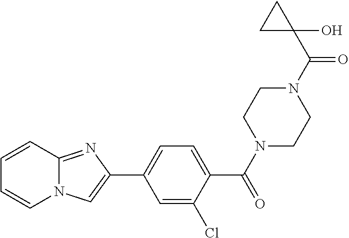 [SMILES: O=C(c1ccc(-c2cn3ccccc3n2)cc1Cl)N1CCN(C(=O)C2(O)CC2)CC1] |
| (4-(1-hydroxycyclopropane-1- carbonyl)piperazin-1-yl)(3- phenoxyphenyl)methanone | 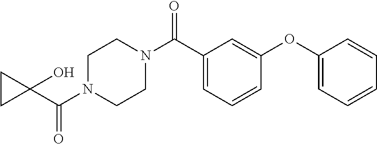 [SMILES: O=C(c1cccc(Oc2ccccc2)c1)N1CCN(C(=O)C2(O)CC2)CC1] |
| (4-(4-(6-fluoroimidazo[1,2- a]pyridin-2-yl)benzoyl)piperazin-1- hydroxycyclopropyl)methanone | 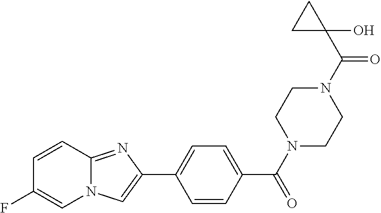 [SMILES: O=C(c1ccc(-c2cn3cc(F)ccc3n2)cc1)N1CCN(C(=O)C2(O)CC2)CC1] |
| (4-(1-hydroxycyclopropane-1- carbonyl)piperazin-1-yl)(4-(6- methoxyimidazo[1,2-a]pyridin-2- yl)phenyl)methanone | 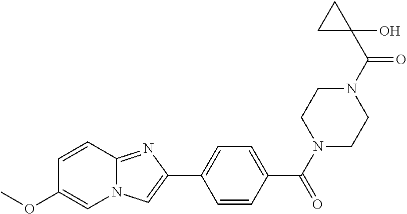 [SMILES: COc1ccc2nc(-c3ccc(C(=O)N4CCN(C(=O)C5(O)CC5)CC4)cc3)cn2c1] |
| N-(4′-(4-(1-hydroxycyclopropane-1- carbonyl)piperazine-1-carbonyl)- [1,1′-biphenyl]-3- yl)cyclopropanecarboxamide | 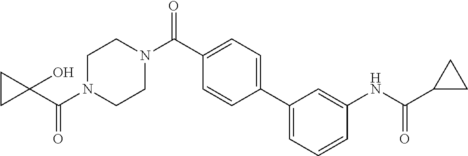 [SMILES: O=C(Nc1cccc(-c2ccc(C(=O)N3CCN(C(=O)C4(O)CC4)CC3)cc2)c1)C1CC1] |
| (S)-(4-(4′-chloro-2′-fluoro-[1,1′- biphenyl]-4-carbonyl)-2- (hydroxymethyl)piperazin-1-yl)(1- hydroxycyclopropyl)methanone | 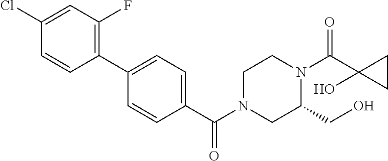 [SMILES: O=C(c1ccc(-c2ccc(Cl)cc2F)cc1)N1CCN(C(=O)C2(O)CC2)[C@H](CO)C1] |
| (4-(3′-cyclopropoxy-[1,1′-biphenyl]- 4-carbonyl)piperazin-1-yl)(1- hydroxycyclopropyl)methanone | 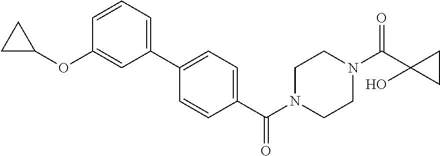 [SMILES: O=C(c1ccc(-c2cccc(OC3CC3)c2)cc1)N1CCN(C(=O)C2(O)CC2)CC1] |
| N-cyclopropyl-4′-(4-(1- hydroxycyclopropane-1- carbonyl)piperazine-1-carbonyl)- [1,1′-biphenyl]-3-sulfonamide | 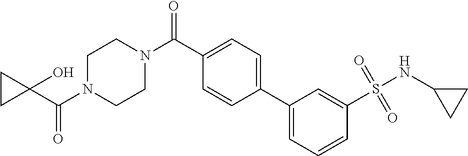 [SMILES: O=C(c1ccc(-c2cccc(S(=O)(=O)NC3CC3)c2)cc1)N1CCN(C(=O)C2(O)CC2)CC1] |
| 5-(4-(4-(1-hydroxycyclopropane-1- carbonyl)piperazine-1- carbonyl)phenyl)-1H-indole-2- carboxamide | 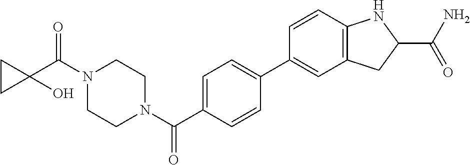 [SMILES: NC(=O)C1Cc2cc(-c3ccc(C(=O)N4CCN(C(=O)C5(O)CC5)CC4)cc3)ccc2N1] |
| (4-(3′-chloro-[1,1′-biphenyl]-4- carbonyl)piperazin-1-yl)(1- hydroxycyclopropyl)methanone | 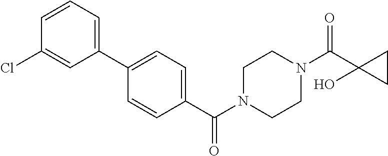 [SMILES: O=C(c1ccc(-c2cccc(Cl)c2)cc1)N1CCN(C(=O)C2(O)CC2)CC1] |
| (4-(4′-chloro-[1,1′-biphenyl]-4- carbonyl)piperazin-1-yl)(1- hydroxycyclopropyl)methanone | 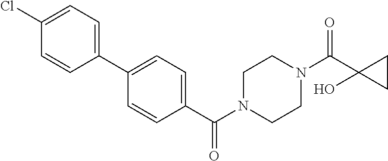 [SMILES: O=C(c1ccc(-c2ccc(Cl)cc2)cc1)N1CCN(C(=O)C2(O)CC2)CC1] |
| (S)-(4-(4′-chloro-2′-fluoro-[1,1′- biphenyl]-4-carbonyl)-3- (hydroxymethyl)piperazin-1-yl)(1- hydroxycyclopropyl)methanone | 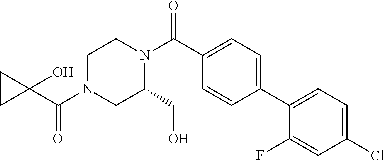 [SMILES: O=C(c1ccc(-c2ccc(Cl)cc2F)cc1)N1CCN(C(=O)C2(O)CC2)C[C@H]1CO] |
| (4-(4-(1H-indol-2- yl)benzoyl)piperazin-1-yl)(1- hydroxycyclopropyl)methanone | 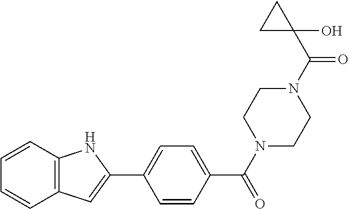 [SMILES: O=C(c1ccc(-c2cc3ccccc3[nH]2)cc1)N1CCN(C(=O)C2(O)CC2)CC1] |
| (4-(4-(3-chloroimidazo[1,2- a]pyridin-2-yl)benzoyl)piperazin-1- yl)(1- hydroxycyclopropyl)methanone | 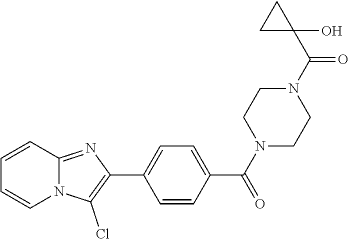 [SMILES: O=C(c1ccc(-c2nc3ccccn3c2Cl)cc1)N1CCN(C(=O)C2(O)CC2)CC1] |
| (4-(1-hydroxycyclopropane-1- carbonyl)piperazin-1-yl)(4-(1- methyl-1H-benzo[d]imidazol-2- yl)phenyl)methanone | 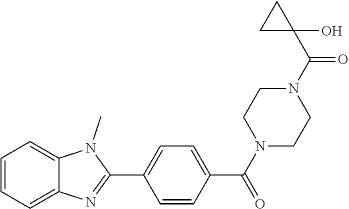 [SMILES: Cn1c(-c2ccc(C(=O)N3CCN(C(=O)C4(O)CC4)CC3)cc2)nc2ccccc21] |
| 3′-chloro-N-cyclopropyl-4′-(4-(1- hydroxycyclopropane-1- carbonyl)piperazine-1-carbonyl)- [1,1′-biphenyl]-3-sulfonamide | 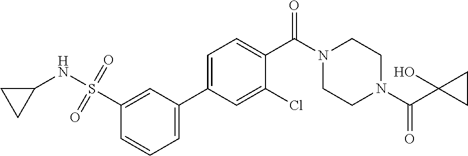 [SMILES: O=C(c1ccc(-c2cccc(S(=O)(=O)NC3CC3)c2)cc1Cl)N1CCN(C(=O)C2(O)CC2)CC1] |
| (4-(2-chloro-4-(furo[3,2-b]pyridin- 5-yl)benzoyl)piperazin-1-yl)(1- hydroxycyclopropyl)methanone | 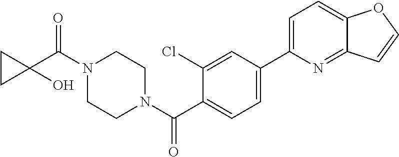 [SMILES: O=C(c1ccc(-c2ccc3occc3n2)cc1Cl)N1CCN(C(=O)C2(O)CC2)CC1] |
| (4-(2-chloro-4-(1-methyl-1H- benzo[d]imidazol-5- yl)benzoyl)piperazin-1-yl)(1- hydroxycyclopropyl)methanone | 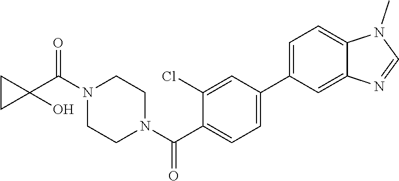 [SMILES: Cn1cnc2cc(-c3ccc(C(=O)N4CCN(C(=O)C5(O)CC5)CC4)c(Cl)c3)ccc21] |
| N-(3′-chloro-4′-(4-(1- hydroxycyclopropane-1- carbonyl)piperazine-1-carbonyl)- [1,1′-biphenyl]-3- yl)cyclopropanesulfonamide | 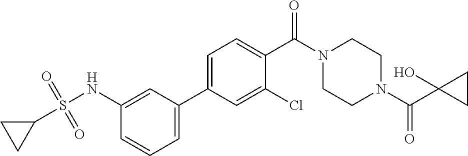 [SMILES: O=C(c1ccc(-c2cccc(NS(=O)(=O)C3CC3)c2)cc1Cl)N1CCN(C(=O)C2(O)CC2)CC1] |
| N-(4′-(4-(1-hydroxycyclopropane-1- carbonyl)piperazine-1-carbonyl)- [1,1′-biphenyl]-3- yl)cyclobutanecarboxamide | 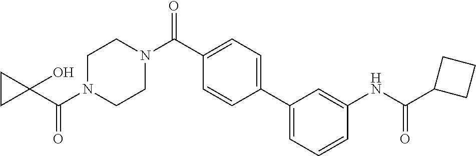 [SMILES: O=C(Nc1cccc(-c2ccc(C(=O)N3CCN(C(=O)C4(O)CC4)CC3)cc2)c1)C1CCC1] |
| (4-(3′-(cyclopropylsulfonyl)-(1,1′- biphenyl]-4-carbonyl)piperazin-1- yl)(1- hydroxycyclopropyl)methanone | 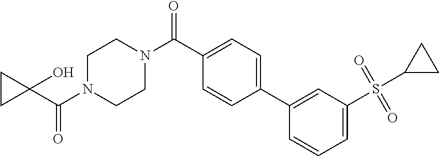 [SMILES: O=C(c1ccc(-c2cccc(S(=O)(=O)C3CC3)c2)cc1)N1CCN(C(=O)C2(O)CC2)CC1] |
| N-(5-(3-chloro-4-(4-(1- hydroxycyclopropane-1- carbonyl)piperazine-1- carbonyl)phenyl)pyridin-3- yl)cyclopropanesulfonamide | 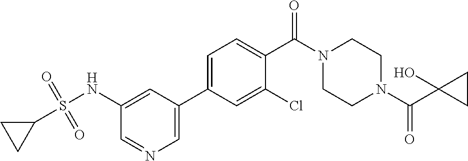 [SMILES: O=C(c1ccc(-c2cncc(NS(=O)(=O)C3CC3)c2)cc1Cl)N1CCN(C(=O)C2(O)CC2)CC1] |
| N-(5-(3-chloro-4-(4-(1- hydroxycyclopropane-1- carbonyl)piperazine-1- carbonyl)phenyl)pyridin-3- yl)cyclopropanecarboxamide | 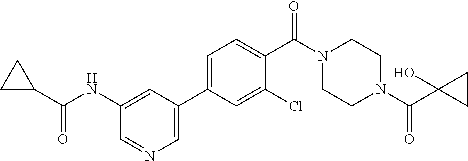 [SMILES: O=C(Nc1cncc(-c2ccc(C(=O)N3CCN(C(=O)C4(O)CC4)CC3)c(Cl)c2)c1)C1CC1] |
| N-(4′-(4-(1-hydroxycyclopropane-1- carbonyl)piperazine-1-carbonyl)- [1,1′-biphenyl]-3-yl)oxetane-3- carboxamide | 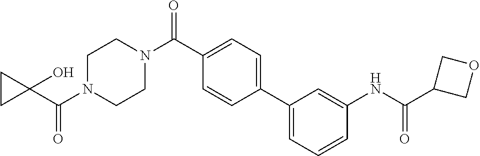 [SMILES: O=C(Nc1cccc(-c2ccc(C(=O)N3CCN(C(=O)C4(O)CC4)CC3)cc2)c1)C1COC1] |
| (4-(3′-(cyclopropylsulfonyl)-[1,1′- biphenyl]-4-carbonyl)piperazin-1- yl)(1-hydroxycyclobutyl)methanone | 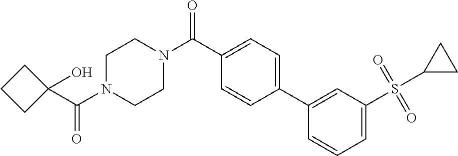 [SMILES: O=C(c1ccc(-c2cccc(S(=O)(=O)C3CC3)c2)cc1)N1CCN(C(=O)C2(O)CCC2)CC1] |
| (4-(3′-(cyclopropylsulfonyl)-[1,1′- biphenyl]-4-carbonyl)piperazin-1- yl)(oxetan-2-yl)methanone | 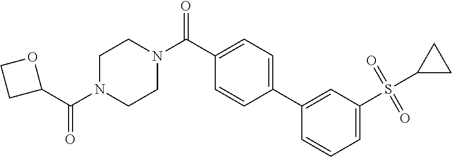 [SMILES: O=C(c1ccc(-c2cccc(S(=O)(=O)C3CC3)c2)cc1)N1CCN(C(=O)C2CCO2)CC1] |
| (4-(4-(3-chloroimidazo[1,2- a]pyridin-2-yl)benzoyl)piperazin-1- yl)(1-hydroxycyclobutyl)methanone | 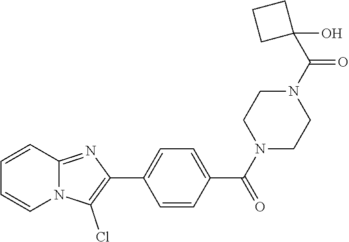 [SMILES: O=C(c1ccc(-c2nc3ccccn3c2Cl)cc1)N1CCN(C(=O)C2(O)CCC2)CC1] |
| (4-(4-(3-chloroimidazo[1,2- a]pyridin-2-yl)benzoyl)piperazin-1- yl)(oxetan-2-yl)methanone | 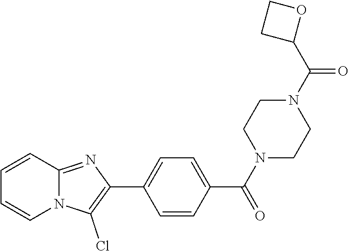 [SMILES: O=C(c1ccc(-c2nc3ccccn3c2Cl)cc1)N1CCN(C(=O)C2CCO2)CC1] |
| (4-(3′-cyclopropoxy-[1,1′-biphenyl]- 4-carbonyl)piperazin-1-yl)(1- hydroxycyclobutyl)methanone | 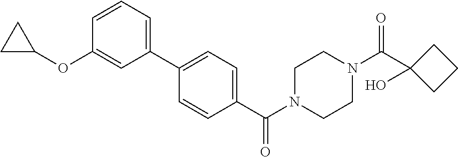 [SMILES: O=C(c1ccc(-c2cccc(OC3CC3)c2)cc1)N1CCN(C(=O)C2(O)CCC2)CC1] |
| (4-(3′-cyclopropoxy-[1,1′-biphenyl]- 4-carbonyl)piperazin-1-yl)(oxetan- 2-yl)methanone | 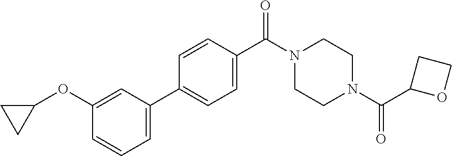 [SMILES: O=C(c1ccc(-c2cccc(OC3CC3)c2)cc1)N1CCN(C(=O)C2CCO2)CC1] |
| N-(4′-(4-(1-hydroxycyclobutane-1- carbonyl)piperazine-1-carbonyl)- [1,1′-biphenyl]-3- yl)cyclopropanecarboxamide | 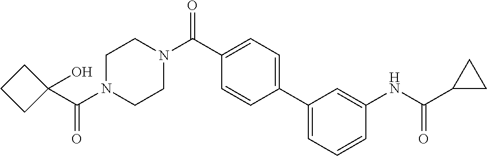 [SMILES: O=C(Nc1cccc(-c2ccc(C(=O)N3CCN(C(=O)C4(O)CCC4)CC3)cc2)c1)C1CC1] |
| N-(4′-(4-(oxetane-2- carbonyl)piperazine-1-carbonyl)- [1,1′-biphenyl]-3- yl)cyclopropanecarboxamide | 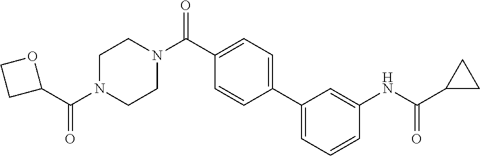 [SMILES: O=C(Nc1cccc(-c2ccc(C(=O)N3CCN(C(=O)C4CCO4)CC3)cc2)c1)C1CC1] |
| 3-(4-(4-(1-hydroxycyclobutane-1- carbonyl)piperazine-1- carbonyl)phenyl)quinolin-2(1H)- one | 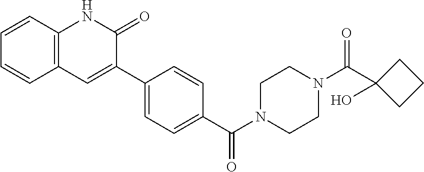 [SMILES: O=C(c1ccc(-c2cc3ccccc3[nH]c2=O)cc1)N1CCN(C(=O)C2(O)CCC2)CC1] |
| 3-(4-(4-(oxetane-2- carbonyl)piperazine-1- carbonyl)phenyl)quinolin-2(1H)- one | 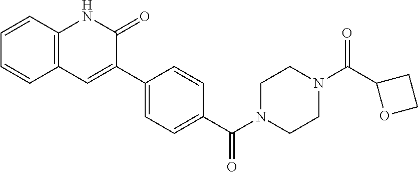 [SMILES: O=C(c1ccc(-c2cc3ccccc3[nH]c2=O)cc1)N1CCN(C(=O)C2CCO2)CC1] |
| 5-(4-(4-(1-hydroxycyclobutane-1- carbonyl)piperazine-1- carbonyl)phenyl)-1H-indole-2- carbonitrile | 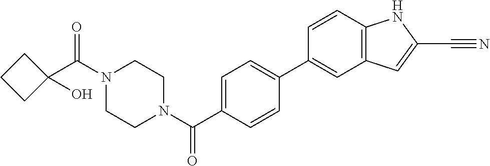 [SMILES: N#Cc1cc2cc(-c3ccc(C(=O)N4CCN(C(=O)C5(O)CCC5)CC4)cc3)ccc2[nH]1] |
| 5-(4-(4-(oxetane-2- carbonyl)piperazine-1- carbonyl)phenyl)-1H-indole-2- carbonitrile | 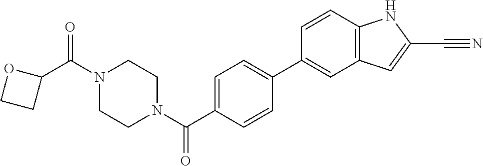 [SMILES: N#Cc1cc2cc(-c3ccc(C(=O)N4CCN(C(=O)C5CCO5)CC4)cc3)ccc2[nH]1] |
| (4-(1-hydroxycyclobutane-1- carbonyl)piperazin-1-yl)(4-(1- methyl-1H-benzo[d]imidazol-5- yl)phenyl)methanone | 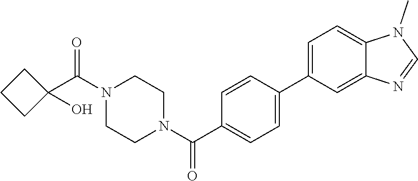 [SMILES: Cn1cnc2cc(-c3ccc(C(=O)N4CCN(C(=O)C5(O)CCC5)CC4)cc3)ccc21] |
| (4-(4-(1-methyl-1H- benzo[d]imidazol-5- yl)benzoyl)piperazin-1-yl)(oxetan-2- yl)methanone | 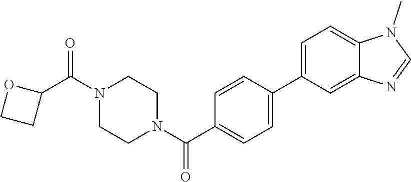 [SMILES: Cn1cnc2cc(-c3ccc(C(=O)N4CCN(C(=O)C5CCO5)CC4)cc3)ccc21] |
| (4-(4-(1,5-dihydroimidazo[1,2- b]pyridazin-6-yl)benzoyl)piperazin- 1-yl)(1- hydroxycyclobutyl)methanone | 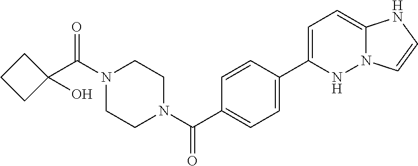 [SMILES: O=C(c1ccc(C2=CC=C3NC=CN3N2)cc1)N1CCN(C(=O)C2(O)CCC2)CC1] |
| (4-(4-(imidazo[1,2-b]pyridazin-6- yl)benzoyl)piperazin-1-yl)(oxetan-2- yl)methanone | 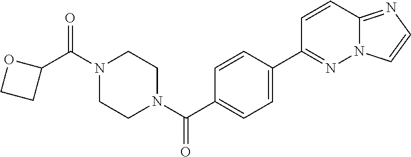 [SMILES: O=C(c1ccc(-c2ccc3nccn3n2)cc1)N1CCN(C(=O)C2CCO2)CC1] |
| (4-(4-(furo[3,2-b]pyridin-5- yl)benzoyl)piperazin-1-yl)(1- hydroxycyclobutyl)methanone | 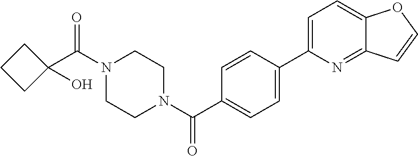 [SMILES: O=C(c1ccc(-c2ccc3occc3n2)cc1)N1CCN(C(=O)C2(O)CCC2)CC1] |
| (4-(4-(furo[3,2-b]pyridin-5- yl)benzoyl)piperazin-1-yl)(oxetan-2- yl)methanone | 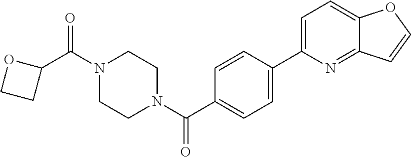 [SMILES: O=C(c1ccc(-c2ccc3occc3n2)cc1)N1CCN(C(=O)C2CCO2)CC1] |
| (4-(4-(1H-indazol-3- yl)benzoyl)piperazin-1-yl)(1- hydroxycyclobutyl)methanone | 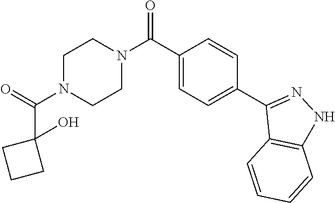 [SMILES: O=C(c1ccc(-c2n[nH]c3ccccc23)cc1)N1CCN(C(=O)C2(O)CCC2)CC1] |
| (4-(4-(1H-indazol-3- yl)benzoyl)piperazin-1-yl)(oxetan-2- yl)methanone | 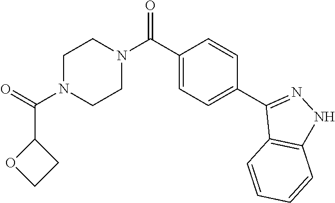 [SMILES: O=C(c1ccc(-c2n[nH]c3ccccc23)cc1)N1CCN(C(=O)C2CCO2)CC1] |
| (4-(4-(1,3-dimethyl-1H-indazol-5- yl)benzoyl)piperazin-1-yl)(1- hydroxycyclobutyl)methanone | 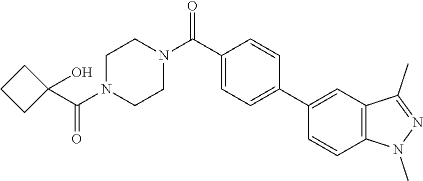 [SMILES: Cc1nn(C)c2ccc(-c3ccc(C(=O)N4CCN(C(=O)C5(O)CCC5)CC4)cc3)cc12] |
| (4-(4-(1,3-dimethyl-1H-indazol-5- yl)benzoyl)piperazin-1-yl)(oxetan-2- yl)methanone | 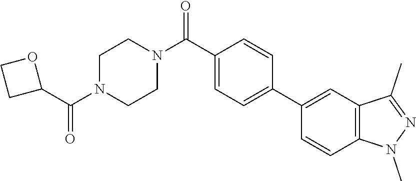 [SMILES: Cc1nn(C)c2ccc(-c3ccc(C(=O)N4CCN(C(=O)C5CCO5)CC4)cc3)cc12] |
| N-(4′-(4-(1-hydroxycyclobutane-1- carbonyl)piperazine-1-carbonyl)- [1,1′-biphenyl]-3- yl)cyclopropanesulfonamide | 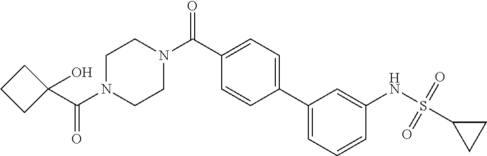 [SMILES: O=C(c1ccc(-c2cccc(NS(=O)(=O)C3CC3)c2)cc1)N1CCN(C(=O)C2(O)CCC2)CC1] |
| N-(4′-(4-(oxetane-2- carbonyl)piperazine-1-carbonyl)- [1,1′-biphenyl]-3- yl)cyclopropanesulfonamide | 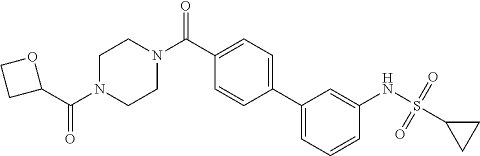 [SMILES: O=C(c1ccc(-c2cccc(NS(=O)(=O)C3CC3)c2)cc1)N1CCN(C(=O)C2CCO2)CC1] |
| (4-(4-(6-fluorobenzo[d]oxazol-2- yl)benzoyl)piperazin-1-yl)(1- hydroxycyclobutyl)methanone | 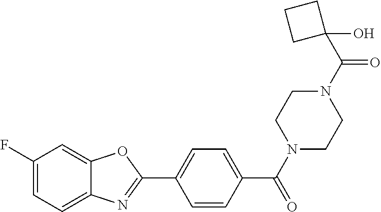 [SMILES: O=C(c1ccc(-c2nc3ccc(F)cc3o2)cc1)N1CCN(C(=O)C2(O)CCC2)CC1] |
| (4-(4-(6-fluorobenzo[d]oxazol-2- yl)benzoyl)piperazin-1-yl)(oxetan-2- yl)methanone | 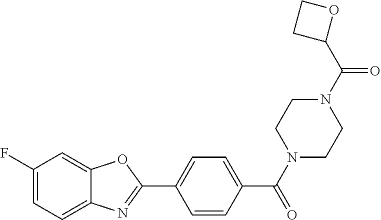 [SMILES: O=C(c1ccc(-c2nc3ccc(F)cc3o2)cc1)N1CCN(C(=O)C2CCO2)CC1] |
| (4-(4-(6-chlorobenzo[d]oxazol-2- yl)benzoyl)piperazin-1-yl)(1- hydroxycyclobutyl)methanone | 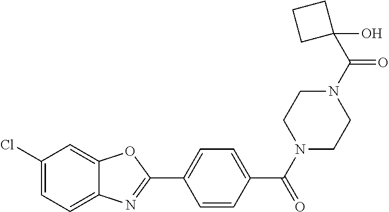 [SMILES: O=C(c1ccc(-c2nc3ccc(Cl)cc3o2)cc1)N1CCN(C(=O)C2(O)CCC2)CC1] |
| (4-(4-(6-chlorobenzo[d]oxazol-2- yl)benzoyl)piperazin-1-yl)(oxetan-2- yl)methanone | 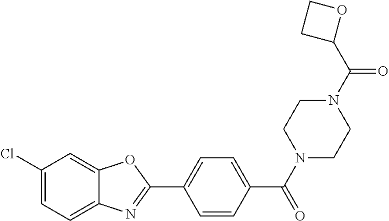 [SMILES: O=C(c1ccc(-c2nc3ccc(Cl)cc3o2)cc1)N1CCN(C(=O)C2CCO2)CC1] |
| 5-(4-(4-(1-hydroxycyclobutane-1- carbonyl)piperazine-1- carbonyl)phenyl)-1H-indole-3- carbonitrile | 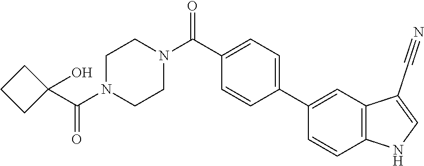 [SMILES: N#Cc1c[nH]c2ccc(-c3ccc(C(=O)N4CCN(C(=O)C5(O)CCC5)CC4)cc3)cc12] |
| 5-(4-(4-(oxetane-2- carbonyl)piperazine-1- carbonyl)phenyl)-1H-indole-3- carbonitrile | 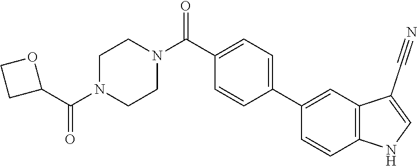 [SMILES: N#Cc1c[nH]c2ccc(-c3ccc(C(=O)N4CCN(C(=O)C5CCO5)CC4)cc3)cc12] |
| (4-(4-(benzo[d]thiazol-2- yl)benzoyl)piperazin-1-yl)(1- hydroxycyclobutyl)methanone | 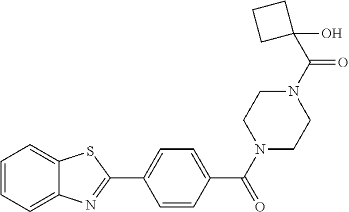 [SMILES: O=C(c1ccc(-c2nc3ccccc3s2)cc1)N1CCN(C(=O)C2(O)CCC2)CC1] |
| (4-(4-(benzo[d]thiazol-2- yl)benzoyl)piperazin-1-yl)(oxetan-2- yl)methanone | 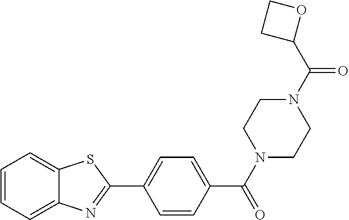 [SMILES: O=C(c1ccc(-c2nc3ccccc3s2)cc1)N1CCN(C(=O)C2CCO2)CC1] |
| (4-(1-hydroxycyclobutane-1- carbonyl)piperazin-1-yl)(4- (quinolin-2-yl)phenyl)methanone | 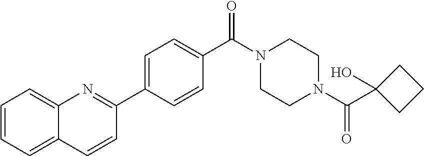 [SMILES: O=C(c1ccc(-c2ccc3ccccc3n2)cc1)N1CCN(C(=O)C2(O)CCC2)CC1] |
| oxetan-2-yl(4-(4-(quinolin-2- yl)benzoyl)piperazin-1- yl)methanone | 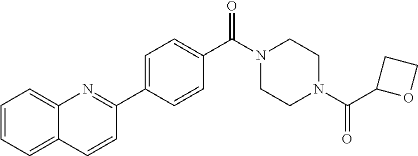 [SMILES: O=C(c1ccc(-c2ccc3ccccc3n2)cc1)N1CCN(C(=O)C2CCO2)CC1] |
| (4-(4-(1H-benzo[d]imidazol-5- yl)benzoyl)piperazin-1-yl)(1- hydroxycyclobutyl)methanone | 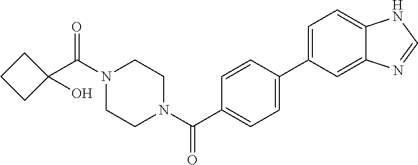 [SMILES: O=C(c1ccc(-c2ccc3[nH]cnc3c2)cc1)N1CCN(C(=O)C2(O)CCC2)CC1] |
| (4-(4-(1H-benzo[d]imidazol-5- yl)benzoyl)piperazin-1-yl)(oxetan-2- yl)methanone | 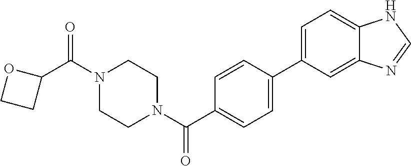 [SMILES: O=C(c1ccc(-c2ccc3[nH]cnc3c2)cc1)N1CCN(C(=O)C2CCO2)CC1] |
| (4-(4-(benzo[d]thiazol-6- yl)benzoyl)piperazin-1-yl)(1- hydroxycyclobutyl)methanone | 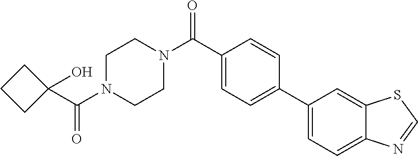 [SMILES: O=C(c1ccc(-c2ccc3ncsc3c2)cc1)N1CCN(C(=O)C2(O)CCC2)CC1] |
| (4-(4-(benzo[d]thiazol-6- yl)benzoyl)piperazin-1-yl)(oxetan-2- yl)methanone | 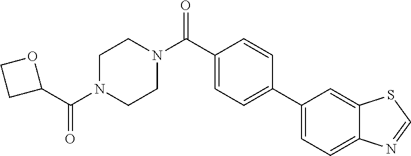 [SMILES: O=C(c1ccc(-c2ccc3ncsc3c2)cc1)N1CCN(C(=O)C2CCO2)CC1] |
| (4-(4-(benzo[d]oxazol-5- yl)benzoyl)piperazin-1-yl)(1- hydroxycyclobutyl)methanone | 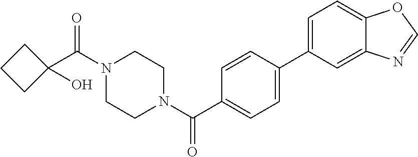 [SMILES: O=C(c1ccc(-c2ccc3ocnc3c2)cc1)N1CCN(C(=O)C2(O)CCC2)CC1] |
| (4-(4-(benzo[d]oxazol-5- yl)benzoyl)piperazin-1-yl)(oxetan-2- yl)methanone | 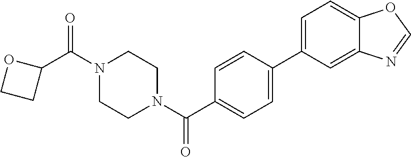 [SMILES: O=C(c1ccc(-c2ccc3ocnc3c2)cc1)N1CCN(C(=O)C2CCO2)CC1] |
| (4-(4-(benzo[d]thiazol-5- yl)benzoyl)piperazin-1-yl)(1- hydroxycyclobutyl)methanone | 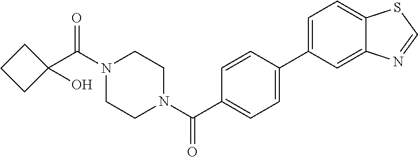 [SMILES: O=C(c1ccc(-c2ccc3scnc3c2)cc1)N1CCN(C(=O)C2(O)CCC2)CC1] |
| (4-(4-(benzo[d]thiazol-5- yl)benzoyl)piperazin-1-yl)(oxetan-2- yl)methanone | 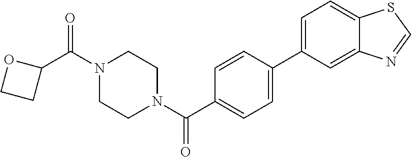 [SMILES: O=C(c1ccc(-c2ccc3scnc3c2)cc1)N1CCN(C(=O)C2CCO2)CC1] |
| (4-(1-hydroxycyclobutane-1- carbonyl)piperazin-1-yl)(4-(1- methyl-1H-indazol-6- yl)phenyl)methanone | 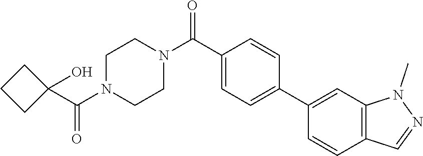 [SMILES: Cn1ncc2ccc(-c3ccc(C(=O)N4CCN(C(=O)C5(O)CCC5)CC4)cc3)cc21] |
| (4-(4-(1-methyl-1H-indazol-6- yl)benzoyl)piperazin-1-yl)(oxetan-2- yl)methanone | 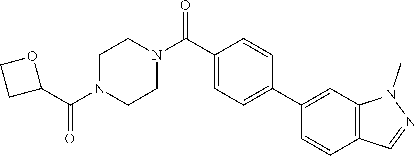 [SMILES: Cn1ncc2ccc(-c3ccc(C(=O)N4CCN(C(=O)C5CCO5)CC4)cc3)cc21] |
| N-cyclopropyl-4′-(4-(1- hydroxycyclobutane-1- carbonyl)piperazine-1-carbonyl)- [1,1′-biphenyl]-3-carboxamide | 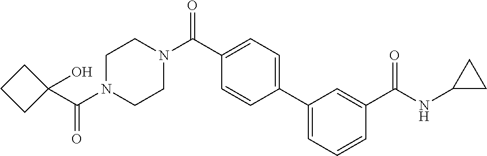 [SMILES: O=C(NC1CC1)c1cccc(-c2ccc(C(=O)N3CCN(C(=O)C4(O)CCC4)CC3)cc2)c1] |
| N-cyclopropyl-4′-(4-(oxetane-2- carbonyl)piperazine-1-carbonyl)- [1,1′-biphenyl]-3-carboxamide | 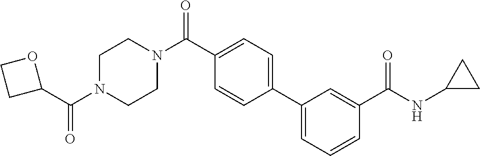 [SMILES: O=C(NC1CC1)c1cccc(-c2ccc(C(=O)N3CCN(C(=O)C4CCO4)CC3)cc2)c1] |
| (4-(1-hydroxycyclobutane-1- carbonyl)piperazin-1-yl)(4-(2- methyl-2H-indazol-6- yl)phenyl)methanone | 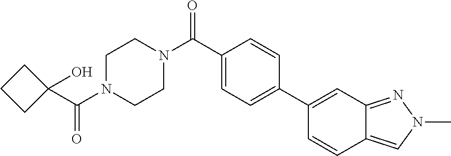 [SMILES: Cn1cc2ccc(-c3ccc(C(=O)N4CCN(C(=O)C5(O)CCC5)CC4)cc3)cc2n1] |
| (4-(4-(2-methyl-2H-indazol-6- yl)benzoyl)piperazin-1-yl)(oxetan-2- yl)methanone | 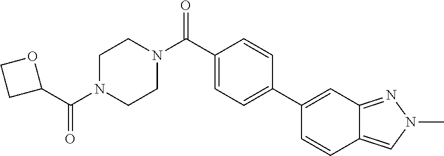 [SMILES: Cn1cc2ccc(-c3ccc(C(=O)N4CCN(C(=O)C5CCO5)CC4)cc3)cc2n1] |
| (4-(4′-chloro-2′-fluoro-[1,1′- biphenyl]-4-carbonyl)piperazin-1- yl)(1-hydroxycyclobutyl)methanone | 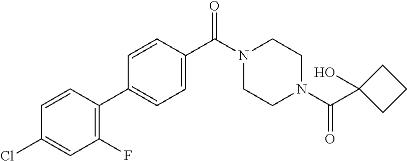 [SMILES: O=C(c1ccc(-c2ccc(Cl)cc2F)cc1)N1CCN(C(=O)C2(O)CCC2)CC1] |
| (4-(4′-chloro-2′-fluoro-[1,1′- biphenyl]-4-carbonyl)piperazin-1- yl)(oxetan-2-yl)methanone | 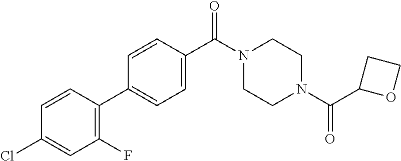 [SMILES: O=C(c1ccc(-c2ccc(Cl)cc2F)cc1)N1CCN(C(=O)C2CCO2)CC1] |
| (4-(5′-chloro-2′-fluoro-[1,1′- biphenyl]-4-carbonyl)piperazin-1- yl)(1-hydroxycyclobutyl)methanone | 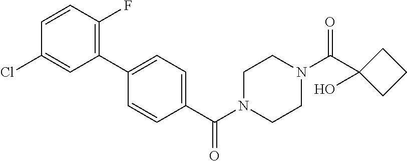 [SMILES: O=C(c1ccc(-c2cc(Cl)ccc2F)cc1)N1CCN(C(=O)C2(O)CCC2)CC1] |
| (4-(5′-chloro-2′-fluoro-[1,1′- biphenyl]-4-carbonyl)piperazin-1- yl)(oxetan-2-yl)methanone | 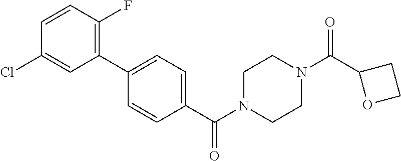 [SMILES: O=C(c1ccc(-c2cc(Cl)ccc2F)cc1)N1CCN(C(=O)C2CCO2)CC1] |
| (4-(1-aminocyclobutane-1- carbonyl)piperazin-1-yl)(3′- (cyclopropylsulfonyl)-[1,1′- biphenyl]-4-yl)methanone | 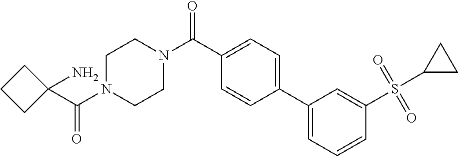 [SMILES: NC1(C(=O)N2CCN(C(=O)c3ccc(-c4cccc(S(=O)(=O)C5CC5)c4)cc3)CC2)CCC1] |
| (4-(1-aminocyclopropane-1- carbonyl)piperazin-1-yl)(3′- (cyclopropylsulfonyl)-[1,1′- biphenyl]-4-yl)methanone | 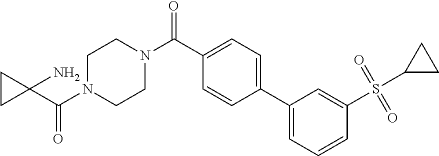 [SMILES: NC1(C(=O)N2CCN(C(=O)c3ccc(-c4cccc(S(=O)(=O)C5CC5)c4)cc3)CC2)CC1] |
| (4-(1-aminocyclobutane-1- carbonyl)piperazin-1-yl)(4-(3- chloroimidazo[1,2-a]pyridin-2- yl)phenyl)methanone | 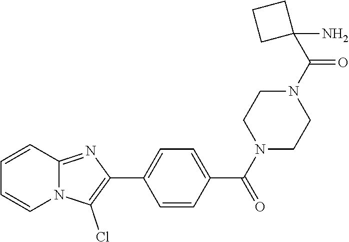 [SMILES: NC1(C(=O)N2CCN(C(=O)c3ccc(-c4nc5ccccn5c4Cl)cc3)CC2)CCC1] |
| (4-(1-aminocyclopropane-1- carbonyl)piperazin-1-yl)(4-(3- chloroimidazo[1,2-a]pyridin-2- yl)phenyl)methanone | 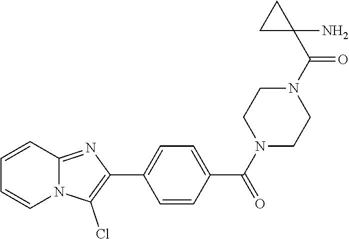 [SMILES: NC1(C(=O)N2CCN(C(=O)c3ccc(-c4nc5ccccn5c4Cl)cc3)CC2)CC1] |
| (4-(1-aminocyclobutane-1- carbonyl)piperazin-1-yl)(3′- cyclopropoxy-[1,1′-biphenyl]-4- yl)methanone | 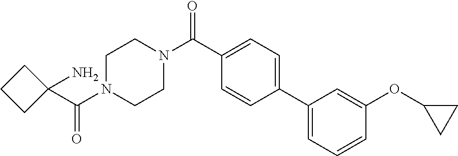 [SMILES: NC1(C(=O)N2CCN(C(=O)c3ccc(-c4cccc(OC5CC5)c4)cc3)CC2)CCC1] |
| (4-(1-aminocyclopropane-1- carbonyl)piperazin-1-yl)(3′- cyclopropoxy-[1,1′-biphenyl]-4- yl)methanone | 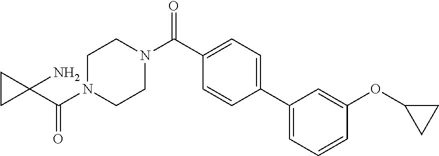 [SMILES: NC1(C(=O)N2CCN(C(=O)c3ccc(-c4cccc(OC5CC5)c4)cc3)CC2)CC1] |
| N-(4′-(4-(1-aminocyclobutane-1- carbonyl)piperazine-1-carbonyl)- [1,1′-biphenyl]-3- yl)cyclopropanecarboxamide | 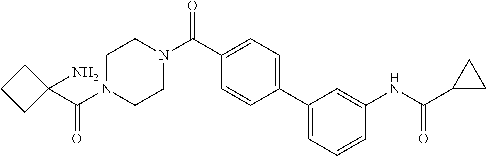 [SMILES: NC1(C(=O)N2CCN(C(=O)c3ccc(-c4cccc(NC(=O)C5CC5)c4)cc3)CC2)CCC1] |
| N-(4′-(4-(1-aminocyclopropane-1- carbonyl)piperazine-1-carbonyl)- [1,1′-biphenyl]-3- yl)cyclopropanecarboxamide | 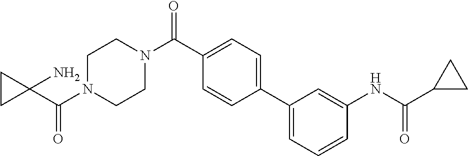 [SMILES: NC1(C(=O)N2CCN(C(=O)c3ccc(-c4cccc(NC(=O)C5CC5)c4)cc3)CC2)CC1] |
| 3-(4-(4-(1-aminocyclobutane-1- carbonyl)piperazine-1- carbonyl)phenyl)quinolin-2(1H)- one | 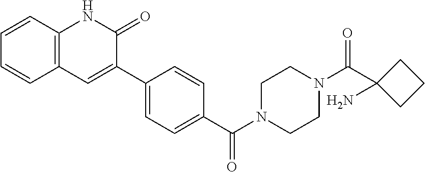 [SMILES: NC1(C(=O)N2CCN(C(=O)c3ccc(-c4cc5ccccc5[nH]c4=O)cc3)CC2)CCC1] |
| 3-(4-(4-(1-aminocyclopropane-1- carbonyl)piperazine-1- carbonyl)phenyl)quinolin-2(1H)- one | 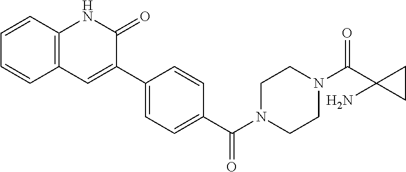 [SMILES: NC1(C(=O)N2CCN(C(=O)c3ccc(-c4cc5ccccc5[nH]c4=O)cc3)CC2)CC1] |
| 5-(4-(4-(1-aminocyclobutane-1- carbonyl)piperazine-1- carbonyl)phenyl)-1H-indole-2- carbonitrile | 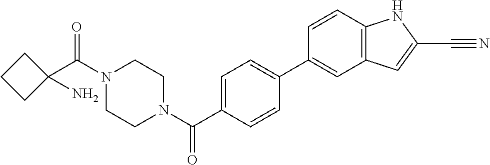 [SMILES: N#Cc1cc2cc(-c3ccc(C(=O)N4CCN(C(=O)C5(N)CCC5)CC4)cc3)ccc2[nH]1] |
| 5-(4-(4-(1-aminocyclopropane-1- carbonyl)piperazine-1- carbonyl)phenyl)-1H-indole-2- carbonitrile | 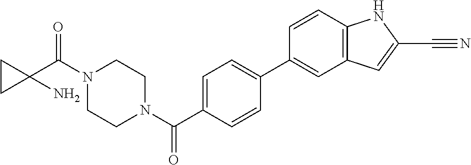 [SMILES: N#Cc1cc2cc(-c3ccc(C(=O)N4CCN(C(=O)C5(N)CC5)CC4)cc3)ccc2[nH]1] |
| (4-(1-aminocyclobutane-1- carbonyl)piperazin-1-yl)(4-(1- methyl-1H-benzo[d]imidazol-5- yl)phenyl)methanone | 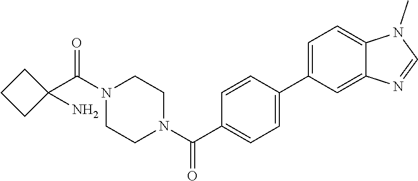 [SMILES: Cn1cnc2cc(-c3ccc(C(=O)N4CCN(C(=O)C5(N)CCC5)CC4)cc3)ccc21] |
| (4-(1-aminocyclopropane-1- carbonyl)piperazin-1-yl)(4-(1- methyl-1H-benzo[d]imidazol-5- yl)phenyl)methanone | 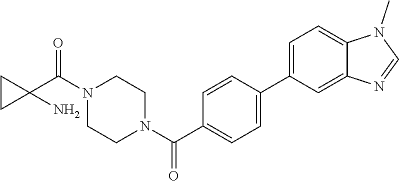 [SMILES: Cn1cnc2cc(-c3ccc(C(=O)N4CCN(C(=O)C5(N)CC5)CC4)cc3)ccc21] |
| (4-(1-aminocyclobutane-1- carbonyl)piperazin-1-yl)(4- (imidazo[1,2-b]pyridazin-6- yl)phenyl)methanone | 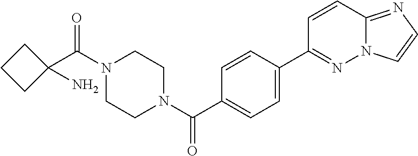 [SMILES: NC1(C(=O)N2CCN(C(=O)c3ccc(-c4ccc5nccn5n4)cc3)CC2)CCC1] |
| (4-(1-aminocyclopropane-1- carbonyl)piperazin-1-yl)(4- (imidazo[1,2-b]pyridazin-6- yl)phenyl)methanone | 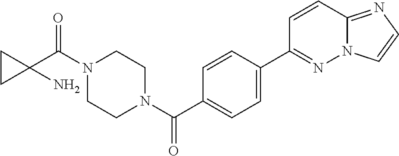 [SMILES: NC1(C(=O)N2CCN(C(=O)c3ccc(-c4ccc5nccn5n4)cc3)CC2)CC1] |
| (4-(1-aminocyclobutane-1- carbonyl)piperazin-1-yl)(4- (furo[3,2-b]pyridin-5- yl)phenyl)methanone | 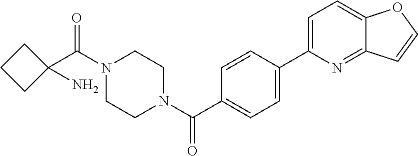 [SMILES: NC1(C(=O)N2CCN(C(=O)c3ccc(-c4ccc5occc5n4)cc3)CC2)CCC1] |
| (4-(1-aminocyclopropane-1- carbonyl)piperazin-1-yl)(4- (furo[3,2-b]pyridin-5- yl)phenyl)methanone | 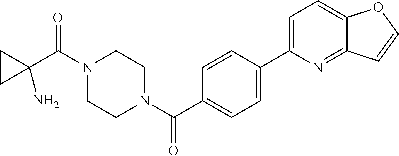 [SMILES: NC1(C(=O)N2CCN(C(=O)c3ccc(-c4ccc5occc5n4)cc3)CC2)CC1] |
| (4-(4-(1H-indazol-3- yl)benzoyl)piperazin-1-yl)(1- aminocyclobutyl)methanone | 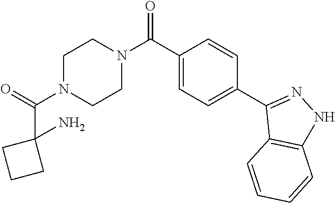 [SMILES: NC1(C(=O)N2CCN(C(=O)c3ccc(-c4n[nH]c5ccccc45)cc3)CC2)CCC1] |
| (4-(4-(1H-indazol-3- yl)benzoyl)piperazin-1-yl)(1- aminocyclopropyl)methanone | 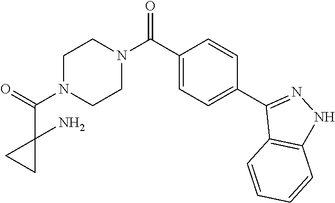 [SMILES: NC1(C(=O)N2CCN(C(=O)c3ccc(-c4n[nH]c5ccccc45)cc3)CC2)CC1] |
| (4-(1-aminocyclobutane-1- carbonyl)piperazin-1-yl)(4-(1,3- dimethyl-1H-indazol-5- yl)phenyl)methanone | 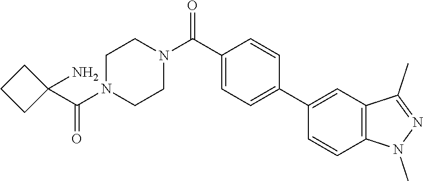 [SMILES: Cc1nn(C)c2ccc(-c3ccc(C(=O)N4CCN(C(=O)C5(N)CCC5)CC4)cc3)cc12] |
| (4-(1-aminocyclopropane-1- carbonyl)piperazin-1-yl)(4-(1,3- dimethyl-1H-indazol-5- yl)phenyl)methanone | 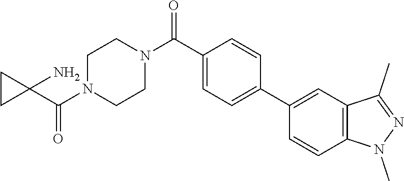 [SMILES: Cc1nn(C)c2ccc(-c3ccc(C(=O)N4CCN(C(=O)C5(N)CC5)CC4)cc3)cc12] |
| N-(4′-(4-(1-aminocyclobutane-1- carbonyl)piperazine-1-carbonyl)- [1,1′-biphenyl]-3- yl)cyclopropanesulfonamide | 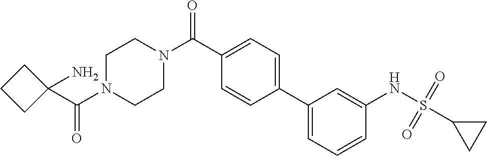 [SMILES: NC1(C(=O)N2CCN(C(=O)c3ccc(-c4cccc(NS(=O)(=O)C5CC5)c4)cc3)CC2)CCC1] |
| N-(4′-(4-(1-aminocyclopropane-1- carbonyl)piperazine-1-carbonyl)- [1,1′-biphenyl]-3- yl)cyclopropanesulfonamide | 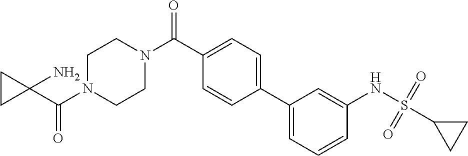 [SMILES: NC1(C(=O)N2CCN(C(=O)c3ccc(-c4cccc(NS(=O)(=O)C5CC5)c4)cc3)CC2)CC1] |
| (4-(1-aminocyclobutane-1- carbonyl)piperazin-1-yl)(4-(6- fluorobenzo[d]oxazol-2- yl)phenyl)methanone | 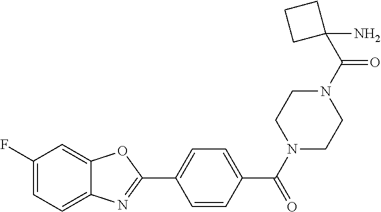 [SMILES: NC1(C(=O)N2CCN(C(=O)c3ccc(-c4nc5ccc(F)cc5o4)cc3)CC2)CCC1] |
| (4-(1-aminocyclopropane-1- carbonyl)piperazin-1-yl)(4-(6- fluorobenzo[d]oxazol-2- yl)phenyl)methanone | 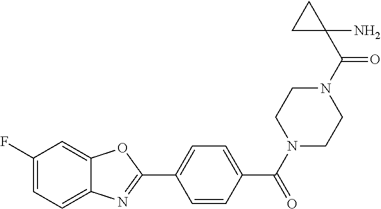 [SMILES: NC1(C(=O)N2CCN(C(=O)c3ccc(-c4nc5ccc(F)cc5o4)cc3)CC2)CC1] |
| (4-(1-aminocyclobutane-1- carbonyl)piperazin-1-yl)(4-(6- chlorobenzo[d]oxazol-2- yl)phenyl)methanone | 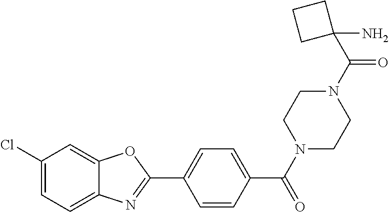 [SMILES: NC1(C(=O)N2CCN(C(=O)c3ccc(-c4nc5ccc(Cl)cc5o4)cc3)CC2)CCC1] |
| (4-(1-aminocyclopropane-1- carbonyl)piperazin-1-yl)(4-(6- chlorobenzo[d]oxazol-2- yl)phenyl)methanone | 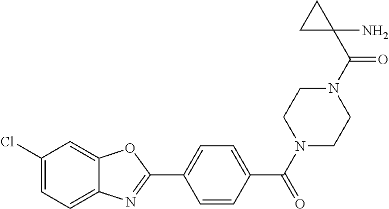 [SMILES: NC1(C(=O)N2CCN(C(=O)c3ccc(-c4nc5ccc(Cl)cc5o4)cc3)CC2)CC1] |
| 5-(4-(4-(1-aminocyclobutane-1- carbonyl)piperazine-1- carbonyl)phenyl)-1H-indole-3- carbonitrile | 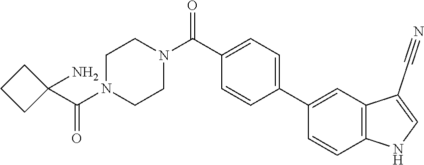 [SMILES: N#Cc1c[nH]c2ccc(-c3ccc(C(=O)N4CCN(C(=O)C5(N)CCC5)CC4)cc3)cc12] |
| 5-(4-(4-(1-aminocyclopropane-1- carbonyl)piperazine-1- carbonyl)phenyl)-1H-indole-3- carbonitrile | 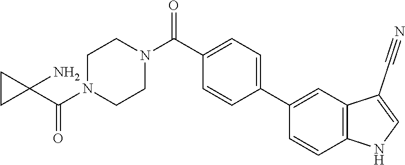 [SMILES: N#Cc1c[nH]c2ccc(-c3ccc(C(=O)N4CCN(C(=O)C5(N)CC5)CC4)cc3)cc12] |
| (4-(1-aminocyclobutane-1- carbonyl)piperazin-1-yl)(4- (benzo[d]thiazol-2- yl)phenyl)methanone | 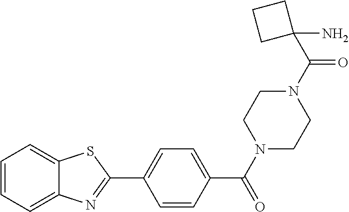 [SMILES: NC1(C(=O)N2CCN(C(=O)c3ccc(-c4nc5ccccc5s4)cc3)CC2)CCC1] |
| (4-(1-aminocyclopropane-1- carbonyl)piperazin-1-yl)(4- (benzo[d]thiazol-2- yl)phenyl)methanone | 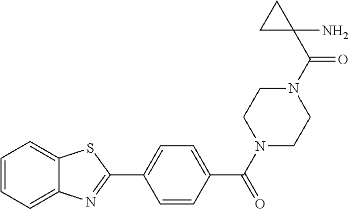 [SMILES: NC1(C(=O)N2CCN(C(=O)c3ccc(-c4nc5ccccc5s4)cc3)CC2)CC1] |
| (4-(1-aminocyclobutane-1- carbonyl)piperazin-1-yl)(4- (quinolin-2-yl)phenyl)methanone | 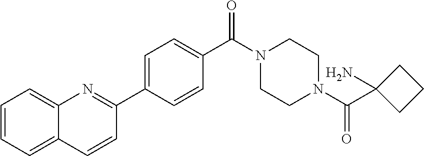 [SMILES: NC1(C(=O)N2CCN(C(=O)c3ccc(-c4ccc5ccccc5n4)cc3)CC2)CCC1] |
| (4-(1-aminocyclopropane-1- carbonyl)piperazin-1-yl)(4- (quinolin-2-yl)phenyl)methanone | 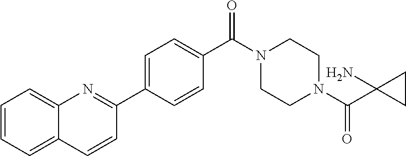 [SMILES: NC1(C(=O)N2CCN(C(=O)c3ccc(-c4ccc5ccccc5n4)cc3)CC2)CC1] |
| (4-(4-(1H-benzo[d]imidazol-5- yl)benzoyl)piperazin-1-yl)(1- aminocyclobutyl)methanone | 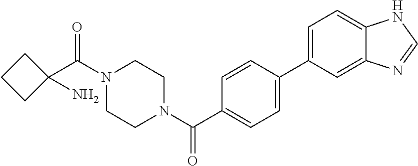 [SMILES: NC1(C(=O)N2CCN(C(=O)c3ccc(-c4ccc5[nH]cnc5c4)cc3)CC2)CCC1] |
| (4-(4-(1H-benzo[d]imidazol-5- yl)benzoyl)piperazin-1-yl)(1- aminocyclopropyl)methanone | 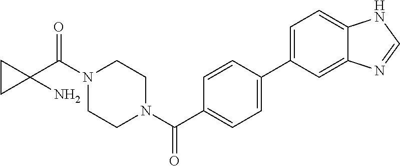 [SMILES: NC1(C(=O)N2CCN(C(=O)c3ccc(-c4ccc5[nH]cnc5c4)cc3)CC2)CC1] |
| (4-(1-aminocyclobutane-1- carbonyl)piperazin-1-yl)(4- (benzo[d]thiazol-6- yl)phenyl)methanone | 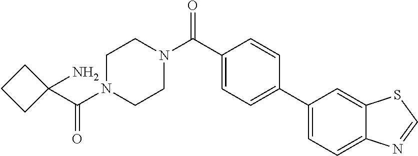 [SMILES: NC1(C(=O)N2CCN(C(=O)c3ccc(-c4ccc5ncsc5c4)cc3)CC2)CCC1] |
| (4-(1-aminocyclopropane-1- carbonyl)piperazin-1-yl)(4- (benzo[d]thiazol-6- yl)phenyl)methanone | 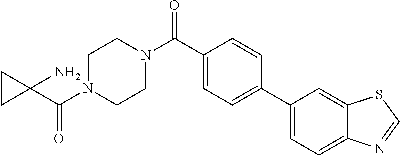 [SMILES: NC1(C(=O)N2CCN(C(=O)c3ccc(-c4ccc5ncsc5c4)cc3)CC2)CC1] |
| (4-(1-aminocyclobutane-1- carbonyl)piperazin-1-yl)(4- (benzo[d]oxazol-5- yl)phenyl)methanone | 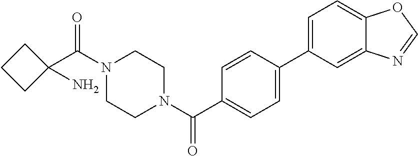 [SMILES: NC1(C(=O)N2CCN(C(=O)c3ccc(-c4ccc5ocnc5c4)cc3)CC2)CCC1] |
| (4-(1-aminocyclopropane-1- carbonyl)piperazin-1-yl)(4- (benzo[d]oxazol-5- yl)phenyl)methanone | 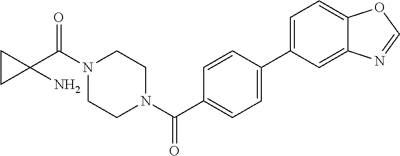 [SMILES: NC1(C(=O)N2CCN(C(=O)c3ccc(-c4ccc5ocnc5c4)cc3)CC2)CC1] |
| (4-(1-aminocyclobutane-1- carbonyl)piperazin-1-yl)(4- (benzo[d]thiazol-5- yl)phenyl)methanone | 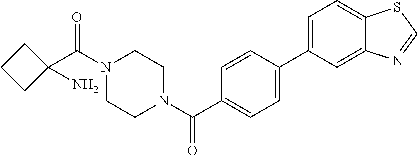 [SMILES: NC1(C(=O)N2CCN(C(=O)c3ccc(-c4ccc5scnc5c4)cc3)CC2)CCC1] |
| (4-(1-aminocyclopropane-1- carbonyl)piperazin-1-yl)(4- (benzo[d]thiazol-5- yl)phenyl)methanone | 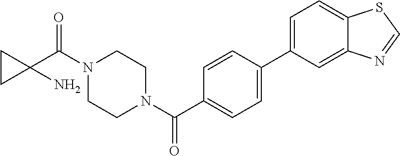 [SMILES: NC1(C(=O)N2CCN(C(=O)c3ccc(-c4ccc5scnc5c4)cc3)CC2)CC1] |
| (4-(1-aminocyclobutane-1- carbonyl)piperazin-1-yl)(4-(1- methyl-1H-indazol-6- yl)phenyl)methanone | 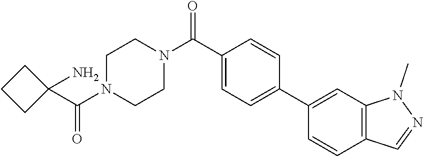 [SMILES: Cn1ncc2ccc(-c3ccc(C(=O)N4CCN(C(=O)C5(N)CCC5)CC4)cc3)cc21] |
| (4-(1-aminocyclopropane-1- carbonyl)piperazin-1-yl)(4-(1- methyl-1H-indazol-6- yl)phenyl)methanone | 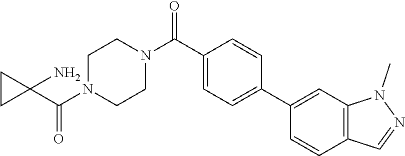 [SMILES: Cn1ncc2ccc(-c3ccc(C(=O)N4CCN(C(=O)C5(N)CC5)CC4)cc3)cc21] |
| 4′-(4-(1-aminocyclobutane-1- carbonyl)piperazine-1-carbonyl)-N- cyclopropyl-[1,1′-biphenyl]-3- carboxamide | 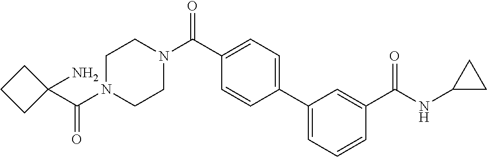 [SMILES: NC1(C(=O)N2CCN(C(=O)c3ccc(-c4cccc(C(=O)NC5CC5)c4)cc3)CC2)CCC1] |
| 4′-(4-(1-aminocyclopropane-1- carbonyl)piperazine-1-carbonyl)-N- cyclopropyl-[1,1′-biphenyl]-3- carboxamide | 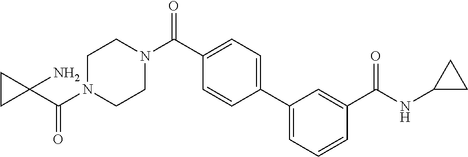 [SMILES: NC1(C(=O)N2CCN(C(=O)c3ccc(-c4cccc(C(=O)NC5CC5)c4)cc3)CC2)CC1] |
| (4-(1-aminocyclobutane-1- carbonyl)piperazin-1-yl)(4-(2- methyl-2H-indazol-6- yl)phenyl)methanone | 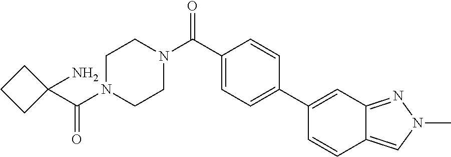 [SMILES: Cn1cc2ccc(-c3ccc(C(=O)N4CCN(C(=O)C5(N)CCC5)CC4)cc3)cc2n1] |
| (4-(1-aminocyclopropane-1- carbonyl)piperazin-1-yl)(4-(2- methyl-2H-indazol-6- yl)phenyl)methanone | 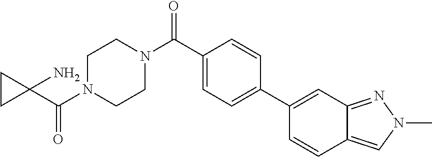 [SMILES: Cn1cc2ccc(-c3ccc(C(=O)N4CCN(C(=O)C5(N)CC5)CC4)cc3)cc2n1] |
| (4-(1-aminocyclobutane-1- carbonyl)piperazin-1-yl)(4′-chloro- 2′-fluoro-1,2,3,4,5,6-hexahydro- [1,1′-biphenyl]-4-yl)methanone | 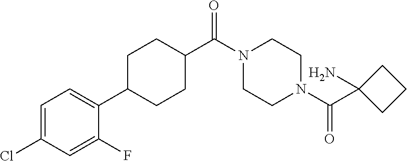 [SMILES: NC1(C(=O)N2CCN(C(=O)C3CCC(c4ccc(Cl)cc4F)CC3)CC2)CCC1] |
| (4-(1-aminocyclopropane-1- carbonyl)piperazin-1-yl)(4′-chloro- 2′-fluoro-[1,1′-biphenyl]-4- yl)methanone | 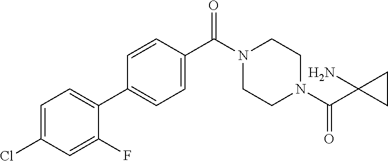 [SMILES: NC1(C(=O)N2CCN(C(=O)c3ccc(-c4ccc(Cl)cc4F)cc3)CC2)CC1] |
| (4-(1-aminocyclobutane-1- carbonyl)piperazin-1-yl)(5′-chloro- 2′-fluoro-[1,1′-biphenyl]-4- yl)methanone | 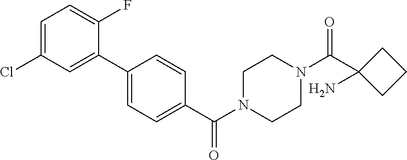 [SMILES: NC1(C(=O)N2CCN(C(=O)c3ccc(-c4cc(Cl)ccc4F)cc3)CC2)CCC1] |
| (4-(1-aminocyclopropane-1- carbonyl)piperazin-1-yl)(5′-chloro- 2′-fluoro-[1,1′-biphenyl]-4- yl)methanone | 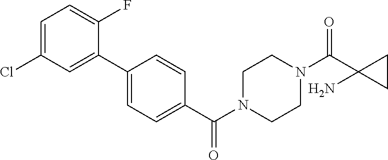 [SMILES: NC1(C(=O)N2CCN(C(=O)c3ccc(-c4cc(Cl)ccc4F)cc3)CC2)CC1] |
| (4-(3′-(cyclopropylsulfonyl)-[1,1′- biphenyl]-4-carbonyl)piperazin-1- yl)(3-hydroxyoxetan-3- yl)methanone | 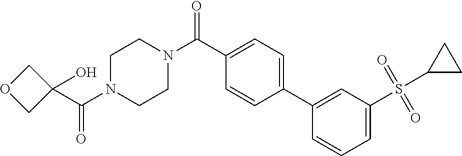 [SMILES: O=C(c1ccc(-c2cccc(S(=O)(=O)C3CC3)c2)cc1)N1CCN(C(=O)C2(O)COC2)CC1] |
| (3-aminooxetan-3-yl)(4-(3′- (cyclopropylsulfonyl)-[1,1′- biphenyl]-4-carbonyl)piperazin-1- yl)methanone | 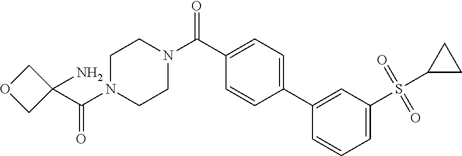 [SMILES: NC1(C(=O)N2CCN(C(=O)c3ccc(-c4cccc(S(=O)(=O)C5CC5)c4)cc3)CC2)COC1] |
| (4-(4-(3-chloroimidazo[1,2- a]pyridin-2-yl)benzoyl)piperazin-1- yl)(3-hydroxyoxetan-3- yl)methanone | 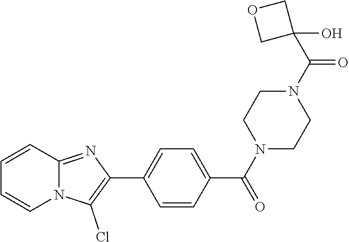 [SMILES: O=C(c1ccc(-c2nc3ccccn3c2Cl)cc1)N1CCN(C(=O)C2(O)COC2)CC1] |
| (3-aminooxetan-3-yl)(4-(4-(3- chloroimidazo[1,2-a]pyridin-2- yl)benzoyl)piperazin-1- yl)methanone | 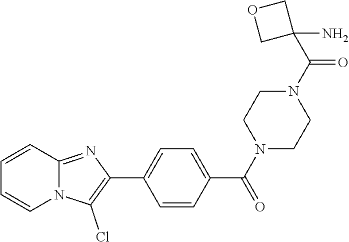 [SMILES: NC1(C(=O)N2CCN(C(=O)c3ccc(-c4nc5ccccn5c4Cl)cc3)CC2)COC1] |
| (4-(3′-cyclopropoxy-[1,1′-biphenyl]- 4-carbonyl)piperazin-1-yl)(3- hydroxyoxetan-3-yl)methanone | 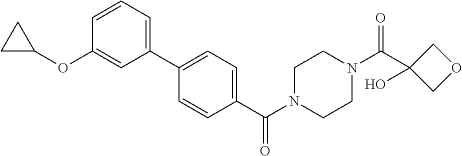 [SMILES: O=C(c1ccc(-c2cccc(OC3CC3)c2)cc1)N1CCN(C(=O)C2(O)COC2)CC1] |
| (3-aminooxetan-3-yl)(4-(3′- cyclopropoxy-[1,1′-biphenyl]-4- carbonyl)piperazin-1-yl)methanone | 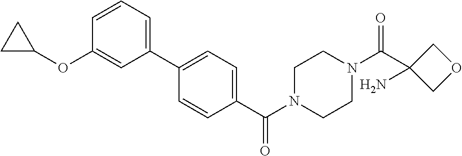 [SMILES: NC1(C(=O)N2CCN(C(=O)c3ccc(-c4cccc(OC5CC5)c4)cc3)CC2)COC1] |
| N-(4′-(4-(3-hydroxyoxetane-3- carbonyl)piperazine-1-carbonyl)- [1,1′-biphenyl]-3- yl)cyclopropanecarboxamide | 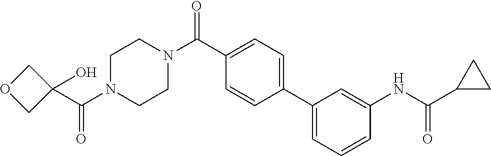 [SMILES: O=C(Nc1cccc(-c2ccc(C(=O)N3CCN(C(=O)C4(O)COC4)CC3)cc2)c1)C1CC1] |
| N-(4′-(4-(3-aminooxetane-3- carbonyl)piperazine-1-carbonyl)- [1,1′-biphenyl]-3- yl)cyclopropanecarboxamide | 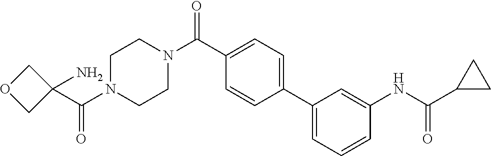 [SMILES: NC1(C(=O)N2CCN(C(=O)c3ccc(-c4cccc(NC(=O)C5CC5)c4)cc3)CC2)COC1] |
| 3-(4-(4-(3-hydroxyoxetane-3- carbonyl)piperazine-1- carbonyl)phenyl)quinolin-2(1H)- one | 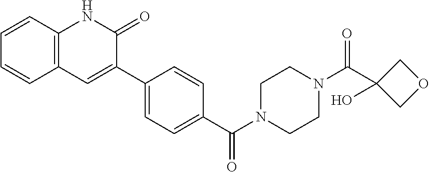 [SMILES: O=C(c1ccc(-c2cc3ccccc3[nH]c2=O)cc1)N1CCN(C(=O)C2(O)COC2)CC1] |
| 3-(4-(4-(3-aminooxetane-3- carbonyl)piperazine-1- carbonyl)phenyl)quinolin-2(1H)- one | 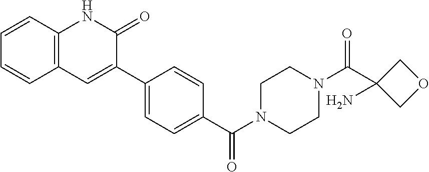 [SMILES: NC1(C(=O)N2CCN(C(=O)c3ccc(-c4cc5ccccc5[nH]c4=O)cc3)CC2)COC1] |
| 5-(4-(4-(3-hydroxyoxetane-3- carbonyl)piperazine-1- carbonyl)phenyl)-1H-indole-2- carbonitrile | 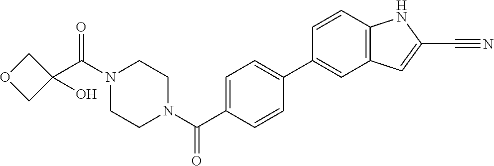 [SMILES: N#Cc1cc2cc(-c3ccc(C(=O)N4CCN(C(=O)C5(O)COC5)CC4)cc3)ccc2[nH]1] |
| 5-(4-(4-(3-aminooxetane-3- carbonyl)piperazine-1- carbonyl)phenyl)-1H-indole-2- carbonitrile | 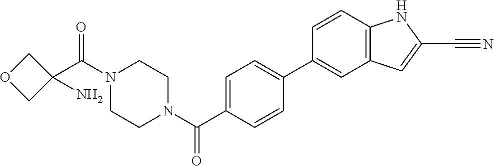 [SMILES: N#Cc1cc2cc(-c3ccc(C(=O)N4CCN(C(=O)C5(N)COC5)CC4)cc3)ccc2[nH]1] |
| (3-hydroxyoxetan-3-yl)(4-(4-(1- methyl-1H-benzo[d]imidazol-5- yl)benzoyl)piperazin-1- yl)methanone | 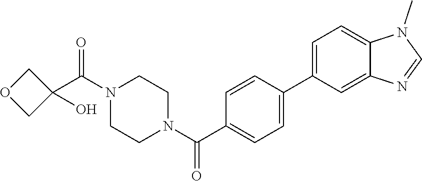 [SMILES: Cn1cnc2cc(-c3ccc(C(=O)N4CCN(C(=O)C5(O)COC5)CC4)cc3)ccc21] |
| (3-aminooxetan-3-yl)(4-(4-(1- methyl-1H-benzo[d]imidazol-5- yl)benzoyl)piperazin-1- yl)methanone | 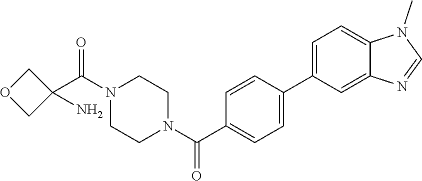 [SMILES: Cn1cnc2cc(-c3ccc(C(=O)N4CCN(C(=O)C5(N)COC5)CC4)cc3)ccc21] |
| (3-hydroxyoxetan-3-yl)(4-(4- (imidazo[1,2-b]pyridazin-6- yl)benzoyl)piperazin-1- yl)methanone | 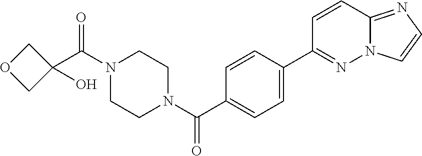 [SMILES: O=C(c1ccc(-c2ccc3nccn3n2)cc1)N1CCN(C(=O)C2(O)COC2)CC1] |
| (3-aminooxetan-3-yl)(4-(4- (imidazo[1,2-b]pyridazin-6- yl)benzoyl)piperazin-1- yl)methanone | 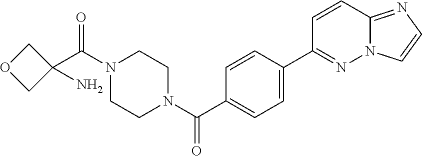 [SMILES: NC1(C(=O)N2CCN(C(=O)c3ccc(-c4ccc5nccn5n4)cc3)CC2)COC1] |
| (4-(4-(furo[3,2-b]pyridin-5- yl)benzoyl)piperazin-1-yl)(3- hydroxyoxetan-3-yl)methanone | 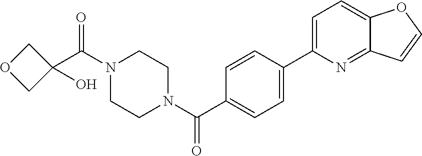 [SMILES: O=C(c1ccc(-c2ccc3occc3n2)cc1)N1CCN(C(=O)C2(O)COC2)CC1] |
| (3-aminooxetan-3-yl)(4-(4- (furo[3,2-b]pyridin-5- yl)benzoyl)piperazin-1-yl)methanone | 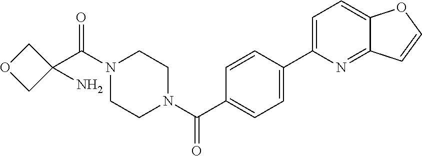 [SMILES: NC1(C(=O)N2CCN(C(=O)c3ccc(-c4ccc5occc5n4)cc3)CC2)COC1] |
| (4-(4-(1H-indazol-3- yl)benzoyl)piperazin-1-yl)(3- hydroxyoxetan-3-yl)methanone | 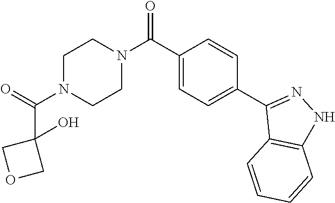 [SMILES: O=C(c1ccc(-c2n[nH]c3ccccc23)cc1)N1CCN(C(=O)C2(O)COC2)CC1] |
| (4-(4-(1H-indazol-3- yl)benzoyl)piperazin-1-yl)(3- aminooxetan-3-yl)methanone | 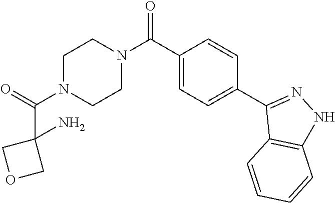 [SMILES: NC1(C(=O)N2CCN(C(=O)c3ccc(-c4n[nH]c5ccccc45)cc3)CC2)COC1] |
| (4-(4-(1,3-dimethyl-1H-indazol-5- yl)benzoyl)piperazin-1-yl)(3- hydroxyoxetan-3-yl)methanone | 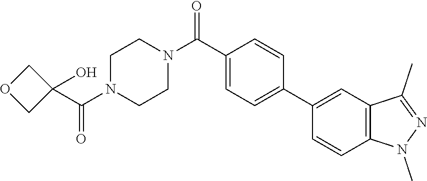 [SMILES: Cc1nn(C)c2ccc(-c3ccc(C(=O)N4CCN(C(=O)C5(O)COC5)CC4)cc3)cc12] |
| (3-aminooxetan-3-yl)(4-(4-(1,3- dimethyl-1H-indazol-5- yl)benzoyl)piperazin-1- yl)methanone | 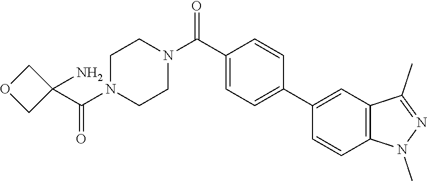 [SMILES: Cc1nn(C)c2ccc(-c3ccc(C(=O)N4CCN(C(=O)C5(N)COC5)CC4)cc3)cc12] |
| N-(4′-(4-(3-hydroxyoxetane-3- carbonyl)piperazine-1-carbonyl)- [1,1′-biphenyl]-3- yl)cyclopropanesulfonamide | 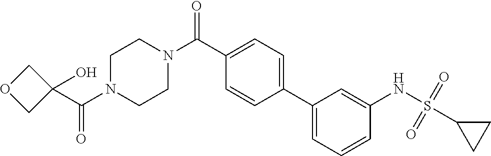 [SMILES: O=C(c1ccc(-c2cccc(NS(=O)(=O)C3CC3)c2)cc1)N1CCN(C(=O)C2(O)COC2)CC1] |
| N-(4′-(4-(3-aminooxetane-3- carbonyl)piperazine-1-carbonyl)- [1,1′-biphenyl]-3- yl)cyclopropanesulfonamide | 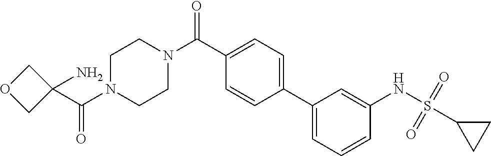 [SMILES: NC1(C(=O)N2CCN(C(=O)c3ccc(-c4cccc(NS(=O)(=O)C5CC5)c4)cc3)CC2)COC1] |
| (4-(4-(6-fluorobenzo[d]oxazol-2- yl)benzoyl)piperazin-1-yl)(3- hydroxyoxetan-3-yl)methanone | 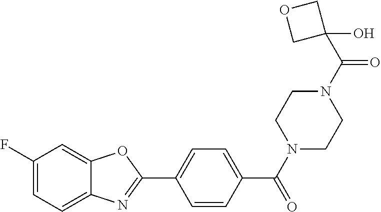 [SMILES: O=C(c1ccc(-c2nc3ccc(F)cc3o2)cc1)N1CCN(C(=O)C2(O)COC2)CC1] |
| (3-aminooxetan-3-yl)(4-(4-(6- fluorobenzo[d]oxazol-2- yl)benzoyl)piperazin-1- yl)methanone | 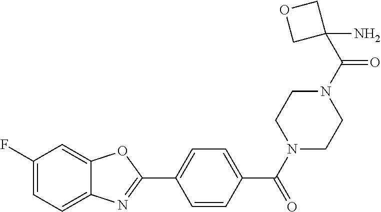 [SMILES: NC1(C(=O)N2CCN(C(=O)c3ccc(-c4nc5ccc(F)cc5o4)cc3)CC2)COC1] |
| (4-(4-(6-chlorobenzo[d]oxazol-2- yl)benzoyl)piperazin-1-yl)(3- hydroxyoxetan-3-yl)methanone | 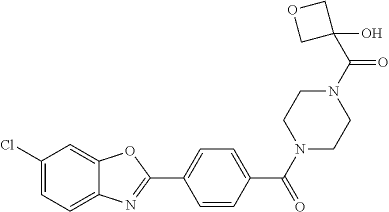 [SMILES: O=C(c1ccc(-c2nc3ccc(Cl)cc3o2)cc1)N1CCN(C(=O)C2(O)COC2)CC1] |
| (3-aminooxetan-3-yl)(4-(4-(6- chlorobenzo[d]oxazol-2- yl)benzoyl)piperazin-1- yl)methanone | 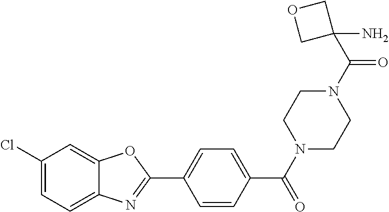 [SMILES: NC1(C(=O)N2CCN(C(=O)c3ccc(-c4nc5ccc(Cl)cc5o4)cc3)CC2)COC1] |
| 5-(4-(4-(3-hydroxyoxetane-3- carbonyl)piperazine-1- carbonyl)phenyl)-1H-indole-3- carbonitrile | 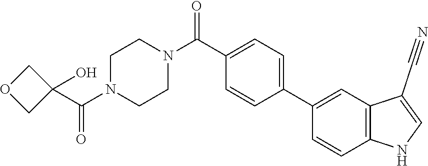 [SMILES: N#Cc1c[nH]c2ccc(-c3ccc(C(=O)N4CCN(C(=O)C5(O)COC5)CC4)cc3)cc12] |
| 5-(4-(4-(3-aminooxetane-3- carbonyl)piperazine-1- carbonyl)phenyl)-1H-indole-3- carbonitrile | 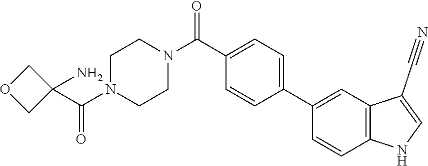 [SMILES: N#Cc1c[nH]c2ccc(-c3ccc(C(=O)N4CCN(C(=O)C5(N)COC5)CC4)cc3)cc12] |
| (4-(4-(benzo[d]thiazol-2- yl)benzoyl)piperazin-1-yl)(3- hydroxyoxetan-3-yl)methanone | 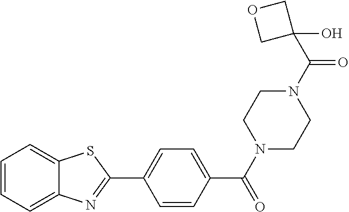 [SMILES: O=C(c1ccc(-c2nc3ccccc3s2)cc1)N1CCN(C(=O)C2(O)COC2)CC1] |
| (3-aminooxetan-3-yl)(4-(4- (benzo[d]thiazol-2- yl)benzoyl)piperazin-1- yl)methanone | 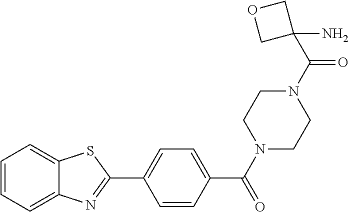 [SMILES: NC1(C(=O)N2CCN(C(=O)c3ccc(-c4nc5ccccc5s4)cc3)CC2)COC1] |
| (3-hydroxyoxetan-3-yl)(4-(4- (quinolin-2-yl)benzoyl)piperazin-1- yl)methanone | 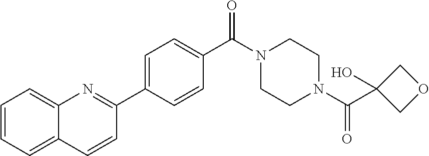 [SMILES: O=C(c1ccc(-c2ccc3ccccc3n2)cc1)N1CCN(C(=O)C2(O)COC2)CC1] |
| (3-aminooxetan-3-yl)(4-(4- (quinolin-2-yl)benzoyl)piperazin-1- yl)methanone | 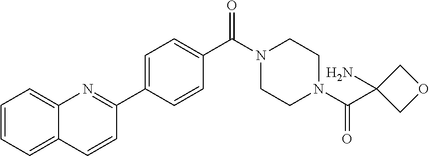 [SMILES: NC1(C(=O)N2CCN(C(=O)c3ccc(-c4ccc5ccccc5n4)cc3)CC2)COC1] |
| (4-(4-(1H-benzo[d]imidazol-5- yl)benzoyl)piperazin-1-yl)(3- hydroxyoxetan-3-yl)methanone | 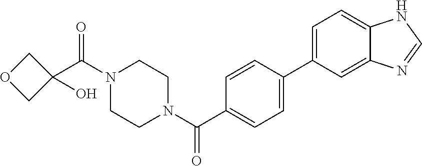 [SMILES: O=C(c1ccc(-c2ccc3[nH]cnc3c2)cc1)N1CCN(C(=O)C2(O)COC2)CC1] |
| (4-(4-(1H-benzo[d]imidazol-5- yl)benzoyl)piperazin-1-yl)(3- aminooxetan-3-yl)methanone | 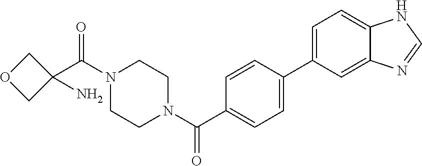 [SMILES: NC1(C(=O)N2CCN(C(=O)c3ccc(-c4ccc5[nH]cnc5c4)cc3)CC2)COC1] |
| (4-(4-(benzo[d]thiazol-6- yl)benzoyl)piperazin-1-yl)(3- hydroxyoxetan-3-yl)methanone | 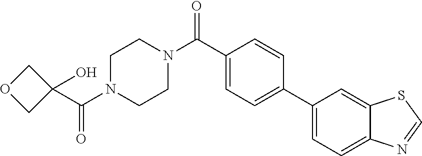 [SMILES: O=C(c1ccc(-c2ccc3ncsc3c2)cc1)N1CCN(C(=O)C2(O)COC2)CC1] |
| (3-aminooxetan-3-yl)(4-(4- (benzo[d]thiazol-6- yl)benzoyl)piperazin-1-yl)methanone | 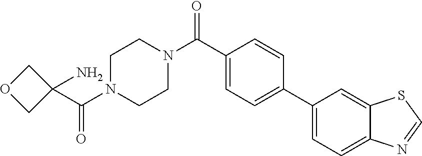 [SMILES: NC1(C(=O)N2CCN(C(=O)c3ccc(-c4ccc5ncsc5c4)cc3)CC2)COC1] |
| (4-(4-(benzo[d]oxazol-5- yl)benzoyl)piperazin-1-yl)(3- hydroxyoxetan-3-yl)methanone | 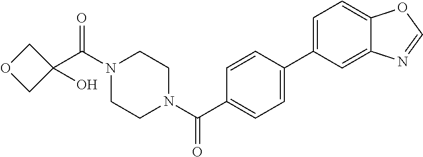 [SMILES: O=C(c1ccc(-c2ccc3ocnc3c2)cc1)N1CCN(C(=O)C2(O)COC2)CC1] |
| (3-aminooxetan-3-yl)(4-(4- (benzo[d]oxazol-5- yl)benzoyl)piperazin-1-yl)methanone | 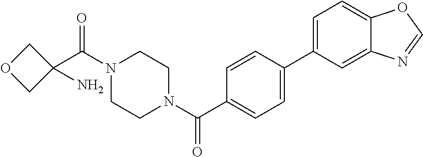 [SMILES: NC1(C(=O)N2CCN(C(=O)c3ccc(-c4ccc5ocnc5c4)cc3)CC2)COC1] |
| (4-(4-(benzo[d]thiazol-5- yl)benzoyl)piperazin-1-yl)(3- hydroxyoxetan-3-yl)methanone | 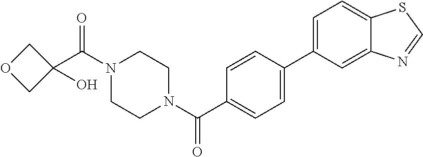 [SMILES: O=C(c1ccc(-c2ccc3scnc3c2)cc1)N1CCN(C(=O)C2(O)COC2)CC1] |
| (3-aminooxetan-3-yl)(4-(4- (benzo[d]thiazol-5- yl)benzoyl)piperazin-1-yl)methanone | 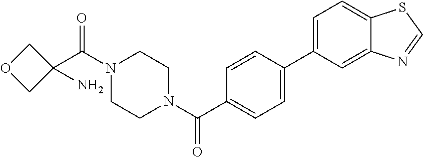 [SMILES: NC1(C(=O)N2CCN(C(=O)c3ccc(-c4ccc5scnc5c4)cc3)CC2)COC1] |
| (3-hydroxyoxetan-3-yl)(4-(4-(1- methyl-1H-indazol-6- yl)benzoyl)piperazin-1- yl)methanone | 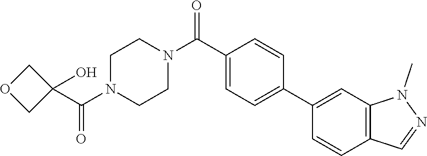 [SMILES: Cn1ncc2ccc(-c3ccc(C(=O)N4CCN(C(=O)C5(O)COC5)CC4)cc3)cc21] |
| (3-aminooxetan-3-yl)(4-(4-(1- methyl-1H-indazol-6- yl)benzoyl)piperazin-1- yl)methanone | 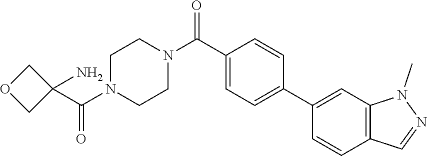 [SMILES: Cn1ncc2ccc(-c3ccc(C(=O)N4CCN(C(=O)C5(N)COC5)CC4)cc3)cc21] |
| N-cyclopropyl-4′-(4-(3- hydroxyoxetane-3- carbonyl)piperazine-1-carbonyl)- [1,1′-biphenyl]-3-carboxamide | 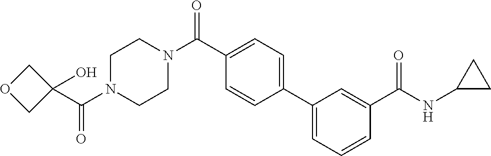 [SMILES: O=C(NC1CC1)c1cccc(-c2ccc(C(=O)N3CCN(C(=O)C4(O)COC4)CC3)cc2)c1] |
| 4′-(4-(3-aminooxetane-3- carbonyl)piperazine-1-carbonyl)-N- cyclopropyl-[1,1′-biphenyl]-3- carboxamide | 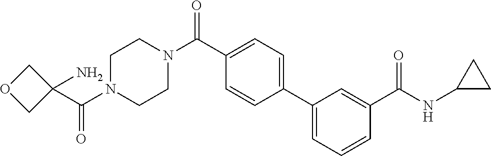 [SMILES: NC1(C(=O)N2CCN(C(=O)c3ccc(-c4cccc(C(=O)NC5CC5)c4)cc3)CC2)COC1] |
| (3-hydroxyoxetan-3-yl)(4-(4-(2- methyl-2H-indazol-6- yl)benzoyl)piperazin-1- yl)methanone | 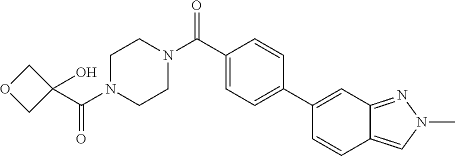 [SMILES: Cn1cc2ccc(-c3ccc(C(=O)N4CCN(C(=O)C5(O)COC5)CC4)cc3)cc2n1] |
| (3-aminooxetan-3-yl)(4-(4-(2- methyl-2H-indazol-6- yl)benzoyl)piperazin-1- yl)methanone | 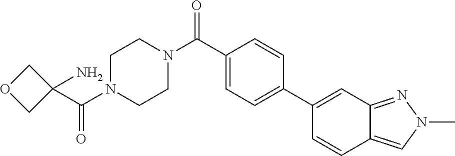 [SMILES: Cn1cc2ccc(-c3ccc(C(=O)N4CCN(C(=O)C5(N)COC5)CC4)cc3)cc2n1] |
| (4-(4′-chloro-2′-fluoro- 1′,2′,3′,4′,5′,6′-hexahydro-[1,1′- biphenyl]-4-carbonyl)piperazin-1- yl)(3-hydroxyoxetan-3- yl)methanone | 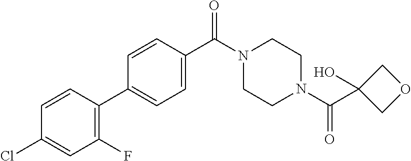 [SMILES: O=C(c1ccc(-c2ccc(Cl)cc2F)cc1)N1CCN(C(=O)C2(O)COC2)CC1] |
| (3-aminooxetan-3-yl)(4-(4′-chloro- 2′-fluoro-[1,1′-biphenyl]-4- carbonyl)piperazin-1-yl)methanone | 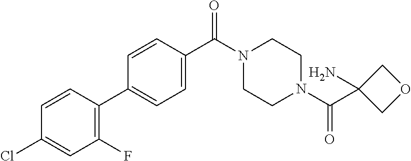 [SMILES: NC1(C(=O)N2CCN(C(=O)c3ccc(-c4ccc(Cl)cc4F)cc3)CC2)COC1] |
| (4-(5′-chloro-2′-fluoro-[1,1′- biphenyl]-4-carbonyl)piperazin-1- yl)(3-hydroxyoxetan-3- yl)methanone | 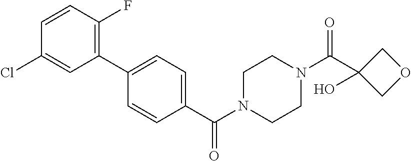 [SMILES: O=C(c1ccc(-c2cc(Cl)ccc2F)cc1)N1CCN(C(=O)C2(O)COC2)CC1] |
| (3-aminooxetan-3-yl)(4-(5′-chloro- 2′-fluoro-[1,1′-biphenyl]-4- carbonyl)piperazin-1-yl)methanone | 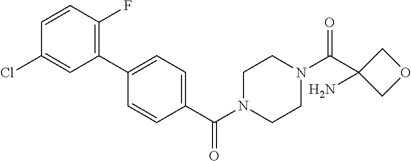 [SMILES: NC1(C(=O)N2CCN(C(=O)c3ccc(-c4cc(Cl)ccc4F)cc3)CC2)COC1] |
| (4-(4-(5-chlorothiazolo[5,4- d]thiazol-2-yl)benzoyl)piperazin-1- hydroxycyclopropyl)methanone | 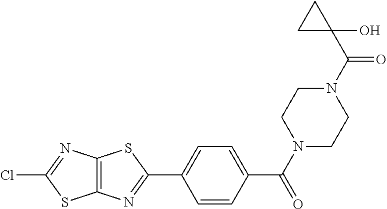 [SMILES: O=C(c1ccc(-c2nc3sc(Cl)nc3s2)cc1)N1CCN(C(=O)C2(O)CC2)CC1] |
| (4-(4′-(2H-tetrazol-5-yl)-[1,1′- biphenyl]-4-carbonyl)piperazin-1- yl)(1- hydroxycyclopropyl)methanone | 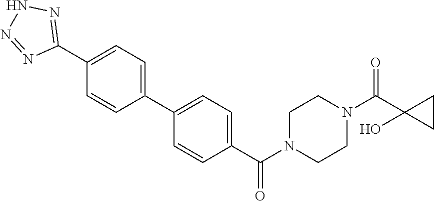 [SMILES: O=C(c1ccc(-c2ccc(-c3nn[nH]n3)cc2)cc1)N1CCN(C(=O)C2(O)CC2)CC1] |
| (4-(1-hydroxycyclopropane-1- carbonyl)piperazin-1-yl)(4- (imidazo[2,1-b][1,3,4]thiadiazol-2- yl)phenyl)methanone | 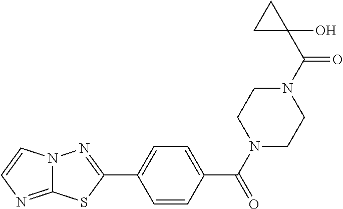 [SMILES: O=C(c1ccc(-c2nn3ccnc3s2)cc1)N1CCN(C(=O)C2(O)CC2)CC1] |
| (4-(4-(6H-thieno[2,3-b]pyrrol-2- yl)benzoyl)piperazin-1-yl)(1- hydroxycyclopropyl)methanone | 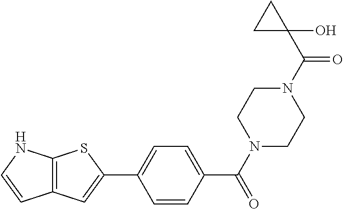 [SMILES: O=C(c1ccc(-c2cc3cc[nH]c3s2)cc1)N1CCN(C(=O)C2(O)CC2)CC1] |
| (4-(4-(4H-thieno[3,2-b]pyrrol-3- yl)benzoyl)piperazin-1-yl)(1- hydroxycyclopropyl)methanone | 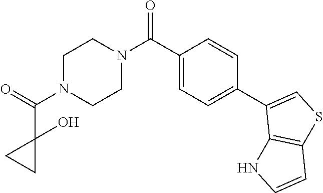 [SMILES: O=C(c1ccc(-c2csc3cc[nH]c23)cc1)N1CCN(C(=O)C2(O)CC2)CC1] |
| 2-(4-(4-(1-hydroxycyclopropane-1- carbonyl)piperazine-1- carbonyl)phenyl)-3H-pyrrolizin-3- one | 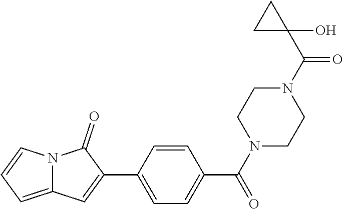 [SMILES: O=C(c1ccc(C2=Cc3cccn3C2=O)cc1)N1CCN(C(=O)C2(O)CC2)CC1] |
| 6-(4-(4-(1-hydroxycyclopropane-1- carbonyl)piperazine-1- carbonyl)phenyl)-3H-pyrrolizin-3- one | 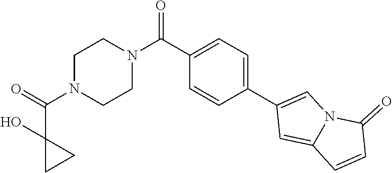 [SMILES: O=C(c1ccc(-c2cc3n(c2)C(=O)C=C3)cc1)N1CCN(C(=O)C2(O)CC2)CC1] |
| (4-(1-hydroxycyclopropane-1- carbonyl)piperazin-1-yl)(4-(1- (methylsulfonyl)-1H-indol-6- yl)phenyl)methanone | 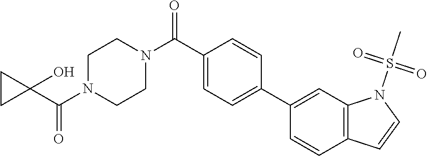 [SMILES: CS(=O)(=O)n1ccc2ccc(-c3ccc(C(=O)N4CCN(C(=O)C5(O)CC5)CC4)cc3)cc21] |
| (4-(1-hydroxycyclopropane-1- carbonyl)piperazin-1-yl)(4′-(1- methyl-1H-pyrazol-5-yl)-[1,1′- biphenyl]-4-yl)methanone | 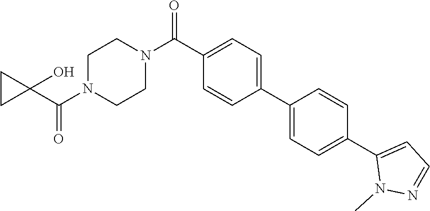 [SMILES: Cn1nccc1-c1ccc(-c2ccc(C(=O)N3CCN(C(=O)C4(O)CC4)CC3)cc2)cc1] |
| (4-(1-hydroxycyclopropane-1- carbonyl)piperazin-1-yl)(4′-(1- methyl-1H-pyrazol-3-yl)-[1,1′- biphenyl]-4-yl)methanone | 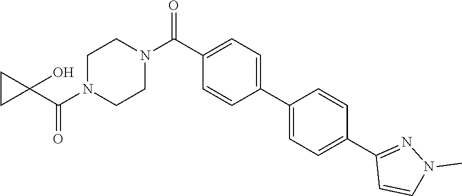 [SMILES: Cn1ccc(-c2ccc(-c3ccc(C(=O)N4CCN(C(=O)C5(O)CC5)CC4)cc3)cc2)n1] |
| (4-(1-hydroxycyclopropane-1- carbonyl)piperazin-1-yl)(4′-(1- methyl-1H-pyrazol-4-yl)-[1,1′- biphenyl]-4-yl)methanone | 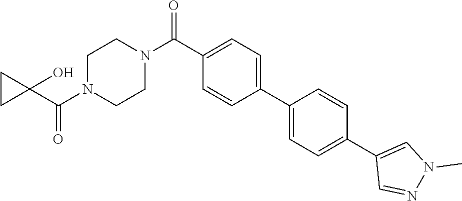 [SMILES: Cn1cc(-c2ccc(-c3ccc(C(=O)N4CCN(C(=O)C5(O)CC5)CC4)cc3)cc2)cn1] |
| (4-(4-(1-ethyl-1H-indazol-5- yl)benzoyl)piperazin-1-yl)(1- hydroxycyclopropyl)methanone | 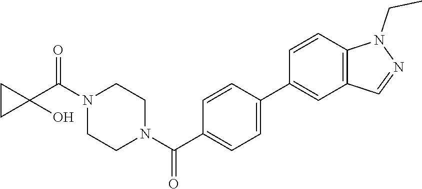 [SMILES: CCn1ncc2cc(-c3ccc(C(=O)N4CCN(C(=O)C5(O)CC5)CC4)cc3)ccc21] |
| (4-(4-(1-cyclobutyl-1H-indazol-5- yl)benzoyl)piperazin-1-yl)(1- hydroxycyclopropyl)methanone | 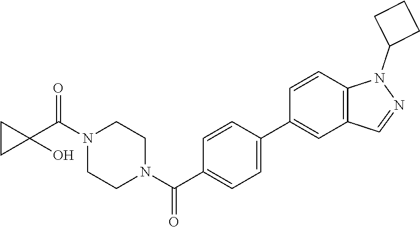 [SMILES: O=C(c1ccc(-c2ccc3c(cnn3C3CCC3)c2)cc1)N1CCN(C(=O)C2(O)CC2)CC1] |
| (4-(1-hydroxycyclopropane-1- carbonyl)piperazin-1-yl)(4-(1- (oxetan-3-yl)-1H-indazol-5- yl)phenyl)methanone | 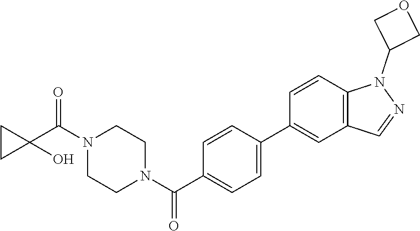 [SMILES: O=C(c1ccc(-c2ccc3c(cnn3C3COC3)c2)cc1)N1CCN(C(=O)C2(O)CC2)CC1] |
| (4-(1-hydroxycyclopropane-1- carbonyl)piperazin-1-yl)(4-(1- isopropyl-1H-indazol-5- yl)phenyl)methanone | 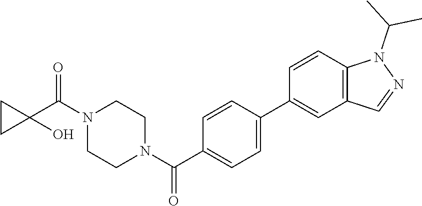 [SMILES: CC(C)n1ncc2cc(-c3ccc(C(=O)N4CCN(C(=O)C5(O)CC5)CC4)cc3)ccc21] |
| (4-(1-hydroxycyclopropane-1- carbonyl)piperazin-1-yl)(4-(1-(2- methoxyethyl)-1H-indazol-5- yl)phenyl)methanone | 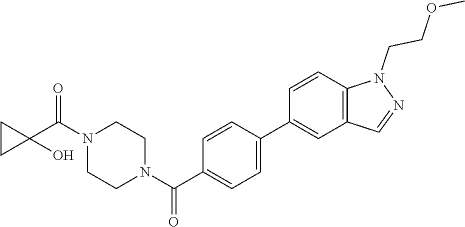 [SMILES: COCCn1ncc2cc(-c3ccc(C(=O)N4CCN(C(=O)C5(O)CC5)CC4)cc3)ccc21] |
| (4-(4-(1-(cyclopropylmethyl)-1H- indazol-5-yl)benzoyl)piperazin-1- yl)(1- hydroxycyclopropyl)methanone | 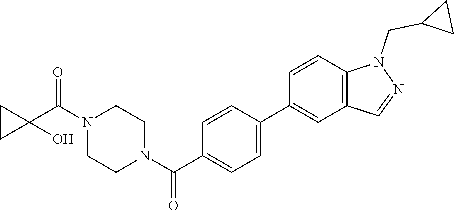 [SMILES: O=C(c1ccc(-c2ccc3c(cnn3CC3CC3)c2)cc1)N1CCN(C(=O)C2(O)CC2)CC1] |
| (4-(1-hydroxycyclopropane-1- carbonyl)piperazin-1-yl)(4-(1- propyl-1H-indazol-5- yl)phenyl)methanone | 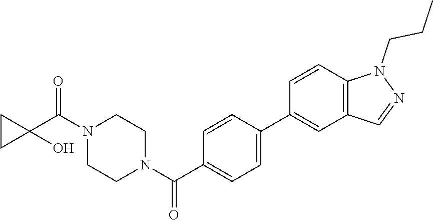 [SMILES: CCCn1ncc2cc(-c3ccc(C(=O)N4CCN(C(=O)C5(O)CC5)CC4)cc3)ccc21] |
| (4-(4-(1-(cyclobutylmethyl)-1H- indazol-5-yl)benzoyl)piperazin-1- yl)(1- hydroxycyclopropyl)methanone | 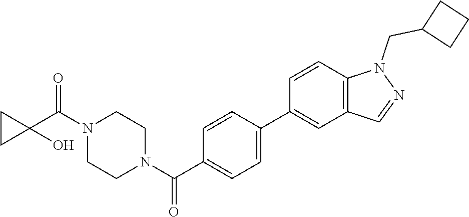 [SMILES: O=C(c1ccc(-c2ccc3c(cnn3CC3CCC3)c2)cc1)N1CCN(C(=O)C2(O)CC2)CC1] |
| (4-(1-hydroxycyclopropane-1- carbonyl)piperazin-1-yl)(4-(1- (oxetan-3-ylmethyl)-1H-indazol-5- yl)phenyl)methanone | 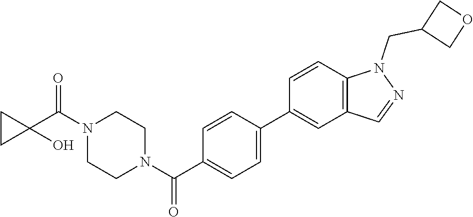 [SMILES: O=C(c1ccc(-c2ccc3c(cnn3CC3COC3)c2)cc1)N1CCN(C(=O)C2(O)CC2)CC1] |
| (4-(1-hydroxycyclopropane-1- carbonyl)piperazin-1-yl)(4-(1-(2- hydroxyethyl)-1H-indazol-5- yl)phenyl)methanone | 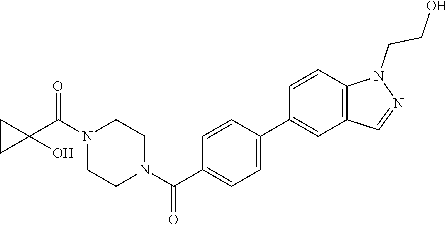 [SMILES: O=C(c1ccc(-c2ccc3c(cnn3CCO)c2)cc1)N1CCN(C(=O)C2(O)CC2)CC1] |
| (4-(4′-(1H-pyrazol-5-yl)-[1,1′- biphenyl]-4-carbonyl)piperazin-1- hydroxycyclopropyl)methanone | 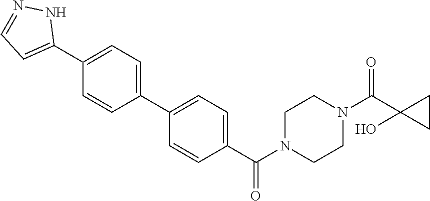 [SMILES: O=C(c1ccc(-c2ccc(-c3ccn[nH]3)cc2)cc1)N1CCN(C(=O)C2(O)CC2)CC1] |
| (4-(4′-(1H-pyrazol-4-yl)-[1,1′- biphenyl]-4-carbonyl)piperazin-1- yl)(1- hydroxycyclopropyl)methanone | 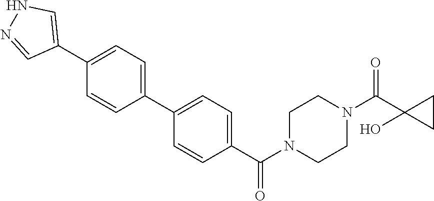 [SMILES: O=C(c1ccc(-c2ccc(-c3cn[nH]c3)cc2)cc1)N1CCN(C(=O)C2(O)CC2)CC1] |
| (4-(1-hydroxycyclopropane-1- carbonyl)piperazin-1-yl)(4′-(2- methyl-2,3-dihydro-1H-1,2,3- triazol-4-yl)-[1,1′-biphenyl]-4- yl)methanone | 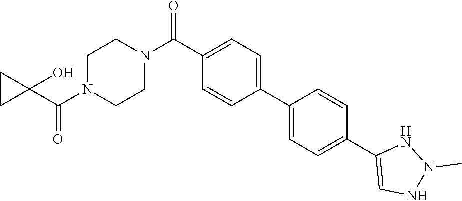 [SMILES: CN1NC=C(c2ccc(-c3ccc(C(=O)N4CCN(C(=O)C5(O)CC5)CC4)cc3)cc2)N1] |
| (4-(1-hydroxycyclopropane-1- carbonyl)piperazin-1-yl)(4′-(1- methyl-1H-1,2,3-triazol-4-yl)-[1,1′- biphenyl]-4-yl)methanone | 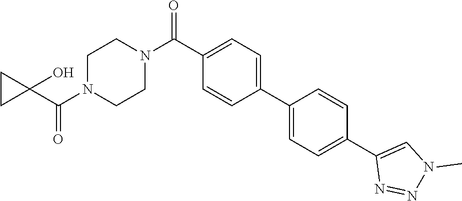 [SMILES: Cn1cc(-c2ccc(-c3ccc(C(=O)N4CCN(C(=O)C5(O)CC5)CC4)cc3)cc2)nn1] |
| (4-(1-hydroxycyclopropane-1- carbonyl)piperazin-1-yl)(4-(1- (methylsulfonyl)-1H-indol-5- yl)phenyl)methanone | 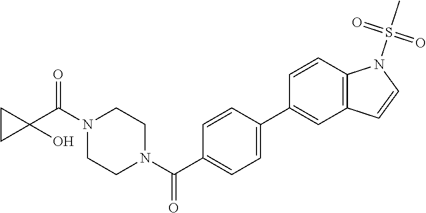 [SMILES: CS(=O)(=O)n1ccc2cc(-c3ccc(C(=O)N4CCN(C(=O)C5(O)CC5)CC4)cc3)ccc21] |
| (4-(4-(1-(cyclopropylsulfonyl)-1H- indol-5-yl)benzoyl)piperazin-1-yl)(1- hydroxycyclopropyl)methanone | 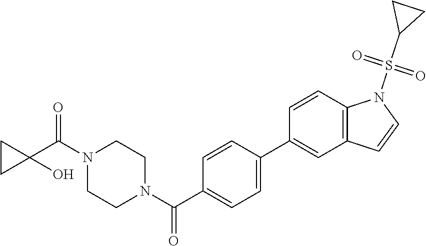 [SMILES: O=C(c1ccc(-c2ccc3c(ccn3S(=O)(=O)C3CC3)c2)cc1)N1CCN(C(=O)C2(O)CC2)CC1] |
| (4-(4-(1-(cyclopropylsulfonyl)-1H- indol-6-yl)benzoyl)piperazin-1-yl)(1- hydroxycyclopropyl)methanone | 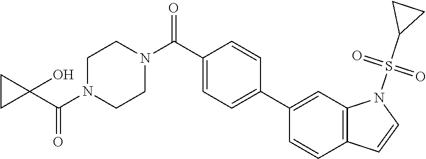 [SMILES: O=C(c1ccc(-c2ccc3ccn(S(=O)(=O)C4CC4)c3c2)cc1)N1CCN(C(=O)C2(O)CC2)CC1] |
| (4-(1-hydroxycyclopropane-1- carbonyl)piperazin-1-yl)(4-(2- (hydroxymethyl)-1H-indol-5- yl)phenyl)methanone | 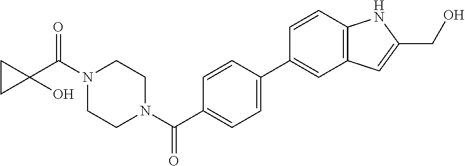 [SMILES: O=C(c1ccc(-c2ccc3[nH]c(CO)cc3c2)cc1)N1CCN(C(=O)C2(O)CC2)CC1] |
| 2-(5-(4-(4-(1-hydroxycyclopropane- 1-carbonyl)piperazine-1- carbonyl)phenyl)-1H-indol-3- yl)acetonitrile | 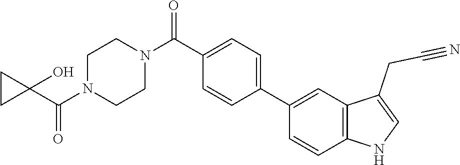 [SMILES: N#CCc1c[nH]c2ccc(-c3ccc(C(=O)N4CCN(C(=O)C5(O)CC5)CC4)cc3)cc12] |
| (4-(1-hydroxycyclopropane-1- carbonyl)piperazin-1-yl)(4-(3-(2- hydroxyethyl)-1H-indol-5- yl)phenyl)methanone | 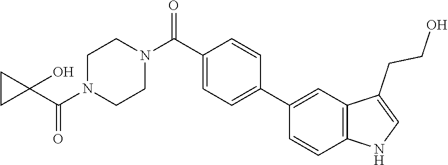 [SMILES: O=C(c1ccc(-c2ccc3[nH]cc(CCO)c3c2)cc1)N1CCN(C(=O)C2(O)CC2)CC1] |
| 5-(4-(4-(1-hydroxycyclopropane-1- carbonyl)piperazine-1- carbonyl)phenyl)benzofuran-2- carbonitrile | 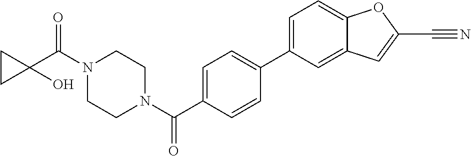 [SMILES: N#Cc1cc2cc(-c3ccc(C(=O)N4CCN(C(=O)C5(O)CC5)CC4)cc3)ccc2o1] |
| (4-(4-(3-amino-1-methyl-1H- indazol-6-yl)benzoyl)piperazin-1- yl)(1- hydroxycyclopropyl)methanone | 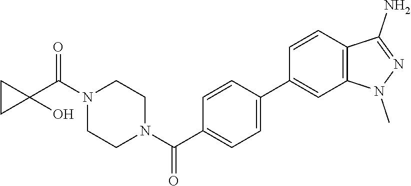 [SMILES: Cn1nc(N)c2ccc(-c3ccc(C(=O)N4CCN(C(=O)C5(O)CC5)CC4)cc3)cc21] |
| (4-(4-(1-amino-2,3- dihydroisoquinolin-6- yl)benzoyl)piperazin-1-yl)(1- hydroxycyclopropyl)methanone | 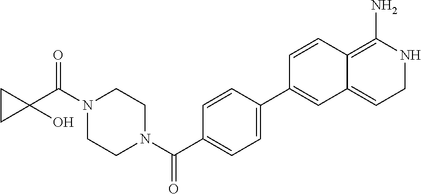 [SMILES: NC1=c2ccc(-c3ccc(C(=O)N4CCN(C(=O)C5(O)CC5)CC4)cc3)cc2=CCN1] |
| (4-(4-(3-aminoisoquinolin-6- yl)benzoyl)piperazin-1-yl)(1- hydroxycyclopropyl)methanone | 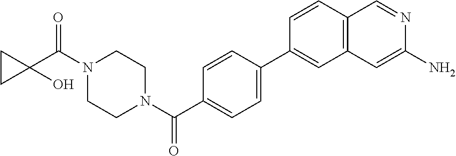 [SMILES: Nc1cc2cc(-c3ccc(C(=O)N4CCN(C(=O)C5(O)CC5)CC4)cc3)ccc2cn1] |
| (4-(4-(1,3-dimethylisoquinolin-6- yl)benzoyl)piperazin-1-yl)(1- hydroxycyclopropyl)methanone | 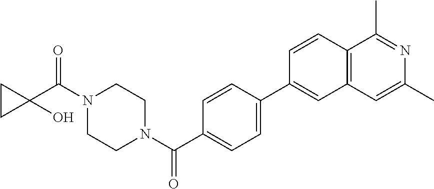 [SMILES: Cc1cc2cc(-c3ccc(C(=O)N4CCN(C(=O)C5(O)CC5)CC4)cc3)ccc2c(C)n1] |
| (4-(1-hydroxycyclopropane-1- carbonyl)piperazin-1-yl)(4-(3- (methoxymethyl)-1-methyl-1H- indazol-6-yl)phenyl)methanone | 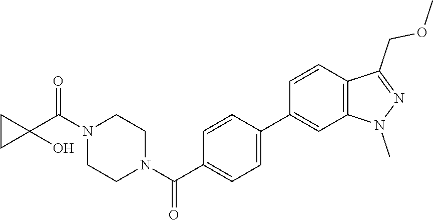 [SMILES: COCc1nn(C)c2cc(-c3ccc(C(=O)N4CCN(C(=O)C5(O)CC5)CC4)cc3)ccc12] |
| (4-(1-hydroxycyclopropane-1- carbonyl)piperazin-1-yl)(4-(3- (hydroxymethyl)-1-methyl-1H- indazol-6-yl)phenyl)methanone | 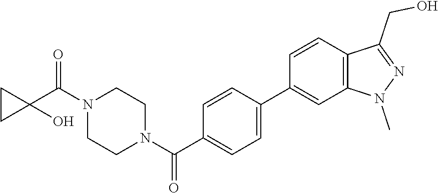 [SMILES: Cn1nc(CO)c2ccc(-c3ccc(C(=O)N4CCN(C(=O)C5(O)CC5)CC4)cc3)cc21] |
| (4-(1-hydroxycyclopropane-1- carbonyl)piperazin-1-yl)(4-(2- methylquinolin-6- yl)phenyl)methanone | 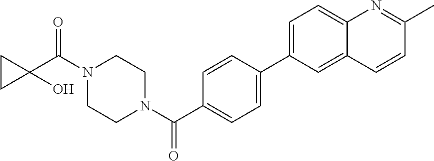 [SMILES: Cc1ccc2cc(-c3ccc(C(=O)N4CCN(C(=O)C5(O)CC5)CC4)cc3)ccc2n1] |
| (4-(4-(2-aminoquinolin-6- yl)benzoyl)piperazin-1-yl)(1- hydroxycyclopropyl)methanone | 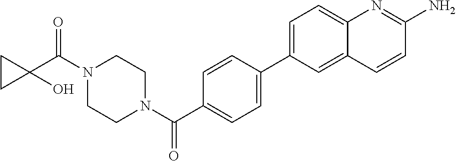 [SMILES: Nc1ccc2cc(-c3ccc(C(=O)N4CCN(C(=O)C5(O)CC5)CC4)cc3)ccc2n1] |
| (4-(4-(1-ethyl-1H-indazol-6- yl)benzoyl)piperazin-1-yl)(1- hydroxycyclopropyl)methanone | 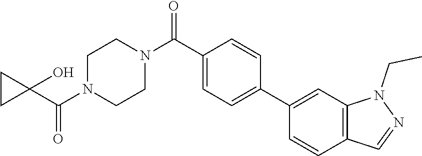 [SMILES: CCn1ncc2ccc(-c3ccc(C(=O)N4CCN(C(=O)C5(O)CC5)CC4)cc3)cc21] |
| (4-(4-(1-cyclobutyl-1H-indazol-6- yl)benzoyl)piperazin-1-yl)(1- hydroxycyclopropyl)methanone | 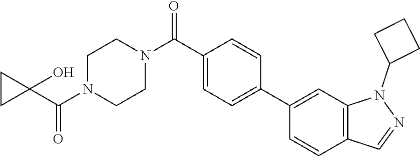 [SMILES: O=C(c1ccc(-c2ccc3cnn(C4CCC4)c3c2)cc1)N1CCN(C(=O)C2(O)CC2)CC1] |
| (4-(1-hydroxycyclopropane-1- carbonyl)piperazin-1-yl)(4-(1- (oxetan-3-yl)-1H-indazol-6- yl)phenyl)methanone | 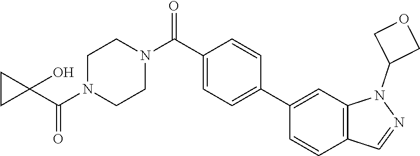 [SMILES: O=C(c1ccc(-c2ccc3cnn(C4COC4)c3c2)cc1)N1CCN(C(=O)C2(O)CC2)CC1] |
| (4-(1-hydroxycyclopropane-1- carbonyl)piperazin-1-yl)(4-(1- isopropyl-1H-indazol-6- yl)phenyl)methanone | 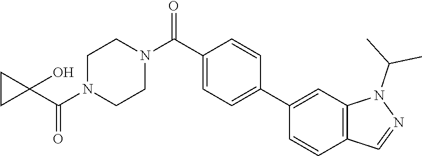 [SMILES: CC(C)n1ncc2ccc(-c3ccc(C(=O)N4CCN(C(=O)C5(O)CC5)CC4)cc3)cc21] |
| (4-(1-hydroxycyclopropane-1- carbonyl)piperazin-1-yl)(4-(1-(2- methoxyethyl)-1H-indazol-6- yl)phenyl)methanone | 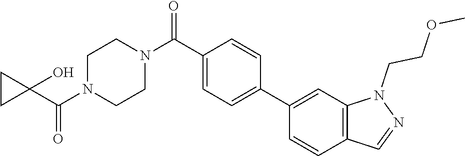 [SMILES: COCCn1ncc2ccc(-c3ccc(C(=O)N4CCN(C(=O)C5(O)CC5)CC4)cc3)cc21] |
| (4-(4-(1-(cyclopropylmethyl)-1H- indazol-6-yl)benzoyl)piperazin-1- yl)(1- hydroxycyclopropyl)methanone | 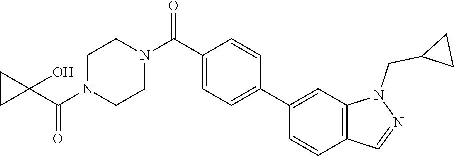 [SMILES: O=C(c1ccc(-c2ccc3cnn(CC4CC4)c3c2)cc1)N1CCN(C(=O)C2(O)CC2)CC1] |
| (4-(1-hydroxycyclopropane-1- carbonyl)piperazin-1-yl)(4-(1- propyl-1H-indazol-6- yl)phenyl)methanone | 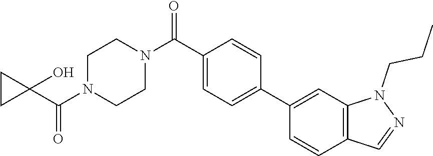 [SMILES: CCCn1ncc2ccc(-c3ccc(C(=O)N4CCN(C(=O)C5(O)CC5)CC4)cc3)cc21] |
| (4-(4-(1-(cyclobutylmethyl)-1H- indazol-6-yl)benzoyl)piperazin-1- yl)(1- hydroxycyclopropyl)methanone | 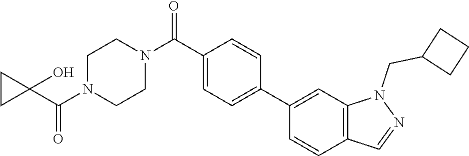 [SMILES: O=C(c1ccc(-c2ccc3cnn(CC4CCC4)c3c2)cc1)N1CCN(C(=O)C2(O)CC2)CC1] |
| (4-(1-hydroxycyclopropane-1- carbonyl)piperazin-1-yl)(4-(1- (oxetan-3-ylmethyl)-1H-indazol-6- yl)phenyl)methanone | 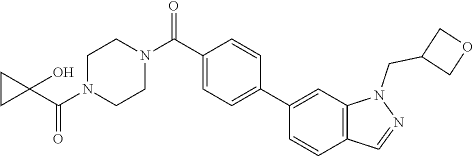 [SMILES: O=C(c1ccc(-c2ccc3cnn(CC4COC4)c3c2)cc1)N1CCN(C(=O)C2(O)CC2)CC1] |
| (4-(1-hydroxycyclopropane-1- carbonyl)piperazin-1-yl)(4-(1-(2- hydroxyethyl)-1H-indazol-6- yl)phenyl)methanone | 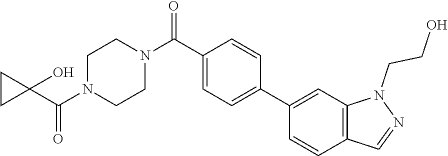 [SMILES: O=C(c1ccc(-c2ccc3cnn(CCO)c3c2)cc1)N1CCN(C(=O)C2(O)CC2)CC1] |
| (4-(4-(1-cyclopropyl-1H-indazol-6- yl)benzoyl)piperazin-1-yl)(1- hydroxycyclopropyl)methanone | 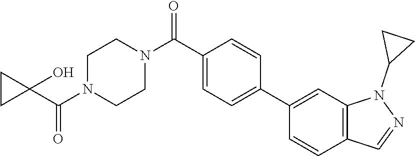 [SMILES: O=C(c1ccc(-c2ccc3cnn(C4CC4)c3c2)cc1)N1CCN(C(=O)C2(O)CC2)CC1] |
| N-(4′-(4-(1-hydroxycyclopropane-1- carbonyl)piperazine-1-carbonyl)- [1,1′-biphenyl]-3-yl)-N- methylcyclopropanecarboxamide | 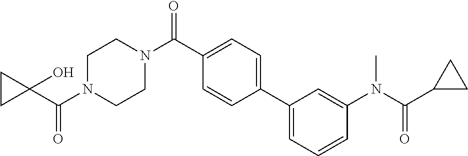 [SMILES: CN(C(=O)C1CC1)c1cccc(-c2ccc(C(=O)N3CCN(C(=O)C4(O)CC4)CC3)cc2)c1] |
| N-(4′-(4-(1-hydroxycyclopropane-1- carbonyl)piperazine-1-carbonyl)- [1,1′-biphenyl]-3-yl)-N- methylcyclobutanecarboxamide | 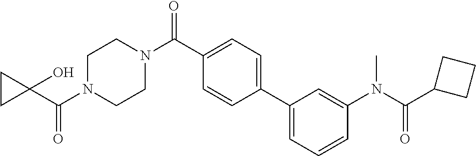 [SMILES: CN(C(=O)C1CCC1)c1cccc(-c2ccc(C(=O)N3CCN(C(=O)C4(O)CC4)CC3)cc2)c1] |
| N-(4′-(4-(1-hydroxycyclopropane-1- carbonyl)piperazine-1-carbonyl)- [1,1′-biphenyl]-3-yl)-N- methylcyclobutanesulfonamide | 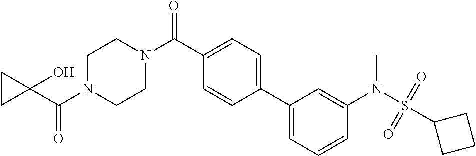 [SMILES: CN(c1cccc(-c2ccc(C(=O)N3CCN(C(=O)C4(O)CC4)CC3)cc2)c1)S(=O)(=O)C1CCC1] |
| N-cyclopropyl-4′-(4-(1- hydroxycyclopropane-1- carbonyl)piperazine-1-carbonyl)-N- methyl-[1,1′-biphenyl]-3- sulfonamide | 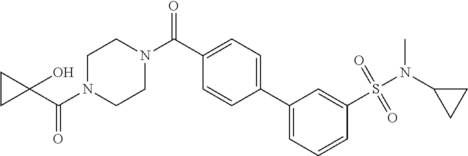 [SMILES: CN(C1CC1)S(=O)(=O)c1cccc(-c2ccc(C(=O)N3CCN(C(=O)C4(O)CC4)CC3)cc2)c1] |
| N-cyclopropyl-4′-(4-(1- hydroxycyclopropane-1- carbonyl)piperazine-1-carbonyl)-N- methyl-[1,1′-biphenyl]-3- carboxamide | 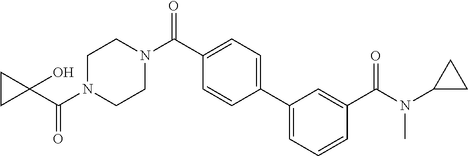 [SMILES: CN(C(=O)c1cccc(-c2ccc(C(=O)N3CCN(C(=O)C4(O)CC4)CC3)cc2)c1)C1CC1] |
| 2-cyclobutyl-N-(4′-(4-(1- hydroxycyclopropane-1- carbonyl)piperazine-1-carbonyl)- [1,1′-biphenyl]-3-yl)acetamide | 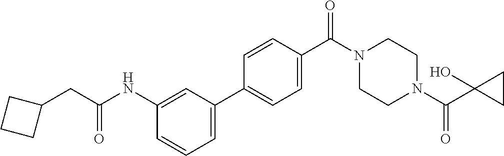 [SMILES: O=C(CC1CCC1)Nc1cccc(-c2ccc(C(=O)N3CCN(C(=O)C4(O)CC4)CC3)cc2)c1] |
| N-(4′-(4-(1-hydroxycyclopropane-1- carbonyl)piperazine-1-carbonyl)- [1,1′-biphenyl]-3-yl)-1- methylazetidine-3-carboxamide | 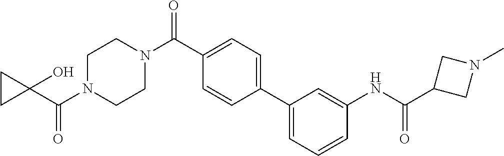 [SMILES: CN1CC(C(=O)Nc2cccc(-c3ccc(C(=O)N4CCN(C(=O)C5(O)CC5)CC4)cc3)c2)C1] |
| N-(4′-(4-(1-hydroxycyclopropane-1- carbonyl)piperazine-1-carbonyl)- [1,1′-biphenyl]-3-yl)-3- methyloxetane-3-carboxamide | 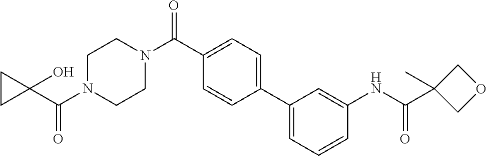 [SMILES: CC1(C(=O)Nc2cccc(-c3ccc(C(=O)N4CCN(C(=O)C5(O)CC5)CC4)cc3)c2)COC1] |
| N-(4′-(4-(1-hydroxycyclopropane-1- carbonyl)piperazine-1-carbonyl)- [1,1′-biphenyl]-3-yl)-2-(oxetan-3- yl)acetamide | 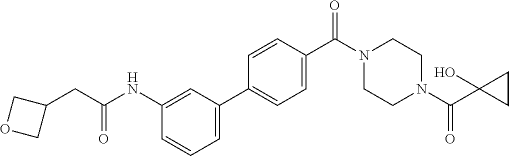 [SMILES: O=C(CC1COC1)Nc1cccc(-c2ccc(C(=O)N3CCN(C(=O)C4(O)CC4)CC3)cc2)c1] |
| 3-fluoro-N-(4′-(4-(1- hydroxycyclopropane-1- carbonyl)piperazine-1-carbonyl)- [1,1′-biphenyl]-3-yl)cyclobutane-1- carboxamide | 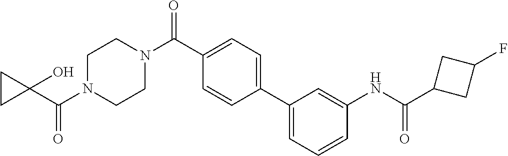 [SMILES: O=C(Nc1cccc(-c2ccc(C(=O)N3CCN(C(=O)C4(O)CC4)CC3)cc2)c1)C1CC(F)C1] |
| 3-ethyl-N-(4′-(4-(1- hydroxycyclopropane-1- carbonyl)piperazine-1-carbonyl)- [1,1′-biphenyl]-3-yl)oxetane-3- carboxamide | 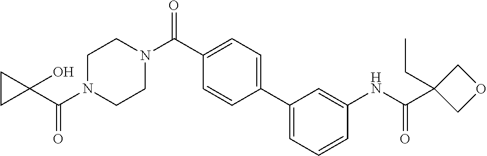 [SMILES: CCC1(C(=O)Nc2cccc(-c3ccc(C(=O)N4CCN(C(=O)C5(O)CC5)CC4)cc3)c2)COC1] |
| N-(4′-(4-(1-hydroxycyclopropane-1- carbonyl)piperazine-1-carbonyl)- [1,1′-biphenyl]-3-yl)propionamide | 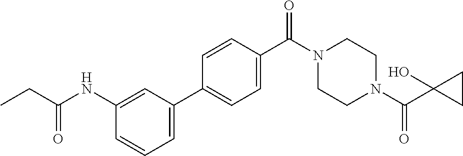 [SMILES: CCC(=O)Nc1cccc(-c2ccc(C(=O)N3CCN(C(=O)C4(O)CC4)CC3)cc2)c1] |
| N-(4′-(4-(1-hydroxycyclopropane-1- carbonyl)piperazine-1-carbonyl)- [1,1′-biphenyl]-3-yl)isobutyramide | 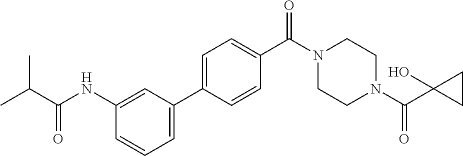 [SMILES: CC(C)C(=O)Nc1cccc(-c2ccc(C(=O)N3CCN(C(=O)C4(O)CC4)CC3)cc2)c1] |
| N-(4′-(4-(1-hydroxycyclopropane-1- carbonyl)piperazine-1-carbonyl)- [1,1′-biphenyl]-3-yl)butyramide | 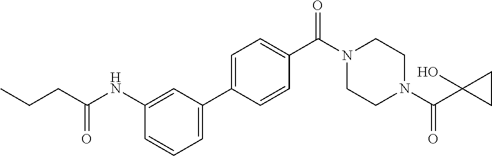 [SMILES: CCCC(=O)Nc1cccc(-c2ccc(C(=O)N3CCN(C(=O)C4(O)CC4)CC3)cc2)c1] |
| N-(4′-(4-(1-hydroxycyclopropane-1- carbonyl)piperazine-1-carbonyl)- [1,1′-biphenyl]-3-yl)-2- methoxyacetamide | 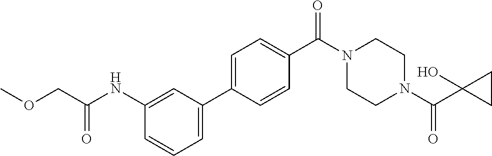 [SMILES: COCC(=O)Nc1cccc(-c2ccc(C(=O)N3CCN(C(=O)C4(O)CC4)CC3)cc2)c1] |
| 2-cyclopropyl-N-(4′-(4-(1- hydroxycyclopropane-1- carbonyl)piperazine-1-carbonyl)- [1,1′-biphenyl]-3-yl)acetamide | 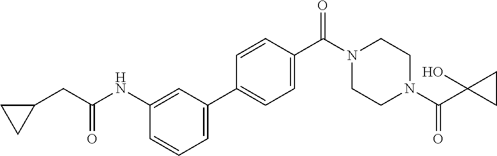 [SMILES: O=C(CC1CC1)Nc1cccc(-c2ccc(C(=O)N3CCN(C(=O)C4(O)CC4)CC3)cc2)c1] |
| 2,2-difluoro-N-(4′-(4-(1- hydroxycyclopropane-1- carbonyl)piperazine-1-carbonyl)- [1,1′-biphenyl]-3-yl)acetamide | 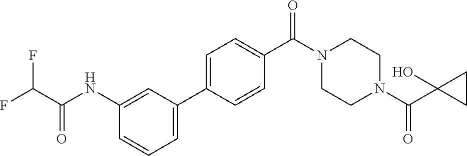 [SMILES: O=C(Nc1cccc(-c2ccc(C(=O)N3CCN(C(=O)C4(O)CC4)CC3)cc2)c1)C(F)F] |
| N-(4′-(4-(1-hydroxycyclopropane-1- carbonyl)piperazine-1-carbonyl)- [1,1′-biphenyl]-3-yl)-1- methylcyclopropane-1- carboxamide | 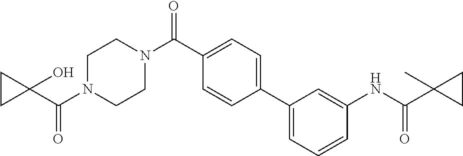 [SMILES: CC1(C(=O)Nc2cccc(-c3ccc(C(=O)N4CCN(C(=O)C5(O)CC5)CC4)cc3)c2)CC1] |
| N-(4′-(4-(1-hydroxycyclopropane-1- carbonyl)piperazine-1-carbonyl)- [1,1′-biphenyl]-3- yl)cyclopentanecarboxamide | 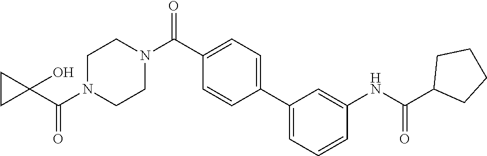 [SMILES: O=C(Nc1cccc(-c2ccc(C(=O)N3CCN(C(=O)C4(O)CC4)CC3)cc2)c1)C1CCCC1] |
| N-(4′-(4-(1-hydroxycyclopropane-1- carbonyl)piperazine-1-carbonyl)- [1,1′-biphenyl]-3- yl)cyclohexanecarboxamide | 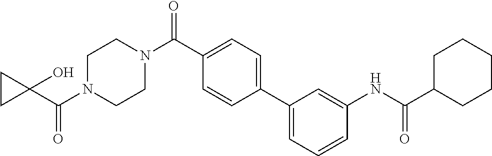 [SMILES: O=C(Nc1cccc(-c2ccc(C(=O)N3CCN(C(=O)C4(O)CC4)CC3)cc2)c1)C1CCCCC1] |
| N-(4′-(4-(1-hydroxycyclopropane-1- carbonyl)piperazine-1-carbonyl)- [1,1′-biphenyl]-3-yl)tetrahydro-2H- pyran-4-carboxamide | 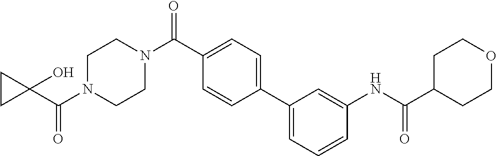 [SMILES: O=C(Nc1cccc(-c2ccc(C(=O)N3CCN(C(=O)C4(O)CC4)CC3)cc2)c1)C1CCOCC1] |
| 1-cyclopropyl-N-(4′-(4-(1- hydroxycyclopropane-1- carbonyl)piperazine-1-carbonyl)- [1,1′-biphenyl]-3- yl)methanesulfonamide | 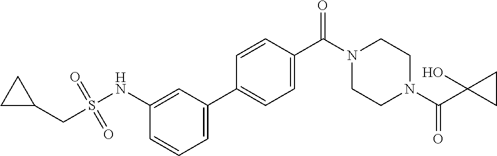 [SMILES: O=C(c1ccc(-c2cccc(NS(=O)(=O)CC3CC3)c2)cc1)N1CCN(C(=O)C2(O)CC2)CC1] |
| N-(4′-(4-(1-hydroxycyclopropane-1- carbonyl)piperazine-1-carbonyl)- [1,1′-biphenyl]-3-yl)-2- methylpropane-1-sulfonamide | 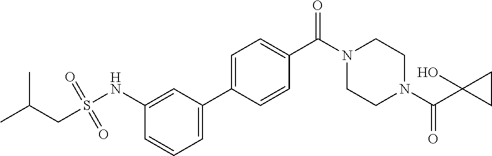 [SMILES: CC(C)CS(=O)(=O)Nc1cccc(-c2ccc(C(=O)N3CCN(C(=O)C4(O)CC4)CC3)cc2)c1] |
| 1,1-difluoro-N-(4′-(4-(1- hydroxycyclopropane-1- carbonyl)piperazine-1-carbonyl)- [1,1′-biphenyl]-3- yl)methanesulfonamide | 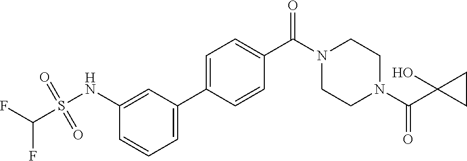 [SMILES: O=C(c1ccc(-c2cccc(NS(=O)(=O)C(F)F)c2)cc1)N1CCN(C(=O)C2(O)CC2)CC1] |
| 3,3,3-trifluoro-N-(4′-(4-(1- hydroxycyclopropane-1- carbonyl)piperazine-1-carbonyl)- [1,1′-biphenyl]-3-yl)propane-1- sulfonamide | 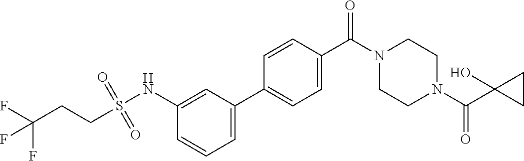 [SMILES: O=C(c1ccc(-c2cccc(NS(=O)(=O)CCC(F)(F)F)c2)cc1)N1CCN(C(=O)C2(O)CC2)CC1] |
| N-(4′-(4-(1-hydroxycyclopropane-1- carbonyl)piperazine-1-carbonyl)- [1,1′-biphenyl]-3-yl)-1- methylcyclopropane-1-sulfonamide | 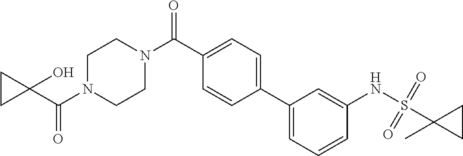 [SMILES: CC1(S(=O)(=O)Nc2cccc(-c3ccc(C(=O)N4CCN(C(=O)C5(O)CC5)CC4)cc3)c2)CC1] |
| N-(4′-(4-(1-hydroxycyclopropane-1- carbonyl)piperazine-1-carbonyl)- [1,1′-biphenyl]-3- yl)cyclobutanesulfonamide | 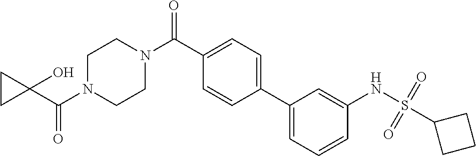 [SMILES: O=C(c1ccc(-c2cccc(NS(=O)(=O)C3CCC3)c2)cc1)N1CCN(C(=O)C2(O)CC2)CC1] |
| N-(4′-(4-(1-hydroxycyclopropane-1- carbonyl)piperazine-1-carbonyl)- [1,1′-biphenyl]-3- yl)tetrahydrofuran-3-sulfonamide | 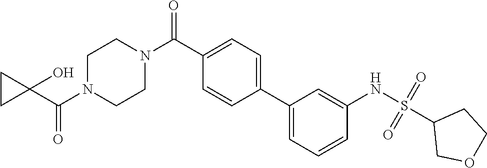 [SMILES: O=C(c1ccc(-c2cccc(NS(=O)(=O)C3CCOC3)c2)cc1)N1CCN(C(=O)C2(O)CC2)CC1] |
| N-(4′-(4-(1-hydroxycyclopropane-1- carbonyl)piperazine-1-carbonyl)- [1,1′-biphenyl]-3- yl)cyclopentanesulfonamide | 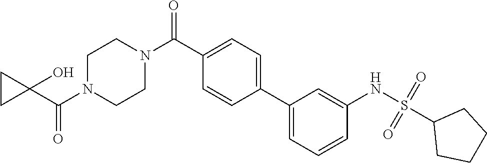 [SMILES: O=C(c1ccc(-c2cccc(NS(=O)(=O)C3CCCC3)c2)cc1)N1CCN(C(=O)C2(O)CC2)CC1] |
| 2,2-difluoro-N-(4′-(4-(1- hydroxycyclopropane-1- carbonyl)piperazine-1-carbonyl)- [1,1′-biphenyl]-3-yl)ethane-1- sulfonamide | 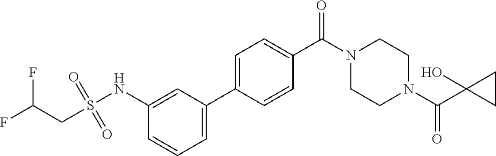 [SMILES: O=C(c1ccc(-c2cccc(NS(=O)(=O)CC(F)F)c2)cc1)N1CCN(C(=O)C2(O)CC2)CC1] |
| N-(4′-(4-(1-hydroxycyclopropane-1- carbonyl)piperazine-1-carbonyl)- [1,1′-biphenyl]-3-yl)-2- methoxyethane-1-sulfonamide | 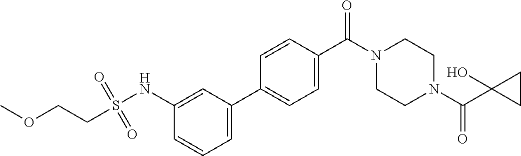 [SMILES: COCCS(=O)(=O)Nc1cccc(-c2ccc(C(=O)N3CCN(C(=O)C4(O)CC4)CC3)cc2)c1] |
| 1-cyclobutyl-N-(4′-(4-(1- hydroxycyclopropane-1- carbonyl)piperazine-1-carbonyl)- [1,1′-biphenyl]-3- yl)methanesulfonamide | 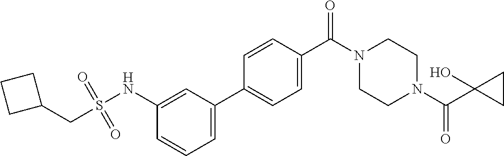 [SMILES: O=C(c1ccc(-c2cccc(NS(=O)(=O)CC3CCC3)c2)cc1)N1CCN(C(=O)C2(O)CC2)CC1] |
| 2-hydroxy-N-(4′-(4-(1- hydroxycyclopropane-1- carbonyl)piperazine-1-carbonyl)- [1,1′-biphenyl]-3-yl)ethane-1- sulfonamide | 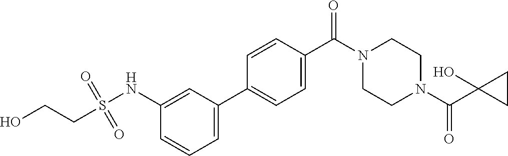 [SMILES: O=C(c1ccc(-c2cccc(NS(=O)(=O)CCO)c2)cc1)N1CCN(C(=O)C2(O)CC2)CC1] |
| N-(4′-(4-(1-hydroxycyclopropane-1- carbonyl)piperazine-1-carbonyl)- [1,1′-biphenyl]-3-yl)tetrahydro-2H- pyran-4-sulfonamide | 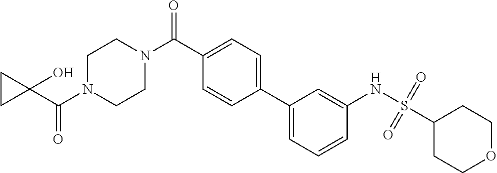 [SMILES: O=C(c1ccc(-c2cccc(NS(=O)(=O)C3CCOCC3)c2)cc1)N1CCN(C(=O)C2(O)CC2)CC1] |
| 2-cyclopropyl-N-(4′-(4-(1- hydroxycyclopropane-1- carbonyl)piperazine-1-carbonyl)- [1,1′-biphenyl]-3-yl)ethane-1- sulfonamide | 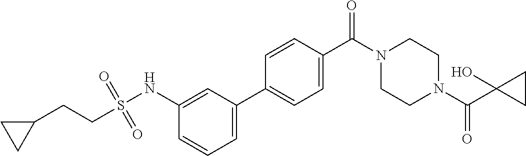 [SMILES: O=C(c1ccc(-c2cccc(NS(=O)(=O)CCC3CC3)c2)cc1)N1CCN(C(=O)C2(O)CC2)CC1] |
| 3,3-difluoro-N-(4′-(4-(1- hydroxycyclopropane-1- carbonyl)piperazine-1-carbonyl)- [1,1′-biphenyl]-3-yl)cyclobutane-1- carboxamide | 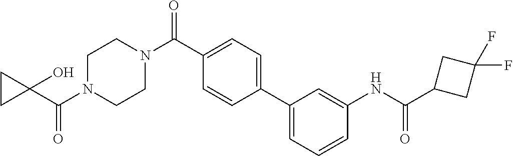 [SMILES: O=C(Nc1cccc(-c2ccc(C(=O)N3CCN(C(=O)C4(O)CC4)CC3)cc2)c1)C1CC(F)(F)C1] |
| (4-(4-(1H-benzo[d]imidazol-4-yl)-2- chlorobenzoyl)piperazin-1-yl)(1- hydroxycyclopropyl)methanone | 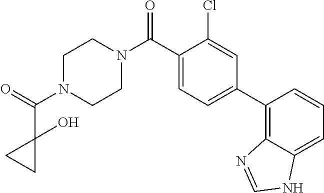 [SMILES: O=C(c1ccc(-c2cccc3[nH]cnc23)cc1Cl)N1CCN(C(=O)C2(O)CC2)CC1] |
| (4-(4-(2-cyclopropyl-2H-indazol-5- yl)benzoyl)piperazin-1-yl)(1- hydroxycyclopropyl)methanone | 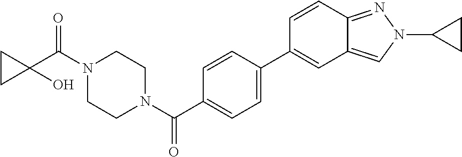 [SMILES: O=C(c1ccc(-c2ccc3nn(C4CC4)cc3c2)cc1)N1CCN(C(=O)C2(O)CC2)CC1] |
| (4-(4-(1-cyclopropyl-1H-indazol-5- yl)benzoyl)piperazin-1-yl)(1- hydroxycyclopropyl)methanone | 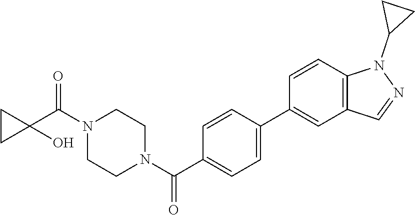 [SMILES: O=C(c1ccc(-c2ccc3c(cnn3C3CC3)c2)cc1)N1CCN(C(=O)C2(O)CC2)CC1] |
| N-(4′-(4-(1-hydroxycyclopropane-1- carbonyl)piperazine-1-carbonyl)- [1,1′-biphenyl]-4- yl)cyclopropanecarboxamide | 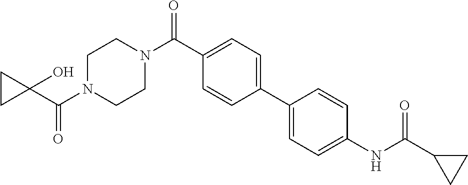 [SMILES: O=C(Nc1ccc(-c2ccc(C(=O)N3CCN(C(=O)C4(O)CC4)CC3)cc2)cc1)C1CC1] |
| N-(4′-(4-(1-hydroxycyclopropane-1- carbonyl)piperazine-1-carbonyl)- [1,1′-biphenyl]-3-yl)-N- methylcyclopropanesulfonamide | 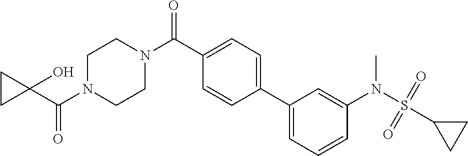 [SMILES: CN(c1cccc(-c2ccc(C(=O)N3CCN(C(=O)C4(O)CC4)CC3)cc2)c1)S(=O)(=O)C1CC1] |
| N-(4′-(4-(1-methoxycyclopropane- 1-carbonyl)piperazine-1-carbonyl)- [1,1′-biphenyl]-3-yl)-N- methylcyclopropanesulfonamide | 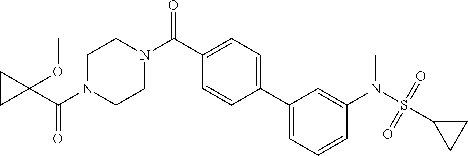 [SMILES: COC1(C(=O)N2CCN(C(=O)c3ccc(-c4cccc(N(C)S(=O)(=O)C5CC5)c4)cc3)CC2)CC1] |
| 1-cyclopropyl-3-(4′-(4-(1- hydroxycyclopropane-1- carbonyl)piperazine-1-carbonyl)- [1,1′-biphenyl]-3-yl)urea | 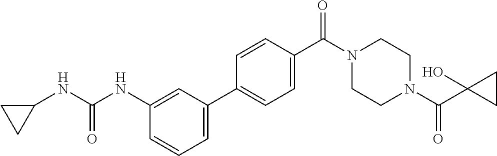 [SMILES: O=C(Nc1cccc(-c2ccc(C(=O)N3CCN(C(=O)C4(O)CC4)CC3)cc2)c1)NC1CC1] |
| 4′-(4-(1-hydroxycyclopropane-1- carbonyl)piperazine-1-carbonyl)- [1,1′-biphenyl]-3-yl cyclopropylcarbamate | 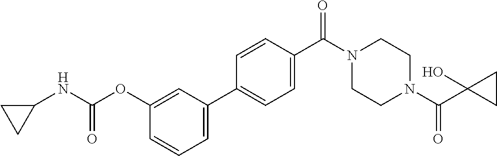 [SMILES: O=C(NC1CC1)Oc1cccc(-c2ccc(C(=O)N3CCN(C(=O)C4(O)CC4)CC3)cc2)c1] |
| N-(4′-(4-(1-hydroxycyclopropane-1- carbonyl)piperazine-1-carbonyl)- [1,1′-biphenyl]-4- yl)cyclopropanesulfonamide | 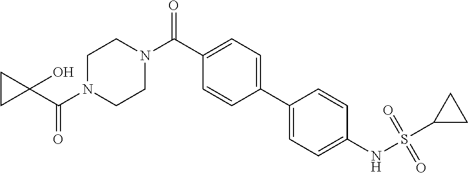 [SMILES: O=C(c1ccc(-c2ccc(NS(=O)(=O)C3CC3)cc2)cc1)N1CCN(C(=O)C2(O)CC2)CC1] |
| N-(3′-chloro-4′-(4-(1- hydroxycyclopropane-1- carbonyl)piperazine-1-carbonyl)- [1,1′-biphenyl]-3- yl)cyclopropanecarboxamide | 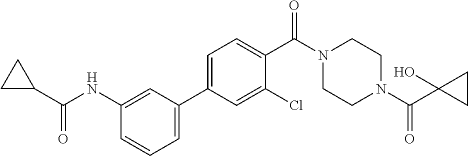 [SMILES: O=C(Nc1cccc(-c2ccc(C(=O)N3CCN(C(=O)C4(O)CC4)CC3)c(Cl)c2)c1)C1CC1] |
| N-(4′-(4-(1-hydroxycyclopropane-1- carbonyl)piperazine-1-carbonyl)- [1,1′-biphenyl]-3- yl)cyclopentanecarboxamide | 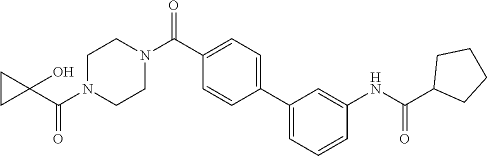 [SMILES: O=C(Nc1cccc(-c2ccc(C(=O)N3CCN(C(=O)C4(O)CC4)CC3)cc2)c1)C1CCCC1] |
| (4-(4′-(1H-pyrazol-4-yl)-[1,1′- biphenyl]-4-carbonyl)piperazin-1- yl)(1- hydroxycyclopropyl)methanone | 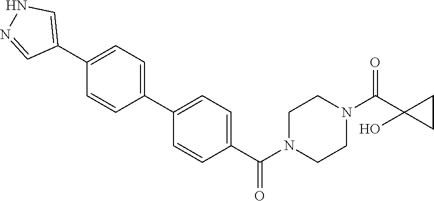 [SMILES: O=C(c1ccc(-c2ccc(-c3cn[nH]c3)cc2)cc1)N1CCN(C(=O)C2(O)CC2)CC1] |
| (4-(4-(3,4-dihydroisoquinolin-2(1H)- yl)benzoyl)piperazin-1-yl)(1- hydroxycyclopropyl)methanone | 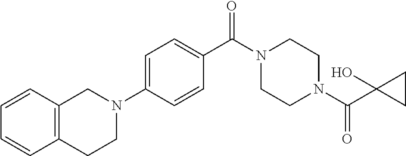 [SMILES: O=C(c1ccc(N2CCc3ccccc3C2)cc1)N1CCN(C(=O)C2(O)CC2)CC1] |
| (4-(1-aminocyclopropane-1- carbonyl)piperazin-1-yl)(4-(1,3- dimethyl-1H-indazol-5-yl )-2- fluorophenyl)methanone | 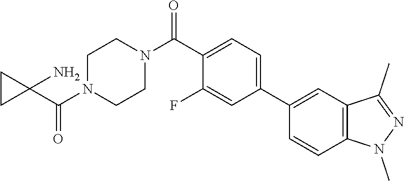 [SMILES: Cc1nn(C)c2ccc(-c3ccc(C(=O)N4CCN(C(=O)C5(N)CC5)CC4)c(F)c3)cc12] |
| (4-(1-hydroxycyclopropane-1- carbonyl)piperazin-1-yl)(4′-(1- methyl-1H-pyrazol-4-yl)-[1,1′- biphenyl]-4-yl)methanone | 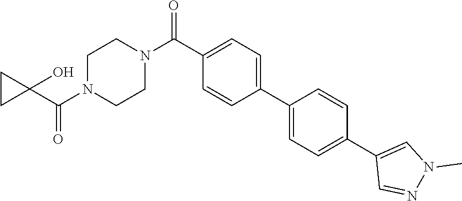 [SMILES: Cn1cc(-c2ccc(-c3ccc(C(=O)N4CCN(C(=O)C5(O)CC5)CC4)cc3)cc2)cn1] |
| (S)-(4-(2-fluoro-4-(1-methyl-1H- benzo[d]imidazol-5-yl)benzoyl)-2- methylpiperazin-1-yl)(1- hydroxycyclopropyl)methanone | 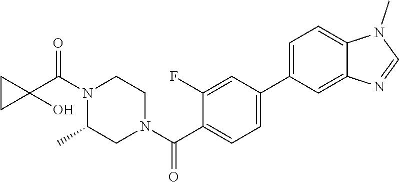 [SMILES: C[C@H]1CN(C(=O)c2ccc(-c3ccc4c(c3)ncn4C)cc2F)CCN1C(=O)C1(O)CC1] |
| (4-(3-chloro-4-(1-methyl-1H- indazol-6-yl)benzoyl)piperazin-1- yl)(1- hydroxycyclopropyl)methanone | 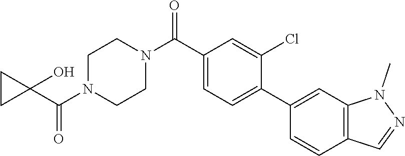 [SMILES: Cn1ncc2ccc(-c3ccc(C(=O)N4CCN(C(=O)C5(O)CC5)CC4)cc3Cl)cc21] |
| (4-(2-fluoro-4-(1-methyl-1H- benzo[d]imidazol-5- yl)benzoyl)piperazin-1-yl)(1- hydroxycyclopropyl)methanone | 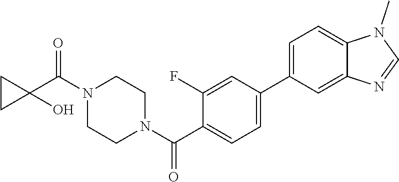 [SMILES: Cn1cnc2cc(-c3ccc(C(=O)N4CCN(C(=O)C5(O)CC5)CC4)c(F)c3)ccc21] |
| (5-chloro-4-(1-methyl-1H- benzo[d]imidazol-5-yl)cyclohexa- 1,5-dien-1-yl)(4-(1- hydroxycyclopropane-1- carbonyl)piperazin-1-yl)methanone | 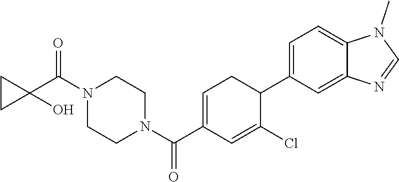 [SMILES: Cn1cnc2cc(C3CC=C(C(=O)N4CCN(C(=O)C5(O)CC5)CC4)C=C3Cl)ccc21] |
| (4-(1-hydroxycyclopropane-1- carbonyl)piperazin-1-yl)(4- (pyrazolo[1,5-a]pyridin-6- yl)phenyl)methanone | 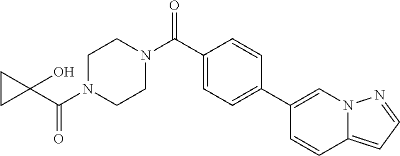 [SMILES: O=C(c1ccc(-c2ccc3ccnn3c2)cc1)N1CCN(C(=O)C2(O)CC2)CC1] |
| (4-(4-(4-fluoroindolin-1- yl)benzoyl)piperazin-1-yl)(1- hydroxycyclopropyl)methanone | 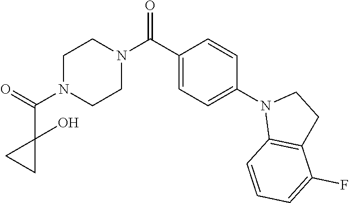 [SMILES: O=C(c1ccc(N2CCc3c(F)cccc32)cc1)N1CCN(C(=O)C2(O)CC2)CC1] |
| (4-(1-hydroxycyclopropane-1- carbonyl)piperazin-1-yl)(4-(3- methyl-1H-indazol-6- yl)phenyl)methanone | 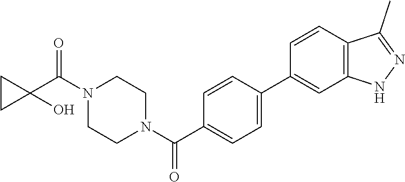 [SMILES: Cc1n[nH]c2cc(-c3ccc(C(=O)N4CCN(C(=O)C5(O)CC5)CC4)cc3)ccc12] |
| (4-(2-fluoro-4-(3-methyl-1H- indazol-7-yl)benzoyl)piperazin-1- hydroxycyclopropyl)methanone | 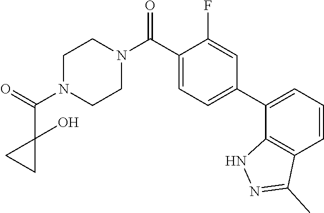 [SMILES: Cc1n[nH]c2c(-c3ccc(C(=O)N4CCN(C(=O)C5(O)CC5)CC4)c(F)c3)cccc12] |
| (S)-(4-(1-hydroxycyclopropane-1- carbonyl)-3-methylpiperazin-1- yl)(4-(1-methyl-1H-indazol-6- yl)phenyl)methanone | 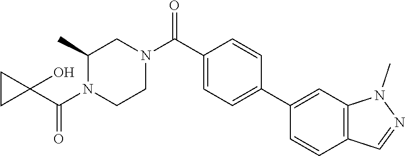 [SMILES: C[C@H]1CN(C(=O)c2ccc(-c3ccc4cnn(C)c4c3)cc2)CCN1C(=O)C1(O)CC1] |
| (4-(3-chloro-4-(2-methyl-2H- indazol-6-yl)benzoyl)piperazin-1- yl)(1- hydroxycyclopropyl)methanone | 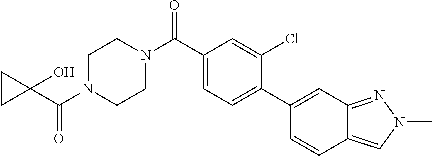 [SMILES: Cn1cc2ccc(-c3ccc(C(=O)N4CCN(C(=O)C5(O)CC5)CC4)cc3Cl)cc2n1] |
| 3,3,3-trifluoro-N-(4′-(4-(1- hydroxycyclopropane-1- carbonyl)piperazine-1-carbonyl)- [1,1′-biphenyl]-3-yl)propane-1- sulfonamide | 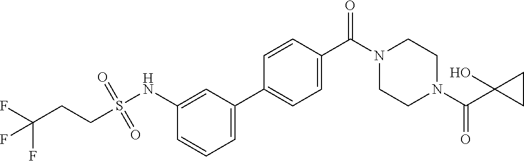 [SMILES: O=C(c1ccc(-c2cccc(NS(=O)(=O)CCC(F)(F)F)c2)cc1)N1CCN(C(=O)C2(O)CC2)CC1] |
| (4-(2-fluoro-4-(1-methyl-1H- indazol-6-yl)benzoyl)piperazin-1- yl)(1- hydroxycyclopropyl)methanone | 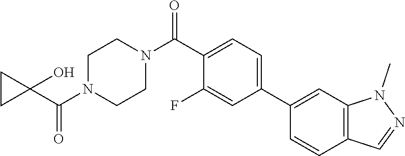 [SMILES: Cn1ncc2ccc(-c3ccc(C(=O)N4CCN(C(=O)C5(O)CC5)CC4)c(F)c3)cc21] |
| (3,3-dimethyl-4-(4-(1-methyl-1H- benzo[d]imidazol-5- yl)benzoyl)piperazin-1-yl)(1- hydroxycyclopropyl)methanone | 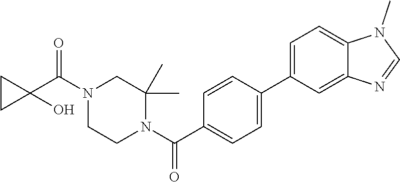 [SMILES: Cn1cnc2cc(-c3ccc(C(=O)N4CCN(C(=O)C5(O)CC5)CC4(C)C)cc3)ccc21] |
| (4-(2,6-difluoro-4-(1-methyl-1H- benzo[d]imidazol-5- yl)benzoyl)piperazin-1-yl)(1- hydroxycyclopropyl)methanone | 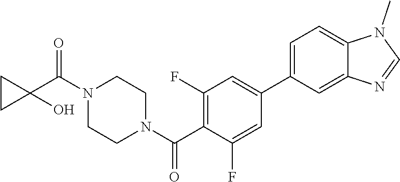 [SMILES: Cn1cnc2cc(-c3cc(F)c(C(=O)N4CCN(C(=O)C5(O)CC5)CC4)c(F)c3)ccc21] |
| (4-(1-aminocyclobutane-1- carbonyl)piperazin-1-yl)(2-fluoro-4- (1-methyl-1H-indazol-6- yl)phenyl)methanone | 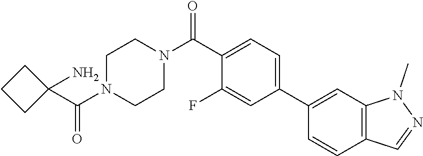 [SMILES: Cn1ncc2ccc(-c3ccc(C(=O)N4CCN(C(=O)C5(N)CCC5)CC4)c(F)c3)cc21] |
| (4-(3-chloro-4-(6- chlorobenzo[d]oxazol-2- yl)benzoyl)piperazin-1-yl)(1- hydroxycyclopropyl)methanone | 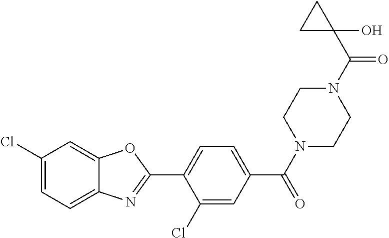 [SMILES: O=C(c1ccc(-c2nc3ccc(Cl)cc3o2)c(Cl)c1)N1CCN(C(=O)C2(O)CC2)CC1] |
| (4-(4-(1,3-dimethyl-1H-indazol-5- yl)-2-fluorobenzoyl)piperazin-1- yl)(1- hydroxycyclopropyl)methanone | 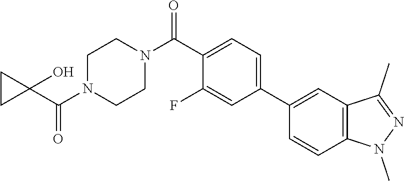 [SMILES: Cc1nn(C)c2ccc(-c3ccc(C(=O)N4CCN(C(=O)C5(O)CC5)CC4)c(F)c3)cc12] |
| N-(4′-(4-(1-hydroxycyclopropane-1- carbonyl)piperazine-1-carbonyl)- [1,1′-biphenyl]-3- yl)cyclobutanesulfonamide | 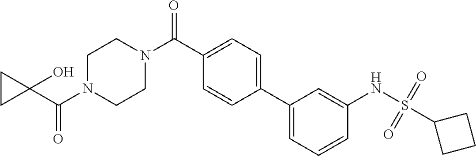 [SMILES: O=C(c1ccc(-c2cccc(NS(=O)(=O)C3CCC3)c2)cc1)N1CCN(C(=O)C2(O)CC2)CC1] |
| (4-(1-hydroxycyclopropane-1- carbonyl)piperazin-1-yl)(4-(indolin- 1-yl)phenyl)methanone | 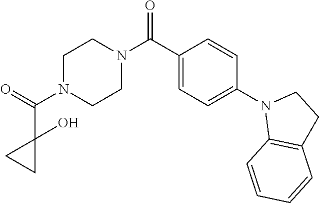 [SMILES: O=C(c1ccc(N2CCc3ccccc32)cc1)N1CCN(C(=O)C2(O)CC2)CC1] |
| (4-(3-chloro-4-(1,3-dimethyl-1H- indazol-5-yl)benzoyl)piperazin-1- yl)(1- hydroxycyclopropyl)methanone | 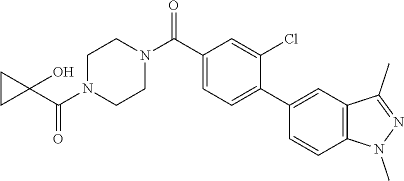 [SMILES: Cc1nn(C)c2ccc(-c3ccc(C(=O)N4CCN(C(=O)C5(O)CC5)CC4)cc3Cl)cc12] |
| (S)-(4-(1-hydroxycyclopropane-1- carbonyl)-3-methylpiperazin-1- yl)(4-(1-methyl-1H- benzo[d]imidazol-5- yl)phenyl)methanone | 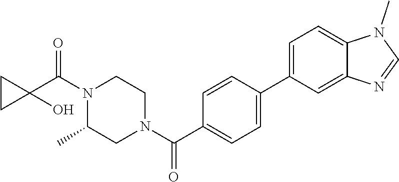 [SMILES: C[C@H]1CN(C(=O)c2ccc(-c3ccc4c(c3)ncn4C)cc2)CCN1C(=O)C1(O)CC1] |
| (4-(2,6-difluoro-4-(2-methyl-2H- indazol-6-yl)benzoyl)piperazin-1- yl)(1- hydroxycyclopropyl)methanone | 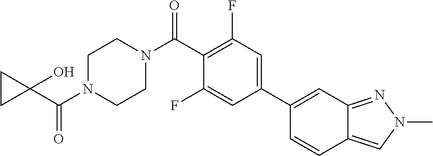 [SMILES: Cn1cc2ccc(-c3cc(F)c(C(=O)N4CCN(C(=O)C5(O)CC5)CC4)c(F)c3)cc2n1] |
| (4-(3′-(5-amino-1l3,2l2-isoxazol-3- yl)-3-fluoro-[1,1′-biphenyl]-4- carbonyl)piperazin-1-yl)(1- hydroxycyclopropyl)methanone | 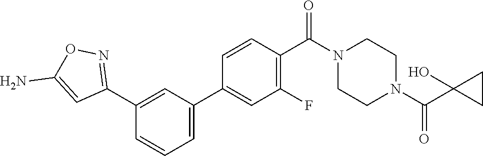 [SMILES: Nc1cc(-c2cccc(-c3ccc(C(=O)N4CCN(C(=O)C5(O)CC5)CC4)c(F)c3)c2)no1] |
| (S)-(4-(1-hydroxycyclopropane-1- carbonyl)-3-methylpiperazin-1- yl)(4-(2-methyl-2H-indazol-6- yl)phenyl)methanone | 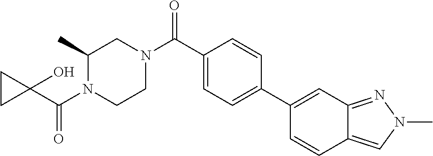 [SMILES: C[C@H]1CN(C(=O)c2ccc(-c3ccc4cn(C)nc4c3)cc2)CCN1C(=O)C1(O)CC1] |
| 1-cyclobutyl-N-(4′-(4-(1- hydroxycyclopropane-1- carbonyl)piperazine-1-carbonyl)- [1,1′-biphenyl]-3- yl)methanesulfonamide | 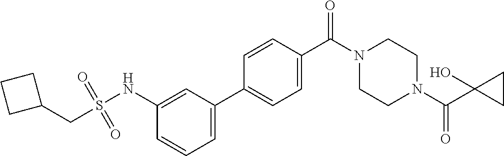 [SMILES: O=C(c1ccc(-c2cccc(NS(=O)(=O)CC3CCC3)c2)cc1)N1CCN(C(=O)C2(O)CC2)CC1] |
| (4-(2-fluoro-4-(7-fluoro-1H-indazol- 3-yl)benzoyl)piperazin-1-yl)(1- hydroxycyclopropyl)methanone | 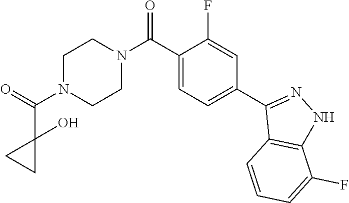 [SMILES: O=C(c1ccc(-c2n[nH]c3c(F)cccc23)cc1F)N1CCN(C(=O)C2(O)CC2)CC1] |
| (S)-(4-(4-(6-chlorobenzo[d]oxazol- 2-yl)benzoyl)-2-methylpiperazin-1- yl)(1- hydroxycyclopropyl)methanone | 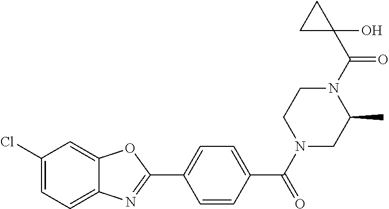 [SMILES: C[C@H]1CN(C(=O)c2ccc(-c3nc4ccc(Cl)cc4o3)cc2)CCN1C(=O)C1(O)CC1] |
| (4-(4-(1H-indol-1- yl)benzoyl)piperazin-1-yl)(1- hydroxycyclopropyl)methanone | 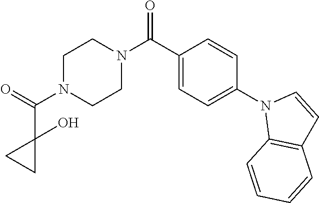 [SMILES: O=C(c1ccc(-n2ccc3ccccc32)cc1)N1CCN(C(=O)C2(O)CC2)CC1] |
| (4-(4-(7-fluoro-1H-indazol-3- yl)benzoyl)piperazin-1-yl)(1- hydroxycyclopropyl)methanone | 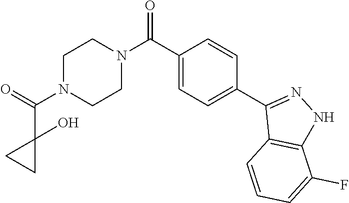 [SMILES: O=C(c1ccc(-c2n[nH]c3c(F)cccc23)cc1)N1CCN(C(=O)C2(O)CC2)CC1] |
| (4-(4-(7-fluoro-3,4- dihydroisoquinolin-2(1H)- yl)benzoyl)piperazin-1-yl)(1- hydroxycyclopropyl)methanone | 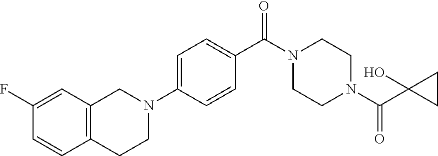 [SMILES: O=C(c1ccc(N2CCc3ccc(F)cc3C2)cc1)N1CCN(C(=O)C2(O)CC2)CC1] |
| (4-(2-fluoro-4-(2-methyl-2H- indazol-6-yl)benzoyl)piperazin-1- yl)(1- hydroxycyclopropyl)methanone | 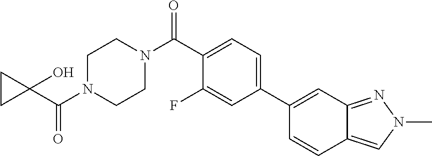 [SMILES: Cn1cc2ccc(-c3ccc(C(=O)N4CCN(C(=O)C5(O)CC5)CC4)c(F)c3)cc2n1] |
| (4-(4-(3-amino-1H-indazol-6- yl)benzoyl)piperazin-1-yl)(1- hydroxycyclopropyl)methanone | 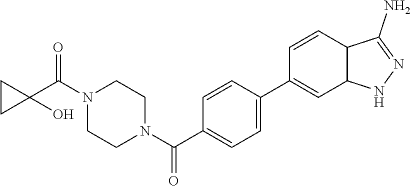 [SMILES: NC1=NNC2C=C(c3ccc(C(=O)N4CCN(C(=O)C5(O)CC5)CC4)cc3)C=CC12] |
| (4-(4-(3-aminobenzo[d]isoxazol-5- yl)benzoyl)piperazin-1-yl)(1- hydroxycyclopropyl)methanone | 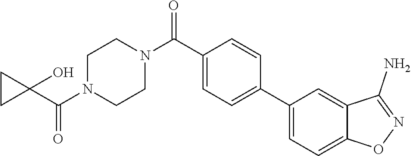 [SMILES: Nc1noc2ccc(-c3ccc(C(=O)N4CCN(C(=O)C5(O)CC5)CC4)cc3)cc12] |
| (4-(1-aminocyclobutane-1- carbonyl)piperazin-1-yl)(4-(1,3- dimethyl-1H-indazol-5-yl)-2- fluorophenyl)methanone | 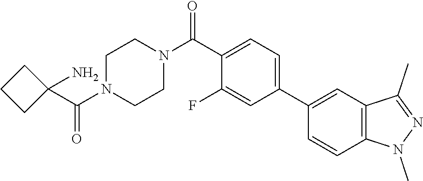 [SMILES: Cc1nn(C)c2ccc(-c3ccc(C(=O)N4CCN(C(=O)C5(N)CCC5)CC4)c(F)c3)cc12] |
| (4-(2-fluoro-4-(3-methyl-1H- indazol-6-yl)benzoyl)piperazin-1- yl)(1- hydroxycyclopropyl)methanone | 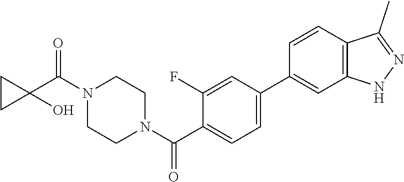 [SMILES: Cc1n[nH]c2cc(-c3ccc(C(=O)N4CCN(C(=O)C5(O)CC5)CC4)c(F)c3)ccc12] |
| ethyl (4′-(4-(1- hydroxycyclopropane-1- carbonyl)piperazine-1-carbonyl)- [1,1′-biphenyl]-3-yl)carbamate | 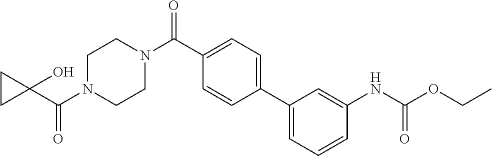 [SMILES: CCOC(=O)Nc1cccc(-c2ccc(C(=O)N3CCN(C(=O)C4(O)CC4)CC3)cc2)c1] |
| (4-(4-(5-chloro-2-methyl-1H- benzo[d]imidazol-7- yl)benzoyl)piperazin-1-yl)(1- hydroxycyclopropyl)methanone | 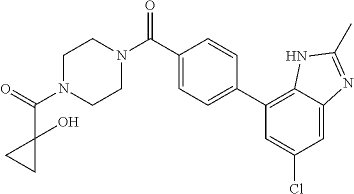 [SMILES: Cc1nc2cc(Cl)cc(-c3ccc(C(=O)N4CCN(C(=O)C5(O)CC5)CC4)cc3)c2[nH]1] |
| (4-(1-hydroxycyclopropane-1- carbonyl)piperazin-1-yl)(4-(1,3,4,9- tetrahydro-2H-pyrido[3,4-b]indol-2- yl)phenyl)methanone | 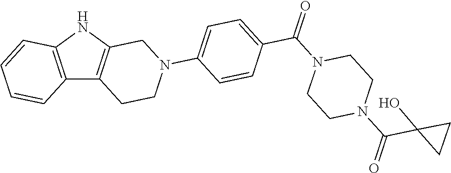 [SMILES: O=C(c1ccc(N2CCc3c([nH]c4ccccc34)C2)cc1)N1CCN(C(=O)C2(O)CC2)CC1] |
| ((2S,6R)-2,6-dimethyl-4-(4-(1- methyl-1H-benzo[d]imidazol-5- yl)benzoyl)piperazin-1-yl)(1- hydroxycyclopropyl)methanone | 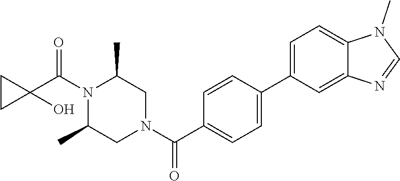 [SMILES: C[C@@H]1CN(C(=O)c2ccc(-c3ccc4c(c3)ncn4C)cc2)C[C@H](C)N1C(=O)C1(O)CC1] |
| (4-(2-chloro-4-(3- chloroimidazo[1,2-a]pyridin-2- yl)benzoyl)piperazin-1-yl)(1- hydroxycyclopropyl)methanone | 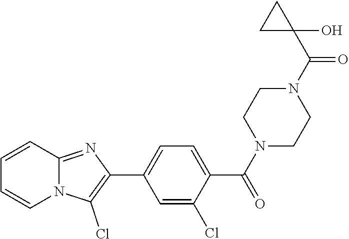 [SMILES: O=C(c1ccc(-c2nc3ccccn3c2Cl)cc1Cl)N1CCN(C(=O)C2(O)CC2)CC1] |
| (4-(4′-chloro-2′-fluoro-[1,1′- biphenyl]-4-carbonyl)-3,3- dimethylpiperazin-1-yl)(1- hydroxycyclopropyl)methanone | 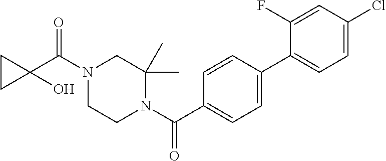 [SMILES: CC1(C)CN(C(=O)C2(O)CC2)CCN1C(=O)c1ccc(-c2ccc(Cl)cc2F)cc1] |
| (4-(2-chloro-4-(1-methyl-1H- indazol-6-yl)benzoyl)piperazin-1- yl)(1- hydroxycyclopropyl)methanone | 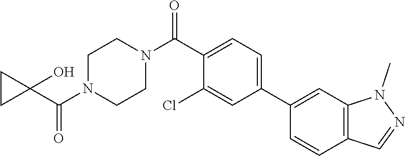 [SMILES: Cn1ncc2ccc(-c3ccc(C(=O)N4CCN(C(=O)C5(O)CC5)CC4)c(Cl)c3)cc21] |
| (S)-(4-(4′-chloro-2′-fluoro-[1,1′- biphenyl]-4-carbonyl)-2- methylpiperazin-1-yl)(1- hydroxycyclopropyl)methanone | 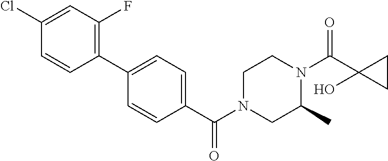 [SMILES: C[C@H]1CN(C(=O)c2ccc(-c3ccc(Cl)cc3F)cc2)CCN1C(=O)C1(O)CC1] |
| (S)-(4-(4-(1,3-dimethyl-1H-indazol- 5-yl)benzoyl)-2-methylpiperazin-1- yl)(1- hydroxycyclopropyl)methanone | 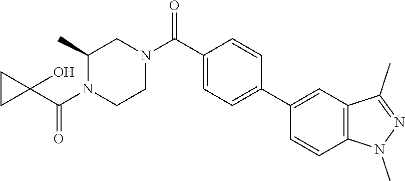 [SMILES: Cc1nn(C)c2ccc(-c3ccc(C(=O)N4CCN(C(=O)C5(O)CC5)[C@@H](C)C4)cc3)cc12] |
| (4-(3′-(5-aminoisoxazol-3-yl)-[1,1′- biphenyl]-4-carbonyl)piperazin-1- yl)(1- hydroxycyclopropyl)methanone | 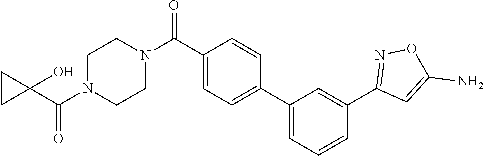 [SMILES: Nc1cc(-c2cccc(-c3ccc(C(=O)N4CCN(C(=O)C5(O)CC5)CC4)cc3)c2)no1] |
| 2-(4′-(4-(1-hydroxycyclopropane-1- carbonyl)piperazine-1-carbonyl)- [1,1′-biphenyl]-3-yl)acetonitrile | 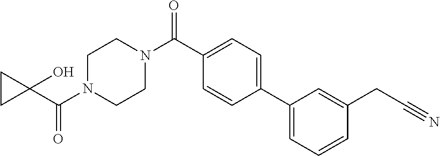 [SMILES: N#CCc1cccc(-c2ccc(C(=O)N3CCN(C(=O)C4(O)CC4)CC3)cc2)c1] |
| N-(4′-(4-(1-hydroxycyclopropane-1- carbonyl)piperazine-1-carbonyl)- [1,1′-biphenyl]-3- yl)cyclopentanesulfonamide | 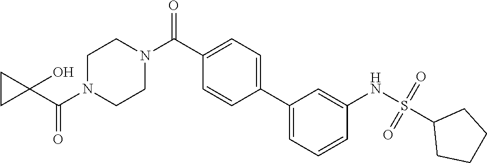 [SMILES: O=C(c1ccc(-c2cccc(NS(=O)(=O)C3CCCC3)c2)cc1)N1CCN(C(=O)C2(O)CC2)CC1] |
| (S)-(4-(1-hydroxycyclopropane-1- carbonyl)-2-methylpiperazin-1- yl)(4-(1-methyl-1H- benzo[d]imidazol-5- yl)phenyl)methanone | 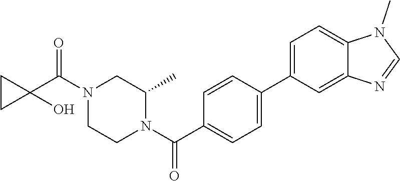 [SMILES: C[C@H]1CN(C(=O)C2(O)CC2)CCN1C(=O)c1ccc(-c2ccc3c(c2)ncn3C)cc1] |
| 1-cyclopropyl-N-(4′-(4-(1- hydroxycyclopropane-1- carbonyl)piperazine-1-carbonyl)- [1,1′-biphenyl]-3- yl)methanesulfonamide | 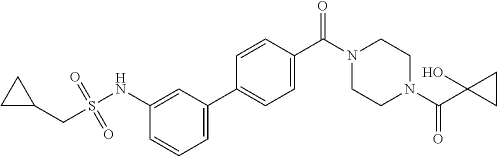 [SMILES: O=C(c1ccc(-c2cccc(NS(=O)(=O)CC3CC3)c2)cc1)N1CCN(C(=O)C2(O)CC2)CC1] |
| (4-(3-chloro-4-(6- fluorobenzo[d]oxazol-2- yl)benzoyl)piperazin-1-yl)(1- hydroxycyclopropyl)methanone | 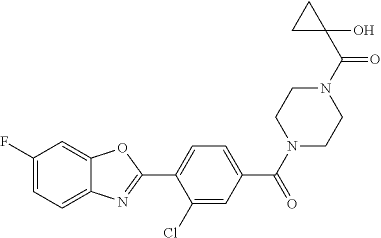 [SMILES: O=C(c1ccc(-c2nc3ccc(F)cc3o2)c(Cl)c1)N1CCN(C(=O)C2(O)CC2)CC1] |
| (4-(2-chloro-4-(2-methyl-2H- indazol-6-yl)benzoyl)piperazin-1- yl)(1- hydroxycyclopropyl)methanone | 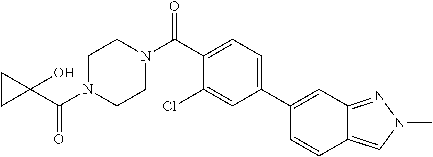 [SMILES: Cn1cc2ccc(-c3ccc(C(=O)N4CCN(C(=O)C5(O)CC5)CC4)c(Cl)c3)cc2n1] |
| (4-(2,6-difluoro-4-(1-methyl-1H- indazol-6-yl)benzoyl)piperazin-1- yl)(1- hydroxycyclopropyl)methanone | 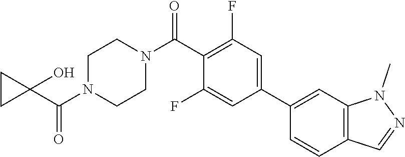 [SMILES: Cn1ncc2ccc(-c3cc(F)c(C(=O)N4CCN(C(=O)C5(O)CC5)CC4)c(F)c3)cc21] |
| (R)-(4-(1-hydroxycyclopropane-1- carbonyl)-2-methylpiperazin-1- yl)(4-(1-methyl-1H- benzo[d]imidazol-5- yl)phenyl)methanone | 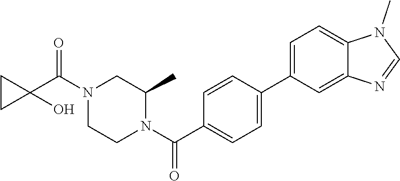 [SMILES: C[C@@H]1CN(C(=O)C2(O)CC2)CCN1C(=O)c1ccc(-c2ccc3c(c2)ncn3C)cc1] |
| N-(6-(4-(4-(1-hydroxycyclopropane- 1-carbonyl)piperazine-1- carbonyl)phenyl)benzo[d]isoxazol- 3-yl)cyclopropanecarboxamide | 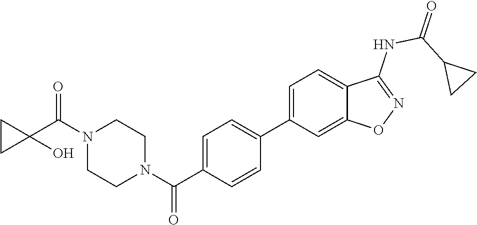 [SMILES: O=C(Nc1noc2cc(-c3ccc(C(=O)N4CCN(C(=O)C5(O)CC5)CC4)cc3)ccc12)C1CC1] |
| ((3S,5R)-3,5-dimethyl-4-(4-(1- methyl-1H-benzo[d]imidazol-5- yl)benzoyl)piperazin-1-yl)(1- hydroxycyclopropyl)methanone | 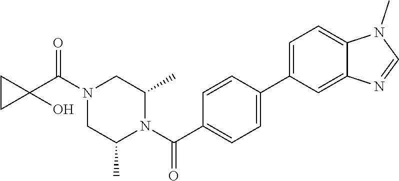 [SMILES: C[C@@H]1CN(C(=O)C2(O)CC2)C[C@H](C)N1C(=O)c1ccc(-c2ccc3c(c2)ncn3C)cc1] |
| isopropyl (4′-(4-(1- hydroxycyclopropane-1- carbonyl)piperazine-1-carbonyl)- [1,1′-biphenyl]-3-yl)carbamate | 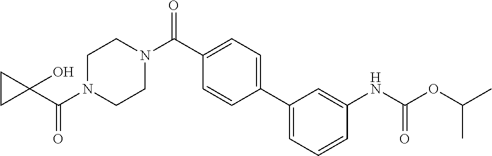 [SMILES: CC(C)OC(=O)Nc1cccc(-c2ccc(C(=O)N3CCN(C(=O)C4(O)CC4)CC3)cc2)c1] |
| (4-(4-(3-amino-1H-indazol-5- yl)benzoyl)piperazin-1-yl)(1- hydroxycyclopropyl)methanone | 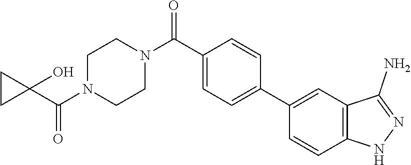 [SMILES: Nc1n[nH]c2ccc(-c3ccc(C(=O)N4CCN(C(=O)C5(O)CC5)CC4)cc3)cc12] |
| (R)-(4-(1-hydroxycyclopropane-1- carbonyl)-3-methylpiperazin-1- yl)(4-(1-methyl-1H- benzo[d]imidazol-5- yl)phenyl)methanone | 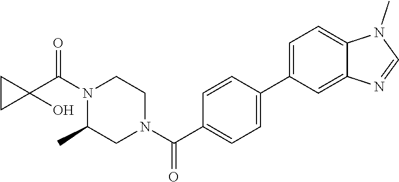 [SMILES: C[C@@H]1CN(C(=O)c2ccc(-c3ccc4c(c3)ncn4C)cc2)CCN1C(=O)C1(O)CC1] |
| 3,3-difluoro-N-(4′-(4-(1- hydroxycyclopropane-1- carbonyl)piperazine-1-carbonyl)- [1,1′-biphenyl]-3-yl)cyclobutane-1- carboxamide | 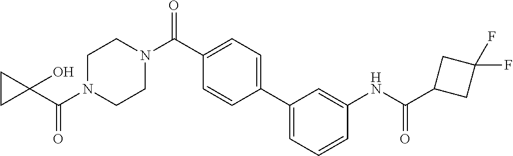 [SMILES: O=C(Nc1cccc(-c2ccc(C(=O)N3CCN(C(=O)C4(O)CC4)CC3)cc2)c1)C1CC(F)(F)C1] |
| ((2S,6R)-4-(4-(6- fluorobenzo[d]oxazol-2-yl)benzoyl)- 2,6-dimethylpiperazin-1-yl)(1- hydroxycyclopropyl)methanone | 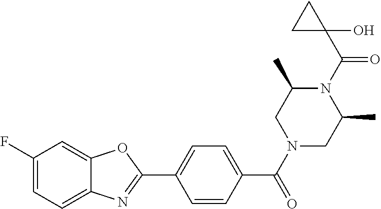 [SMILES: C[C@@H]1CN(C(=O)c2ccc(-c3nc4ccc(F)cc4o3)cc2)C[C@H](C)N1C(=O)C1(O)CC1] |
| (4-(1-hydroxycyclopropane-1- carbonyl)piperazin-1-yl)(4-(1-(2- hydroxyethyl)-1H- benzo[d]imidazol-6- yl)phenyl)methanone | 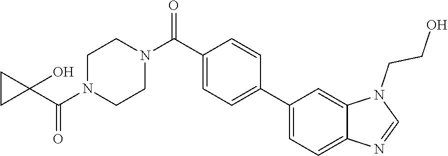 [SMILES: O=C(c1ccc(-c2ccc3ncn(CCO)c3c2)cc1)N1CCN(C(=O)C2(O)CC2)CC1] |
| (4-(4-(1H-benzo[d]imidazol-4-yl)-2- chlorobenzoyl)piperazin-1-yl)(1- hydroxycyclopropyl)methanone | 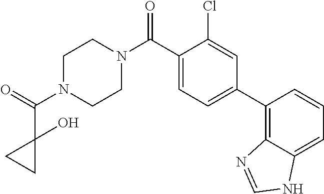 [SMILES: O=C(c1ccc(-c2cccc3[nH]cnc23)cc1Cl)N1CCN(C(=O)C2(O)CC2)CC1] |
| (4-(4-(5-fluoroindolin-1- yl)benzoyl)piperazin-1-yl)(1- hydroxycyclopropyl)methanone | 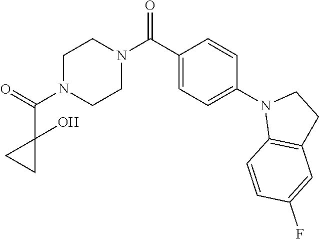 [SMILES: O=C(c1ccc(N2CCc3cc(F)ccc32)cc1)N1CCN(C(=O)C2(O)CC2)CC1] |
| (4-(4-(3-chloroimidazo[1,2- a]pyridin-2-yl)-2- fluorobenzoyl)piperazin-1-yl)(1- hydroxycyclopropyl)methanone | 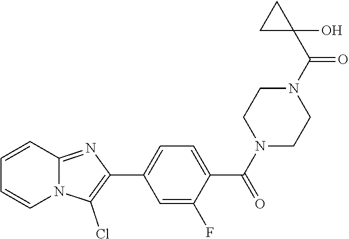 [SMILES: O=C(c1ccc(-c2nc3ccccn3c2Cl)cc1F)N1CCN(C(=O)C2(O)CC2)CC1] |
| (4-(4′-chloro-2′,3-difluoro-[1,1′- biphenyl]-4-carbonyl)piperazin-1- yl)(1- hydroxycyclopropyl)methanone | 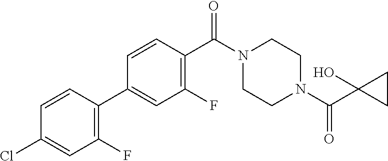 [SMILES: O=C(c1ccc(-c2ccc(Cl)cc2F)cc1F)N1CCN(C(=O)C2(O)CC2)CC1] |
| (4-(2,4′-dichloro-2′-fluoro-[1,1′- biphenyl]-4-carbonyl)piperazin-1- yl)(1- hydroxycyclopropyl)methanone | 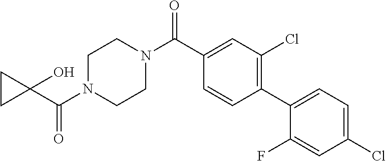 [SMILES: O=C(c1ccc(-c2ccc(Cl)cc2F)c(Cl)c1)N1CCN(C(=O)C2(O)CC2)CC1] |
| (4-(2-chloro-4-(6- chlorobenzo[d]oxazol-2- yl)benzoyl)piperazin-1-yl)(1- hydroxycyclopropyl)methanone | 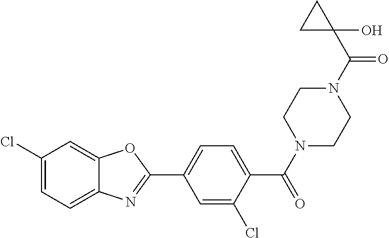 [SMILES: O=C(c1ccc(-c2nc3ccc(Cl)cc3o2)cc1Cl)N1CCN(C(=O)C2(O)CC2)CC1] |
| (4-(4-(5-fluoroisoindolin-2- yl)benzoyl)piperazin-1-yl)(1- hydroxycyclopropyl)methanone | 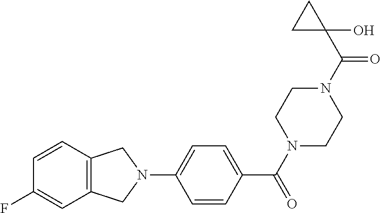 [SMILES: O=C(c1ccc(N2Cc3ccc(F)cc3C2)cc1)N1CCN(C(=O)C2(O)CC2)CC1] |
| (4-(1-hydroxycyclopropane-1- carbonyl)piperazin-1-yl)(4-(3- methyl-1H-indazol-4- yl)phenyl)methanone | 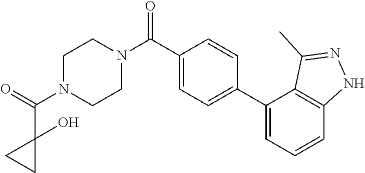 [SMILES: Cc1n[nH]c2cccc(-c3ccc(C(=O)N4CCN(C(=O)C5(O)CC5)CC4)cc3)c12] |
| (4-(1-hydroxycyclopropane-1- carbonyl)piperazin-1-yl)(4-(6- methoxybenzo[d]oxazol-2- yl)phenyl)methanone | 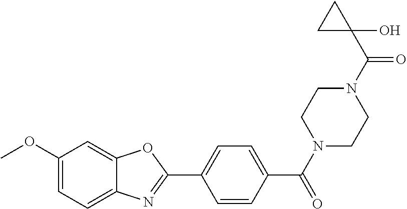 [SMILES: COc1ccc2nc(-c3ccc(C(=O)N4CCN(C(=O)C5(O)CC5)CC4)cc3)oc2c1] |
| (4-(1-hydroxycyclopropane-1- carbonyl)piperazin-1-yl)(4-(3- methyl-1H-indazol-7- yl)phenyl)methanone | 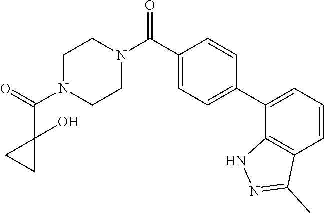 [SMILES: Cc1n[nH]c2c(-c3ccc(C(=O)N4CCN(C(=O)C5(O)CC5)CC4)cc3)cccc12] |
| (R)-(4-(4′-chloro-2′-fluoro-[1,1′- biphenyl]-4-carbonyl)-2- methylpiperazin-1-yl)(1- hydroxycyclopropyl)methanone | 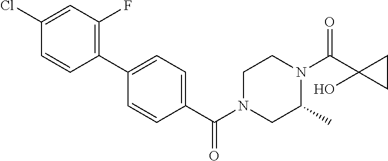 [SMILES: C[C@@H]1CN(C(=O)c2ccc(-c3ccc(Cl)cc3F)cc2)CCN1C(=O)C1(O)CC1] |
| 1-(4′-(4-(1-hydroxycyclopropane-1- carbonyl)piperazine-1-carbonyl)- [1,1′-biphenyl]-3-yl)cyclobutane-1- carbonitrile | 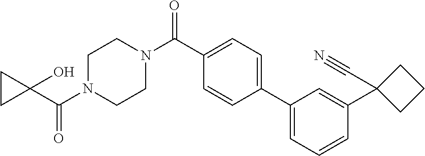 [SMILES: N#CC1(c2cccc(-c3ccc(C(=O)N4CCN(C(=O)C5(O)CC5)CC4)cc3)c2)CCC1] |
| (4-(2-fluoro-4-(6- fluorobenzo[d]oxazol-2- yl)benzoyl)piperazin-1-yl)(1- hydroxycyclopropyl)methanone | 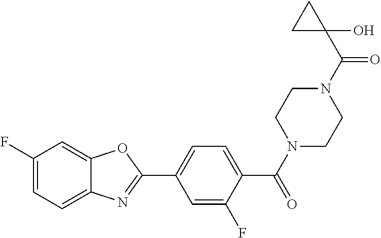 [SMILES: O=C(c1ccc(-c2nc3ccc(F)cc3o2)cc1F)N1CCN(C(=O)C2(O)CC2)CC1] |
| (4-(1-hydroxycyclopropane-1- carbonyl)piperazin-1-yl)(4-(1,3,4,5- tetrahydro-2H-pyrido[4,3-b]indol-2- yl)phenyl)methanone | 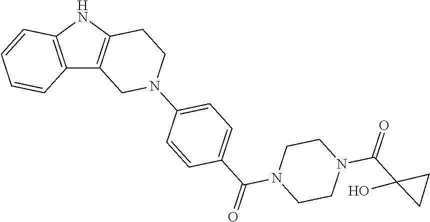 [SMILES: O=C(c1ccc(N2CCc3[nH]c4ccccc4c3C2)cc1)N1CCN(C(=O)C2(O)CC2)CC1] |
| (4-(1-hydroxycyclopropane-1- carbonyl)piperazin-1-yl)(4-(2- methyl-1H-indol-1- yl)phenyl)methanone | 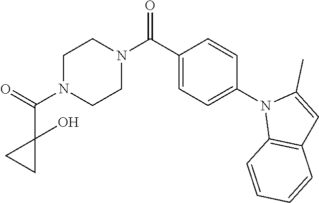 [SMILES: Cc1cc2ccccc2n1-c1ccc(C(=O)N2CCN(C(=O)C3(O)CC3)CC2)cc1] |
| (S)-(4-(1-aminocyclopropane-1- carbonyl)-3-methylpiperazin-1- yl)(4-(1-methyl-1H- benzo[d]imidazol-5- yl)phenyl)methanone | 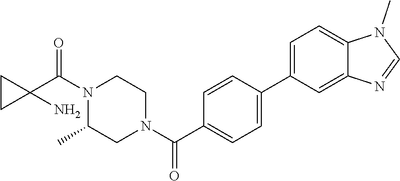 [SMILES: C[C@H]1CN(C(=O)c2ccc(-c3ccc4c(c3)ncn4C)cc2)CCN1C(=O)C1(N)CC1] |
| (4-(3-fluoro-4-(1-methyl-1H- benzo[d]imidazol-5- yl)benzoyl)piperazin-1-yl)(1- hydroxycyclopropyl)methanone | 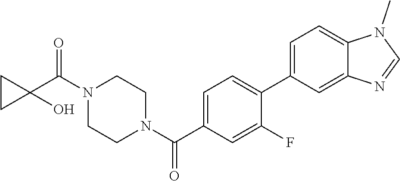 [SMILES: Cn1cnc2cc(-c3ccc(C(=O)N4CCN(C(=O)C5(O)CC5)CC4)cc3F)ccc21] |
| (4-(1-aminocyclobutane-1- carbonyl)piperazin-1-yl)(2-fluoro-4- (1-methyl-1H-benzo[d]imidazol-5- yl)cyclohex-1-en-1-yl)methanone | 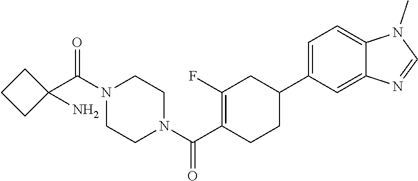 [SMILES: Cn1cnc2cc(C3CCC(C(=O)N4CCN(C(=O)C5(N)CCC5)CC4)=C(F)C3)ccc21] |
| (4-(3-fluoro-4-(1-methyl-1H- indazol-6-yl)benzoyl)piperazin-1- yl)(1- hydroxycyclopropyl)methanone | 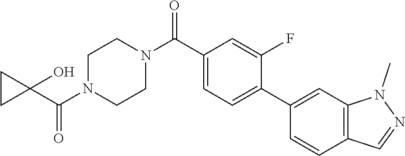 [SMILES: Cn1ncc2ccc(-c3ccc(C(=O)N4CCN(C(=O)C5(O)CC5)CC4)cc3F)cc21] |
| (S)-(4-(4-(6-fluorobenzo[d]oxazol-2- yl)benzoyl)-2-methylpiperazin-1- hydroxycyclopropyl)methanone | 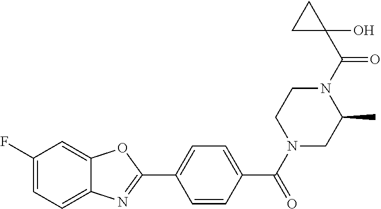 [SMILES: C[C@H]1CN(C(=O)c2ccc(-c3nc4ccc(F)cc4o3)cc2)CCN1C(=O)C1(O)CC1] |
| (4-(4-(4-fluoroisoindolin-2- yl)benzoyl)piperazin-1-yl)(1- hydroxycyclopropyl)methanone | 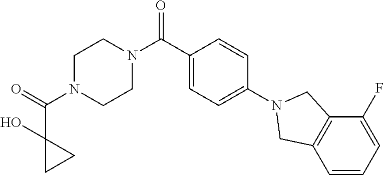 [SMILES: O=C(c1ccc(N2Cc3cccc(F)c3C2)cc1)N1CCN(C(=O)C2(O)CC2)CC1] |
| (4-(2-chloro-3′- (cyclopropylsulfonyl)-[1,1′- biphenyl]-4-carbonyl)piperazin-1- yl)(1- hydroxycyclopropyl)methanone | 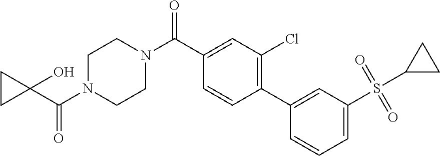 [SMILES: O=C(c1ccc(-c2cccc(S(=O)(=O)C3CC3)c2)c(Cl)c1)N1CCN(C(=O)C2(O)CC2)CC1] |
| (4-(4-(7-fluoroindolin-1- yl)benzoyl)piperazin-1-yl)(1- hydroxycyclopropyl)methanone | 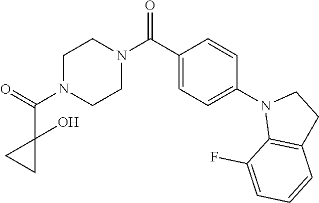 [SMILES: O=C(c1ccc(N2CCc3cccc(F)c32)cc1)N1CCN(C(=O)C2(O)CC2)CC1] |
| (4-(4-(3-cyclopropyl-1H-indazol-6- yl)benzoyl)piperazin-1-yl)(1- hydroxycyclopropyl)methanone | 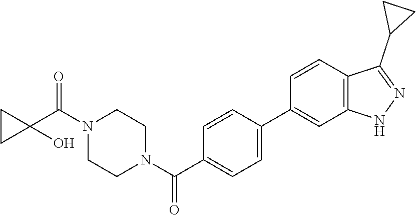 [SMILES: O=C(c1ccc(-c2ccc3c(C4CC4)n[nH]c3c2)cc1)N1CCN(C(=O)C2(O)CC2)CC1] |
| (4-(4-(6-fluorobenzo[d]oxazol-2- yl)benzoyl)-3,3-dimethylpiperazin- hydroxycyclopropyl)methanone | 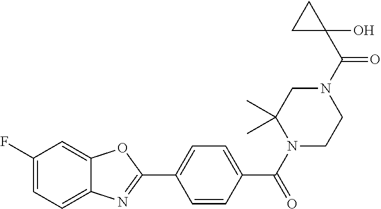 [SMILES: CC1(C)CN(C(=O)C2(O)CC2)CCN1C(=O)c1ccc(-c2nc3ccc(F)cc3o2)cc1] |
| (4-(1-hydroxycyclopropane-1- carbonyl)piperazin-1-yl)(4-(3- methyl-1H-indazol-1- yl)phenyl)methanone | 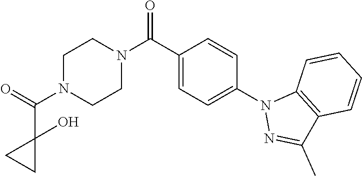 [SMILES: Cc1nn(-c2ccc(C(=O)N3CCN(C(=O)C4(O)CC4)CC3)cc2)c2ccccc12] |
| (4-(1-aminocyclopropane-1- carbonyl)piperazin-1-yl)(2-fluoro-4- (3-methyl-1H-indazol-7- yl)phenyl)methanone | 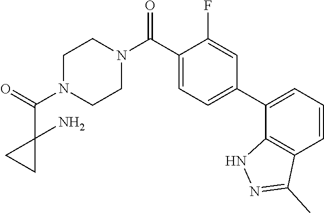 [SMILES: Cc1n[nH]c2c(-c3ccc(C(=O)N4CCN(C(=O)C5(N)CC5)CC4)c(F)c3)cccc12] |
| (4-(1-hydroxycyclopropane-1- carbonyl)piperazin-1-yl)(4-(1- (methylsulfonyl)-1H-indol-6- yl)phenyl)methanone | 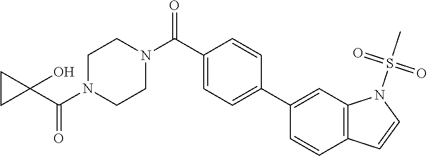 [SMILES: CS(=O)(=O)n1ccc2ccc(-c3ccc(C(=O)N4CCN(C(=O)C5(O)CC5)CC4)cc3)cc21] |
| (4-(1-aminocyclopropane-1- carbonyl)piperazin-1-yl)(4-(1- methyl-1H-benzo[d]imidazol-5- yl)phenyl)methanone | 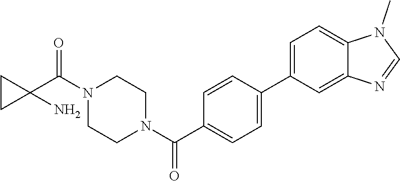 [SMILES: Cn1cnc2cc(-c3ccc(C(=O)N4CCN(C(=O)C5(N)CC5)CC4)cc3)ccc21] |
| (4-(1-hydroxycyclopropane-1- carbonyl)piperazin-1-yl)(4-(2- methylindolin-1- yl)phenyl)methanone | 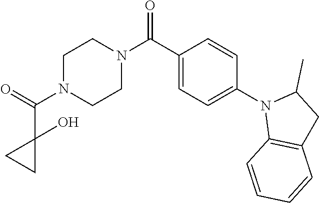 [SMILES: CC1Cc2ccccc2N1c1ccc(C(=O)N2CCN(C(=O)C3(O)CC3)CC2)cc1] |
| (4-(4-(5-fluoro-1H- benzo[d][1,2,3]triazol-6- yl)benzoyl)piperazin-1-yl)(1- hydroxycyclopropyl)methanone | 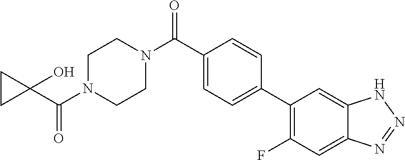 [SMILES: O=C(c1ccc(-c2cc3[nH]nnc3cc2F)cc1)N1CCN(C(=O)C2(O)CC2)CC1] |
| (4-(2,5′-dichloro-2′-fluoro-[1,1′- biphenyl]-4-carbonyl)piperazin-1- yl)(1- hydroxycyclopropyl)methanone | 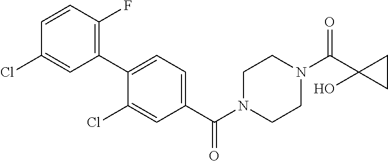 [SMILES: O=C(c1ccc(-c2cc(Cl)ccc2F)c(Cl)c1)N1CCN(C(=O)C2(O)CC2)CC1] |
| ((2S,6R)-4-(4′-chloro-2′-fluoro-[1,1′- biphenyl]-4-carbonyl)-2,6- dimethylpiperazin-1-yl)(1- hydroxycyclopropyl)methanone | 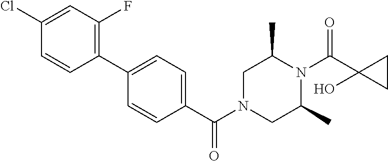 [SMILES: C[C@@H]1CN(C(=O)c2ccc(-c3ccc(Cl)cc3F)cc2)C[C@H](C)N1C(=O)C1(O)CC1] |
| N-(4′-(4-(1-hydroxycyclopropane-1- carbonyl)piperazine-1-carbonyl)- [1,1′-biphenyl]-3-yl)tetrahydro-2H- pyran-4-sulfonamide | 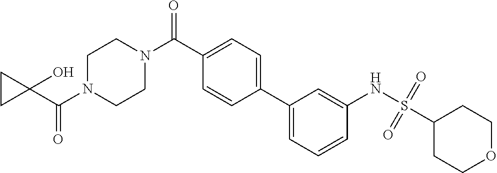 [SMILES: O=C(c1ccc(-c2cccc(NS(=O)(=O)C3CCOCC3)c2)cc1)N1CCN(C(=O)C2(O)CC2)CC1] |
| (4-(1-aminocyclopropane-1- carbonyl)piperazin-1-yl)(4-(1- methyl-1H-indazol-6- yl)phenyl)methanone | 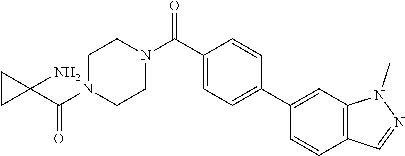 [SMILES: Cn1ncc2ccc(-c3ccc(C(=O)N4CCN(C(=O)C5(N)CC5)CC4)cc3)cc21] |
| (4-(1-hydroxycyclopropane-1- carbonyl)piperazin-1-yl)(4-(7- methoxy-3,4-dihydroisoquinolin- 2(1H)-yl)phenyl)methanone | 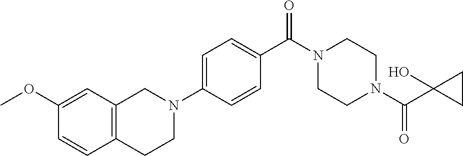 [SMILES: COc1ccc2c(c1)CN(c1ccc(C(=O)N3CCN(C(=O)C4(O)CC4)CC3)cc1)CC2] |
| 1-(3′-fluoro-4′-(4-(1- hydroxycyclopropane-1- carbonyl)piperazine-1-carbonyl)- [1,1′-biphenyl]-3-yl)cyclopropane- 1-carbonitrile | 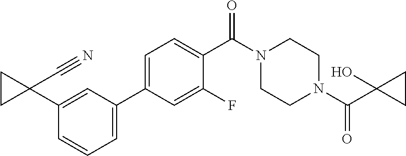 [SMILES: N#CC1(c2cccc(-c3ccc(C(=O)N4CCN(C(=O)C5(O)CC5)CC4)c(F)c3)c2)CC1] |
| (4-(2-fluoro-4-(2-methyl-1H-indol- 1-yl)benzoyl)piperazin-1-yl)(1- hydroxycyclopropyl)methanone | 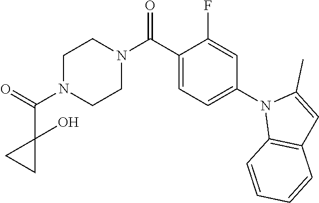 [SMILES: Cc1cc2ccccc2n1-c1ccc(C(=O)N2CCN(C(=O)C3(O)CC3)CC2)c(F)c1] |
| (4-(4-(1H-benzo[d]imidazol-1- yl)benzoyl)piperazin-1-yl)(1- hydroxycyclopropyl)methanone | 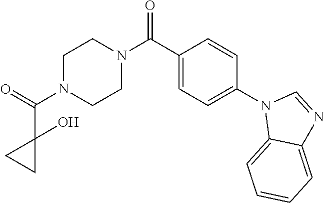 [SMILES: O=C(c1ccc(-n2cnc3ccccc32)cc1)N1CCN(C(=O)C2(O)CC2)CC1] |
| 1-(4′-(4-(1-hydroxycyclopropane-1- carbonyl)piperazine-1-carbonyl)- [1,1′-biphenyl]-3-yl)cyclopropane- 1-carbonitrile | 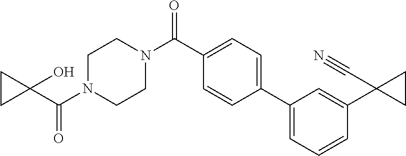 [SMILES: N#CC1(c2cccc(-c3ccc(C(=O)N4CCN(C(=O)C5(O)CC5)CC4)cc3)c2)CC1] |
| (4-(2-fluoro-4-(3-methyl-1H- indazol-1-yl)benzoyl)piperazin-1- yl)(1- hydroxycyclopropyl)methanone | 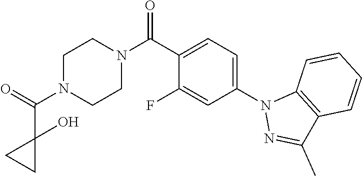 [SMILES: Cc1nn(-c2ccc(C(=O)N3CCN(C(=O)C4(O)CC4)CC3)c(F)c2)c2ccccc12] |
| (4-(1-aminocyclopropane-1- carbonyl)piperazin-1-yl)(2-fluoro-4- (1-methyl-1H-indazol-6- yl)phenyl)methanone | 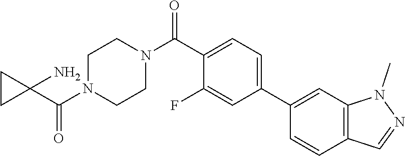 [SMILES: Cn1ncc2ccc(-c3ccc(C(=O)N4CCN(C(=O)C5(N)CC5)CC4)c(F)c3)cc21] |
| 2-(4-(4-(1-hydroxycyclopropane-1- carbonyl)piperazine-1- carbonyl)phenyl)-1,2,3,4- tetrahydroisoquinoline-7- carbonitrile | 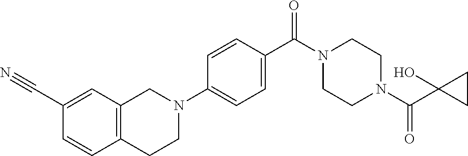 [SMILES: N#Cc1ccc2c(c1)CN(c1ccc(C(=O)N3CCN(C(=O)C4(O)CC4)CC3)cc1)CC2] |
| (4-(1-hydroxycyclopropane-1- carbonyl)piperazin-1-yl)(4-(1-(2- hydroxyethyl)-1H- benzo[d]imidazol-5- yl)phenyl)methanone | 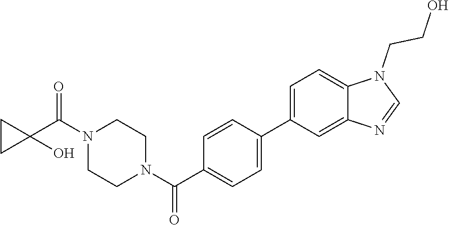 [SMILES: O=C(c1ccc(-c2ccc3c(c2)ncn3CCO)cc1)N1CCN(C(=O)C2(O)CC2)CC1] |
| (R)-(4-(4-(6-fluorobenzo[d]oxazol-2- yl)benzoyl)-2-methylpiperazin-1- hydroxycyclopropyl)methanone | 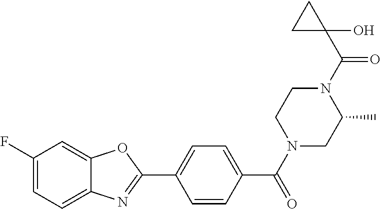 [SMILES: C[C@@H]1CN(C(=O)c2ccc(-c3nc4ccc(F)cc4o3)cc2)CCN1C(=O)C1(O)CC1] |
| (4-(4-(1H-indazol-1- yl)benzoyl)piperazin-1-yl)(1- hydroxycyclopropyl)methanone | 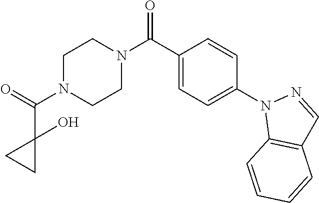 [SMILES: O=C(c1ccc(-n2ncc3ccccc32)cc1)N1CCN(C(=O)C2(O)CC2)CC1] |
| (4-(3,5′-dichloro-2′-fluoro-[1,1′- biphenyl]-4-carbonyl)piperazin-1- yl)(1- hydroxycyclopropyl)methanone | 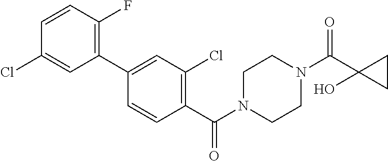 [SMILES: O=C(c1ccc(-c2cc(Cl)ccc2F)cc1Cl)N1CCN(C(=O)C2(O)CC2)CC1] |
| (4-(4-(1,3-dimethyl-1H-indazol-5- yl)-3-fluorobenzoyl)piperazin-1- yl)(1- hydroxycyclopropyl)methanone | 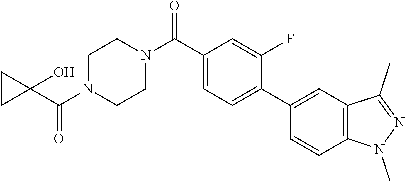 [SMILES: Cc1nn(C)c2ccc(-c3ccc(C(=O)N4CCN(C(=O)C5(O)CC5)CC4)cc3F)cc12] |
| (4-(4′-chloro-2′-fluoro-[1,1′- biphenyl]-4-carbonyl)-4,7- diazaspiro[2.5]octan-7-yl)(1- hydroxycyclopropyl)methanone | 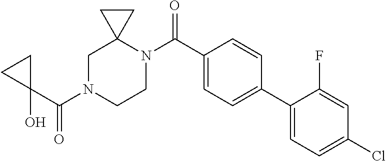 [SMILES: O=C(c1ccc(-c2ccc(Cl)cc2F)cc1)N1CCN(C(=O)C2(O)CC2)CC12CC2] |
| (4-(1-aminocyclopropane-1- carbonyl)piperazin-1-yl)(2-fluoro-4- (7-fluoro-1H-indazol-3- yl)phenyl)methanone | 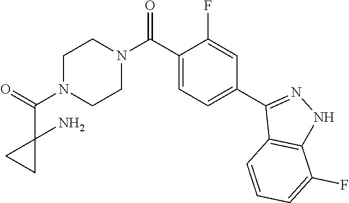 [SMILES: NC1(C(=O)N2CCN(C(=O)c3ccc(-c4n[nH]c5c(F)cccc45)cc3F)CC2)CC1] |
| 6-(1-(4-(1-hydroxycyclopropane-1- carbonyl)piperazine-1- carbonyl)piperidin-4-yl)-2- naphthonitrile | 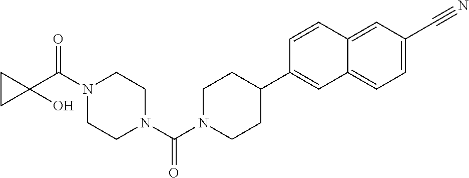 [SMILES: N#Cc1ccc2cc(C3CCN(C(=O)N4CCN(C(=O)C5(O)CC5)CC4)CC3)ccc2c1] |
| 1-(4′-(4-(1-hydroxycyclopropane-1- carbonyl)piperazine-1-carbonyl)- [1,1′-biphenyl]-3-yl)imidazolidin-2- one | 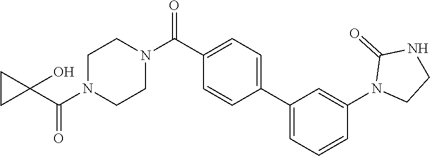 [SMILES: O=C(c1ccc(-c2cccc(N3CCNC3=O)c2)cc1)N1CCN(C(=O)C2(O)CC2)CC1] |
| (4-(1-aminocyclopropane-1- carbonyl)piperazin-1-yl)(2,6- difluoro-4-(2-methyl-2H-indazol-6- yl)phenyl)methanone | 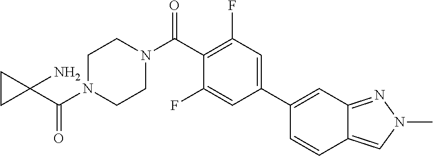 [SMILES: Cn1cc2ccc(-c3cc(F)c(C(=O)N4CCN(C(=O)C5(N)CC5)CC4)c(F)c3)cc2n1] |
| (4-(4-(1,3-dimethyl-1H-indazol-5- yl)benzoyl)piperazin-1-yl)(oxetan-2- yl)methanone | 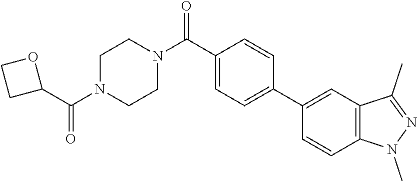 [SMILES: Cc1nn(C)c2ccc(-c3ccc(C(=O)N4CCN(C(=O)C5CCO5)CC4)cc3)cc12] |
| (4-(1-aminocyclopropane-1- carbonyl)piperazin-1-yl)(2-fluoro-4- (2-methyl-2H-indazol-6- yl)phenyl)methanone | 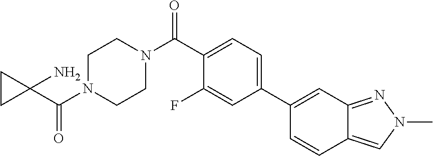 [SMILES: Cn1cc2ccc(-c3ccc(C(=O)N4CCN(C(=O)C5(N)CC5)CC4)c(F)c3)cc2n1] |
| (4-(3′-(5-amino-1H-1,2,4-triazol-3- yl)-[1,1′-biphenyl]-4- carbonyl)piperazin-1-yl)(1- hydroxycyclopropyl)methanone | 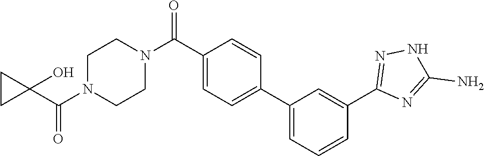 [SMILES: Nc1nc(-c2cccc(-c3ccc(C(=O)N4CCN(C(=O)C5(O)CC5)CC4)cc3)c2)n[nH]1] |
| (7-(1-hydroxycyclopropane-1- carbonyl)-4,7-diazaspiro[2.5]octan- 4-yl)(4-(1-methyl-1H- benzo[d]imidazol-5- yl)phenyl)methanone | 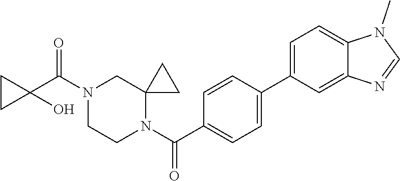 [SMILES: Cn1cnc2cc(-c3ccc(C(=O)N4CCN(C(=O)C5(O)CC5)CC45CC5)cc3)ccc21] |
| (4-(1-hydroxycyclopropane-1- carbonyl)piperazin-1-yl)(4-(2- methyl-1H-indol-3- yl)phenyl)methanone | 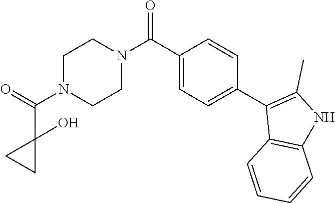 [SMILES: Cc1[nH]c2ccccc2c1-c1ccc(C(=O)N2CCN(C(=O)C3(O)CC3)CC2)cc1] |
| (4-(1-hydroxycyclopropane-1- carbonyl)piperazin-1-yl)(4-(2- methyl-1H-benzo[d]imidazol-5- yl)phenyl)methanone | 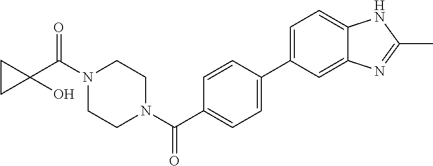 [SMILES: Cc1nc2cc(-c3ccc(C(=O)N4CCN(C(=O)C5(O)CC5)CC4)cc3)ccc2[nH]1] |
| (4-(4-(6-fluorobenzo[d]oxazol-2- yl)benzoyl)-4,7- diazaspiro[2.5]octan-7-yl)(1- hydroxycyclopropyl)methanone | 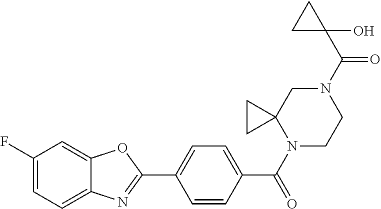 [SMILES: O=C(c1ccc(-c2nc3ccc(F)cc3o2)cc1)N1CCN(C(=O)C2(O)CC2)CC12CC2] |
| (4-(4-(6-fluoro-1-methyl-1H- benzo[d][1,2,3]triazol-5- yl)benzoyl)piperazin-1-yl)(1- hydroxycyclopropyl)methanone | 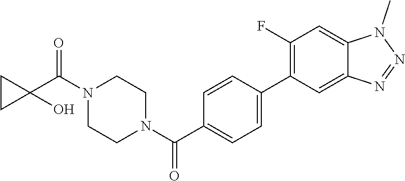 [SMILES: Cn1nnc2cc(-c3ccc(C(=O)N4CCN(C(=O)C5(O)CC5)CC4)cc3)c(F)cc21] |
| (4-(1-hydroxycyclobutane-1- carbonyl)piperazin-1-yl)(4-(1- methyl-1H-benzo[d]imidazol-5- yl)phenyl)methanone | 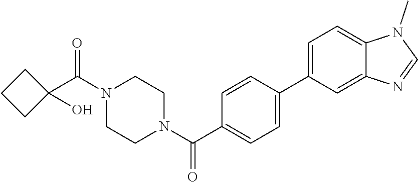 [SMILES: Cn1cnc2cc(-c3ccc(C(=O)N4CCN(C(=O)C5(O)CCC5)CC4)cc3)ccc21] |
| 6-chloro-4-(4-(4-(1- hydroxycyclopropane-1- carbonyl)piperazine-1- carbonyl)phenyl)-1,3-dihydro-2H- benzo[d]imidazol-2-one | 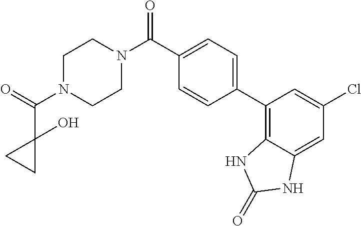 [SMILES: O=C(c1ccc(-c2cc(Cl)cc3[nH]c(=O)[nH]c23)cc1)N1CCN(C(=O)C2(O)CC2)CC1] |
| (4-(1-hydroxycyclopropane-1- carbonyl)piperazin-1-yl)(4-(2- (hydroxymethyl)-1H- benzo[d]imidazol-5- yl)phenyl)methanone | 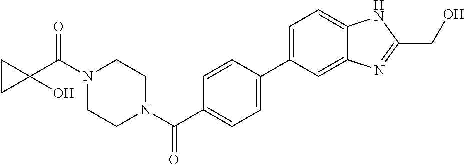 [SMILES: O=C(c1ccc(-c2ccc3[nH]c(CO)nc3c2)cc1)N1CCN(C(=O)C2(O)CC2)CC1] |
| (4-(4-(3-aminobenzo[d]isoxazol-5- yl)benzoyl)piperazin-1-yl)(1- aminocyclopropyl)methanone | 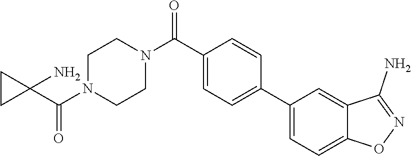 [SMILES: Nc1noc2ccc(-c3ccc(C(=O)N4CCN(C(=O)C5(N)CC5)CC4)cc3)cc12] |
| (4-(4-(2-cyclopropyl-2H-indazol-5- yl)benzoyl)piperazin-1-yl)(1- hydroxycyclopropyl)methanone | 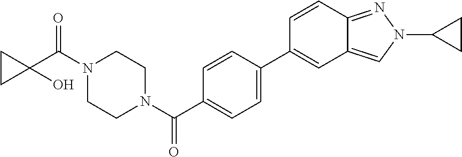 [SMILES: O=C(c1ccc(-c2ccc3nn(C4CC4)cc3c2)cc1)N1CCN(C(=O)C2(O)CC2)CC1] |
| (4-(4-(5,8-dihydro-1,7- naphthyridin-7(6H)-yl)-2- fluorobenzoyl)piperazin-1-yl)(1- hydroxycyclopropyl)methanone | 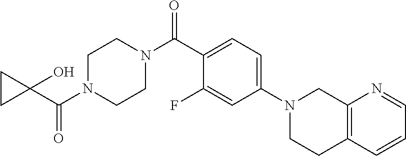 [SMILES: O=C(c1ccc(N2CCc3cccnc3C2)cc1F)N1CCN(C(=O)C2(O)CC2)CC1] |
| (4-(1-hydroxycyclopropane-1- carbonyl)piperazin-1-yl)(4-(3- methyl-3,4-dihydroisoquinolin- 2(1H)-yl)phenyl)methanone | 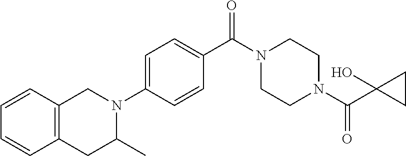 [SMILES: CC1Cc2ccccc2CN1c1ccc(C(=O)N2CCN(C(=O)C3(O)CC3)CC2)cc1] |
| (4-(1-hydroxycyclopropane-1- carbonyl)piperazin-1-yl)(4- (isoindolin-2-yl)phenyl)methanone | 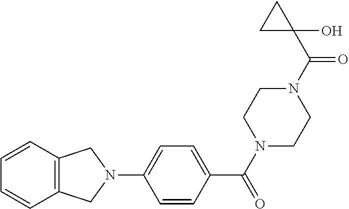 [SMILES: O=C(c1ccc(N2Cc3ccccc3C2)cc1)N1CCN(C(=O)C2(O)CC2)CC1] |
| (R)-(4-(4′-chloro-2′-fluoro-[1,1′- biphenyl]-4-carbonyl)-3- methylpiperazin-1-yl)(1- hydroxycyclopropyl)methanone | 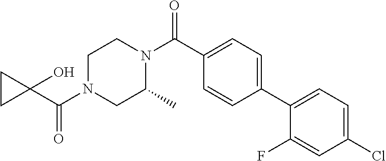 [SMILES: C[C@@H]1CN(C(=O)C2(O)CC2)CCN1C(=O)c1ccc(-c2ccc(Cl)cc2F)cc1] |
| (4-(1-hydroxycyclopropane-1- carbonyl)piperazin-1-yl)(4-(3- methyl-1H-pyrazolo[3,4-b]pyridin- 5-yl)phenyl)methanone | 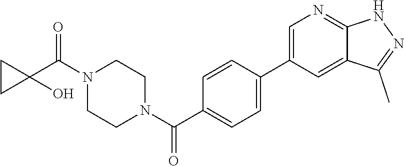 [SMILES: Cc1n[nH]c2ncc(-c3ccc(C(=O)N4CCN(C(=O)C5(O)CC5)CC4)cc3)cc12] |
| (4-(1-aminocyclopropane-1- carbonyl)piperazin-1-yl)(4-(3- methyl-1H-indazol-6- yl)phenyl)methanone | 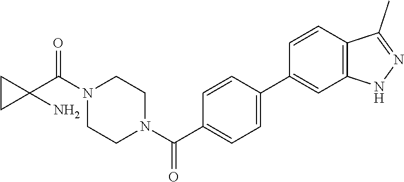 [SMILES: Cc1n[nH]c2cc(-c3ccc(C(=O)N4CCN(C(=O)C5(N)CC5)CC4)cc3)ccc12] |
| (4-(1-aminocyclopropane-1- carbonyl)piperazin-1-yl)(2-fluoro-4- (1-methyl-1H-benzo[d]imidazol-5- yl)phenyl)methanone | 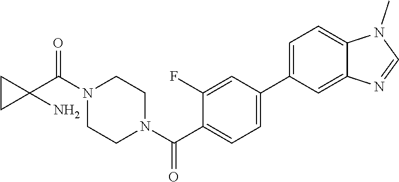 [SMILES: Cn1cnc2cc(-c3ccc(C(=O)N4CCN(C(=O)C5(N)CC5)CC4)c(F)c3)ccc21] |
| (4-(4-(3-hydroxy-1-methyl-1H- indazol-6-yl)benzoyl)piperazin-1- yl)(1- hydroxycyclopropyl)methanone | 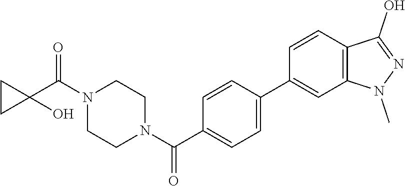 [SMILES: Cn1nc(O)c2ccc(-c3ccc(C(=O)N4CCN(C(=O)C5(O)CC5)CC4)cc3)cc21] |
| (4-(4-(3,4-dihydroquinolin-1(2H)- yl)benzoyl)piperazin-1-yl)(1- hydroxycyclopropyl)methanone | 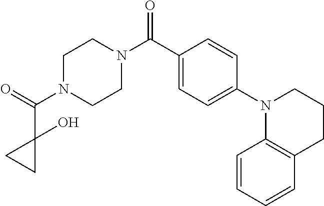 [SMILES: O=C(c1ccc(N2CCCc3ccccc32)cc1)N1CCN(C(=O)C2(O)CC2)CC1] |
| (4-(1-aminocyclopropane-1- carbonyl)piperazin-1-yl)(4-(3- methyl-1H-indazol-7- yl)phenyl)methanone | 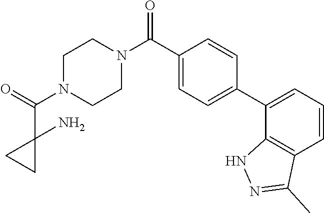 [SMILES: Cc1n[nH]c2c(-c3ccc(C(=O)N4CCN(C(=O)C5(N)CC5)CC4)cc3)cccc12] |
| (4-(4-(6,7-dihydrothieno[3,2- c]pyridin-5(4H)- yl)benzoyl)piperazin-1-yl)(1- hydroxycyclopropyl)methanone | 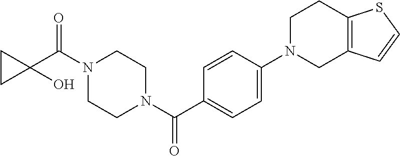 [SMILES: O=C(c1ccc(N2CCc3sccc3C2)cc1)N1CCN(C(=O)C2(O)CC2)CC1] |
| (4-(1-aminocyclobutane-1- carbonyl)piperazin-1-yl)(4-(1- methyl-1H-indazol-6- yl)phenyl)methanone | 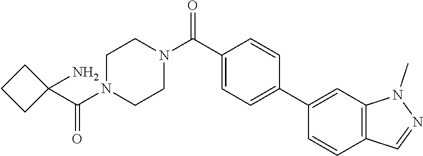 [SMILES: Cn1ncc2ccc(-c3ccc(C(=O)N4CCN(C(=O)C5(N)CCC5)CC4)cc3)cc21] |
| (4-(3′-(2-aminothiazol-4-yl)-[1,1′- biphenyl]-4-carbonyl)piperazin-1- yl)(1- hydroxycyclopropyl)methanone | 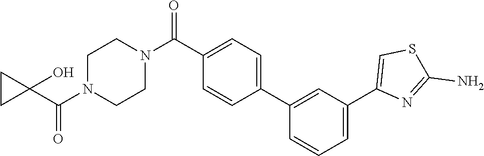 [SMILES: Nc1nc(-c2cccc(-c3ccc(C(=O)N4CCN(C(=O)C5(O)CC5)CC4)cc3)c2)cs1] |
| (4-(1-aminocyclobutane-1- carbonyl)piperazin-1-yl)(4-(1,3- dimethyl-1H-indazol-5- yl)phenyl)methanone | 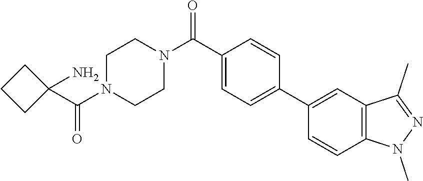 [SMILES: Cc1nn(C)c2ccc(-c3ccc(C(=O)N4CCN(C(=O)C5(N)CCC5)CC4)cc3)cc12] |
| (4-(3′-(cyclopropylsulfonyl)-3- fluoro-[1,1′-biphenyl]-4- carbonyl)piperazin-1-yl)(1- hydroxycyclopropyl)methanone | 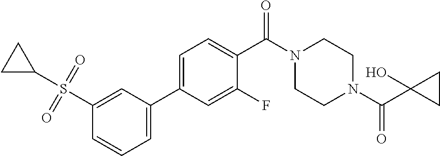 [SMILES: O=C(c1ccc(-c2cccc(S(=O)(=O)C3CC3)c2)cc1F)N1CCN(C(=O)C2(O)CC2)CC1] |
| (4-(4-(6-fluoroquinazolin-2- yl)benzoyl)piperazin-1-yl)(1- hydroxycyclopropyl)methanone | 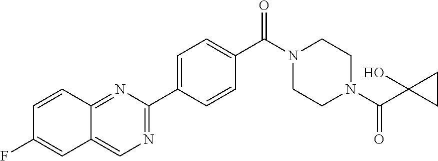 [SMILES: O=C(c1ccc(-c2ncc3cc(F)ccc3n2)cc1)N1CCN(C(=O)C2(O)CC2)CC1] |
| N-(5-(4-(4-(1-hydroxycyclopropane- 1-carbonyl)piperazine-1- carbonyl)phenyl)benzo[d]isoxazol- 3-yl)cyclopropanecarboxamide | 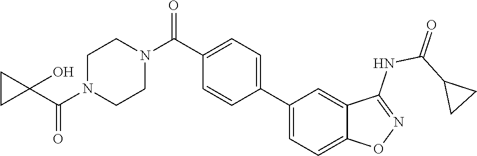 [SMILES: O=C(Nc1noc2ccc(-c3ccc(C(=O)N4CCN(C(=O)C5(O)CC5)CC4)cc3)cc12)C1CC1] |
| 4-(4-(4-(1-hydroxycyclopropane-1- carbonyl)piperazine-1- carbonyl)phenyl)-1-methylindolin- 2-one | 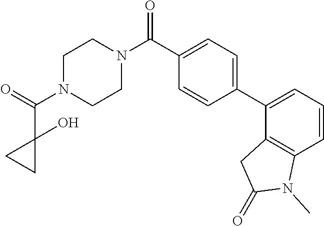 [SMILES: CN1C(=O)Cc2c(-c3ccc(C(=O)N4CCN(C(=O)C5(O)CC5)CC4)cc3)cccc21] |
| (R)-(4-(4-(6-fluorobenzo[d]oxazol-2- yl)benzoyl)-3-methylpiperazin-1- hydroxycyclopropyl)methanone | 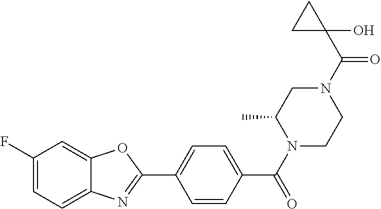 [SMILES: C[C@@H]1CN(C(=O)C2(O)CC2)CCN1C(=O)c1ccc(-c2nc3ccc(F)cc3o2)cc1] |
| (4-(1-hydroxycyclopropane-1- carbonyl)piperazin-1-yl)(4-(2- methyl-1H-benzo[d]imidazol-1- yl)phenyl)methanone | 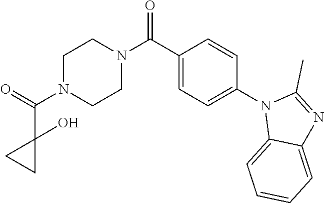 [SMILES: Cc1nc2ccccc2n1-c1ccc(C(=O)N2CCN(C(=O)C3(O)CC3)CC2)cc1] |
| (4-(1-aminocyclopropane-1- carbonyl)piperazin-1-yl)(5′-chloro- 2′-fluoro-[1,1′-biphenyl]-4- yl)methanone | 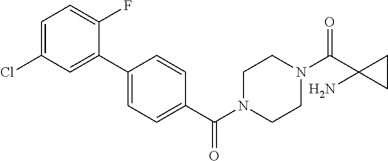 [SMILES: NC1(C(=O)N2CCN(C(=O)c3ccc(-c4cc(Cl)ccc4F)cc3)CC2)CC1] |
| (4-(3-fluoro-4-(2-methyl-2H- indazol-6-yl)benzoyl)piperazin-1- yl)(1- hydroxycyclopropyl)methanone | 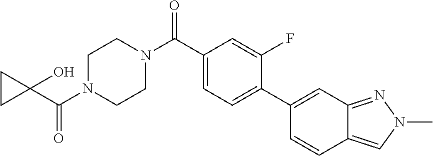 [SMILES: Cn1cc2ccc(-c3ccc(C(=O)N4CCN(C(=O)C5(O)CC5)CC4)cc3F)cc2n1] |
| 4′-(4-(1-hydroxycyclopropane-1- carbonyl)piperazine-1-carbonyl)-N- methyl-[1,1′-biphenyl]-3- carboxamide | 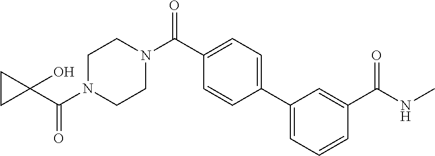 [SMILES: CNC(=O)c1cccc(-c2ccc(C(=O)N3CCN(C(=O)C4(O)CC4)CC3)cc2)c1] |
| (4-(3′-(5-amino-1H-pyrazol-3-yl)- [1,1′-biphenyl]-4- carbonyl)piperazin-1-yl)(1- hydroxycyclopropyl)methanone | 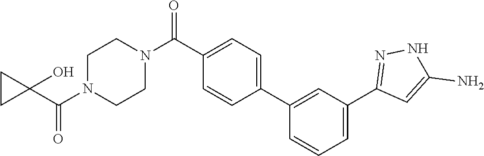 [SMILES: Nc1cc(-c2cccc(-c3ccc(C(=O)N4CCN(C(=O)C5(O)CC5)CC4)cc3)c2)n[nH]1] |
| (S)-(4-(4-(5,8-dihydro-1,7- naphthyridin-7(6H)-yl)benzoyl)-2- methylpiperazin-1-yl)(1- hydroxycyclopropyl)methanone | 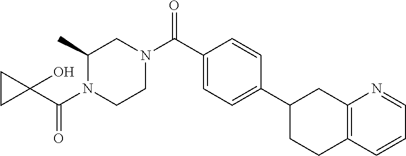 [SMILES: C[C@H]1CN(C(=O)c2ccc(C3CCc4cccnc4C3)cc2)CCN1C(=O)C1(O)CC1] |
| (S)-(4-(4′-chloro-2′-fluoro-[1,1′- biphenyl]-4-carbonyl)-3- methylpiperazin-1-yl)(1- hydroxycyclopropyl)methanone | 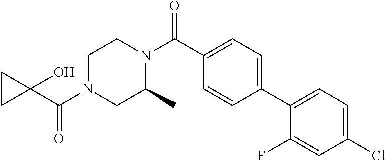 [SMILES: C[C@H]1CN(C(=O)C2(O)CC2)CCN1C(=O)c1ccc(-c2ccc(Cl)cc2F)cc1] |
| 2-((4′-(4-(1-hydroxycyclopropane-1- carbonyl)piperazine-1-carbonyl)- 1′,2′,3′,4′,5′,6′-hexahydro-[1,1′- biphenyl]-3-yl)oxy)-N- methylacetamide | 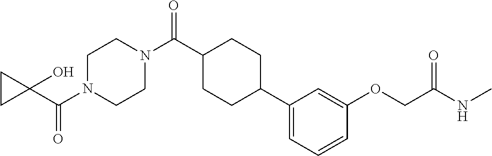 [SMILES: CNC(=O)COc1cccc(C2CCC(C(=O)N3CCN(C(=O)C4(O)CC4)CC3)CC2)c1] |
| (4-(4-(1-cyclopropyl-1H-indazol-5- yl)benzoyl)piperazin-1-yl)(1- hydroxycyclopropyl)methanone | 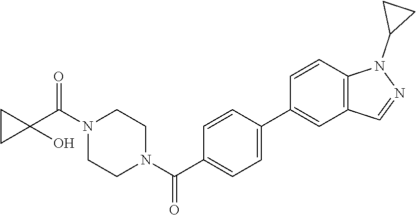 [SMILES: O=C(c1ccc(-c2ccc3c(cnn3C3CC3)c2)cc1)N1CCN(C(=O)C2(O)CC2)CC1] |
| (4-(4-(1-methyl-1H- benzo[d]imidazol-5- yl)benzoyl)piperazin-1-yl)(oxetan-2- yl)methanone | 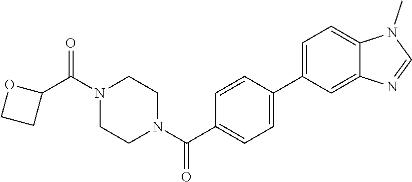 [SMILES: Cn1cnc2cc(-c3ccc(C(=O)N4CCN(C(=O)C5CCO5)CC4)cc3)ccc21] |
| (4-(1- (hydroxymethyl)cyclopropane-1- carbonyl)piperazin-1-yl)(4-(1- methyl-1H-benzo[d]imidazol-5- yl)phenyl)methanone | 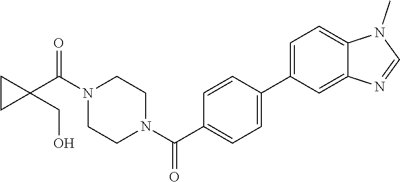 [SMILES: Cn1cnc2cc(-c3ccc(C(=O)N4CCN(C(=O)C5(CO)CC5)CC4)cc3)ccc21] |
| (4-(1-aminocyclobutane-1- carbonyl)piperazin-1-yl)(4-(1- methyl-1H-benzo[d]imidazol-5- yl)phenyl)methanone | 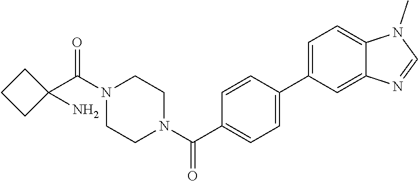 [SMILES: Cn1cnc2cc(-c3ccc(C(=O)N4CCN(C(=O)C5(N)CCC5)CC4)cc3)ccc21] |
| (4-(3-chloro-4-(3- chloroimidazo[1,2-a]pyridin-2- yl)benzoyl)piperazin-1-yl)(1- hydroxycyclopropyl)methanone | 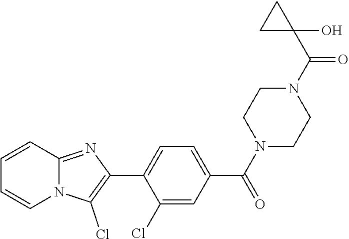 [SMILES: O=C(c1ccc(-c2nc3ccccn3c2Cl)c(Cl)c1)N1CCN(C(=O)C2(O)CC2)CC1] |
| (R)-(4-(1-hydroxycyclopropane-1- carbonyl)-3- (hydroxymethyl)piperazin-1-yl)(4- (1-methyl-1H-benzo[d]imidazol-5- yl)phenyl)methanone | 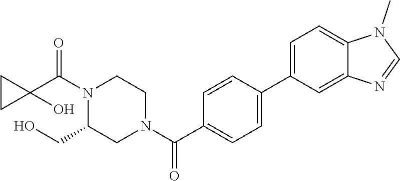 [SMILES: Cn1cnc2cc(-c3ccc(C(=O)N4CCN(C(=O)C5(O)CC5)[C@@H](CO)C4)cc3)ccc21] |
| (4-(1-hydroxycyclopropane-1- carbonyl)piperazin-1-yl)(4-(1,2,4,5- tetrahydro-3H-benzo[d]azepin-3- yl)phenyl)methanone | 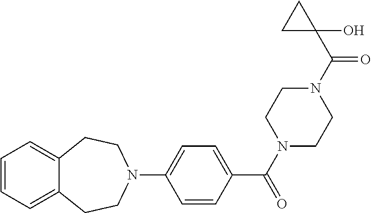 [SMILES: O=C(c1ccc(N2CCc3ccccc3CC2)cc1)N1CCN(C(=O)C2(O)CC2)CC1] |
| ((3S,5R)-4-(1-aminocyclopropane-1- carbonyl)-3,5-dimethylpiperazin-1- yl)(4-(6-fluorobenzo[d]oxazol-2- yl)phenyl)methanone | 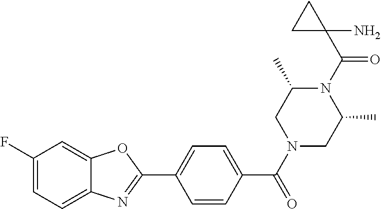 [SMILES: C[C@@H]1CN(C(=O)c2ccc(-c3nc4ccc(F)cc4o3)cc2)C[C@H](C)N1C(=O)C1(N)CC1] |
| (S)-(4-(1-aminocyclopropane-1- carbonyl)-3-methylpiperazin-1- yl)(4-(6-fluorobenzo[d]oxazol-2- yl)phenyl)methanone | 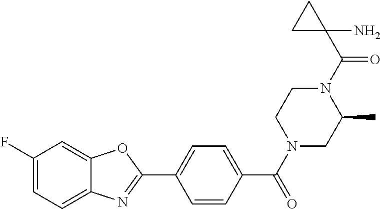 [SMILES: C[C@H]1CN(C(=O)c2ccc(-c3nc4ccc(F)cc4o3)cc2)CCN1C(=O)C1(N)CC1] |
| (4-(1-hydroxycyclopropane-1- carbonyl)piperazin-1-yl)(3′-(5- methyl-1,3,4-thiadiazol-2-yl)-[1,1′- biphenyl]-4-yl)methanone | 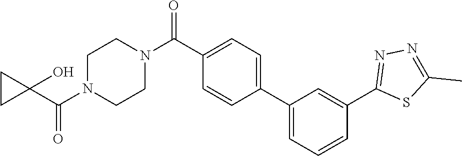 [SMILES: Cc1nnc(-c2cccc(-c3ccc(C(=O)N4CCN(C(=O)C5(O)CC5)CC4)cc3)c2)s1] |
| (4-(1-hydroxycyclopropane-1- carbonyl)piperazin-1-yl)(4-(3- methyl-2H-indazol-2- yl)phenyl)methanone | 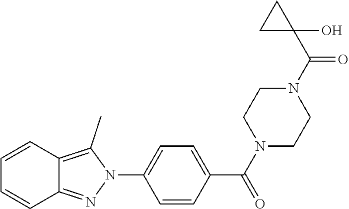 [SMILES: Cc1c2ccccc2nn1-c1ccc(C(=O)N2CCN(C(=O)C3(O)CC3)CC2)cc1] |
| 2-(4′-(4-(1-hydroxycyclopropane-1- carbonyl)piperazine-1-carbonyl)- [1,1′-biphenyl]-3-yl)-2- methylpropanenitrile | 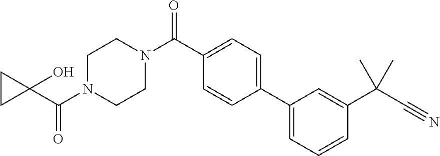 [SMILES: CC(C)(C#N)c1cccc(-c2ccc(C(=O)N3CCN(C(=O)C4(O)CC4)CC3)cc2)c1] |
| (S)-(4-(3′-(cyclopropylsulfonyl)- 1,2,3,4,5,6-hexahydro-[1,1′- biphenyl]-4-carbonyl)-2- methylpiperazin-1-yl)(1- hydroxycyclopropyl)methanone | 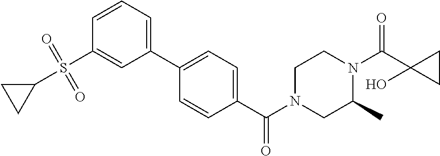 [SMILES: C[C@H]1CN(C(=O)c2ccc(-c3cccc(S(=O)(=O)C4CC4)c3)cc2)CCN1C(=O)C1(O)CC1] |
| 1-(4′-(4-(1-hydroxycyclopropane-1- carbonyl)piperazine-1-carbonyl)- [1,1′-biphenyl]-3-yl)pyrrolidin-2- one | 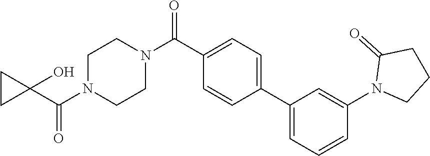 [SMILES: O=C(c1ccc(-c2cccc(N3CCCC3=O)c2)cc1)N1CCN(C(=O)C2(O)CC2)CC1] |
| (4-(3′-(5-amino-1l3,2l2-isoxazol-3- yl)-3-fluoro-[1,1′-biphenyl]-4- carbonyl)piperazin-1-yl)(1- aminocyclopropyl)methanone | 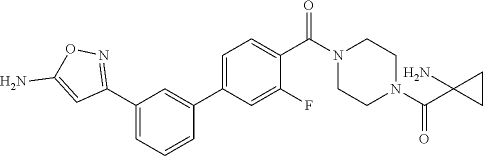 [SMILES: Nc1cc(-c2cccc(-c3ccc(C(=O)N4CCN(C(=O)C5(N)CC5)CC4)c(F)c3)c2)no1] |
| 6-(4-(4-(1-hydroxycyclopropane-1- carbonyl)piperazine-1- carbonyl)phenyl)-N- methylpicolinamide | 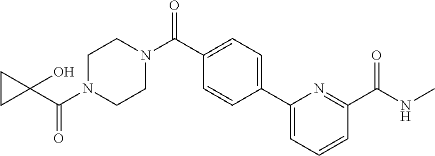 [SMILES: CNC(=O)c1cccc(-c2ccc(C(=O)N3CCN(C(=O)C4(O)CC4)CC3)cc2)n1] |
| (S)-(4-(4-(6-fluorobenzo[d]oxazol-2- yl)benzoyl)-3-methylpiperazin-1- hydroxycyclopropyl)methanone | 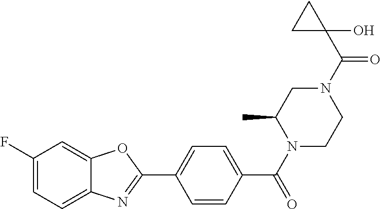 [SMILES: C[C@H]1CN(C(=O)C2(O)CC2)CCN1C(=O)c1ccc(-c2nc3ccc(F)cc3o2)cc1] |
| 4-(4′-(4-(1-hydroxycyclopropane-1- carbonyl)piperazine-1-carbonyl)- [1,1′-biphenyl]-3-yl)tetrahydro-2H- pyran-4-carbonitrile | 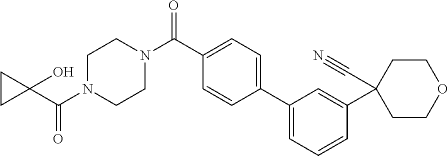 [SMILES: N#CC1(c2cccc(-c3ccc(C(=O)N4CCN(C(=O)C5(O)CC5)CC4)cc3)c2)CCOCC1] |
| (4-(1-hydroxycyclopropane-1- carbonyl)piperazin-1-yl)(3′-(5- methyl-1,2,4-oxadiazol-3-yl)-[1,1′- biphenyl]-4-yl)methanone | 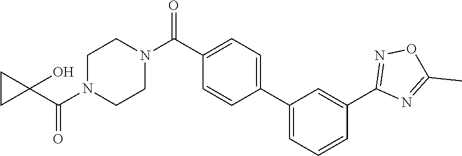 [SMILES: Cc1nc(-c2cccc(-c3ccc(C(=O)N4CCN(C(=O)C5(O)CC5)CC4)cc3)c2)no1] |
| 5-(4′-(4-(1-hydroxycyclopropane-1- carbonyl)piperazine-1-carbonyl)- [1,1′-biphenyl]-3-yl)-5- methylimidazolidine-2,4-dione | 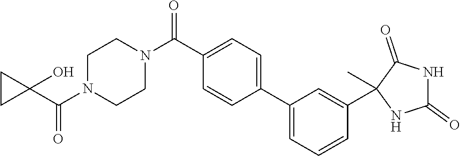 [SMILES: CC1(c2cccc(-c3ccc(C(=O)N4CCN(C(=O)C5(O)CC5)CC4)cc3)c2)NC(=O)NC1=O] |
| (4-(1-aminocyclopropane-1- carbonyl)piperazin-1-yl)(2-fluoro-4- (2-methyl-1H-indol-1- yl)phenyl)methanone | 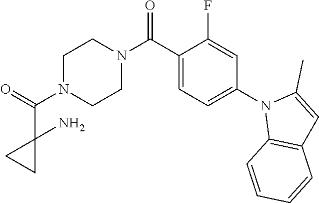 [SMILES: Cc1cc2ccccc2n1-c1ccc(C(=O)N2CCN(C(=O)C3(N)CC3)CC2)c(F)c1] |
| (4-(4-(3-aminobenzo[d]isoxazol-6- yl)benzoyl)piperazin-1-yl)(1- hydroxycyclopropyl)methanone | 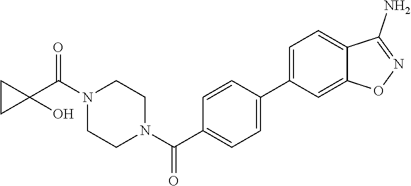 [SMILES: Nc1noc2cc(-c3ccc(C(=O)N4CCN(C(=O)C5(O)CC5)CC4)cc3)ccc12] |
| (4-(1-aminocyclobutane-1- carbonyl)piperazin-1-yl)(2-fluoro-4- (2-methyl-2H-indazol-6- yl)phenyl)methanone | 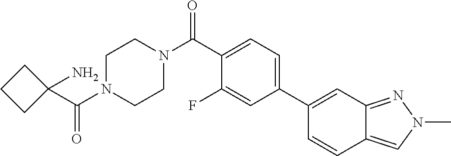 [SMILES: Cn1cc2ccc(-c3ccc(C(=O)N4CCN(C(=O)C5(N)CCC5)CC4)c(F)c3)cc2n1] |
| (4-(3′-(1,1-dioxidoisothiazolidin-2- yl)-[1,1′-biphenyl]-4- carbonyl)piperazin-1-yl)(1- hydroxycyclopropyl)methanone | 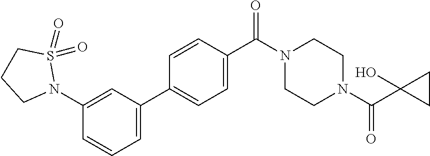 [SMILES: O=C(c1ccc(-c2cccc(N3CCCS3(=O)=O)c2)cc1)N1CCN(C(=O)C2(O)CC2)CC1] |
| (4-(4-(6-chlorobenzo[d]oxazol-2-yl)- 3-fluorobenzoyl)piperazin-1-yl)(1- hydroxycyclopropyl)methanone | 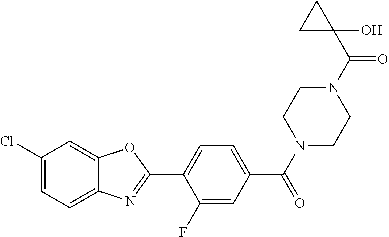 [SMILES: O=C(c1ccc(-c2nc3ccc(Cl)cc3o2)c(F)c1)N1CCN(C(=O)C2(O)CC2)CC1] |
| (4-(1-hydroxycyclopropane-1- carbonyl)piperazin-1-yl)(4-(1,3,4,5- tetrahydro-2H-benzo[c]azepin-2- yl)phenyl)methanone | 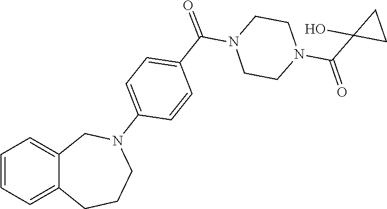 [SMILES: O=C(c1ccc(N2CCCc3ccccc3C2)cc1)N1CCN(C(=O)C2(O)CC2)CC1] |
| (4-(4-(3-chloroimidazo[1,2- a]pyridin-2-yl)-3- fluorobenzoyl)piperazin-1-yl)(1- hydroxycyclopropyl)methanone | 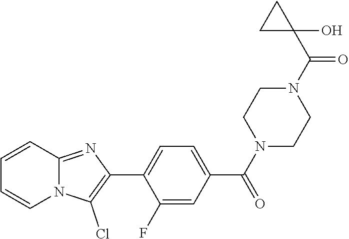 [SMILES: O=C(c1ccc(-c2nc3ccccn3c2Cl)c(F)c1)N1CCN(C(=O)C2(O)CC2)CC1] |
| 1-(4′-(4-(1-aminocyclopropane-1- carbonyl)piperazine-1-carbonyl)- [1,1′-biphenyl]-3-yl)cyclopropane- 1-carbonitrile | 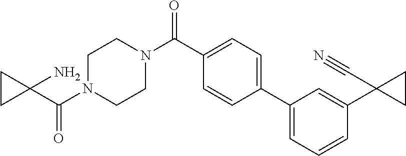 [SMILES: N#CC1(c2cccc(-c3ccc(C(=O)N4CCN(C(=O)C5(N)CC5)CC4)cc3)c2)CC1] |
| (4-(3-fluoro-4-(6- fluorobenzo[d]oxazol-2- yl)benzoyl)piperazin-1-yl)(1- hydroxycyclopropyl)methanone | 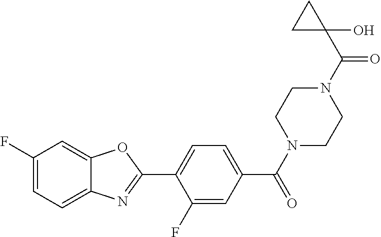 [SMILES: O=C(c1ccc(-c2nc3ccc(F)cc3o2)c(F)c1)N1CCN(C(=O)C2(O)CC2)CC1] |
| (4-(1-hydroxycyclopropane-1- carbonyl)piperazin-1-yl)(4-(2- methyl-2H-benzo[d][1,2,3]triazol-5- yl)phenyl)methanone | 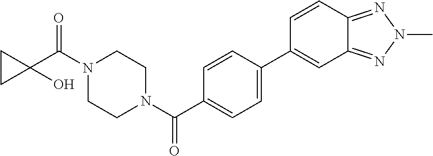 [SMILES: Cn1nc2ccc(-c3ccc(C(=O)N4CCN(C(=O)C5(O)CC5)CC4)cc3)cc2n1] |
| (S)-(4-(1-aminocyclopropane-1- carbonyl)-2-methylpiperazin-1- yl)(4-(1-methyl-1H- benzo[d]imidazol-5- yl)phenyl)methanone | 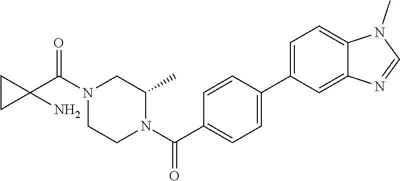 [SMILES: C[C@H]1CN(C(=O)C2(N)CC2)CCN1C(=O)c1ccc(-c2ccc3c(c2)ncn3C)cc1] |

or a pharmaceutically acceptable salt thereof.

### Pharmaceutical Compositions

In some embodiments, the present disclosure provides methods for treating glioblastoma, comprising administering a pharmaceutical composition comprising a provided compound optionally in combination with a pharmaceutically acceptable excipient (e.g. carrier).

Suitable pharmaceutical compositions include optical isomers, diastereomers, or pharmaceutically acceptable salts of the compounds disclosed herein. For example, in some embodiments, pharmaceutical compositions include a pharmaceutically acceptable salt. A compound included in the pharmaceutical composition may be covalently attached to a pharmaceutically acceptable carrier. Alternatively, the inventive compound included in the pharmaceutical composition is not covalently linked to a pharmaceutically acceptable carrier.

A “pharmaceutically acceptable carrier,” as used herein refers to pharmaceutical excipients, for example, pharmaceutically, physiologically, acceptable organic, or inorganic carrier substances suitable for enteral or parenteral application which do not deleteriously react with the compounds used in accordance with the provided methods. Suitable pharmaceutically acceptable carriers include water, salt solutions (such as Ringer's solution), alcohols, oils, gelatins and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like which do not deleteriously react with the compounds used in accordance with the provided methods.

Compounds can be administered alone or can be coadministered to a patient along with one or more other drugs. Coadministration is meant to include simultaneous or sequential administration of the compounds individually or in combination (more than one compound). In some embodiments, the preparations are combined with other active substances (e.g. to reduce metabolic degradation).

### Formulations

In some embodiments, the present disclosure provides methods for treating glioblastoma, comprising administering of a formulation of a compound or composition described herein. Suitable formulations can be prepared and administered in a wide variety of oral, parenteral, and topical dosage forms. In some embodiments, provided compounds are administered by injection (e.g. intravenously, intramuscularly, intracutaneously, subcutaneously, intraduodenally, or intraperitoneally). In some embodiments, compounds described herein are administered by inhalation, for example, intranasally. In some embodiments, provided compounds are administered transdermally. It is also envisioned that multiple routes of administration (e.g., intramuscular, oral, transdermal) can be used to administer the compounds of the invention. The present invention also provides pharmaceutical compositions comprising one or more provided compounds and one or more pharmaceutically acceptable carriers or excipients.

Pharmaceutically acceptable carriers can be either solid or liquid. Solid form preparations include powders, tablets, pills, capsules, cachets, suppositories, and dispersible granules. In some embodiments, a solid carrier is one or more substances, which may also act as diluents, flavoring agents, binders, preservatives, tablet disintegrating agents, or an encapsulating material.

In some embodiments, when a composition is a powder, a carrier is a finely divided solid in a mixture with the finely divided active component. In some embodiments, when a composition is formulated for a tablet, an active component is mixed with the carrier having the necessary binding properties in suitable proportions and compacted in the shape and size desired.

In some embodiments, powders and tablets contain from 5% to 70% of the active compound. Suitable carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. In some embodiments, the composition is formulated for a cachet or lozenge. In some embodiments, tablets, powders, capsules, pills, cachets, and/or lozenges are used as solid dosage forms suitable for oral administration.

In some embodiments, for preparing suppositories, a low melting wax, such as a mixture of fatty acid glycerides or cocoa butter, is first melted and the active component is dispersed homogeneously therein. The molten homogeneous mixture is then poured into convenient sized molds, allowed to cool and solidify.

Liquid form preparations include solutions, suspensions, and emulsions, for example, water or water/propylene glycol solutions. In some embodiments, for parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. Compounds useful in provided methods can also be incorporated into liposomes or administered via transdermal pumps or patches. Pharmaceutical admixtures suitable for use in the present invention include those described, for example, in Pharmaceutical Sciences (17th Ed., Mack Pub. Co., Easton, Pa.) and WO 96/05309, each of which is hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations intended for conversion shortly before use to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

In some embodiments, pharmaceutical compositions are in unit dosage form. In such form the composition is subdivided into unit doses containing appropriate quantities of the active component. The unit dosage form can be a packaged preparation, the package containing discrete quantities of a pharmaceutical composition, such as packeted tablets, capsules, and powders in vials or ampoules. In some embodiments, the unit dosage form is a capsule, tablet, cachet, or lozenge itself, or it is the appropriate number of any of these in packaged form.

The quantity of active component in a unit dosage form may be varied or adjusted from 0.1 mg to 10000 mg, more typically 1.0 mg to 1000 mg, most typically 10 mg to 500 mg, according to the particular application and the potency of the active component. In some embodiments, provided compositions contain other compatible therapeutic agents.

Some compounds may have limited solubility in water and may require a surfactant or other appropriate co-solvent in the composition. Such co-solvents include: Polysorbate 20, 60 and 80, Pluronic F-68, F-84 and P-103, cyclodextrin, and polyoxyl 35 castor oil. Such co-solvents are typically employed at a level between about 0.01% and about 2% by weight.

In some embodiments, viscosity greater than that of simple aqueous solutions may be desirable to decrease variability in dispensing the formulations, to decrease physical separation of components of a suspension or emulsion of formulation and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

Compositions used in provided methods may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

### Combinations

In some embodiments, a provided method further comprises administration of an additional therapeutic agent. In some embodiments, the present disclosure provides methods for treating glioblastoma, comprising administering a FASN inhibitor in combination with additional therapeutic agents. In one embodiment, the additional therapeutic agent is a BRAF inhibitor. In another embodiment, the additional therapeutic agent is a PD1/PDL1 inhibitor.

In some embodiments, a BRAF inhibitor is GDC-0879, SB590885; Encorafenib (LGX818); RAF265 (CHIR-265); Dabrafenib (GSK2118436); TAK-632; PLX-4720; CEP-32496; Sorafenib Tosylate; Sorafenib; Vemurafenib (PLX4032, RG7204); AZ 628; or vemurafenib.

In some embodiments, a PD1/PDL1 inhibitor is selected from the group consisting of PD1 inhibitors Pembrolizumab, and Nivolumab; and PDL1 inhibitors Atezolizumab, MEDI4736, Avelumab, and PDR001.

In some embodiments, the additional therapeutic agent is Temozolomide.

The invention thus provides, in a further aspect, a combination comprising a compound described herein or a pharmaceutically acceptable salt thereof together with at least one additional therapeutic agent. In an exemplary embodiment, the additional therapeutic agent is a compound of the invention.

When a compound described herein is used in combination with a second therapeutic agent active against the same disease state, the dose of each compound may differ from that when the compound is used alone. Appropriate doses will be readily appreciated by those skilled in the art. It will be appreciated that the amount of a compound required for use in treatment will vary with the nature of the condition being treated and the age and the condition of the patient and will be ultimately at the discretion of the attendant physician.

### Effective Dosage

Pharmaceutical compositions of the present disclosure include compositions wherein the active ingredient is contained in a therapeutically effective amount, i.e., in an amount effective to achieve its intended purpose. The actual amount effective for a particular application will depend, inter alia, on the condition being treated.

The dosage and frequency (single or multiple doses) of compound administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds of the invention.

For any compound described herein, the therapeutically effective amount can be initially determined from cell culture assays. Target concentrations will be those concentrations of active compound(s) that are capable of killing parasites and/or controlling their growth or reproduction as measured, for example, using the methods described.

Therapeutically effective amounts for use in humans may be determined from animal models. For example, a dose for humans can be formulated to achieve a concentration that has been found to be effective in animals. The dosage in humans can be adjusted by monitoring kinase inhibition and adjusting the dosage upwards or downwards, as described above. Therapeutically effective amounts for use in animals (e.g., cattle) may be determined from animal models (e.g., mouse models).

Dosages may be varied depending upon the requirements of the patient and the compound being employed. The dose administered to a patient, in the context of the present invention, should be sufficient to effect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached. In some embodiments, the dosage range is 0.001% to 10% w/v. In some embodiments, the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the individual's disease state.

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### EXAMPLES

The following are illustrative, but non-limiting, examples of certain embodiments of the present invention.

### Example 1

### Enzymatic Assay for FASN Inhibitors

Assays were performed in a 384-well black plate to measure the inhibition of FASN activity by individual compounds disclosed herein. An aliquot of 250 nL of compound was incubated with 10 μL of 40 nM FASN enzyme in assay buffer (50 mM HEPES (pH=7.3), 0.5 mM EDTA, 1 mM ascorbate, 100 mM sodium chloride and 0.04% TritonX-100) in each well at 25° C. for 60 minutes. After the plate was centrifuged briefly, 10 μL of substrate mix (20 μM acetyl-CoA, 60 μM malonyl-CoA, and 100 μM NADPH in assay buffer) was added to each well. The reaction was maintained at 25° C. for 90 minutes. The reaction was then quenched by adding 10 μL of 90 μM 7-diethylamino-3-(4′-maleimidylpheynyl)-4-methylcoumarin in 50/50 ethanol/water solution. The assay plate was incubated at 25° C. for 15 minutes and read on a plate reader with excitation and emission wavelengths at 360 nm and 530 nm, respectively. The IC₅₀ of a given compound was calculated by fitting the dose response curve with a four-parameter logistic equation. Data shown below in Table B, where compounds having an activity designated as “A” provided an IC₅₀<100 nm and compounds having an activity designated as “B” provided an IC₅₀ of 100 nm-1.00 μM.

### Example 2

### Determination of Brain Exposure in Balb/c Nude Mice Following Oral Administration

Balb/c nude mice (body weight: 20-30 g; age range: approximately 6-8 weeks) were purchased from a qualified provider and acclimated for 5-7 days. The dosing formulation (dose: 5 mg/kg; concentration: 0.5 mg/mL; vehicle: PEG400:ethanol (v/v 9:1)) was prepared on the day of dosing and administered via oral gavage (dose volume: 10 mL/kg) to the mice (n=3 animals per time point). At the desired time point (2 or 8 h post dose) approximately 0.03 mL of blood sample was collected via venipuncture of peripheral veins into Heparin Na tubes and immediately placed on ice. After gently reverting the tube for approximately 10 seconds, the blood sample was centrifuged at 4,000×g for 5 minutes at 4° C. Supernatant was transferred into a micro-centrifuge tube and kept in a −80° C. freezer. Prior to brain collection, mice were fully exsanguinated via the following procedure: the chest cavity was opened, the ventricle was cut and a gentle iv saline flush (saline flush volume ˜20 mL) was performed with the animal placed head down at a 45 degree angle to facilitate blood removal. Brain samples were collected at the desired time point (2 or 8 h post dose), quick frozen in an ice box, and kept at −75±15° C. All brain samples were weighed and homogenated with buffer (brain weight (g) to PBS volume (mL) ratio of 1:4) before analysis. The actual concentration is the detected value multiplied by the dilution factor. For plasma sample analysis, plasma samples were quantified against a standard curve in plasma of Balb/c nude mice using an LC-MS/MS Method. For brain sample analysis, brain samples were quantified against a standard curve in brain homogenate of Balb/c nude mice using an LC-MS/MS Method. Each standard curve covered at least 6 calibration points including lower limit of quantification (LLOQ).

TABLE B

| | FASN | Brain | Plasma | [Brain]/ |
|---|---|---|---|---|
| | enzyme | (μM) | (μM) | [Plasma] |
| ID | IC₅₀ | at 2, 8 h | at 2, 8 h | at 2, 8 h |
| Compound 1 | B | 1.3, 0.40 | 7.3, 2.1 | 0.18, 0.20 |
| Compound 2 | A | 0.3, 0.01 | 1.4, 0.06 | 0.20, 0.21 |
| Compound 3 | B | 1.8, 0.89 | 7.8, 3.3 | 0.23, 0.27 |
| Compound 4 | A | 1.6, 0.17 | 10.6, 1.1 | 0.15, 0.16 |
| Compound 5 | B | 1.4, 0.62 | 8.4, 3.3 | 0.17, 0.19 |
| Compound 6 | B | 0.042 | 0.396 | 0.105 |
| Compound 7 | B | 0.013 | 0.238 | 0.05 |
| Compound 8 | B | 0.038 | 0.309 | 0.12 |
| Compound 9 | A | 0.03, 0.007 | 5.2, 0.6 | 0.006, 0.012 |
| Compound 10 | A | 0.04, 0.004 | 4.4, 1.0 | 0.007, 0.004 |
| Compound 11 | A | 0.02, 0.0 | 4.4, 0.6 | 0.005, 0.000 |
| Compound 12 | B | 0.02, 0.0 | 1.5, 0.2 | 0.014, 0.000 |
| Compound 13 | A | 0.28, 0.12 | 5.8, 3.1 | 0.05, 0.04 |

### Example 3

### Inhibition of GSC Neurosphere Formation

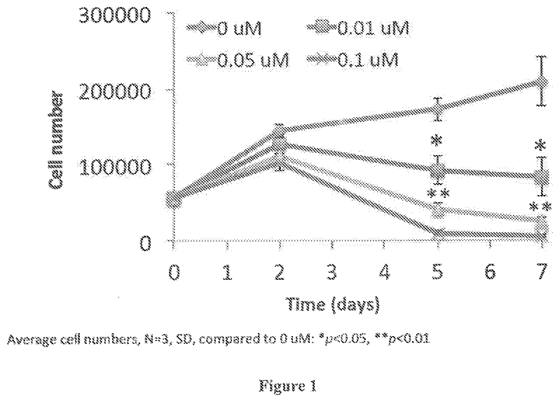

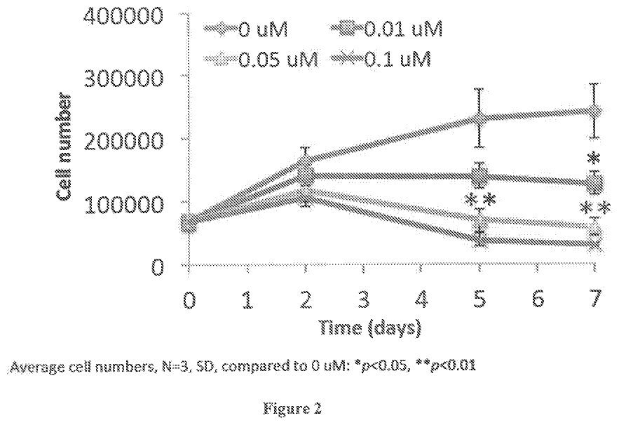

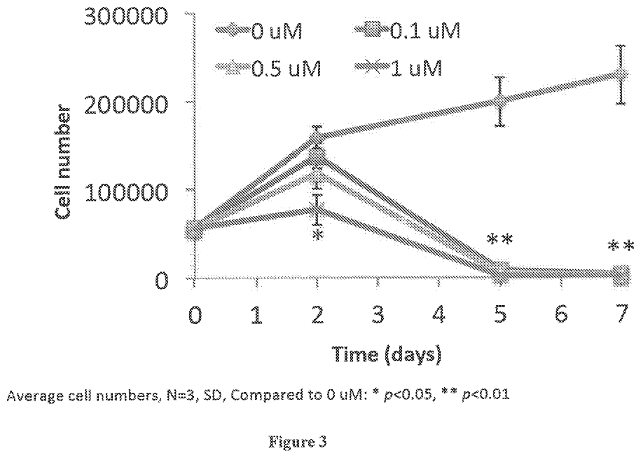

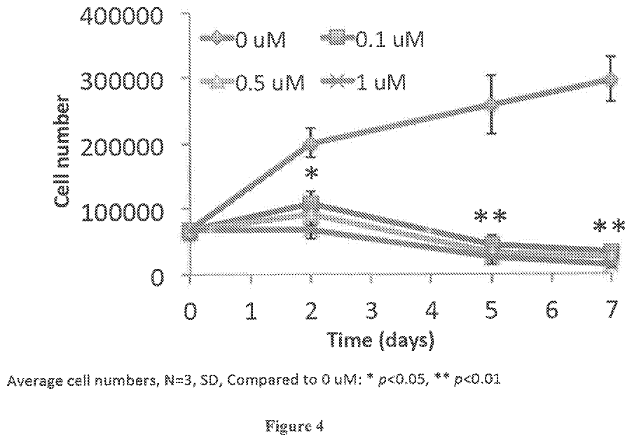

The Example demonstrates that a FASN inhibitor blocked the proliferation of neurosphere growth by inhibiting FASN activity. Briefly, glioma stem cells (GSC827 or GSC923) were grown as neurospheres in serum free neural stem cell medium supplemented with basic fibroblast growth factor (bFGF) and epidermal growth factor (EGF) in the presence of 0.01-1 uM of test compound for 7 days, and the viability of the neurospheres was monitored by counting viable cells at day 2, 5, and 7. Test compound inhibited neurosphere formation in GSC827 and GSC923 with an estimated IC50 of 10-50 nM. Applicant observed that it took more than five days to maximize the antiproliferative activity in both lines. The antiproliferative potency of the compound in the two GSC neurosphere growth assays was comparable to potency from FASN inhibition. See FIGS. 1-4.

### Example 4

### Efficacy Study in a Patient-Derived Orthotopic Glioblastoma PDX Mouse Model

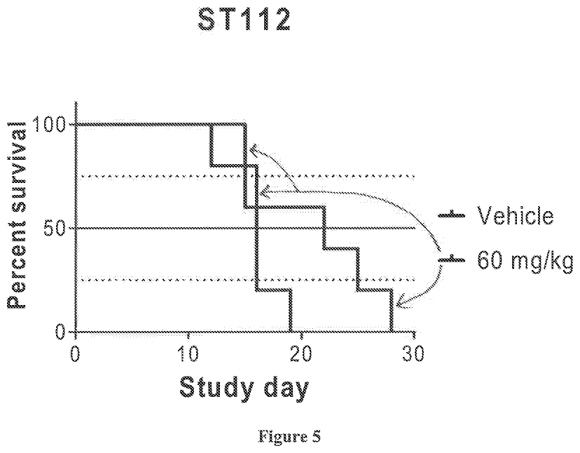

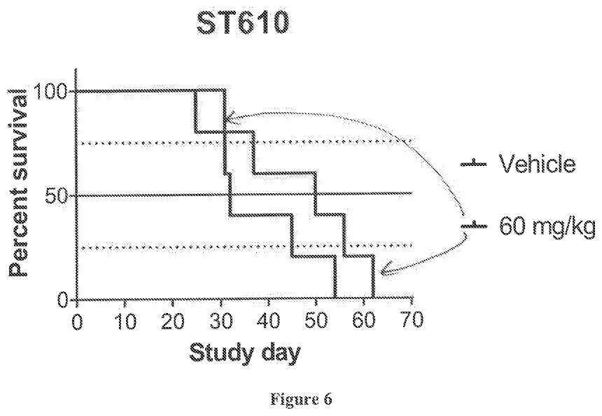

This Example demonstrates the antitumor activity of test compounds resulting in the benefit of extended extension of median survival. Mice were implanted by stereotactic injection of 2×10⁵ ST112 or ST610 patient-derived GBM cells in 10 μL into 1.5 mm right of the sutura saggitalis and 0.5 mm posterior to the bregma using a micro-drill in 10 week old female NMRI nude mice (stock from Janvier Labs, France). Tumors were grown until reaching a mean value of 2.0 mm³ as determined by T2-weighted MR imaging (MRI). The tumor-bearing animals were assigned to 2 groups (n=5 each); Vehicle (80% de-ionized water, 10% Kolliphor® EL, 10% propylene glycol) or test compound 60 mg/kg BID every other day. T2-weighted MRI was used to monitor tumor volume 14 and 28 days after treatment start. Animals were treated until >20% BW loss or morbidity as defined by clinical scoring. See FIGS. 5-6.

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1. A method for treating glioblastoma comprising the step of administering to a patient in need thereof a therapeutically effective amount of compound 1 or a pharmaceutically acceptable salt thereof:

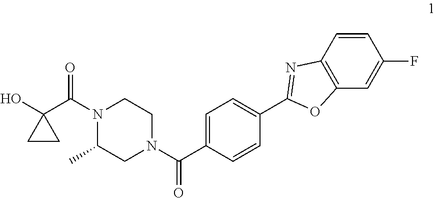 [SMILES: C[C@H]1CN(C(=O)c2ccc(-c3nc4ccc(F)cc4o3)cc2)CCN1C(=O)C1(O)CC1]

2-20. (canceled)

21. The method of claim 1, wherein the compound 1 crosses the blood brain barrier.

22. (canceled)

23. The method of claim 1, wherein the compound 1, or the pharmaceutically acceptable salt thereof, is administered orally.

24. The method of claim 1, wherein the compound 1, or the pharmaceutically acceptable salt thereof, is administered as a pharmaceutical composition comprising the compound 1, or the pharmaceutically acceptable salt thereof, and a pharmaceutically acceptable excipient.

25. The method of claim 21, wherein the compound 1, or the pharmaceutically acceptable salt thereof, is administered orally.

26. The method of claim 21, wherein the compound 1, or the pharmaceutically acceptable salt thereof, is administered as a pharmaceutical composition comprising the compound 1, or the pharmaceutically acceptable salt thereof, and a pharmaceutically acceptable excipient.

27. The method of claim 23, wherein the compound 1, or the pharmaceutically acceptable salt thereof, is administered as a pharmaceutical composition comprising the compound 1, or the pharmaceutically acceptable salt thereof, and a pharmaceutically acceptable excipient.

28. The method of claim 25, wherein the compound 1, or the pharmaceutically acceptable salt thereof, is administered as a pharmaceutical composition comprising the compound 1, or the pharmaceutically acceptable salt thereof, and a pharmaceutically acceptable excipient.

29. A method for inhibiting FASN in the brain of a subject, the method comprising the step of administering to a subject in need thereof a pharmaceutical composition comprising compound 1 or a pharmaceutically acceptable salt thereof:

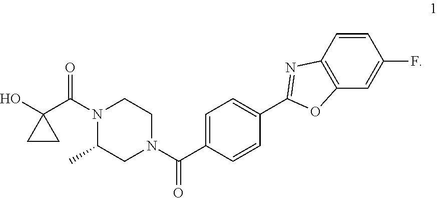 [SMILES: C[C@H]1CN(C(=O)c2ccc(-c3nc4ccc(F)cc4o3)cc2)CCN1C(=O)C1(O)CC1]

30. The method of claim 29, wherein the composition comprising compound 1, or the pharmaceutically acceptable salt thereof, is orally administered to the subject.
